(19) United States
(12) Patent Application Publication
(10) Pub. No.: US 2009/0318396 A1
(43) Pub. Date: Dec. 24, 2009

(54) CORTICOSTEROID LINKED BETA-AGONIST COMPOUNDS FOR USE IN THERAPY

(75) Inventors: William R. Baker, Bellevue, WA (US); Musong Kim, Bothell, WA (US); Alexander Rudolph, Snoqualmie, WA (US); Marcin Stasiak, Seattle, WA (US); Josh Van Veldhuizen, Seattle, WA (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/481,364

(22) Filed: Jun. 9, 2009

Related U.S. Application Data
(60) Provisional application No. 61/153,518, filed on Feb. 18, 2009.
(60) Provisional application No. 61/060,388, filed on Jun. 10, 2008.

(51) Int. Cl. A61K 31/58; C07J 71/00; A61P 11/00

(52) U.S. Cl. 514/171; 540/67; 514/174

(57) ABSTRACT

New chemical entities which comprise corticosteroids and phosphorylated β-agonists for use in therapy and compositions comprising and processes for preparing the same.

### RELATED APPLICATIONS

This claims priority to U.S. provisional application No. 61/153,518, filed 18 Feb. 2009 and U.S. provisional application No. 61/060,388, filed 10 Jun. 2008.

### FIELD OF THF INVENTION

The instant invention relates to new chemical entities which comprise corticosteroids and phosphorylated β-agonists for use in therapy and compositions comprising and processes for preparing the same.

### BACKGROUND OF THF INVENTION

Asthma is a chronic inflammatory disease of the airways produced by the infiltration of pro-inflammatory cells, mostly eosinophils and activated T-lymphocytes (Poston, Am. Rev. Respir. Dis., 145 (4 Pt 1), 918-921, 1992; Walker, J. Allergy Clin. Immunol, 88 (6), 935-42, 1991) into the bronchial mucosa and submucosa. The secretion of potent chemical mediators, including cytokines, by these proinflammatory cells alters mucosal permeability, mucus production, and causes smooth muscle contraction. All of these factors lead to an increased reactivity of the airways to a wide variety of irritant stimuli (Kaliner, “Bronchial asthma, Immunologic diseases” E. M. Samter, Boston, Little, Brown and Company: 117-118, 1988).

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. Allergy Clin. Immunol, 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10^{th} edition, 2001). A solution to systemic side effects is to deliver steroid drugs directly to the site of inflammation. Inhaled corticosteroids (ICS) have been developed to mitigate the severe adverse effects of oral steroids. While ICS are very effective in controlling inflammation in asthma, they too are not precisely delivered to the optimal site of action in the lungs and produce unwanted side effects in the mouth and pharynx (candidiasis, sore throat, dysphonia).

Combinations of inhaled β₂-adrenoreceptor agonist bronchodilators such as formoterol or salmeterol with ICS's are also used to treat both the bronchoconstriction and the inflammation associated with asthma and COPD (Symbicort® and Advair®, respectively). However, these combinations have the side effects of both the ICS's and the β₂-adrenoreceptor agonist because of systemic absorption (tachycardia, ventricular dysrhythmias, hypokalemia) primarily because neither agent is delivered exclusively to the optimal sites of action in the lungs. In consideration of all problems and disadvantages connected with the adverse side effect profile of ICS and of β-agonists it would be highly advantageous to provide a drug which masks the pharmacological properties of both steroids and β-agonists until such a drug reaches the optimal site of action.

Phenylphosphate based mutual prodrugs of corticosteroids and β₂-agonists have been described by Baker (WO/2006/138212) wherein the component drugs are released at the site of action in the lungs.

### SUMMARY OF THF INVENTION

In one aspect, the instant invention comprises new compounds which are useful as therapeutic agents. The compounds generally comprise a corticosteroid moiety and a phosphorylated β-agonist moiety. The compounds of the invention are believed to be useful for treating conditions and diseases for which corticosteroids and β-agonists, particularly β₂-agonists, are employed. Specific examples of such conditions include pulmonary inflammation and bronchoconstriction in diseases such as asthma, bronchitis (including chronic bronchitis or bronchiectasis) and COPD.

In one aspect, the invention comprises compounds of Formula I-1:

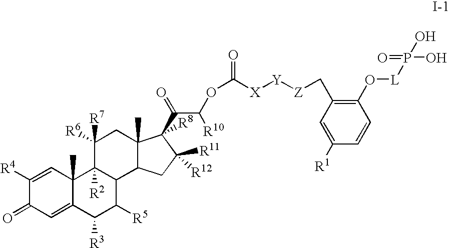

and pharmaceutically acceptable salts thereof,
wherein:
R¹ is

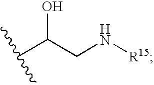

each R², R³, R⁴, and R⁵ are, independently, H, C₁-C₄alkyl or halo;
R⁶ and R⁷ are, independently, H or OH; or R⁶ and R⁷ taken together with the carbon to which they are attached form a >C═O group;
R⁸ is H, OH, O(CO)R⁹, or O(CO)OR⁹;
each R⁹ is, independently, C₁-C₄alkyl;
each R¹⁰ and R¹¹ is, independently, H or C₁-C₄alkyl;
R¹² is H, OH, or R⁹; or R¹¹ and R¹² taken together with the carbon to which they are attached form a >═CH₂ group; or R¹² and R⁸ taken together with the carbons to which they are attached form a 1,3-dioxolane ring represented by formula B

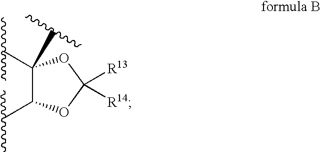

each R¹³ and R¹⁴ are, independently, H, optionally substituted C₁-C₁₀alkyl, optionally substituted C₂-C₁₀alkenyl, optionally substituted C₂-C₁₀alkynyl, optionally substituted C₃-C₁₀ carbocyclyl, optionally substituted C₆-C₁₀ aryl, or optionally substituted heteroaryl;
R¹⁵ is optionally substituted C₁-C₁₂alkyl, arylalkyl, substituted arylalkyl, or optionally substituted carbocyclyl wherein 1-3 carbon atoms of said optionally substituted C₁-C₁₂alkyl, arylalkyl, substituted arylalkyl or optionally substituted carbocyclyl may be replaced by O, S, N(H), or N(C₁-C₄alkyl);
X is a bond, O, S, N(H), N(C₁-C₄alkyl), optionally substituted C₁-C₁₀alkylene, optionally substituted C₂-C₁₀alkenylene, optionally substituted C₂-C₁₀alkynylene, optionally substituted C₆-C₁₀ arylene, optionally substituted heterocyclene, optionally substituted heteroarylene or optionally substituted C₃-C₁₀ carbocyclene;
Y is a bond, optionally substituted C₁-C₁₀alkylene, optionally substituted C₂-C₁₀alkenylene, optionally substituted C₂-C₁₀alkynylene, optionally substituted C₃-C₁₀ carbocyclene, optionally substituted C₆-C₁₀ arylene, or optionally substituted heteroarylene; wherein one or more carbon atoms of said C₁-C₁₀alkylene or C₃-C₁₀ carbocyclene is, optionally, replaced by O, S, N(H), N(C₁-C₄alkyl), —N(H)C(O)—, —N(C₁-C₄alkyl)C(O)—, —C(O)N(H), or —C(O)N(C₁-C₄alkyl)-;
Z is ^{⊕}(NR¹⁷R¹⁸)A⁽⁻⁾, N(O)R¹⁷ (N-oxide), S(O) (sulfoxide), S(═O)₂, ^{⊕}(SR¹⁷)A⁽⁻⁾, a heterocyclene comprising ^{⊕}(NR¹⁷)A⁽⁻⁾ or ^{⊕}SA⁽⁻⁾, or a heteroarylene comprising a ^{⊕}NA⁽⁻⁾; wherein when Z is said heterocyclene or said heteroarylene the group represented by

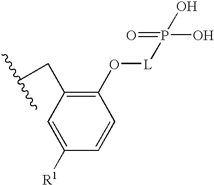

in Formula I is directly bonded to a ^{⊕} NR¹⁷ or ^{⊕}S of said heterocyclene or a ^{⊕}N of said heteroarylene;
L is a bond or —(CH₂O)—;
each R¹⁷ and R¹⁸ are, independently, optionally substituted C₁-C₁₀alkyl, optionally substituted C₂-C₁₀alkenyl, optionally substituted C₂-C₁₀alkynyl, optionally substituted C₃-C₁₀ carbocyclyl, optionally substituted C₆-C₁₀ aryl, or optionally substituted heteroaryl; or 17 and R¹⁸ taken together with the nitrogen to which they are attached form a heterocyclic ring comprising 3-7 carbon atoms wherein one or more carbon atoms of said heterocyclic ring is, optionally, replaced by O, S, N(H), or N(C₁-C₄alkyl); and
A⁽⁻⁾ is a pharmaceutically acceptable negative counterion.

In another aspect, the invention provides a compound of Formula I:

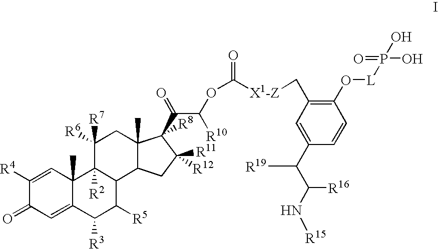

or a pharmaceutically acceptable salt thereof,
wherein:
R¹⁵ is a side chain radical of a β-agonist;
R¹⁶ is H, methyl or ethyl;
R¹⁹ is H, F, OH or methyl;
each R², R³, R⁴, and R⁵ are independently H, C₁-C₄alkyl or halo;
R⁶ and R⁷ are independently H or OH; or R⁶ and R⁷ taken together with the carbon to which they are attached form a >C═O group;
R⁸ is H, OH, O(CO)R⁹, or O(CO)OR⁹;
each R⁹ is independently C₁-C₄alkyl;
each R¹⁰ and R¹¹ is independently H or C₁-C₄alkyl;
R¹² is H, OH, or C₁-C₄alkyl; or
R¹¹ and R¹² taken together with the carbon to which they are attached form a >═CH₂ group; or
R¹² and R⁸ taken together with the carbons to which they are attached form a 1,3-dioxolane ring represented by formula B:

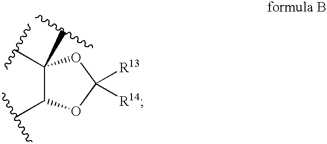

wherein one of R¹³ and R¹⁴ is H, methyl or ethyl and the other is H, C₁-C₁₀alkyl, C₂-C₁₀alkenyl, C₂-C₁₀alkynyl, optionally substituted C₃-C₁₀ carbocyclyl or optionally substituted 5-6 ring atom heterocycle wherein one or two ring atoms are selected from N, O and S, and wherein said carbocyclyl and said heterocyclyl are each optionally substituted 1, 2 or 3 times with a substituent selected from halo, C₁-C₄alkyl, and O—C₁-C₄alkyl;
Z is N(H), N(C₁-C₁₀alkyl), ^{⊕}(NR¹⁷R¹⁸)A⁽⁻⁾, N(O)R¹⁷ (N-oxide), S(O) (sulfoxide), S(═O)₂, ^{⊕}(SR¹⁷)A⁽⁻⁾, or a 4-9 ring atom heterocyclene wherein one ring atom is N, ^{⊕}(N)A⁽⁻⁾, ^{⊕}(N(C₁-C₆alkyl))A⁽⁻⁾ or ^{⊕}SA⁽⁻⁾ and the β-agonist moiety

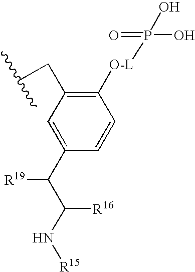

in Formula I is bonded to the N, ^{⊕}N, ^{⊕}N(C₁-C₆alkyl) or ^{⊕}S atom of the heterocyclene;
X¹ is selected from a bond,
   C₁-C₁₂alkylene, C₂-C₁₂alkenylene, C₂-C₁₂alkynylene,
   O—C₁-C₁₂alkylene, O—C₂-C₁₂alkenylene, O—C₂-C₁₂alkynylene,
   S—C₁-C₁₂alkylene, S—C₂-C₁₂alkenylene, S—C₂-C₂alkynylene,
   N(H)—C₁-C₁₂alkylene, N(H)—C₂-C₁₂alkenylene, N(H)—C₂-C₁₂alkynylene,
   N(C₁-C₆alkyl)-C₁-C₁₂alkylene, N(C₁-C₆alkyl)-C₂-C₁₂alkenylene, N(C₁-C₆alkyl)-C₂-C₁₂alkynylene,
   C₃-C₇-carbocyclene, C₃-C₇-carbocyclene-C₁-C₆alkylene, heterocyclene, heterocyclene-C₁-C₆alkylene, heterocyclene-C(O), wherein said heterocyclene is a 3-9 ring atom heterocyclene wherein 1 or 2 ring atoms are selected from N, O and S,
   C₁-C₆alkylene-O—C₁-C₆alkylene, C₁-C₆alkylene-S—C₁-C₆alkylene, C₁-C₆alkylene-N(H)—C₁-C₆alkylene, C₁-C₆alkylene-N(C₁-C₃alkyl)-C₁-C₆alkylene,
   C₁-C₆alkylene-C₃-C₇-carbocyclene-C₁-C₆alkylene, C₁-C₆alkylene-heterocyclene-C₁-C₆alkylene, wherein said heterocyclene is a 3-9 ring atom heterocyclene wherein 1 or 2 ring atoms are selected from N, O and S,
   C₁-C₁₂alkylene-O, C₁-C₁₂alkylene-S, C₁-C₁₂alkylene-N(H), C₁-C₁₂alkylene-N(C₁-C₆alkyl), C₁-C₈alkylene-N(H)C(O), C₁-C₈alkylene-N(C₁-C₄alkyl)C(O), C₁-C₈alkylene-C(O)N(H), C₁-C₈alkylene-C(O)N(C₁-C₄alkyl),
   CH-AA, and C(H)(AA)-N(H)C(O), wherein AA is a proteinogenic amino acid side chain;
   wherein each alkyl, alkylene, alkenylene, and alkynylene is optionally substituted 1 or 2 times with a substituent independently selected from halo, OH, OCH₃, NH₂, N(H)CH₃, and N(CH₃)₂, and each carbocyclene and heterocyclene is optionally substituted 1, 2 or 3 times with a substituent independently selected from halo, and C₁-C₄alkyl;
wherein when Z is N(H), N(C₁-C₆alkyl), ^{⊕}(NR¹⁷R¹⁸)A⁽⁻⁾N(O)R¹⁷ (N-oxide), S(O) (sulfoxide), S(═O)₂, or ^{⊕}(SR¹⁷)A⁽⁻⁾, then X¹ is neither a bond nor bound to Z through O, S, N(H), N(C₁-C₆alkyl), N(H)C(O), N(C₁-C₄alkyl)C(O), C(O)N(H) or C(O)N(C₁-C₄alkyl);
wherein each R¹⁷ and R¹⁸ are, independently, C₁-C₆alkyl, C₁-C₆alkenyl, C₁-C₆alkynyl, or C₃-C₇-carbocycle, wherein said alkyl, alkenyl, alkynyl is optionally substituted 1, 2 or 3 times with a substituent independently selected from halo, OH, and ═O, and the carbocycle is optionally substituted 1, 2 or 3 times with a substituent independently selected from halo, C₁-C₄alkyl, OH, and ═O;
L is a bond or —(CH₂O)—; and
A⁽⁻⁾ is a pharmaceutically acceptable negative counterion.

According to one embodiment, the compound of Formula I is defined wherein R¹⁵ is C₁-C₆alkyl;
C₆-C₁₀-carbocycle optionally substituted 1 or 2 times with halo, C₁-C₄alkyl, O—C₁-C₄alkyl, O—(CH₂)₄—NH₂, O—(CH₂)₄—N(H)C₁-C₄alkyl, O—(CH₂)₄—N(C₁-C₄alkyl)₂, O—C₁-C₄alkyl-C(O)—NH₂, O—C₁-C₄alkyl-C(O)—N(H)C₁-C₄alkyl. O—C₁-C₄alkyl-C(O)—N(C₁-C₄alkyl)₂,
or a group represented by formula i, ii, iii, iv, v, vi, vii, viii, or ix:
i: C₆alkylene-O—R²¹-Ph⁴;
ii: C₂-C₃alkylene-Ph¹-O—R²¹-Ph⁴;
iii: C₂-C₃alkylene-Ph¹-N(H)—R²²-Ph²;
iv: C₂-C₃alkylene-Het-(R²³)-Ph³;
v: C₂-C₃alkylene-Ph¹—CO—C₂alkylene-C(O)N(H)—C₁-C₄alkylene-Ph³;
vi: C₂-C₃alkylene-Ph³;
vii: C₂-C₃alkylene-S(O)₂—C₂-C₄alkylene-O—C₂-C₄alkylene-Ph³;
viii: C₃-C₆alkylene-Ph¹-C₁₀-C₁₂alkylene-C(O)N(H)—C₁₀-C₁₂ bicyclic carbocycle;
ix: C₃-C₆alkylene-Het-Ph⁴;
   wherein:
   R²¹ is C₂-C₆alkylene wherein one carbon of said alkylene is optionally replaced by O;
   Ph⁴ is phenyl optionally substituted 1 or 2 times by halo, N(H)C(O)NH₂ or S-cyclopentyl,
   Ph¹ is phenylene;
   R²² is a bond or C₁-C₂alkylene optionally substituted once by OH or NH₂;
   Ph² is phenyl optionally substituted 1 or 2 times by O-methyl, —OCH₂C(CH₃)₂CH₂NH₂, —SO₂—NH(C₆H₃)(CH₃)(C₇H₁₅) or

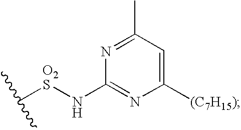

Het is 4-10 ring atom heterocyclene wherein 1, 2 or 3 ring atoms is/are N, O or S (e.g., triazole, indolene or benzodioxylene) optionally substituted once by methyl;
R²³ is a C₂-C₄alkylene wherein one carbon of said alkylene is optionally replaced by O, or CO—C₂alkylene-C(O)N(H)—C₂-C₄alkylene; and
Ph³ is phenyl optionally substituted 1 or 2 times by halo or O-methyl.

In another aspect, the invention provides a compound of Formula II:

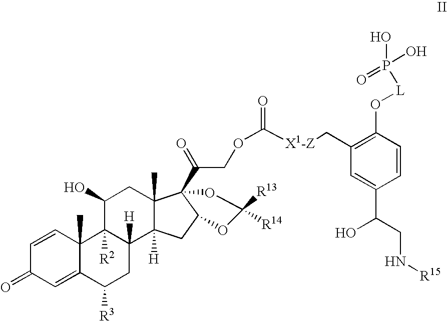

or a pharmaceutically acceptable salt thereof, wherein all variables are as defined above.

In another aspect, the invention provides a compound of Formula III:

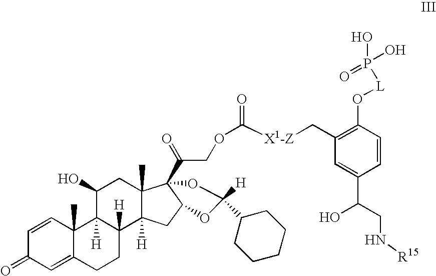

or a pharmaceutically acceptable salt thereof, wherein all variables are as defined above.

In another aspect, the invention provides a pharmaceutical composition comprising an effective amount of a compound of Formula I-1, I, II or III, or a pharmaceutically acceptable salt thereof, in combination with a pharmaceutically acceptable excipient, diluent or carrier. In one embodiment, the composition further comprises a therapeutically active agent selected from anti-inflammatory agents, anticholinergic agents, β-agonists, antiinfective agents and antihistamines.

In another aspect, the invention provides a method comprising administering to a human, an effective amount of a compound of Formula I-1, I, II or III, or a pharmaceutically acceptable salt thereof.

In another aspect, the invention provides a method for the treatment of pulmonary inflammation or bronchoconstriction in a human in need thereof, comprising administering to said human an effective amount of a compound of Formula I-1, I, II or III, or a pharmaceutically acceptable salt thereof.

In another aspect, the invention provides a method for the treatment of a disease associated with reversible airway obstruction, asthma, COPD, bronchiectasis or emphysema in a human in need thereof comprising administering to the human an effective amount of a compound of Formula I-1, I, II or III, or a pharmaceutically acceptable salt thereof.

In another aspect, the invention provides a method for delivering an effective amount of a steroid and a β-agonist to the lung of a human. The method comprises delivering an effective amount of a compound of Formula I-1, I, II or III, or a pharmaceutically acceptable salt thereof to the lung of the human, wherein a phosphate group of the compound is cleaved by an endogenous enzyme and an ester group of the compound is cleaved by an endogenous esterase or chemically by hydrolysis to deliver the steroid and the β-agonist.

In another aspect, the invention provides a compound of Formula I-1, I, II or III, or a pharmaceutically acceptable salt thereof for use as a medicament.

In another aspect, the invention provides a compound of Formula I-1, I, II or III, or a pharmaceutically acceptable salt thereof for use in the treatment of pulmonary inflammation or bronchoconstriction in a human.

In another aspect, the invention provides a compound of Formula I-1, I, II or III, or a pharmaceutically acceptable salt thereof for use in the treatment of a disease associated with reversible airway obstruction, asthma, COPD, bronchiectasis, or emphysema in a human.

In another aspect, the invention provides the use of a compound of Formula I-1, I, II or III, or a pharmaceutically acceptable salt thereof for the manufacture of a medicament for the treatment of pulmonary inflammation or bronchoconstriction in a human.

In another aspect, the invention provides the use of a compound of Formula I-1, I, II or III, or a pharmaceutically acceptable salt thereof for the manufacture of a medicament for the treatment of a disease associated with reversible airway obstruction, asthma, COPD, bronchiectasis, or emphysema in a human.

In another aspect, the invention provides a composition comprising a compound of Formula I-1, I, II or III, or a pharmaceutically acceptable salt thereof for use in the preparation of a medicament for the treatment of pulmonary inflammation or bronchoconstriction in a human.

In another aspect, the invention provides a composition comprising a compound of Formula I-1, I, II or III, or a pharmaceutically acceptable salt thereof for use in the preparation of a medicament for the treatment of reversible airway obstruction, asthma, COPD, bronchiectasis, or emphysema in a human.

In another aspect, the invention provides processes and novel intermediates which are useful for preparing the compounds of Formula I-1, I, II, III and pharmaceutically acceptable salts thereof.

In another aspect, the present invention includes compounds of Formula I-1, I, II, III and pharmaceutically acceptable salts thereof and all racemates, enantiomers, diastereomers, tautomers, polymorphs, pseudopolymorphs and amorphous forms thereof.

### DETAILED DESCRIPTION OF THF INVENTION

Headings are employed throughout the disclosure solely for ease of reference and are in no way to be construed as indicating that all subject matter in the passages below a particular heading constitute the sole disclosure relevant to the topic.

### DEFINITIONS

Unless stated otherwise, the following terms and phrases as used herein are intended to have the following meanings:

When trade names are used herein, applicants intend to independently include the trade name product and the active pharmaceutical ingredient(s) of the trade name product.

As used herein, “a compound of the invention” means a compound of Formula I-1, I, II, or III or a salt, particularly a pharmaceutically acceptable salt thereof.

“A compound of Formula I” means a compound having the structural formula designated herein as Formula I. Compounds of Formula I include solvates and hydrates as well as any amorphous and crystalline (polymorphic) forms thereof. In those embodiments wherein a compound of Formula I includes one or more chiral centers, the phrase is intended to encompass each individual stereoisomer including optical isomers (enantiomers and diastereomers) and geometric isomers (cis-/trans-isomerism) and mixtures of stereoisomers. Similarly, with respect to other compounds referred to herein, such as compounds of Formula I-1, Formula II, Formula III and isolatable intermediates, the phrase “a compound of Formula (number)” means a compound of that formula and solvates and hydrates as well as amorphous and crystalline (polymorphic) forms thereof and stereoisomers (where compounds include a chiral center) thereof.

“Alkyl” is linear or branched hydrocarbon containing normal, secondary, or tertiary carbon atoms and having 1 to 12 carbon atoms (i.e., C₁-C₁₂alkyl), typically 1 to 10 carbon atoms (i.e., C₁-C₁₀alkyl), or more typically, 1 to 6 carbon atoms (i.e., C₁-C₆alkyl), unless the number of carbon atoms is otherwise specified. When the compound of Formula I-1, I, II or III includes more than one alkyl, the alkyls may be the same or different. Examples of suitable alkyl groups include, but are not limited to, methyl (Me, —CH₃), ethyl (Et, —CH₂CH₃), 1-propyl (n-Pr, i-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)₂), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃) “Alkenyl” is a linear or branched hydrocarbon containing normal, secondary, or tertiary carbon atoms with at least one site of unsaturation, i.e. a carbon-carbon, sp² double bond and having 2 to 12 carbon atoms (i.e., C₂-C₁₂alkenyl), or more typically, 2 to 6 carbon atoms (i.e., C₂-C₆alkenyl) unless the number of carbon atoms is otherwise specified. When the compound of Formula I-1, I, II, or III includes more than one alkenyl, the alkenyls may be the same or different. Examples of suitable alkenyl groups include, but are not limited to, ethenyl or vinyl (—CH═CH₂), propenyl or allyl (—CH₂CH═CH₂), and 5-hexenyl (—CH₂CH₂CH₂CH₉CH═CH₂).

“Alkynyl” is a linear or branched hydrocarbon containing normal, secondary, or tertiary carbon atoms with at least one site of unsaturation, i.e. a carbon-carbon, Sp triple bond and having 2 to 12 carbon atoms (i.e., C₂-C₁₂alkyne), or more typically 2 to 6 carbon atoms (i.e., C₂-C₆alkynyl) unless the number of carbon atoms is otherwise specified. When the compound of Formula I-1, I, II or III includes more than one alkynyl, the alkynyls may be the same or different. Examples of suitable alkynyl groups include, but are not limited to, ethynyl (—C≡CH), propargyl (—CH₂C≡CH), and the like.

“Alkylene” refers to a saturated, branched or straight chain hydrocarbon radical having two monovalent radical centers derived by the removal of two hydrogen atoms from the same or two different carbon atoms of a parent alkane, and having 1 to 10 carbon atoms, or more typically 1 to 6 carbon atoms, unless the number of carbon atoms is otherwise specified. When the compound of Formula I-1, I, II or III includes more than one alkylene, the alkylenes may be the same or different. Typical alkylene radicals include, but are not limited to, methylene (—CH₂—), 1,1-ethyl (—CH(CH₃)—), 1,2-ethyl (—CH₂CH₂—), 1,1-propyl (—CH(CH₂CH₃)—), 1,2-propyl (—CH₂CH(CH₃)—), 1,3-propyl (—CH₂CH₂CH₂—), 1,4-butyl (—CH₂CH₂CH₂CH₂—), and the like.

“Alkenylene” refers to an unsaturated, branched or straight chain hydrocarbon radical having two monovalent radical centers derived by the removal of two hydrogen atoms from the same or two different carbon atoms of a parent alkene. For example, and alkenylene group can have 1 to 10 carbon atoms, or more typically 1 to 6 carbon atoms. When the compound of Formula I-1, I, II or III includes more than one alkenylene the alkenylenes may be the same or different. Typical alkenylene radicals include, but are not limited to, 1,2-ethylene (—CH═CH—).

“Alkynylene” refers to an unsaturated, branched or straight chain hydrocarbon radical having two monovalent radical centers derived by the removal of two hydrogen atoms from the same or two different carbon atoms of a parent alkyne, and having 1 to 10 carbon atoms, or 1 to 6 carbon atoms, unless the number of carbon atoms is otherwise specified. When the compound of Formula I-1, I, II or III includes more than one alkynylene, the alkynylene may be the same or different. Typical alkynylene radicals include, but are not limited to, acetylene (—C≡C—), propargyl (—CH₂CH₂CH₂C≡C—), and 4-pentynyl (—CH₂CH₂CH₂C≡C—).

“Carbocycle” or “carbocyclyl” refers to a saturated (i.e., cycloalkyl), partially unsaturated (e.g., cycloakenyl, cycloalkadienyl, etc.) or aromatic ring (i.e., aryl ring) having 3 to 7 carbon atoms as a monocycle, 7 to 12 carbon atoms as a bicycle, including spiro-fused rings, and up to about 20 carbon atoms as a polycycle, unless the number of carbon atoms is otherwise specified (e.g., “C₃-C₆ carbocycle”). Monocyclic carbocycles typically have 3 to 6 ring atoms, and in one embodiment, 5 or 6 ring atoms. Bicyclic carbocycles typically have 7 to 12 ring atoms, e.g., arranged as a bicyclo [4,5], [5,5], [5,6] or [6,6] system, or 9 or 10 ring atoms arranged as a bicyclo [5,6] or [6,6] system, or spiro-fused rings. Non-limiting examples of monocyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and phenyl. Non-limiting examples of bicyclo carbocycles includes naphthyl, dihydronaphthyl, tetrahydronaphthyl, indenyl, and indanyl. In one embodiment, “carbocycle” refers to a saturated, partially unsaturated or aromatic ring which is monocyclic and having from 3 to 7 carbon atoms or which is bicyclic and having from 7 to 12 carbon atoms. In those embodiments wherein the compound of Formula I-1, I, II or III includes more than one carbocycle, the carbocycles may be the same or different.

“Aryl” refers to a subset of carbocycles, namely those carbocycles which are an aromatic hydrocarbon radical derived by the removal of one hydrogen atom from a single carbon atom of an optionally substituted parent aromatic ring system and having 6 to 14 carbon atoms, or more typically 6 to 12 carbon atoms. Typical aryl groups include, but are not limited to, radicals derived from benzene (e.g., phenyl), naphthalene, and the like. In one embodiment, “aryl” is phenyl. In those embodiments wherein the compound of Formula I-1, I, II or III includes more than one aryl, the aryls may be the same or different.

“Arylalkyl” refers to an acyclic alkyl radical in which one of the hydrogen atoms bonded to a carbon atom, typically a terminal or sp³ carbon atom, is replaced with an aryl that is optionally substituted. Typical arylalkyl groups include, but are not limited to, benzyl, 2-phenylethan-1-yl, naphthylmethyl, 2-naphthylethan-1-yl, and the like. The arylalkyl group can comprise 7 to 26 carbon atoms, and more typically 7 to 18 carbon atoms, e.g., the alkyl moiety is 1 to 12 carbon atoms, more typically 1 to 6 carbon atoms, and the aryl moiety is 6 to 14, more typically 6 to 12 carbon atoms.

“Carbocyclene” refers to a saturated (i.e., cycloalkylene), partially unsaturated (e.g., cycloakenylene, cycloalkadienylene, etc.) or aromatic radical as described for “carbocycle” having two monovalent radical centers derived by the removal of two hydrogen atoms from the same or two different carbon atoms of a parent carbocycle. In those embodiments wherein the compound of Formula I-1, I, II or III includes more than one carbocyclene, the carbocyclenes may be the same or different.

“Heterocycle” or “heterocyclyl” are described in Paquette, Leo A.; Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. As used herein, “heterocycle” and “heterocyclyl” are synonymous and refer to a “carbocycle” as defined herein, having 3 to 7 ring atoms as a monocycle, 7 to 12 ring atoms as a bicycle, and up to about 20 ring atoms as a polycycle wherein 1, 2, 3, or 4 carbon ring atoms have been replaced with a heteroatom selected from O, N, and S. The terms “heterocycle” or “heterocyclyl” includes saturated rings, partially unsaturated rings, and aromatic rings (i.e., heterocycle and heterocyclyl includes as a subset heteroaromatic or “heteroaryl” rings).

In one particular embodiment, ⁴“heterocycle” or “heterocyclyl” refers to saturated, partially unsaturated or aromatic monocyclic carbocycles of 4, 5 or 6 ring atoms wherein 1, 2 or 3 of the ring atoms is/are a heteroatom independently selected from N, O and S, and saturated, partially unsaturated or aromatic bicyclic carbocycles of 9 or 10 ring atoms wherein 1, 2, 3 or 4 of the ring atoms is/are a heteroatom independently selected from N, O and S.

In those embodiments wherein the compounds of Formula I-1, I, II, or III include more than one heterocycle, the heterocycles may be the same or different.

Examples of heterocycles include but are not limited to pyridyl, dihydropyridyl, piperidyl, thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, β-carbolinyl, phenanthridinyl, acridinyl, pyrimidinyl, phenanthrolinyl, farazanyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, and bis-tetrahydrofuranyl:

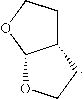

Heterocyclyl groups may be bound through any available ring carbon or ring heteroatom. By way of example and not limitation, carbon bonded heterocycles are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heterocycles are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or β-carboline. Still more typically, nitrogen bonded heterocycles include 1-aziridyl, 1-azetedyl, 1-pyrrolyl, 1-imidazolyl, 1-pyrazolyl, and 1-piperidinyl.

“Heteroaryl” refers to a subset of heterocycles, namely monocyclic and bicyclic fused aromatic heterocycles as defined herein. Non-limiting examples of heteroaryl rings include all of aromatic heterocycles listed above, and particularly pyridinyl, pyrrolyl, oxazolyl, indolyl, isoindolyl, purinyl, furanyl, thienyl, benzofuranyl, benzothiophenyl, imidazolyl, thiazolyl, isoxazolyl, pyrazolyl, isothiazolyl, quinolyl, isoquinolyl, pyridazyl, pyrimidyl, pyrazyl, etc. In those embodiments wherein the compounds of Formula I-1, I, II, or III include more than one heteroaryl, the heteroaryls may be the same or different.

“Heterocyclene” refers to a bivalent heterocycle as defined herein. For example, heterocyclenes include:

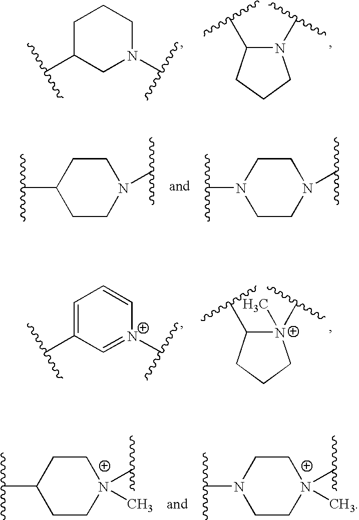

In those embodiments wherein the compounds of Formula I-1, I, II, or III include more than one heterocyclene, the heterocyclenes may be the same or different.

“Heteroarylene” refers to a bivalent, aromatic heterocycle as defined herein. In those embodiments wherein the compounds of Formula I-1, I, II or III include more than one heteroarylene, the heteroarylenes may be the same or different.

“Heteroarylalkyl” refers to an alkyl group, as defined herein, in which a hydrogen atom of the alkyl has been replaced with a heteroaryl as defined herein. Non-limiting examples of heteroarylalkyl include: —CH₂-pyridinyl, —CH₂-pyrrolyl, —CH₂-oxazolyl, —CH₂-indolyl, —CH₂-isoindolyl, —CH₂-purinyl, —CH₂-furanyl, —CH₂-thienyl, —CH₂-benzofuranyl, —CH₂-benzothiophenyl, —CH₂-carbazolyl, —CH₂-imidazolyl, —CH₂-thiazolyl, —CH₂-isoxazolyl, —CH₂-pyrazolyl, —CH₂-isothiazolyl, —CH₂-quinolyl, —CH₂-isoquinolyl, —CH₂-pyridazyl, —CH₂-pyrimidyl, —CH₂-pyrazyl, —CH(CH₃)-pyridinyl, —CH(CH₃)-pyrrolyl, —CH(CH₃)-oxazolyl, —CH(CH₃)-indolyl, —CH(CH₃)-isoindolyl, —CH(CH₃)-purinyl, —CH(CH₃)-furanyl, —CH(CH₃)-thienyl, —CH(CH₃)-benzofuranyl, —CH(CH₃)— benzothiophenyl, —CH(CH₃)-carbazolyl, —CH(CH₃)-imidazolyl, —CH(CH₃)-thiazolyl, —CH(CH₃)-isoxazolyl, —CH(CH₃)-pyrazolyl, —CH(CH₃)-isothiazolyl, —CH(CH₃)-quinolyl, —CH(CH₃)-isoquinolyl, —CH(CH₃)-pyridazyl, —CH(CH₃)-pyrimidyl, —CH(CH₃)-pyrazyl, etc.

The term “optionally substituted” in reference to a particular moiety of the compound of Formula I-1, I, II or III (e.g., an optionally substituted aryl group) refers to a moiety having 0, 1, 2, or more substituents, more particularly 0, 1 or 2 substituents, unless otherwise indicated. In reference to alkyl, alkylene, aryl, alkoxy, carbocyclyl, and carbocyclene, typical substituents include, but are not limited to, halogen (halo) (i.e., F, Cl, Br, or I), C₁-C₆alkyl, ═O, —OR, —SR, —SR₂⁺A⁽⁻⁾, —NR₂, —N⁺R₃A⁽⁻⁾, ═NR, —CN, —NO₂, —NHC(═O)R, —NHC(═O)NR₂, —C(═O)R, —C(═O)NR₂, —S(═O)₂OH, —S(═O)₂NY, —S(═O)R, —OP(═O)(OR)₂, —P(═O)(OR)₂, —C(O)OR, —C(S)OR, —C(O)SR, and —C(═NR)NRR, wherein R is H or C₁-C₆alkyl. Unless otherwise indicated, when the term “substituted” is used in conjunction with groups which have multiple available sites for substitution, two or more moieties capable of substitution, the substituents can be attached to any available C or heteroatom.

“Linker” or “link” means a chemical moiety comprising a covalent bond or a chain of atoms.

The term “prodrug” as used herein refers to any compound that when administered to a biological system generates the drug substance, i.e., active ingredient, as a result of spontaneous chemical reactions), enzyme catalyzed chemical reaction(s), photolysis, and/or metabolic chemical reaction(s). A prodrug is thus a covalently modified analog or latent form of a therapeutically active compound.

### Compounds

One skilled in the art will recognize that substituents and other moieties of the compounds of Formula I-1, I, II or III should be selected in order to avoid embodiments which would be recognized by one of ordinary skill in the art as obviously inoperative. In one embodiment the substituents and other moieties are selected in order to provide a compound which is sufficiently stable to provide a pharmaceutically active compound. Compounds of Formula I-1, I, II or III which have such stability are contemplated as falling within the scope of the present invention.

In some chemical structure representations where carbon atoms do not have a sufficient number of variables attached to produce a valence of four, the remaining carbon substituents needed to provide a valence of four should be assumed to be hydrogen. For example,

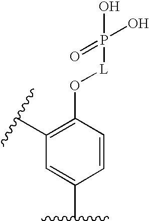

has the same meaning as

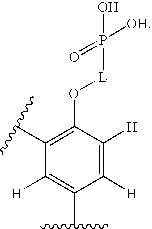

Similarly, in some chemical structures where a bond is drawn without specifying the terminal group, such bond is indicative of a methyl group, as is conventional in the art. Thus,

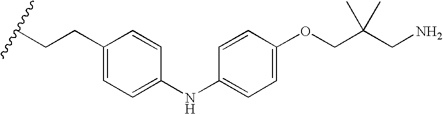

is the same as

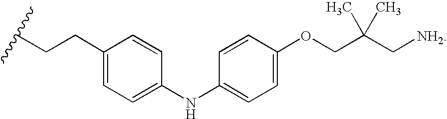

For ease of reference, the constituent moieties of the compounds of Formula I may be referred to herein from time to time as follows:

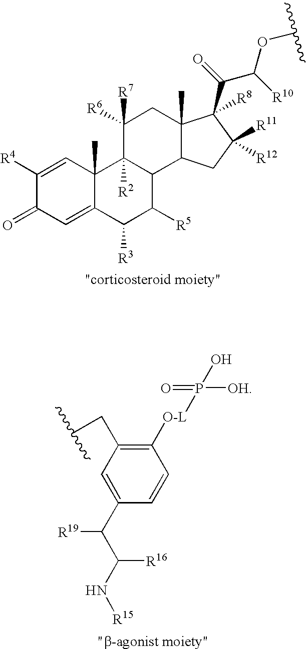

Thus, in one aspect, the invention comprises a compound of Formula I-1:

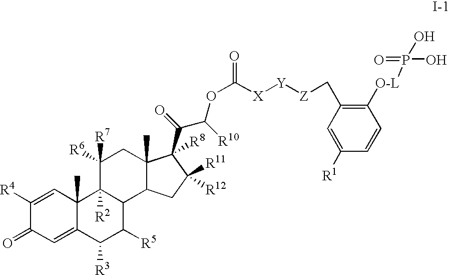

or a pharmaceutically acceptable salt thereof,
wherein:
R¹ is

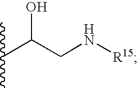

each R², R³, R⁴, and R⁵ are, independently, H, C₁-C₄alkyl or halo;
R⁶ and R⁷ are, independently, H or OH; or R⁶ and R⁷ taken together with the carbon to which they are attached form a >C═O group;
R⁸ is H, OH, O(CO)R⁹, or O(CO)OR⁹;
each R⁹ is, independently, C₁-C₄alkyl;
each R¹⁰ and R¹ is, independently, H or C₁-C₄alkyl;
R¹² is H, OH, or R⁹; or R¹¹ and R¹² taken together with the carbon to which they are attached form a >═CH₂ group; or R¹² and R⁸ taken together with the carbons to which they are attached form a 1,3-dioxolane ring represented by formula B

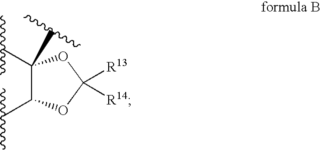

each R¹³ and R¹⁴ are, independently, H, optionally substituted C₁-C₁₀alkyl, optionally substituted C₂-C₁₀alkenyl, optionally substituted C₂-C₁₀alkynyl, optionally substituted C₃-C₁₀ carbocyclyl, optionally substituted C₆-C₁₀ aryl, or optionally substituted heteroaryl;
R¹⁵ is optionally substituted C₁-C₁₂alkyl, arylalkyl, substituted arylalkyl, or optionally substituted carbocyclyl wherein 1-3 carbon atoms of said optionally substituted C₁-C₁₂alkyl, arylalkyl, substituted arylalkyl or optionally substituted carbocyclyl may be replaced by O, S, N(H), or N(C₁-C₄alkyl);
X is a bond, O, S, N(H), N(C₁-C₄alkyl), optionally substituted C₁-C₁₀alkylene, optionally substituted C₂-C₁₀alkenylene, optionally substituted C₂-C₁₀alkynylene, optionally substituted C₆-C₁₀ arylene, optionally substituted heterocyclene, optionally substituted heteroarylene or optionally substituted C₃-C₁₀ carbocyclene;
Y is a bond, optionally substituted C₁-C₁₀alkylene, optionally substituted C₂-C₁₀alkenylene, optionally substituted C₂-C₁₀alkynylene, optionally substituted C₃-C₁₀ carbocyclene, optionally substituted C₆-C₁₀ arylene, or optionally substituted heteroarylene; wherein one or more carbon atoms of said C₁-C₁₀alkylene or C₃-C₁₀ carbocyclene is, optionally, replaced by O, S, N(H), N(C₁-C₄alkyl), —N(H)C(O)—, —N(C₁-C₄alkyl)C(O)—, —C(O)N(H), or CC(O)N(C₁-C₄alkyl)-;
Z is ^{⊕}(NR¹⁷R¹⁸)A⁽⁻⁾, N(O)R¹⁷ (N-oxide), S(O) (sulfoxide), S(═O)₂, ^{⊕}(SR¹⁷)A⁽⁻⁾, a heterocyclene comprising ^{⊕}(NR¹⁷)A⁽⁻⁾ or (SA⁽⁻⁾, or a heteroarylene comprising a ^{⊕}NA⁽⁻⁾; wherein when Z is said heterocyclene or said heteroarylene the group represented by

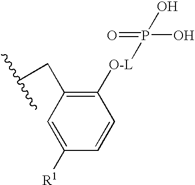

in Formula I-1 is directly bonded to a ^{⊕}NR¹⁷ or ^{⊕}S of said heterocyclene or a ^{⊕}N of said heteroarylene;
L is a bond or —(CH₂O)—;
each R¹⁷ and R¹⁸ are, independently, optionally substituted C₁-C₁₀alkyl, optionally substituted C₂-C₁₀alkenyl, optionally substituted C₂-C₁₀alkynyl, optionally substituted C₃-C₁₀ carbocyclyl, optionally substituted C₆-C₁₀ aryl, or optionally substituted heteroaryl; or R¹⁷ and R¹⁸ taken together with the nitrogen to which they are attached form a heterocyclic ring comprising 3-7 carbon atoms wherein one or more carbon atoms of said heterocyclic ring is, optionally, replaced by O, S, N(H), or N(C₁-C₄alkyl); and
A⁽⁻⁾ is a pharmaceutically acceptable negative counterion.

The compounds of Formula I-1 comprise a charged phosphate group and a highly polarized N or S group creating a highly polar molecule that has high affinity for lung cell surfaces, lung DNA and protein thus minimizing systemic absorption.

When X in Formula I-1 is a bond, it is intended that the carbonyl group in Formula I-1 is directly attached to Y. When Y is a bond, it is intended that X in Formula I-1 is directly attached to Z. When each X and Y of Formula I-1 are both a bond, it is intended that the carbonyl group is directly attached to Z. Similarly, when L is a bond, it is intended that the aryl oxygen is directly attached to the P atom.

In one embodiment of Formula I-1, X is a bond, O, S, N(H), N(C₁-C₄alkyl), optionally substituted C₁-C₁₀alkylene, optionally substituted C₂-C₁₀alkenylene, optionally substituted C₂-C₁₀alkynylene, optionally substituted C₆-C₁) arylene, optionally substituted heterocyclene, optionally substituted heteroarylene or optionally substituted C₃-C₁₀ carbocyclene. In one embodiment, X is a bond. In another embodiment, X is O.

In another embodiment, X is S. In another embodiment, X is N(H) or N(C₁-C₄alkyl). In another embodiment, X is optionally substituted C₁-C₆alkylene. In another embodiment, X is optionally substituted C₂-C₄alkenylene. In another embodiment, X is optionally substituted C₂-C₄alkynylene. In another embodiment, X is optionally substituted C₆ arylene. In another embodiment, X is optionally substituted heterocyclene. In another embodiment, X is optionally substituted heteroarylene. In another embodiment, X is optionally substituted C₃-C₁₀ carbocyclene.

In another embodiment of Formula I-1, Y is a bond, optionally substituted C₁-C₁₀alkylene, optionally substituted C₂-C₁₀alkenylene, optionally substituted C₂-C₁₀alkynylene, optionally substituted C₃-C₁₀ carbocyclene, optionally substituted C₆-C₁₀ arylene, or optionally substituted heteroarylene; wherein one or more carbon atoms of said C₁-C₁₀alkylene or C₃-C₁₀ carbocyclene is, optionally, replaced by O, S, N(H), N(C₁-C₄alkyl), —N(H)—C(O)—, —N(C₁-C₄alkyl)-C(O)—, —C(O)N(H)— or —C(O)N(C₁-C₄alkyl)-. In a preferred embodiment, Y is a bond. In another preferred embodiment, Y is optionally substituted C₁-C₆alkylene. In another preferred embodiment, Y is optionally substituted C₁-C₆alkylene wherein a carbon atom of said C₁-C₆alkylene is replaced by —N(H)—C(O)—, —N(C₁-C₄alkyl)-C(O)—, C(O)N(H)— or C(O)N(C₁-C₄alkyl)-. In another preferred embodiment, Y is C₂-C₄alkenylene or C₂-C₄alkynylene.

In another embodiment of Formula I-1, Z is ^{⊕}(NR¹⁷R¹⁸)A⁽⁻⁾, N(O)R¹⁷(N-oxide), S(O) (sulfoxide), S(O)₂, ^{⊕}(SR¹⁷)A⁽⁻⁾, a heterocyclene comprising ^{⊕}(NR⁷)A⁽⁻⁾ or ^{⊕}SA⁽⁻⁾, or a heteroarylene comprising a ^{⊕}NA⁽⁻⁾; wherein when Z is said heterocyclene or said heteroarylene the group represented by

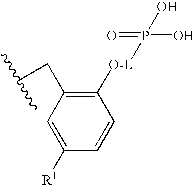

in Formula I-1 is directly bonded to a ^{⊕}NR⁷ or ^{⊕}S of said heterocyclene or a ^{⊕}N of said heteroarylene. As represented in Formula I-1, Z is a highly polarized center comprising a nitrogen atom or a sulfur atom that may bear a positive charge. In another embodiment, Z is ^{⊕}(NR¹⁷R¹⁸)A⁽⁻⁾. In another embodiment, Z is ^{⊕}(NR¹⁷R¹⁸)A⁽⁻⁾ and R¹⁷ and R¹⁸ are independently methyl or ethyl. In another embodiment, Z is N(O)R¹⁷ (N-oxide). In another embodiment, Z is 3 (SR¹⁷)A⁽⁻⁾. In another embodiment, Z is a heterocyclene comprising ^{⊕}(NR¹⁷)A⁽⁻⁾ wherein the group represented by

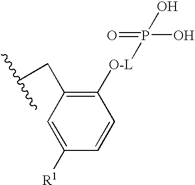

in Formula I-1 is bonded to ^{⊕}NR¹⁷. In another embodiment Z is S(O) (sulfoxide). In another embodiment, Z is S(═O)₂. In another embodiment, Z is a heterocyclene comprising ^{⊕}SA⁽⁻⁾ wherein the group represented by

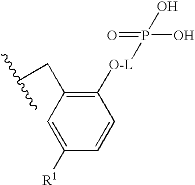

in Formula I-1 is bonded to ^{⊕}S.

In another embodiment, Z is heteroarylene comprising a ^{⊕}NA⁽⁻⁾ wherein the group represented by

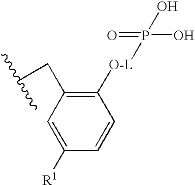

in Formula I-1 is bonded to a ^{⊕}N of said heteroarylene.

In a preferred embodiment of Formula I-1, X is a bond, Y is C₁-C₆alkylene, and Z is ^{⊕}(NR¹⁷R¹⁸)A⁽⁻⁾. In another preferred embodiment, X is a bond, Y is C₁-C₆alkylene, and Z is ^{⊕}(NR¹⁷R¹⁸)A⁽⁻⁾, wherein each R¹⁷ and R¹⁸ is independently methyl or ethyl. In another preferred embodiment, X is O, Y is C₁-C₆alkylene, and Z is ^{⊕}(NR¹⁷R¹⁸)A⁽⁻⁾, wherein each R¹⁷ and R¹⁸ is independently methyl or ethyl. In another preferred embodiment X is optionally substituted C₆ arylene, Y is C₁-C₆alkylene, and Z is ^{⊕}(NR¹⁷R¹⁸)A⁽⁻⁾, wherein each R¹⁷ and R¹⁸ is independently methyl or ethyl. In another preferred embodiment, each X and Y is a bond and Z is heteroarylene comprising a ^{⊕}NA⁽⁻⁾. In another preferred embodiment, X is a bond, Y is C₁-C₆alkylene and Z is heteroarylene comprising a ^{⊕}NA⁽⁻⁾. In another preferred embodiment, X is a bond, Y is C₂-C₄alkenylene or C₂-C₄alkynylene, and Z is heteroarylene comprising a ^{⊕}NA⁽⁻⁾. In another preferred embodiment, each X and Y is a bond and Z is heterocyclene comprising ^{⊕}(NR¹⁷R¹⁸)A⁽⁻⁾ wherein R¹⁷ is methyl or ethyl. In another preferred embodiment, X is N(H) or N(C₁-C₄alkyl), Y is C₁-C₆alkylene, and Z is ^{⊕}(NR¹⁷R¹⁸)A⁽⁻⁾, wherein each R¹⁷ and R¹⁸ is independently methyl or ethyl.

In one preferred embodiment, invention comprises compounds of Formula I:

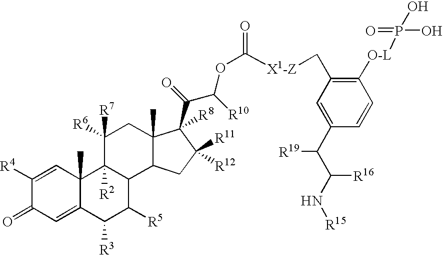

or a pharmaceutically acceptable salt thereof,
wherein:
R¹⁵ is a side chain radical of a β-agonist;

R¹⁶ is H, methyl or ethyl;
R¹⁹ is H, F, OH or methyl;
each R², R³, R⁴, and R⁵ are independently H, C₁-C₄alkyl or halo;
R⁶ and R⁷ are independently H or OH; or R⁶ and R⁷ taken together with the carbon to which they are attached form a >C═O group;
R⁸ is H, OH, O(CO)R⁹, or O(CO)OR⁹;
each R⁹ is independently C₁-C₄alkyl;
each R¹⁰ and R¹¹ is independently H or C₁-C₄alkyl;
R¹² is H, OH, or C₁-C₄alkyl; or
R¹¹ and R¹² taken together with the carbon to which they are attached form a >═CH, group; or
R¹² and R⁸ taken together with the carbons to which they are attached form a 1,3-dioxolane ring represented by formula B:

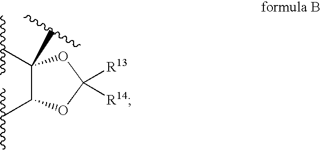

wherein one of R¹³ and R¹⁴ is H, methyl or ethyl and the other is H, C₁-C₁₀alkyl, C₂-C₁₀alkenyl, C₂-C₁₀alkynyl, optionally substituted C₃-C₁₀ carbocyclyl or optionally substituted 5-6 ring atom heterocycle wherein one or two ring atoms are selected from N, O and S, and wherein said carbocyclyl and said heterocyclyl are each optionally substituted 1, 2 or 3 times with a substituent selected from halo, C₁-C₄alkyl, and O—C₁-C₄alkyl;
Z is N(H), N(C₁-C₆alkyl), ^{⊕}(NR¹⁷R¹⁸)A⁽⁻⁾, N(O)R¹⁷ (N-oxide), S(O) (sulfoxide), S(═O)₂, ^{⊕}(SR¹⁷)A⁽⁻⁾, or a 4-9 ring atom heterocyclene wherein one ring atom is N, ^{⊕}(N)A⁽⁻⁾, ^{⊕}(N(C₁-C₆alkyl))A⁽⁻⁾ or ^{⊕}SA⁽⁻⁾, and the β-agonist moiety

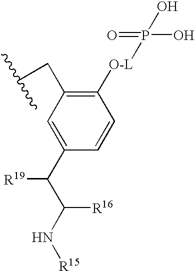

in Formula I is bonded to the N, ^{⊕}N, ^{⊕}N(C₁-C₆alkyl) or ^{⊕}S atom of the heterocyclene;
X¹ is selected from a bond,
   C₁-C₂alkylene, C₂-C₁₂alkenylene, C₂-C₁₂alkynylene,
   O—C₁-C₁₂alkylene, O—C₂-C₂alkenylene, O—C₂-C₁₂alkynylene,
   S—C₁-C₁₂alkylene, S—C₂-C₁₂alkenylene, S—C₆-C₁₂alkynylene,
   N(H)—C₁-C₁₂alkylene, N(H)—C₂-C₁₂alkenylene, N(H)—C₂-C₁₂alkynylene,
   N(C₁-C₆alkyl)-C₁-C₁₂alkylene, N(C₁-C₆alkyl)-C₂-C₁₂alkenylene, N(C₁-C₆alkyl)-C₂-C₁₂alkynylene,
   C₃-C₇-carbocyclene, C₃-C₇-carbocyclene-C₁-C₆alkylene, heterocyclene, heterocyclene-C₁-C₆alkylene, heterocyclene-C(O), wherein said heterocyclene is a 3-9 ring atom heterocyclene wherein 1 or 2 ring atoms are selected from N, O and S,
   C₁-C₆alkylene-O—C₁-C₆alkylene, C₁-C₆alkylene-S—C₁-C₆alkylene, C₁-C₆alkylene-N(H)—C₆-C₆alkylene, C₁-C₆alkylene-N(C₁-C₃alkyl)-C₁-C₆alkylene,
   C₁-C₆alkylene-C₃-C₇-carbocyclene-C₁-C₆alkylene, C₁-C₆alkylene-heterocyclene-C₁-C₆alkylene, wherein said heterocyclene is a 3-9 ring atom heterocyclene wherein 1 or 2 ring atoms are selected from N, O and S,
   C₁-C₁₂alkylene-O, C₁-C₁₂alkylene-S, C₁-C₁₂alkylene-N(H), C₁-C₂alkylene-N(C₁-C₆alkyl), C₁-C₈alkylene-N(H)C(O), C₁-C₈alkylene-N(C₁-C₄alkyl)C(O), C₁-C₈alkylene-C(O)N(H), C₁-C₈alkylene-C(O)N(C₁-C₄alkyl),
   CH-AA, and C(H)(AA)-N(H)C(O), wherein AA is a proteinogenic amino acid side chain;
   wherein each alkyl, alkylene, alkenylene, and alkynylene is optionally substituted 1 or 2 times with a substituent independently selected from halo, OH. OCH₃, NH₂, N(H)CH₃, and N(CH₃)₂, and each carbocyclene and heterocyclene is optionally substituted 1, 2 or 3 times with a substituent independently selected from halo, and C₁-C₄alkyl;
wherein when Z is N(H), N(C₁-C₆alkyl), ^{⊕}(NR¹⁷R¹⁸)A⁽⁻⁾, N(O)R¹⁷ (N-oxide), S(O) (sulfoxide), S(═O)₂, or ^{⊕}(SR¹⁷)A⁽⁻⁾, then X¹ is neither a bond nor bound to Z through O, S, N(H), N(C₁-C₆alkyl), N(H)C(O), N(C₁-C₄alkyl)C(O), C(O)N(H) or C(O)N(C₁-C₄alkyl);
wherein each R¹⁷ and R¹⁸ are, independently, C₁-C₆alkyl, C₁-C₆alkenyl, C₁-C₆alkynyl, or C₃-C₇-carbocycle, wherein said alkyl, alkenyl, alkynyl is optionally substituted 1, 2 or 3 times with a substituent independently selected from halo, OH, and ═O, and the carbocycle is optionally substituted 1, 2 or 3 times with a substituent independently selected from halo, C₁-C₄alkyl, OH, and —O;
L is a bond or —(CH₂O)—; and
A⁽⁻⁾ is a pharmaceutically acceptable negative counterion.

In Formula I, and also Formulas II and III disclosed below, when XI is a bond, it is intended that the carbonyl group is directly attached to Z. Similarly, when L is a bond, it is intended that the benzyl oxygen is directly attached to the P atom.

For the sake of brevity, the description of embodiments below may reference “compounds of Formula I”. It should be understood that the definitions of variables and embodiments thereof apply equally to the same variable in compounds of Formula I-1, II and III as if the disclosure referenced all of “Formulas I-1, I, II and III.”

In Formula I, R¹⁵ is a side chain radical of a β-agonist. β-agonists which may provide the requisite side chain radical R¹⁵ are known in the art and include a variety of chemical structures. Suitable side chain radicals of a β-agonist may for example be derived from β-agonist compounds such as those disclosed in Brown et al., Bioorg. Med. Chem Letters 17 (2007) 6188-6191; Bioorg. Med Chem Letters 18 (2008) 1280-1283; and Glossop et al., Annual Reports in Medicinal Chemistry 41 (2006) 237-248. In one embodiment, the side chain radical of a β-agonist is a side chain radical of a selective β₂-agonist.

Specific examples of known β-agonists from which the side chain radical R¹⁵ may be derived include but are not limited to the following compounds:

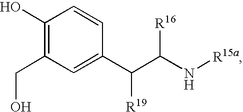

wherein R^{15a} is
t-butyl;
isopropyl;
—(CH₂)₆—O—(CH₂)₄-phenyl;

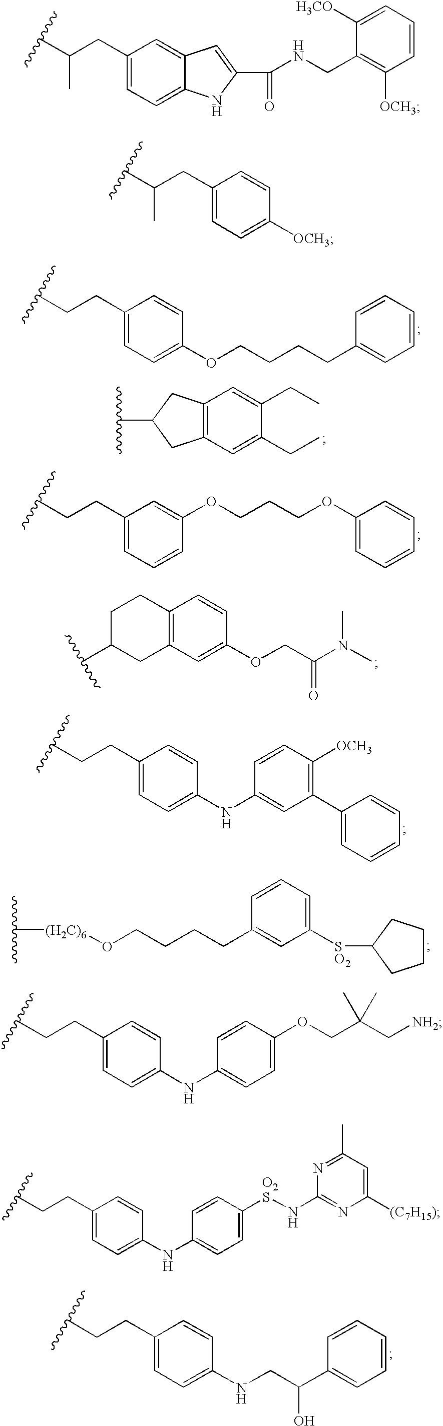

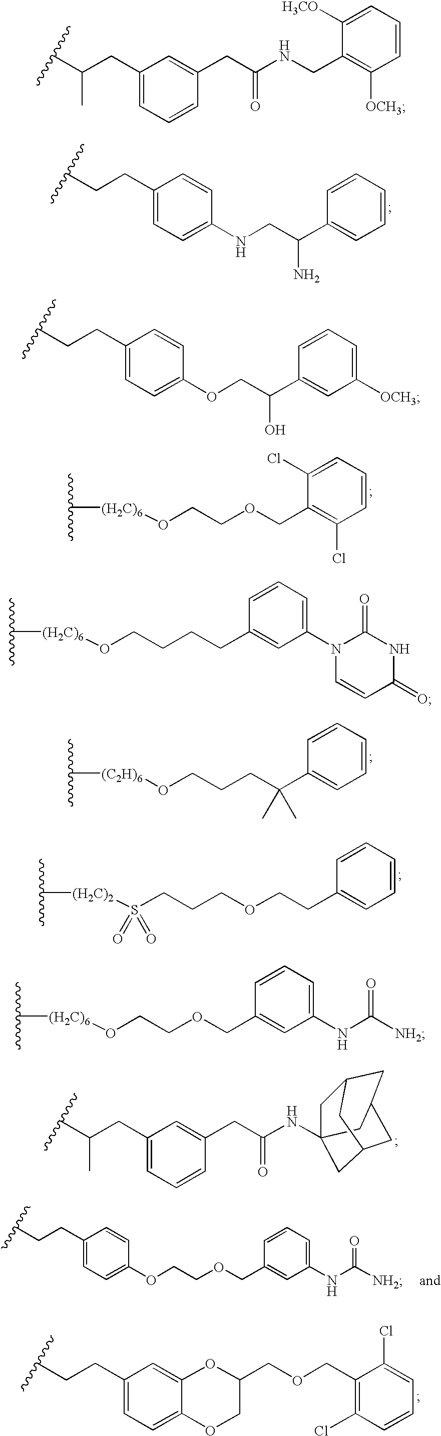

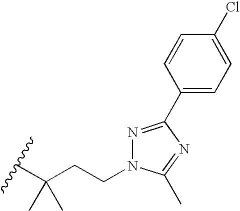

or any subset thereof.

In a particular embodiment, R¹⁵ is
C₁-C₆alkyl;
C₆-C₁₀-carbocycle optionally substituted 1 or 2 times with halo, C₁-C₄alkyl, O—C₁-C₄alkyl, O—(CH₂)₄—NH₂, O—(CH₂)₄—N(H)C₁-C₄alkyl, O—(CH₂)₄—N(C₁-C₄alkyl)₂, O—C₁-C₄alkyl-C(O)—NH₂, O—C₁-C₄alkyl-C(O)—N(H)C₁-C₄alkyl, O—C₁-C₄alkyl-C(O)—N(C₁-C₄alkyl)₂,
or a group represented by formula i, ii, iii, iv, v, vi, vii, viii or ix:
i: C₆alkylene-O—R²¹-Ph⁴;
ii: C₂-C₃alkylene-Ph¹-O—R²¹-Ph⁴;
iii: C₂-C₃alkylene-Ph¹-N(H)—R²²-Ph²;
iv: C₂-C₃alkylene-Het-(R²³)-Ph³;
v: C₂-C₃alkylene-Ph¹-CO—C₂alkylene-C(O)N(H)—C₁-C₄alkylene-Ph³;
vi: C₂-C₃alkylene-Ph³;
vii: C₂-C₃alkylene-S(O)₂—C₁-C₄alkylene-O—C₂-C₄alkylene-Ph³;
viii: C₃-C₆alkylene-Ph¹-C₁₀-C₁₂alkylene-C(O)N(H)—C₁₀-C₁₂ bicyclic carbocycle;
ix: C₃-C₆alkylene-Het-Ph⁴;
   wherein:
   R²¹ is C₂-C₆alkylene wherein one carbon of said alkylene is optionally replaced by O;
   Ph⁴ is phenyl optionally substituted 1 or 2 times by halo, N(H)C(O)NH₂ or S-cyclopentyl,
   Ph¹ is phenylene;
   R²² is a bond or C₁-C₂alkylene optionally substituted once by OH or NH—;
   Ph² is phenyl optionally substituted 1 or 2 times by O-methyl, —OCH₂C(CH₃)₂CH₂NH₂, —SO₂—NH(C₆H₃)(CH₃)(C₇H₁₅), or

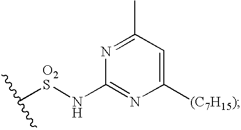

Het is 4-10 ring atom heterocyclene wherein 1, 2 or 3 ring atoms is/are N, O or S (e.g., indolene or benzodioxylene);
R²³ is a C₂-C₄alkylene wherein one carbon of said alkylene is optionally replaced by O or —C₁-C₂alkylene-C(O)N(H)—C₂-C₄alkylene; and
Ph³ is phenyl optionally substituted 1 or 2 times by halo or O-methyl.

In one embodiment, R¹⁵ is C₁-C₆alkyl. More particularly R¹⁵ is C₃-C₄alkyl. In one particular embodiment, R¹⁵ is isopropyl or t-butyl.

In one embodiment, R¹⁵ is C₆-C₁₀ carbocycle optionally substituted 1 or 2 times with C₁-C₄alkyl, O—C₁-C₄alkyl, or O—C₁-C₄alkyl-C(O)—NH₂, or any subset thereof. In one embodiment, R⁵ is C₉-C₁₀ carbocycle optionally substituted 1 or 2 times with C₁-C₄alkyl, O—C₁-C₄alkyl, or O—C₁-C₄alkyl-C(O)—NH₂, or any subset thereof. In one embodiment, R¹⁵ is

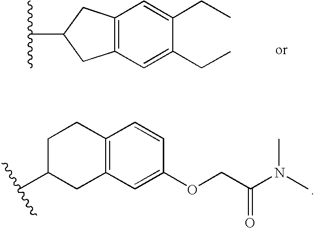

In one embodiment, R¹⁵ is a group represented by formula i: C₆alkylene-O—R²¹-Ph⁴. In one embodiment R¹⁵ is a group represented by formula i and R²¹ is C₄alkylene. In one particular embodiment, R¹⁵ is a group represented by formula i and R²¹ is C₄alkylene and Ph⁴ is phenyl, particularly unsubstituted phenyl. According to one preferred embodiment, R¹⁵ is —(CH₂)₆—O—(CH₂)₄-phenyl, i.e.,

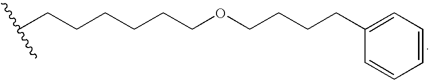

In one embodiment R¹⁵ is a group represented by formula i and i<21 is C₄alkylene wherein one C is replaced by O; more particularly, R²¹ is —(CH₂)₂—O—CH₂—. In one particular embodiment R²¹ is —(CH₂)₂—O—CH₂— and Ph⁴ is phenyl optionally substituted 1 or 2 times with halo, particularly Cl, or 1 time with —N(H)—C(O)—NH₂.

In one embodiment R¹⁵ is a group represented by formula ii: C₂-C₃alkylene-Ph¹-O—R²″-Ph⁴. In one embodiment R¹⁵ is a group represented by formula ii and R²¹ is C₄alkylene wherein one C is optionally replaced by O and Ph⁴ is unsubstituted phenyl. In one particular embodiment R¹⁵ is a group represented by formula ii and R²¹ is —(CH₂)₄— or —(CH₂)₂—O—CH₂— and Ph⁴ is unsubstituted phenyl.

In one embodiment R¹⁵ is a group represented by formula iii: C₂-C₃alkylene-Ph¹-N(H)—R²²-Ph². In one embodiment R¹⁵ is a group represented by formula iii and R²² is a bond or C₂alkylene substituted once by OH or NH₂. In one embodiment R¹⁵ is a group represented by formula iii, R²² is a bond and ph² is phenyl substituted by O-methyl and unsubstituted phenyl or Ph² is phenyl substituted by —OCH₂C(CH₃)₂CH₂NH₉ In one embodiment R¹⁵ is a group represented by formula iii, R²² is C₂ₐₖylene substituted once by OH or NH₂, and Ph² is unsubstituted phenyl.

In one embodiment R¹⁵ is a group represented by formula iv: C₂-C₃alkylene-Het-(R²³)-ph³. In one embodiment R¹⁵ is a group represented by formula iv and Het is a 9 or 10 ring atom heterocyclene wherein 1 or 2 ring atoms is N, O or S. In one embodiment, R¹⁵ is a group represented by formula iv and Het is indolene or benzodioxolene. In one embodiment, R¹⁵ is a group represented by formula iv and R²³ is —CH₂—O—CH₂— or —C(O)N(H)—C₂—. In one embodiment, R¹⁵ is a group represented by formula iv and Ph³ is unsubstituted phenyl, phenyl substituted twice by halo (particularly Cl) or O-methyl, or any subset thereof.

In one embodiment R¹⁵ is a group represented by formula v: C₂-C₃alkylene-Ph¹-CO—C₂alkylene-C(O)N(H)—C₁₋₄alkylene-Ph³. In one embodiment, R¹⁵ is a group represented by formula v and Ph³ is phenyl substituted twice by halo (particularly Cl) or O-methyl. In one embodiment, R¹⁵ is C₂-C₃alkylene-Ph¹-CH₂—C(O)N(H)—CH₂-Ph³.

In one embodiment R¹⁵ is a group represented by formula vi: C₂-C₃alkylene-Ph³. Tin one embodiment, R is a group represented by formula vi and Ph³ is phenyl substituted once by O-methyl.

In one embodiment, R¹⁵ is a group represented by formula vii: C₂-C₃alkylene-S(O)₂—C₂₋₄alkylene-O—C₂₋₄alkylene-Ph³. In one embodiment, R¹⁵ is a group represented by formula vii and Ph³ is unsubstituted phenyl.

In one embodiment, R¹⁵ is a group represented by formula viii: C₃-C₆alkylene-Ph¹-CO—C₂alkylene-C(O)N(H)—C₁₀-C₁₂ bicyclic carbocycle. In one embodiment, R¹⁵ is a group represented by formula viii-a: (branched) C₃ₐₗₖylene-Ph¹-CH₂C(O)N(H)-adamantyl.

In one embodiment, R¹⁵ is a group represented by formula ix: C₃-C₆alkylene-Het-Ph⁴. In one embodiment, R¹⁵ is a group represented by formula ix wherein Het is a 5 or 6 ring atom heterocyclene wherein 1, 2 or 3 atoms are N and the remaining atoms are C, wherein said heterocyclene is optionally substituted once by methyl and Ph⁴ is halo-substituted, particularly Cl-substituted phenyl.

In one particular embodiment, R¹⁵ is selected from:

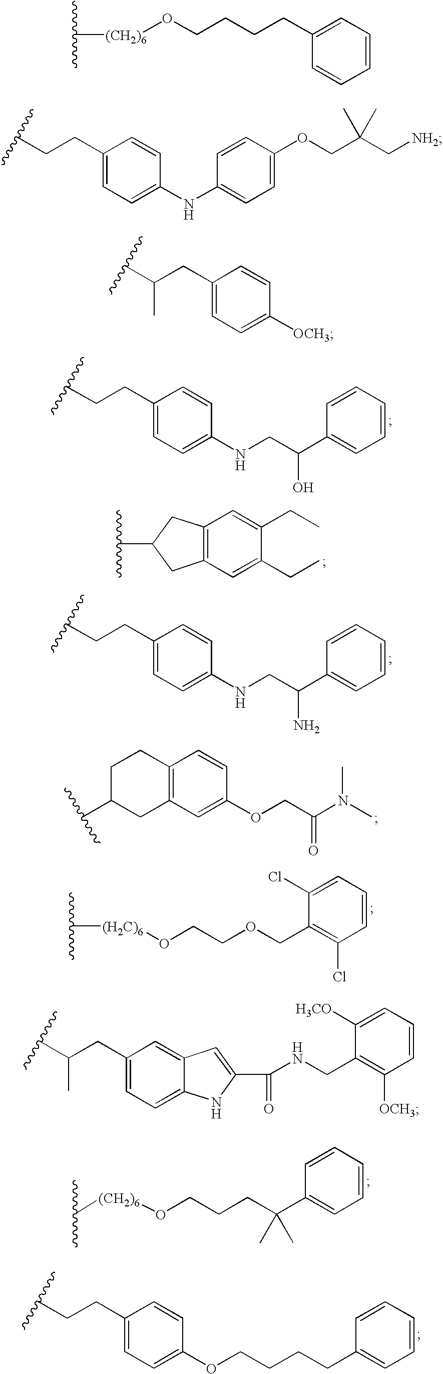

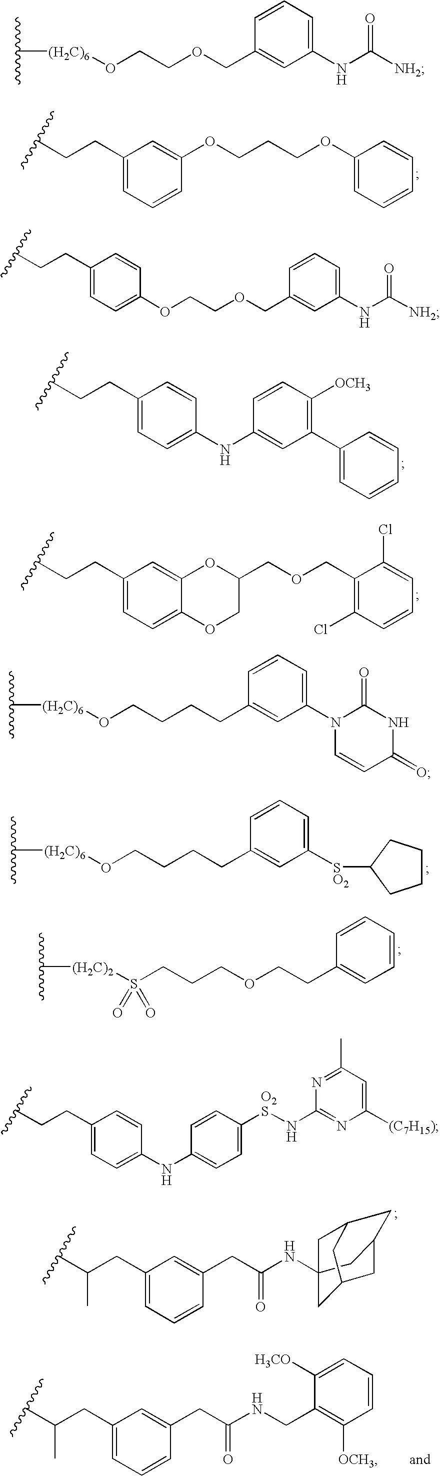

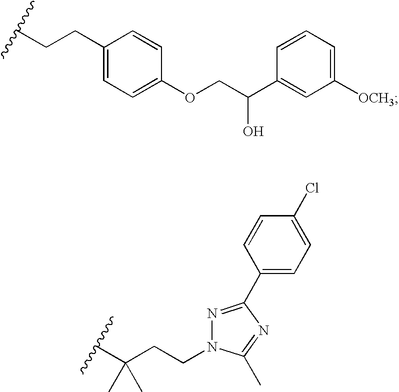

or any subset thereof wherein the wavy bond indicates the point of attachment.

In one preferred embodiment, R¹⁵ is selected from

t-butyl, isopropyl:

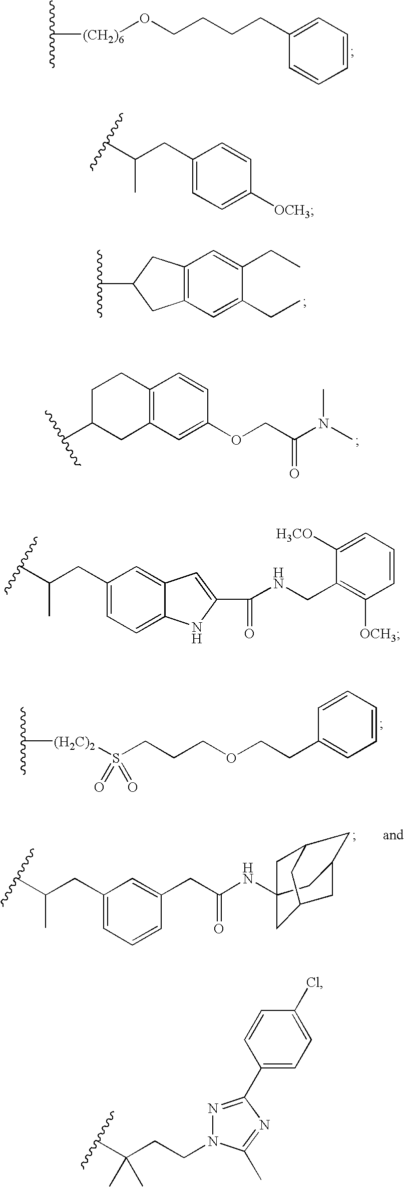

or any subset thereof

In one preferred embodiment, R¹⁵ is

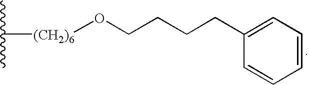

In one preferred embodiment, R¹⁵ is

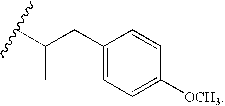

In one preferred embodiment: R¹⁵ is

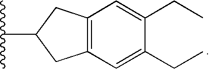

In one preferred embodiments R¹⁵ is

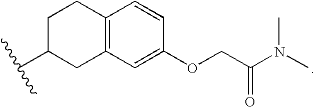

In one preferred embodiment, R¹⁵ is

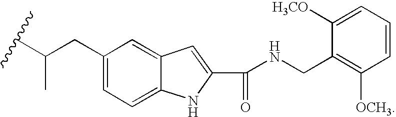

In one preferred embodiment, R¹⁵ is

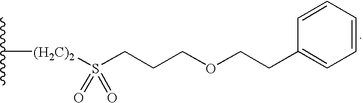

In one preferred embodiment, R¹⁵ is

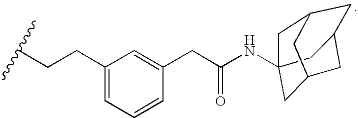

In one preferred embodiment, R¹⁵ is

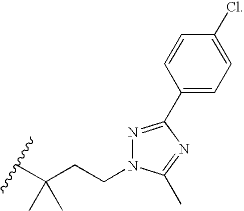

In one embodiment R¹⁶ is H or methyl. In one preferred embodiment, R¹⁶ is H.

In one preferred embodiment, R¹⁹ is OH.

In addition to the β-agonist moiety, the compounds of Formula I also include a corticosteroid moiety:

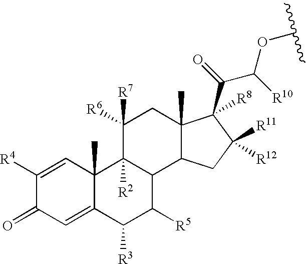

wherein
each of R², R³, R⁴, and R⁵ are independently H, C₁-C₄alkyl or halo;
R⁶ and R⁷ are independently H or OH; or R⁶ and R⁷ taken together with the carbon to which they are attached form a >C═O group;
R⁸ is H, OH, O(CO)R⁹, or O(CO)OR⁹;
each R⁹ is independently C₁-C₄alkyl;
each R¹⁰ and R¹¹ is independently H or C₁-C₄alkyl;
R¹² is H, OH, or C₁-C₄alkyl; or
R¹¹ and R¹² taken together with the carbon to which they are attached form a >═CH₂ group; or
R¹² and R⁸ taken together with the carbons to which they are attached form a 1,3-dioxolane ring represented by formula B:

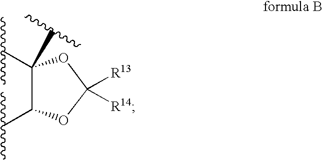

wherein one of R³ and R¹⁴ is H, methyl or ethyl and the other is H, C₁-C₁₀alkyl, C₂-C₁₀alkenyl, C₂-C₁₀alkynyl, optionally substituted C₃-C₁₀ carbocyclyl or optionally substituted 5-6 ring atom heterocycle wherein one or two ring atoms are selected from N, O and S, and wherein said carbocyclyl and said heterocyclyl are each optionally substituted 1, 2 or 3 times with a substituent selected from halo, C₁-C₄alkyl, and O—C₁-C₄alkyl.

In one embodiment each of R², R³, R⁴, and R⁵ are independently H, methyl, F or Cl, or any subset thereof. In one preferred embodiment R², R³, R⁴, and R⁵ are H. In one embodiment R⁴ and R⁵ are H and R² and R³ are H, F, Cl or methyl. In one embodiment R⁴ and R¹⁵ are H, R² is H, F or Cl and R¹³ is H. F or methyl. In one particular embodiment R⁴ and R⁵ are H and R² and R¹³ are H or F. In one particular embodiment R⁴ and R⁵ are H and R² and R³ are F. In one particular embodiment R⁴ and R⁵ are H, R² is H and R³ is F or R² is F and R³ is H.

In one particular embodiment R⁶ and R⁷ taken together with the carbon to which they are attached form a >C═O group. In one preferred embodiment R¹⁶ is H and R⁷ is OH.

In one embodiment R⁸ is H, OH, O(CO)CH₂CH₃, O(CO)OCH₃, or O(CO)CH₂CH₃, or any subset thereof.

In one embodiment R¹⁰ is H. In one particular embodiment R¹⁰ and R¹¹ are H. In one embodiment R¹⁰ is H and R¹¹ is methyl.

In one embodiment R¹² is H, OH, or methyl. In one particular embodiment R¹² is H or methyl, more particularly H.

In one embodiment R¹¹ and R¹² taken together with the carbon to which they are attached form a >═CH, group.

In one preferred embodiment R¹² and R⁸ taken together with the carbons to which they are attached form a 1,3-dioxolane ring represented by formula B:

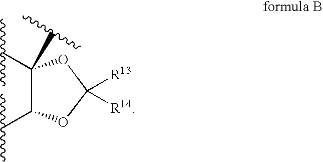

In one embodiment wherein R¹² and R⁸ form a ring represented by formula B, one of R¹³ and R¹⁴ is H, methyl or ethyl and the other is H, C₁-C₁₀alkyl, C₂-C₁₀alkenyl, C₂-C₁₀alkynyl, optionally substituted C₃-C₁₀ carbocyclyl or optionally substituted 5-6 ring atom heterocycle wherein one or two ring atoms are selected from N, O and S, or any subset thereof, wherein the carbocyclyl and heterocyclyl are each optionally substituted 1, 2 or 3 times with a substituent selected from halo, C₁-C₄alkyl, and O—C₁-C₄alkyl. In one embodiment wherein R¹² and R⁸ form a ring represented by formula B, one of R¹³ and R¹⁴ is H, methyl or ethyl and the other is H, C₁-C₁₀alkyl, C₂-C₁₀alkenyl, C₂-C₁₀alkynyl, or optionally substituted C₃-C₁₀ carbocyclyl, wherein the carbocyclyl is optionally substituted 1, 2 or 3 times with a substituent selected from halo, C₁-C₄alkyl, and O—C₁-C₄alkyl. In one embodiment one of R¹³ and R¹⁴ is H, methyl or ethyl and the other is H, C₁-C₁₀alkyl, or C₃-C₁₀carbocyclyl, or any subset thereof. In one embodiment one of R¹³ and R¹⁴ is H, methyl or ethyl and the other is H, C₁-C₄alkyl, or C₃-C₆ cycloalkyl, or any subset thereof, more particularly cyclohexyl. In one embodiment one of R¹³ and R¹⁴ is H or methyl, more particularly H, and the other is H, C₁-C₄alkyl, or C₃-C₆ cycloalkyl, or any subset thereof, more particularly cyclohexyl. In one embodiment R¹³ and R¹⁴ are each methyl. In one embodiment R¹³ is H and R¹⁴ is propyl. In one preferred embodiment R¹³ is H and R¹⁴ is cyclohexyl.

In a particular embodiment the corticosteroid moiety is

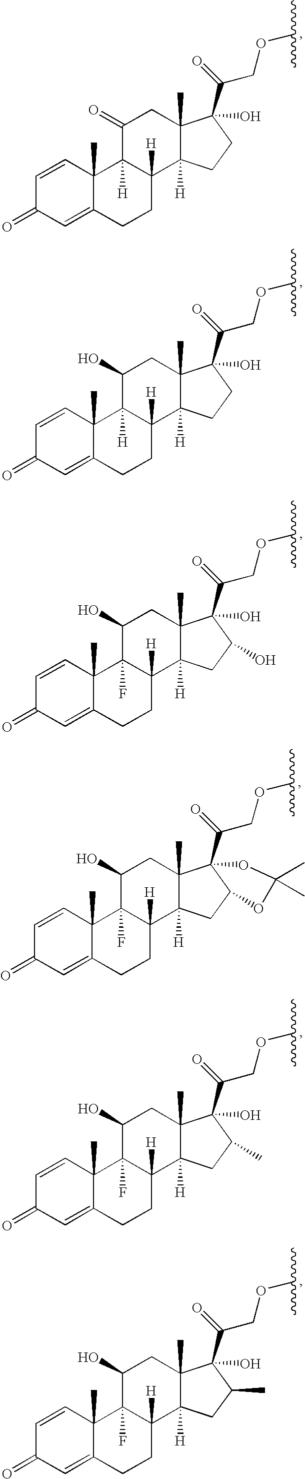

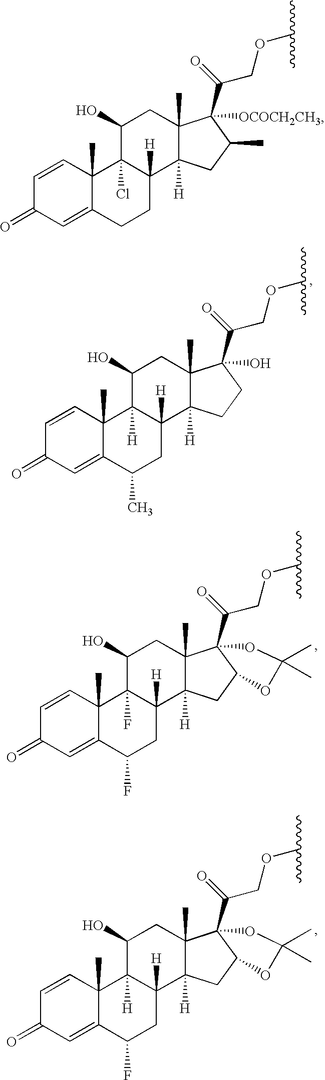

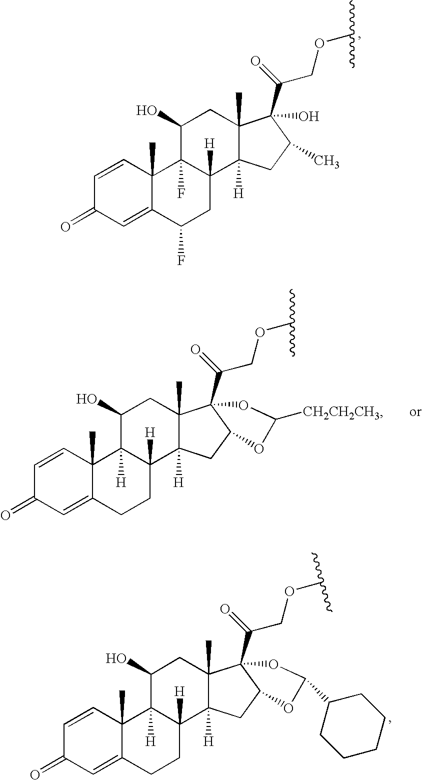

or any subset thereof.

In one preferred embodiment the corticosteroid moiety is

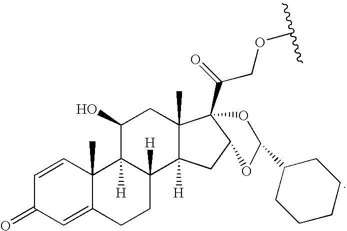

The selection of variables X¹ and Z should be made in view of each other in order to avoid embodiments which are clearly unstable or inoperative based upon the knowledge of those skilled in the art of organic chemistry. For this purpose, Z has been defined such that when Z is NH, N(C₁-C₆alkyl), ^{⊕}(NR¹⁷R¹⁸)A⁽⁻⁾, N(O)R¹⁷ (N-oxide), S(O) (sulfoxide), S(═O)₂, or ^{⊕}(SR⁷)A⁽⁻⁾, then X¹ is neither a bond nor a group bound to Z through O, S, N(H), N(C₁-C₆alkyl), N(H)C(O), N(C₁-C₄alkyl)C(O), C(O)N(H) or C(O)N(C₁-C₄alkyl).

In one embodiment the compounds of the invention are defined wherein Z is ^{⊕}(NR¹⁷R¹⁸)A⁽⁻⁾, ^{⊕}(SR¹⁷)A⁽⁻⁾, or a 4-9 ring atom heterocyclene wherein one ring atom is ^{⊕}(N)A⁽⁻⁾, O(N(C₁-C₆alkyl))A⁽⁻⁾, or ^{⊕}SA⁽⁻⁾, or any subset thereof, and the β-agonist moiety

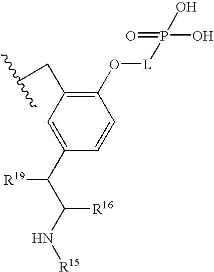

is bonded to the ^{⊕}N, ^{⊕}N(C₁-C₆alkyl) or ^{⊕}S atom of the heterocyclene.

It is to be understood that in all embodiments wherein Z is a heterocyclene, one ring atom is ^{⊕}(N)A⁽⁻⁾, ^{⊕}(N(C₁-C₆alkyl))A⁽⁻⁾ or ^{⊕}SA⁽⁻⁾, up to one other ring atom is N, O or S and all remaining ring atoms are carbon. It is to be understood that in all embodiments wherein Z is a heterocyclene X¹ is bound to any suitable carbon or heteroatom of the heterocyclene except the ^{⊕}N, ^{⊕}N(C₁-C₆alkyl), or ^{⊕}S to which the β-agonist moiety is bound.

In one particular embodiment Z is ^{⊕}(NR¹⁷R¹⁸)A⁽⁻⁾ or a 4-9 ring atom heterocyclene wherein one ring atom is ^{⊕}(N)A⁽⁻⁾, ^{⊕}(N(C₁-C₆alkyl))A⁽⁻⁾ or ^{⊕}SA⁽⁻⁾, up to one other ring atom is N, O or S, all other ring atoms are carbon, and the β-agonist moiety is bonded to ^{⊕}N, ^{⊕}N(C₁-C₆alkyl) or ^{⊕}S, or any subset thereof.

In one particular embodiment Z is ^{⊕}(NR¹⁷R¹⁸)A⁽⁻⁾ or a 5-6 ring atom heterocyclene wherein one ring atom is ^{⊕}(N)A⁽⁻⁾ or ^{⊕}(N(C₁-C₆alkyl))A⁽⁻⁾, up to one other ring atom is N, O or S, all other ring atoms are carbon, and the β-agonist moiety is bonded to ^{⊕}N, ^{⊕}N(C₁-C₆alkyl), or any subset thereof.

In one preferred embodiment Z is ^{⊕}(NR¹⁷R¹⁸)A⁽⁻⁾. In another preferred embodiment Z is a 5-6 ring atom heterocyclene wherein one ring atom is (N)A⁽⁻⁾ or ^{⊕}(N(C₁-C₆alkyl))A⁽⁻⁾, up to one other ring atom is N, O or S, all other ring atoms are carbon, and the β-agonist moiety is bonded to ^{⊕}N or ^{⊕}N(C₁-C₆alkyl). In another preferred embodiment, Z is a 6 ring atom heteroarylene wherein one ring atom is ^{⊕}(N)A⁽⁻⁾, up to one other ring atom is N, O or S, all other ring atoms are carbon, and the β-agonist moiety is bonded to ^{⊕}N. In another preferred embodiment, Z is a 5-6 ring atom saturated or partially unsaturated, non-aromatic, heterocyclene wherein one ring atom is ^{⊕}(N(CH₃))A⁽⁻⁾, up to one other ring atom is N, O or S, all other ring atoms are carbon, and the β-agonist moiety is bonded to E) N(CH₃).

In the embodiments wherein Z is ^{⊕}(NR¹⁷R¹⁸)A⁽⁻⁾ or ^{⊕}(SR¹⁷)A⁽⁻⁾, R¹⁷ and R¹⁸ are each independently, C₁-C₆alkyl, C₁-C₆alkenyl, C₁-C₆alkynyl, or C₃-C₇-carbocycle, or any subset thereof, wherein said alkyl, alkenyl, alkynyl is optionally substituted 1, 2 or 3 times, more particularly 1 or 2 times with halo (particularly F, Cl or Br), OH and ═O and the carbocycle is optionally substituted 1, 2 or 3 times, more particularly 1 or 2 times, with a substituent selected from halo (particularly F, Cl or Br), C₁-C₄alkyl, OH, and ═O. In one embodiment R¹⁷ and R¹⁸ are each independently, unsubstituted C₁-C₆alkyl, unsubstituted C₁-C₆alkenyl, unsubstituted C₁-C₆alkynyl, or unsubstituted C₃-C₇-carbocycle, or any subset thereof. In one particular embodiment, R¹⁷ and R¹⁸ are each independently, unsubstituted C₁-C₆alkyl, cyclopropyl, cyclopentyl or cyclohexyl, or any subset thereof. In one particular embodiment, R¹⁷ and R¹⁸ are each independently methyl, ethyl, propyl, isopropyl, t-butyl, cyclopropyl, cyclopentyl or cyclohexyl, or any subset thereof. In one preferred embodiment, R¹⁷ and R¹⁸ are each independently methyl, ethyl, propyl, or isopropyl, more particularly methyl or ethyl. In one preferred embodiment, R¹⁷ and R¹⁸ are the same.

In one embodiment X¹ is a bond. In another embodiment X¹ is selected from
C₁-C₁₂alkylene, C₂-C₁₂alkenylene, C₂-C₁₂alkynylene,
O—C₁-C₂alkylene, O—C₂-C₁₂alkenylene, O—C₂-C₂alkynylene,
S—C₁-C₁₂alkylene, S—C₂-C₁₂alkenylene, S—C₂-C₁₂alkynylene,
N(H)—C₁-C₁₂alkylene, N(H)—C₂-C₁₂alkenylene, N(H)—C₂-C₁₂alkynylene,
N(C₁-C₆alkyl)-C₁-C₁₂alkylene, N(C₁-C₆alkyl)-C₁₂-C₁₂alkenylene, N(C₁-C₆alkyl)-C₂-C₁₂alkynylene,
C₃-C₇-carbocyclene, C₃-C₇-carbocyclene-C₁-C₆alkylene, heterocyclene, heterocyclene-C₁-C₆alkylene, heterocyclene-C(O), wherein said heterocyclene is a 3-9 ring atom heterocyclene wherein 1 or 2 ring atoms are selected from N, O and S,
C₁-C₆alkylene-O—C₁-C₆alkylene, C₁-C₆alkylene-S—C₁-C₆alkylene, C₁-C₆alkylene-N(H)—C₁-C₆alkylene, C₁-C₆alkylene-N(C₁-C₃alkyl)-C₁-C₆alkylene, C₁-C₆alkylene-C₃-C₇carbocyclene-C₁-C₆alkylene, C₁-C₆alkylene-heterocyclene-C₁-C₆alkylene, wherein said heterocyclene is a 3-9 ring atom heterocyclene wherein 1 or 2 ring atoms are selected from N, O and S,
C₁-C₁₂alkylene-O, C₁-C₁₂alkylene-S, C₁-C₁₂alkylene-N(H), C₁-C₁₂alkylene-N(C₁-C₆alkyl), C₁-C₈alkylene-N(H)C(O), C₁-C₈alkylene-N(C₁-C₄alkyl)C(O), C₁-C₈alkylene-C(O)N(H) and C₁-C₈alkylene-C(O)N(C₁-C₄alkyl), or any subset thereof;
CH₂-AA, and C(H)(AA)-N(H)C(O), wherein AA is a proteinogenic amino acid side chain;
wherein each alkyl, alkylene, alkenylene, and alkynylene is optionally substituted 1 or 2 times with a substituent independently selected from halo, OH, OCH₃, NH₂, N(H)CH₃, and N(CH₃)₂, or any subset thereof, and each carbocyclene and heterocyclene is optionally substituted 1, 2 or 3 times with a substituent independently selected from halo, and C₁-C₄alkyl, or any subset thereof.

In all instances where X¹ is alkylene or a group including alkylene (e.g., C₁-C₈alkylene-N(H)C(O)—) the alkylene may be linear or branched. In one embodiment X¹ is a group including branched alkylene.

In the heterocyclene of X¹, 1 or 2 ring atoms is a heteroatom independently selected from N, O and S.

When X¹ is CH-AA or C(H)(AA)-N(H)C(O), the proteinogenic amino acid side chain is selected from arginine, lysine, serine and threonine radicals. In one embodiment, X¹ is

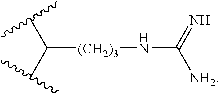

In one particular embodiment, X¹ is selected from
C₁-C₆alkylene, C₂-C₆alkenylene, C₂-C₆alkynylene,
O—C₁-C₆alkylene, O—C₂-C₆alkenylene, O—C₂-C₆alkynylene,
S—C₁-C₆alkylene, S—C₂-C₆alkenylene, S—C₂-C₆alkynylene,
N(H)—C₁-C₆alkylene, N(H)—C₂-C₆alkenylene, N(H)—C₂-C₆alkynylene,
N(C₁-C₄alkyl)-C₁-C₆alkylene, N(C₁-C₄alkyl)-C₂-C₆alkenylene, N(C₁-C₄alkyl)-C₂-C₆alkynylene,
C₃-C₆-carbocyclene, C₃-C₆-carbocyclene-C₁-C₄alkylene, 5-9 ring atom heterocyclene, 5-9 ring atom heterocyclene-C₁-C₄alkylene, 5-9 ring atom heterocyclene-C(O), wherein 1 or 2 ring atoms of said heterocyclene is/are selected from N, O and S,
C₁-C₃alkylene-O—C₁-C₃alkylene, C₁-C₃alkylene-S—C₁-C₃alkylene, C₁-C₃alkylene-N(H)—C₁-C₃alkylene, C₁-C₃alkylene-N(C₁-C₃alkyl)-C₁-C₃alkylene,
C₁-C_alkylene-C₃-C₆-carbocyclene-C₁-C₃alkylene, C₁-C₃ alkylene, 5-9 ring atom heterocyclene-C₁-C₃alkylene, wherein 1 or 2 ring atoms of said heterocyclene is/are selected from N, O and S,
C₁-C₆alkylene-O, C₁-C₆alkylene-S, C₁-C₆alkylene-N(H), C₁-C₆alkylene-N(C₁-C₃alkyl), C₁-C₄alkylene-N(H)C(O), C₁-C₄alkylene-N(C₁-C₃alkyl)C(O), C₁-C₄alkylene-C(O)N(H) and C₁-C₄alkylene-C(O)N(C₁-C₃alkyl), or any subset thereof;
wherein each alkyl, alkylene, alkenylene, and alkynylene is optionally substituted 1 or 2 times with a substituent independently selected from halo, OH, OCH₃, NH₂, N(H)CH₃, and N(CH₃)₂, or any subset thereof, and each carbocyclene and heterocyclene is optionally substituted 1, 2 or 3 times with a substituent independently selected from halo, and C₁-C₄alkyl, or any subset thereof.

In one embodiment, X¹ is selected from
C₁-C₆alkylene, C₂-C₆alkenylene, C₂-C₆alkynylene,
O—C₁-C₆alkylene, S—C₁-C₆alkylene, N(H)—C₁-C₆alkylene, N(H)—C₂-C₆alkenylene, N(C₁-C₄alkyl)-C₁-C₆alkylene,
C₃-C₆-carbocyclene, C₃-C₆-carbocyclene-C₁-C₄alkylene, 5-6 ring atom heterocyclene, 5-6 ring atom heterocyclene-C₁-C₄alkylene, 5-6 ring atom heterocyclene-C(O), wherein 1 or 2 ring atoms of said heterocyclene is/are selected from N, O and S,
C₁-C₃alkylene-O—C₁-C₃alkylene, C₁-C₃alkylene-N(H)—C₁-C₃alkylene, C₁-C₃alkylene-N(C₁-C₃alkyl)-C₁-C₃alkylene,
C₁-C₆alkylene-O, C₁-C₆alkylene-S, C₁-C₆alkylene-N(H), C₁-C₆alkylene-N(C₁-C₃alkyl), C₁-C₄alkylene-N(H)C(O)—, C₁-C₄alkylene-C(O)N(H), and C₁-C₄alkylene-C(O)N(C₃-C₃alkyl), or any subset thereof;
wherein each alkyl, alkylene, alkenylene, and alkynylene is optionally substituted 1 or 2 times with a substituent independently selected from halo, OH, OCH₃, NH₂, N(H)CH₃, and N(CH₃)₂, or any subset thereof, and each carbocyclene and heterocyclene is optionally substituted 1, 2 or 3 times with a substituent independently selected from halo, and C₁-C₄alkyl, or any subset thereof.

In one embodiment X¹ is selected from
C₁-C₆alkylene, C₂-C₆alkenylene, C₂-C₆alkynylene,
O—C₁-C₆alkylene, S—C₁-C₆alkylene, N(H)—C₁-C₆alkylene, N(H)—C₂-C₆alkenylene, N(C₁-C₄alkyl)-C₁-C₆alkylene,
C₃-C₆-carbocyclene, C₃-C₆-carbocyclene-C₁-C₄alkylene, and
C₁-C₄alkylene-N(H)C(O), or any subset thereof;
wherein each alkyl, alkylene, alkenylene, and alkynylene is optionally substituted 1 or 2 times with a substituent independently selected from halo, OH, OCH₃, NH₂, N(H)CH₃, and N(CH₃)₂, or any subset thereof, and each carbocyclene and heterocyclene is optionally substituted 1, 2 or 3 times with a substituent independently selected from halo, and C₁-C₄alkyl, or any subset thereof.

In one particular embodiment X¹ is selected from
C₁-C₆alkylene, C₂-C₆alkenylene, C₂-C₆alkynylene,
O—C₁-C₆alkylene, N(H)—C₁-C₆alkylene, N(C₁-C₄alkyl)-C₁-C₆alkylene, phenylene, C₃-C₆-carbocyclene-C₁-C₄alkylene, and C₁-C₄alkylene-N(H)C(O), or any subset thereof;
wherein each alkyl, alkylene, alkenylene, and alkynylene is optionally substituted 1 or 2 times with a substituent independently selected from halo, OH, OCH₃, NH₂, N(H)CH₃, and N(CH₃)₂, or any subset thereof, and each carbocyclene and heterocyclene is optionally substituted 1, 2 or 3 times with a substituent independently selected from halo, and C₁-C₄alkyl, or any subset thereof.

In one particular embodiment X¹ is selected from
C₁-C₆alkylene, C₂-C₆alkenylene, C₂-C₆alkynylene,
O—C₁-C₆alkylene, N(H)—C₁-C₆alkylene, N(C₁-C₄alkyl)C₁-C₆alkylene, phenylene, C₃-C₆-carbocyclene-C₁-C₄alkylene, and C₁-C₄alkylene-N(H)C(O), or any subset thereof;
wherein each alkyl, alkylene, alkenylene, and alkynylene is optionally substituted 1 or 2 times with a substituent independently selected from halo, OH, OCH₃, NH₂, N(H)CH₃, and N(CH₃)₂, or any subset thereof and each carbocyclene and heterocyclene is optionally substituted 1, 2 or 3 times with a substituent independently selected from halo, and C₁-C₄alkyl, or any subset thereof.

In any of the foregoing embodiments any or all of the alkyl, alkylene, alkenylene, alkynylene, carbocyclene and heterocyclene of X¹ may be unsubstituted.

In one preferred embodiment X¹ is selected from a bond, —CH₂—, —CH₂CH₂—, —(CH₂)₃—, —CH(CH₃)—, —CH(CH₃)CH₂—, —CH═CH—, —O—CH₂—, —O—CH₂CH₂—, —O—CH(CH₃)CH₁₂—, —N(H)—CH₂—, —N(H)—CH₂CH₂—, —N(CH₃)—CH₂—, —N(CH₃)—CH₂CH₂—, phenylene, -cyclopropylene-CH₂—, -cyclopentylene-CH₂—, -cyclohexylene-CH₂—, phenylene-CH₂—, —CH₂—N(H)C(O)—, —CH(CH₃)—N(H)C(O)—, and —CH(CH(CH₃)₂)—N(H)C(O)—, or any subset thereof.

In one particular embodiment Z is ^{⊕}(NR¹⁷R¹⁸)A⁽⁻⁾ and X¹ is selected from
C₁-C₆alkylene, C₂-C₆alkenylene, C₂-C₆alkynylene,
O—C₁-C₆alkylene, S—C₁-C₆alkylene, N(H)—C₁-C₆alkylene, N(H)—C₂-C₆alkenylene, N(C₁-C₄alkyl)-C₁-C₆alkylene,
C₃-C₆-carbocyclene, C₃-C₆-carbocyclene-C₁-C₄alkylene, 5-6 ring atom heterocyclene, 5-6 ring atom heterocyclene-C₁-C₄alkylene, 5-6 ring atom heterocyclene-C(O), wherein 1 or 2 ring atoms of said heterocyclene is/are selected from N, O and S,
C₁-C₃alkylene-O—C₁-C₃alkylene, C₁-C₃alkylene-N(H)—C₁-C₃alkylene, C₁-C₃alkylene-N(C₁-C₃alkyl)-C₁-C₃alkylene, or any subset thereof,
wherein each alkyl, alkylene, alkenylene, and alkynylene is optionally substituted 1 or 2 times with a substituent independently selected from halo, OH, OCH₃, NH₂, N(H)CH₃, and N(CH₃)₂, or any subset thereof, and each carbocyclene and heterocyclene is optionally substituted 1, 2 or 3 times with a substituent independently selected from halo, and C₁-C₄alkyl, or any subset thereof.

In one preferred embodiment Z is ^{⊕}(NR¹⁷R¹⁸)A⁽⁻⁾ and X¹ is selected from C₁-C₆alkylene, C₂-C₆alkenylene, C₂-C₆alkynylene, O—C₁-C₆alkylene, N(H)—C₁-C₆alkylene, N(C₁-C₄alkyl)-C₁-C₆alkylene, phenylene, and C₃-C₆-carbocyclene-C₁-C₄alkylene, or any subset thereof, wherein each alkyl, alkylene, alkenylene, alkynylene, carbocyclene and phenylene of X¹ are unsubstituted.

In one embodiment Z is a 5-9 ring atom heterocyclene wherein one ring atom is ^{⊕}(N)A⁽⁻⁾ and ^{⊕}(N(C₁-C₆alkyl))A⁽⁻⁾, up to one other ring atom is N, O or S, all other ring atoms are carbon, the β-agonist moiety is bound to ^{⊕}N and O N(C₁-C₆alkyl), and X¹ is selected from a bond, C₁-C₆alkylene, C₂-C₆alkenylene, C₂-C₆alkynylene,
O—C₁-C₆alkylene, S—C₁-C₆alkylene, N(H)—C₁-C₆alkylene, N(C₁-C₄alkyl)-C₁-C₆alkylene,
C₃-C₆-carbocyclene, C₃-C₆carbocyclene-C₁-C₄alkylene,
C₁-C₃alkylene-O—C₁-C₃alkylene, C₁-C₃alkylene-N(H)—C₁-C₃alkylene, C₁-C₃alkylene-N(C₁-C₃alkyl)-C₁-C₃alkylene;
C₁-C₆alkylene-O, C₁-C₆alkylene-S, C₁-C₆alkylene-N(H), C₁-C₆alkylene-N(C₁-C₃alkyl), C₁-C₄alkylene-N(H)C(O), C₁-C₄alkylene-C(O)N(H), and C₁-C₄alkylene-C(O)N(C₁-C₃alkyl),
or any subset thereof;
wherein each alkyl alkylene, alkenylene, and alkynylene is optionally substituted 1 or 2 times with a substituent independently selected from halo, OH, OCH₃, NH₂, N(H)CH₃, and N(CH₃)₂, or any subset thereof, and each carbocyclene and heterocyclene is optionally substituted 1, 2 or 3 times with a substituent independently selected from halo, and C₁-C₄alkyl, or any subset thereof.

In one embodiment Z is a 5-9 ring atom heterocyclene wherein one ring atom is ^{⊕}(N)A⁽⁻⁾ or ^{⊕}(N(C₁-C₆alkyl))A⁽⁻⁾, up to one other ring atom is N, O or S, all other ring atoms are carbon, the β-agonist moiety is bound to ^{⊕}N or ^{⊕}N(C₁-C₆alkyl), and X¹ is selected from a bond, C₁-C₆alkylene, C₁-C₆alkenylene, C₃-C₆-carbocyclene, C₃-C₆-carbocyclene-C₁-C₄alkylene, and C₁-C₄alkylene-N(H)C(O), or any subset thereof, wherein each alkyl, alkylene, alkenylene, alkynylene, carbocyclene and phenylene of X¹ are unsubstituted.

In one embodiment Z is a 5-6 ring atom heterocyclene wherein one ring atom is ^{⊕}(N)A⁽⁻⁾ or ^{⊕}(N(C₁-C₆alkyl))A⁽⁻⁾, up to one other ring atom is N, O or S, all other ring atoms are carbon, the β-agonist moiety is bound to ^{⊕}N or ^{⊕}N(C₁-C₆alkyl), and ^{⊕}N is selected from a bond, C₁-C₆alkylene, C₂-C₆alkenylene, C₃-C₆-carbocyclene, C₃-C₆-carbocyclene-C₁-C₄alkylene, and C₁-C₄alkylene-N(H)C(O), or any subset thereof, wherein each alkyl, alkylene, alkenylene, alkynylene, carbocyclene and phenylene of X¹ are unsubstituted.

The counterion, A⁽⁻⁾, is typically an anion of a pharmaceutically acceptable inorganic acid addition salt, such as chloride, bromide, iodide, hydroxide, sulfate, phosphate, or an anion from a salt derived from pharmaceutically acceptable organic acids such as acetic acid, oxalic acid, tartaric acid, succinic acid, maleic acid, fumaric acid, succinic acid, gluconic acid, citric acid, malic acid, ascorbic acid, benzoic acid, isethionic acid, lactobionic acid, tannic acid, palmitic acid, alginic acid, polyglutamic acid, naphthalenesulfonic acid, methanesulfonic acid, p-toluenesulfonic acid, benzenesulfonic acid, naphthalenedisulfonic acid, naphthalene-1,5-disulfonic acid, polygalacturonic acid, malonic acid, sulfosalicylic acid, glycolic acid, 2-hydroxy-3-naphthoate, 1-hydroxy-2-naphthoate (xinafoate), pannoate, salicylic acid, stearic acid, phthalic acid, mandelic acid, lactic acid, ethanesulfonic acid, lysine, arginine, glutamic acid, glycine, serine, threonine, alanine, isoleucine, leucine and the like. In one embodiment, the counterion A⁽⁻⁾ is selected from chloride, bromide, sulfate, phosphate, acetate, tartrate, fumarate, or xinafoate, or any subset thereof. Preferred anions include those from inorganic or organic acid salts which are either acceptable for use in inhaled products and/or known or believed to minimize pulmonary irritation. In one embodiment, A⁽⁻⁾ is selected from chloride, bromide, sulfate, acetate, tartrate, fumarate and xinafoate, or any subset thereof. In one particular embodiment, A⁽⁻⁾ is chloride. In one particular embodiment, A⁽⁻⁾ is sulfate. In one particular embodiment, A⁽⁻⁾ is acetate. In one particular embodiment, A⁽⁻⁾ is tartrate. In one particular embodiment, A⁽⁻⁾ is fumarate. In one particular embodiment, A⁽⁻⁾ is xinafoate. In one particular embodiment, A⁽⁻⁾ is succinate.

In one preferred embodiment L is a bond. In another embodiment L is —CH₂O—.

In one embodiment the invention comprises compounds of Formula II

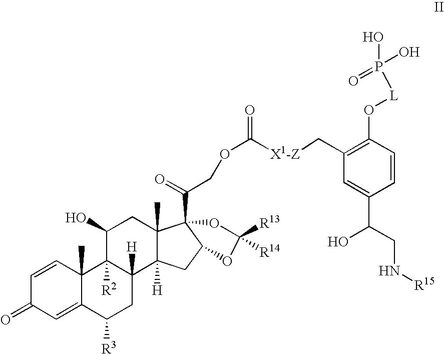

and pharmaceutically acceptable salts thereof wherein all variables are defined as for Formula I, including all embodiments thereof.

In one embodiment R² and R³ are H or F. In one preferred embodiment R² and R³ are H.

In one embodiment R² and R³ are F. In one embodiment R² is H and R³ is F or R² is F and R³ is H.

In one embodiment one of R¹³ and R¹⁴ is H or methyl and the other is H, C₁-C₁₀alkyl, or C₃-C₁₀ carbocyclyl, more particularly C₃-C₆ carbocycle. In one embodiment one of R¹³ and R¹⁴ is H or methyl and the other is H, C₁-C₄alkyl, or C₃-C₆ cycloalkyl, more particularly cyclohexyl. In one embodiment R¹³ and R¹⁴ are each methyl. In one embodiment R¹³ is H and R¹⁴ is propyl. In one preferred embodiment R³ is H and R¹⁴ is cyclohexyl.

In one embodiment R² and R³ are H, R¹³ is H and R¹⁴ is propyl or cyclohexyl. In one preferred embodiment R² and R³ are H, R¹³ is H and R¹⁴ is cyclohexyl. In one embodiment R² and R³ are H or F, and R¹³ and R¹⁴ are methyl. In one embodiment R² and R³ are F, and R¹³ and R¹⁴ are methyl. In one embodiment R² is H, R³ is F, and R³ and R⁴ are methyl.

Specific embodiments, including particular and preferred embodiments of R¹⁵, X¹, Z and L are as described above for compounds of Formula I. For the sake of brevity, the disclosure of those embodiments, including particular and preferred embodiments is not repeated. Any of the previously disclosed embodiments, particular embodiments and preferred embodiments of R¹⁵, X¹, Z and L are contemplated for combination with the foregoing embodiments (including particular and preferred embodiments) of R², R³, R¹³, and R¹⁴.

In one preferred embodiment the invention comprises compounds of Formula III:

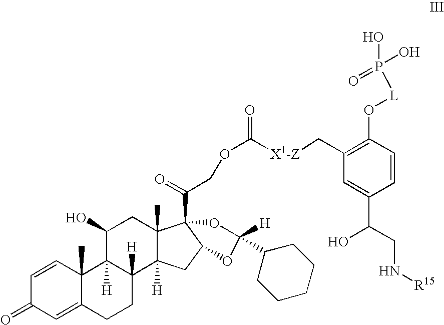

and pharmaceutically acceptable salts thereof, wherein all variables are defined as for Formula I, including all embodiments thereof.

Specific embodiments, including particular and preferred embodiments of R¹⁵, X¹, Z and L are as described above for compounds of Formula I. For the sake of brevity, the disclosure of those embodiments, including particular and preferred embodiments is not repeated. Any of the previously disclosed embodiments, particular embodiments and preferred embodiments of R¹⁵, X¹, Z and L are contemplated for combination with the foregoing embodiments (including particular and preferred embodiments) of R², R³, and R¹³, and R¹⁴.

It is to be understood that the present invention includes all combinations and subsets of the particular groups defined hereinabove in the compounds of the invention. Specific examples of compounds of the invention include those recited in the Examples and free base and pharmaceutically acceptable salts thereof.

Specific examples of compounds of the invention include the compounds set forth in the examples below (and free base and pharmaceutically acceptable salt forms thereof) as well as the following additional compounds.

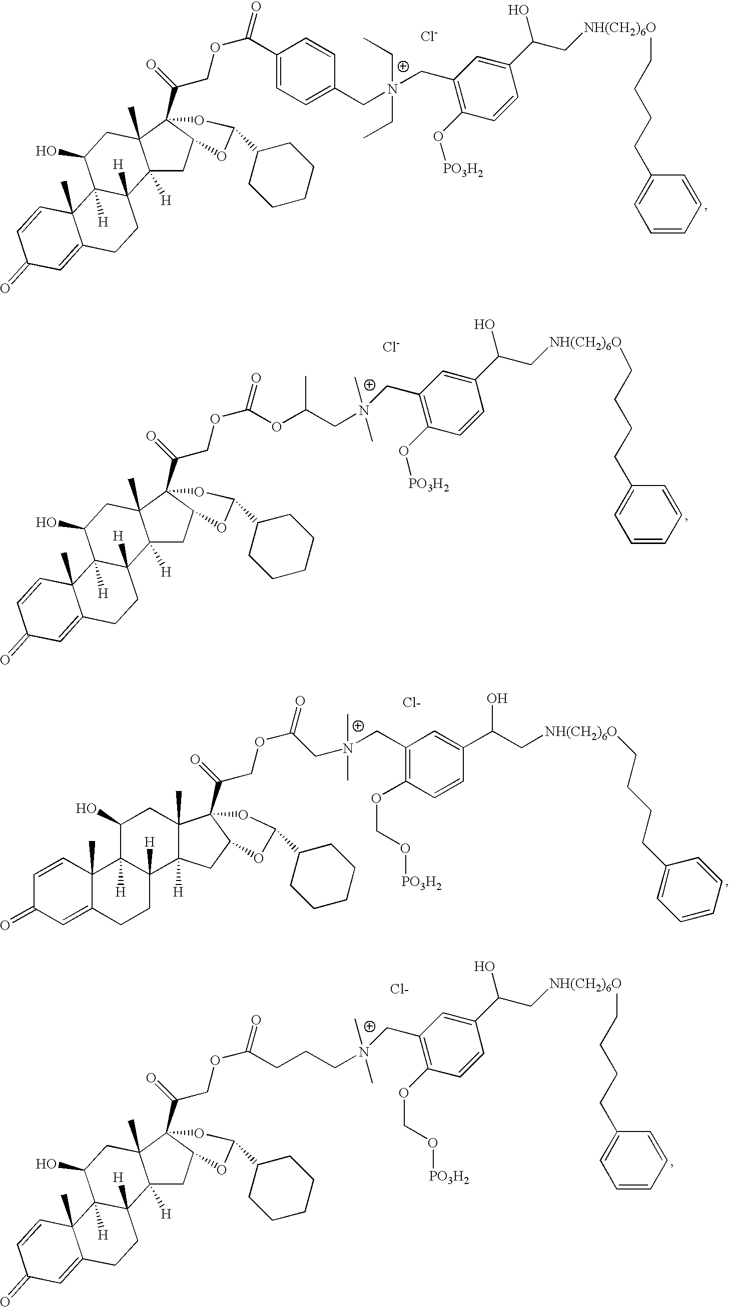

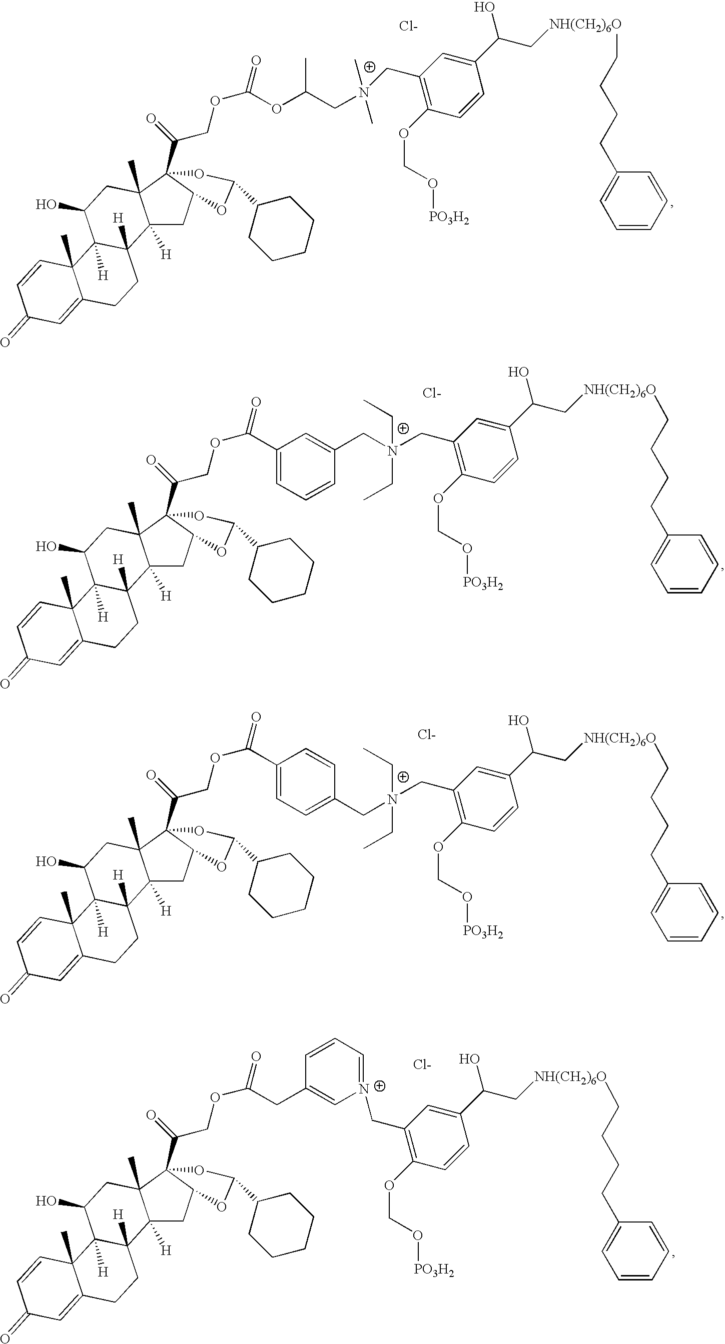

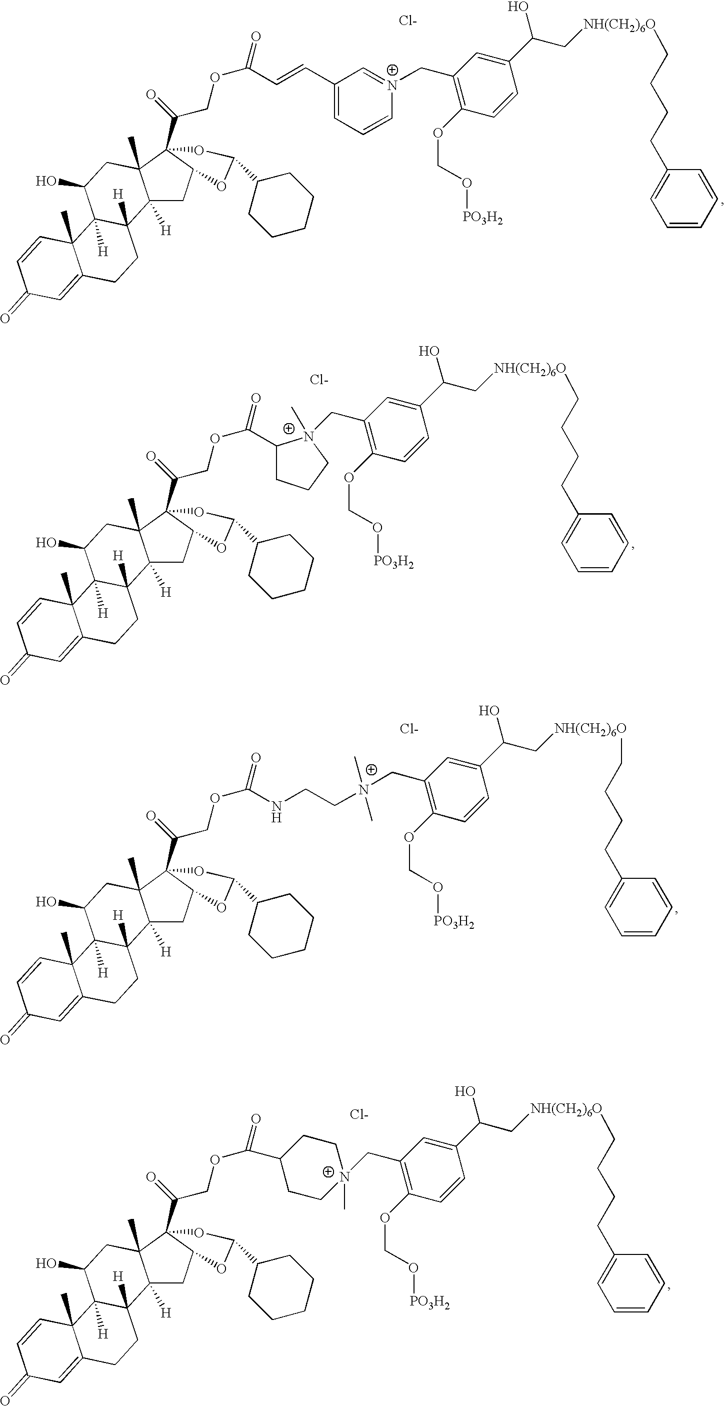

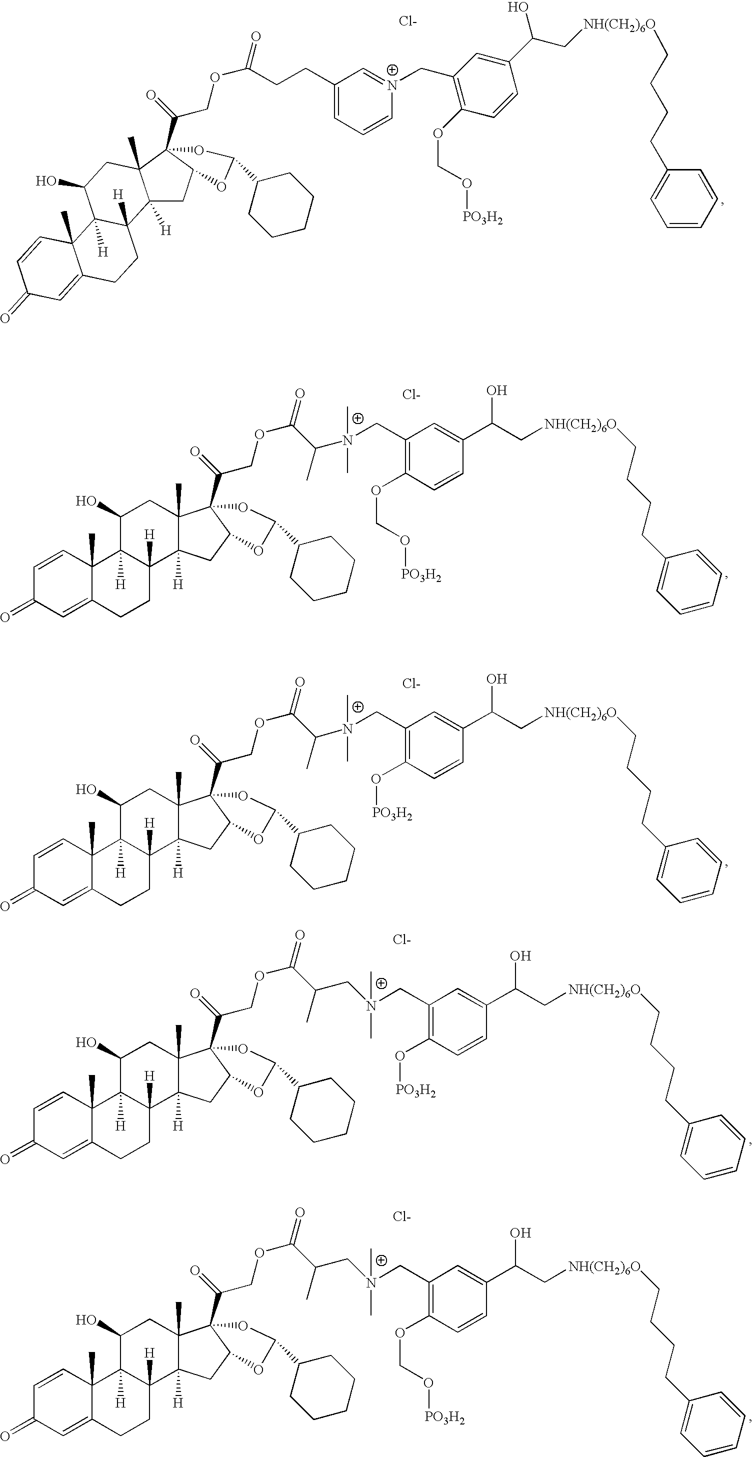

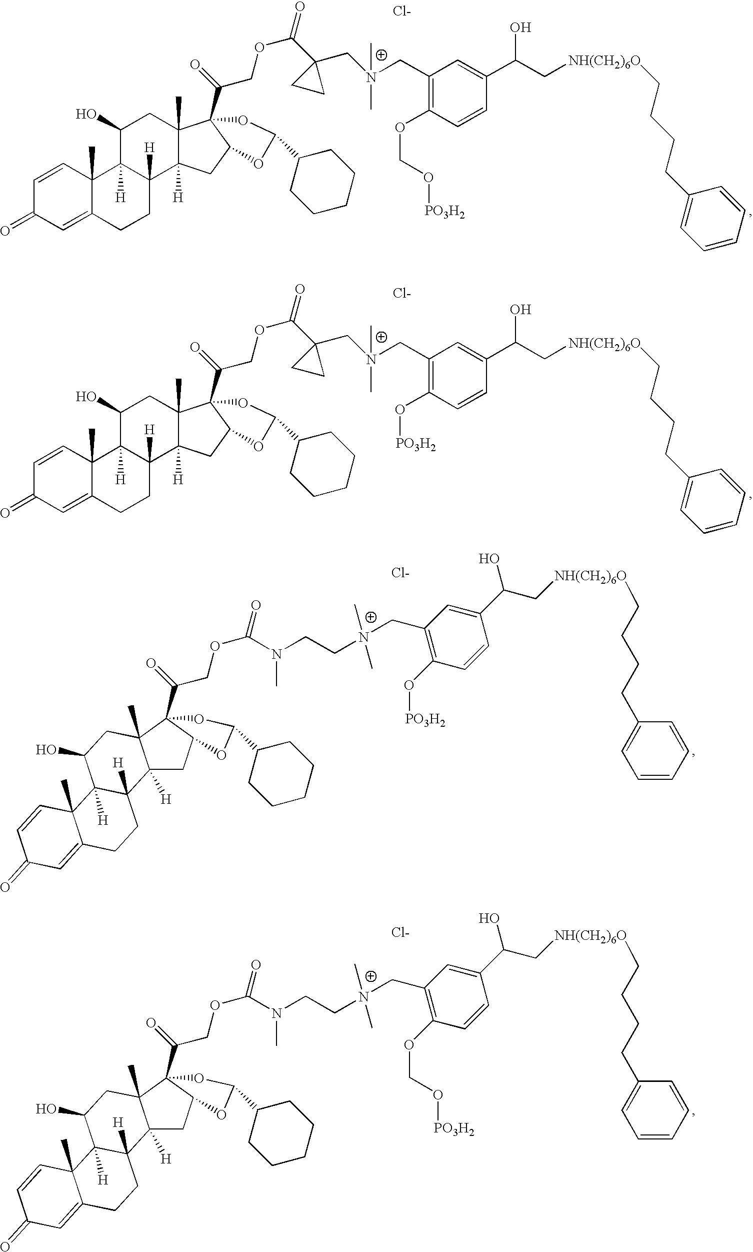

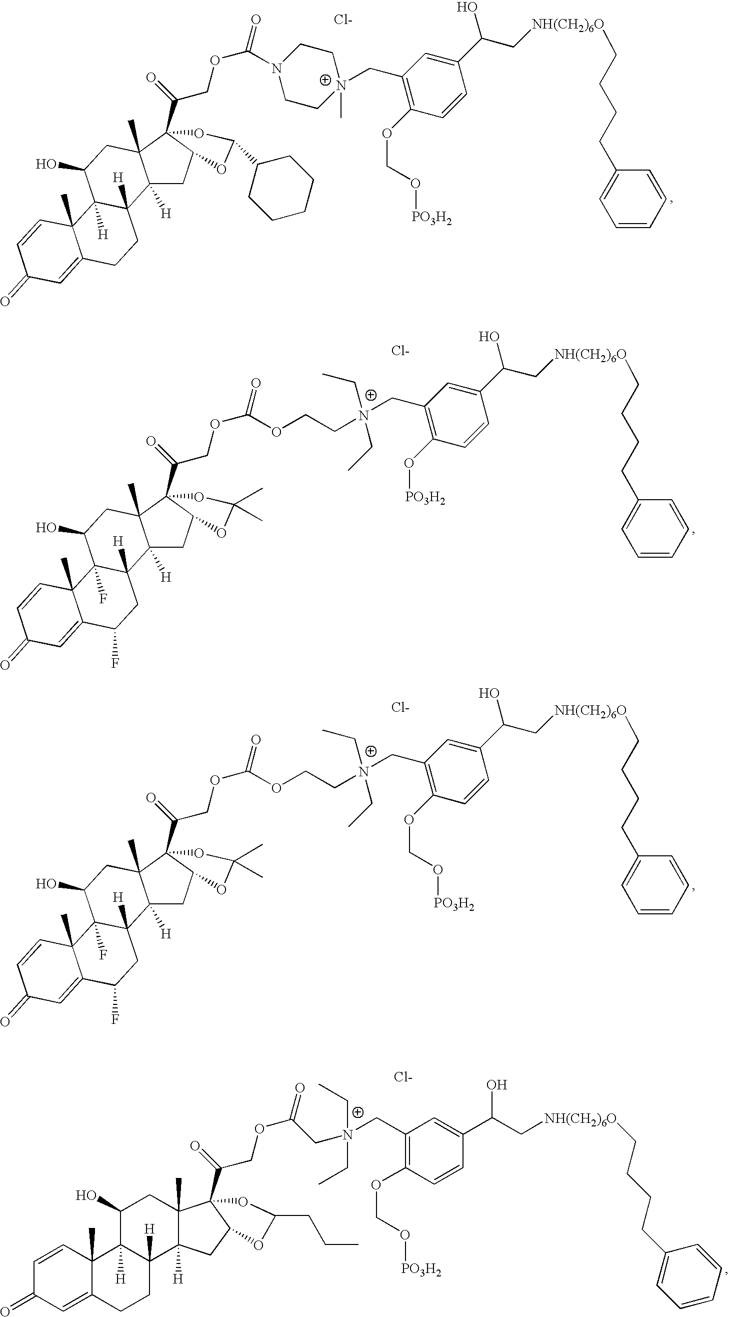

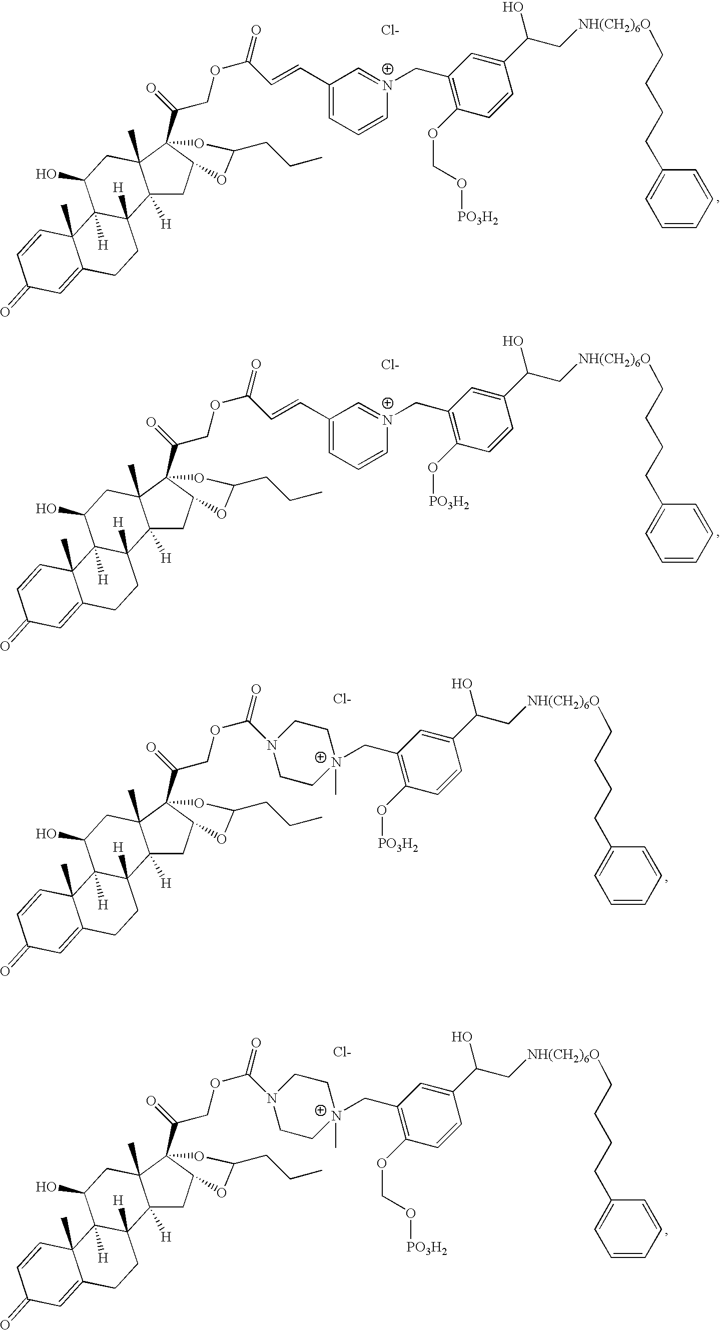

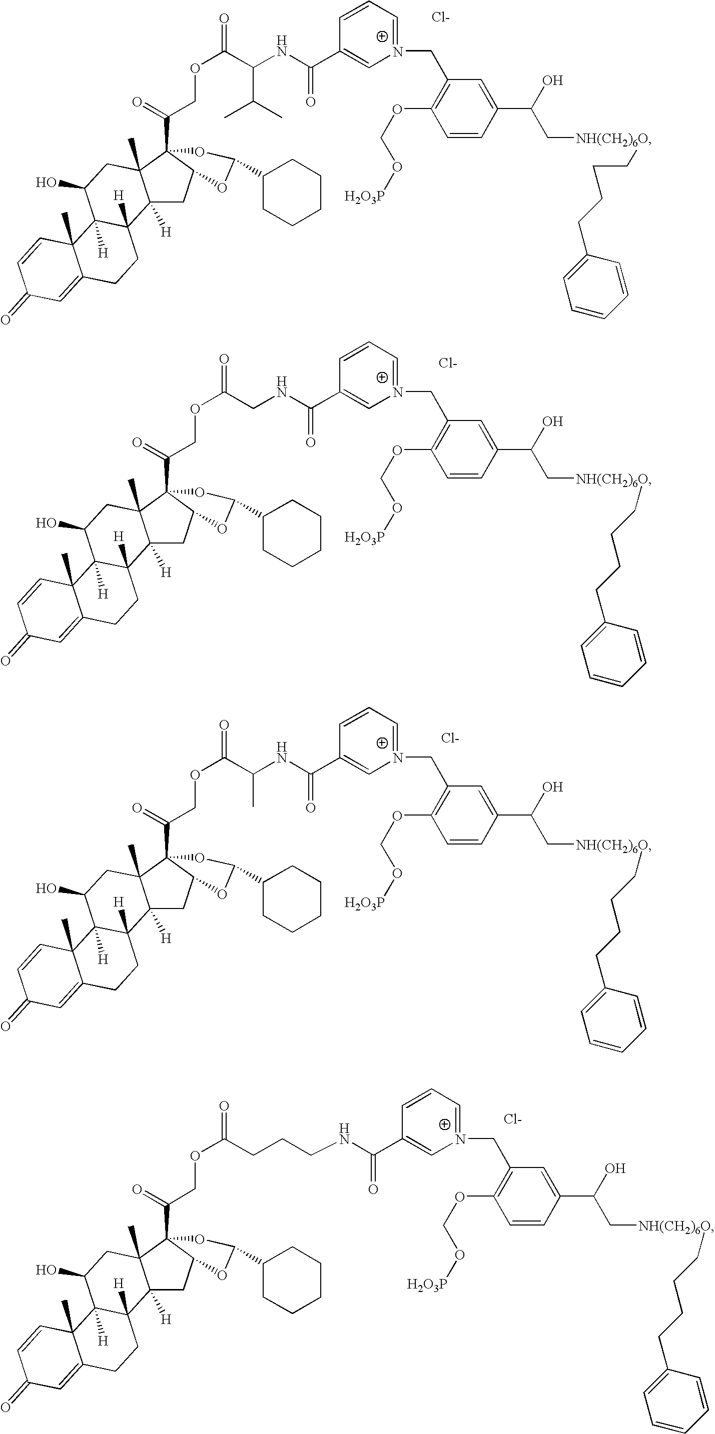

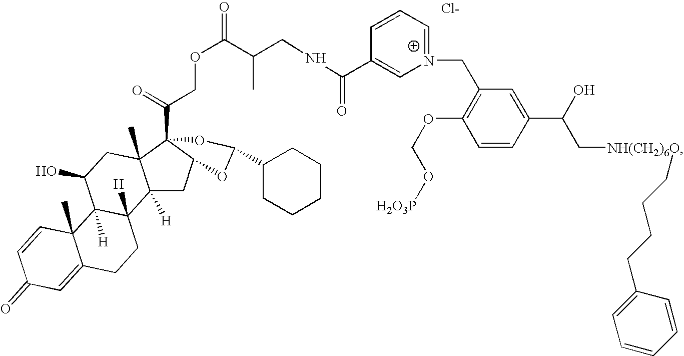

and pharmaceutically acceptable salts thereof, or any subset thereof.

In one preferred embodiment, the compounds of the invention are selected from
1-[5-[1-Hydroxy-2-[6-(4-phenylbutoxy)hexylamino]ethyl]-2-phosphonooxybenzyl]-3-[2-[[11β,16α]-[16,17-((R)-cyclohexylmethylene)bis(oxy)]-11-hydroxypregna-1,4-diene-3,20-dion-21-oxy]carbonyl]ethen-1-yl]pyridinium chloride

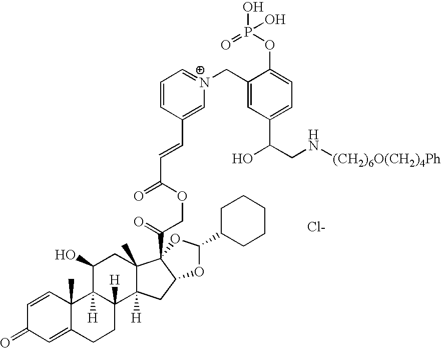

[5-[1-hydroxy-2-[6-(4-phenylbutoxy)hexylamino]ethyl]-2-phosphonooxybenzyl]-(diethyl)-[[11β,16α]-[[15,16-((R)-cyclohexylmethylene)bis(oxy)]-11-hydroxypregna-1,4-diene-3,20-dion-21-yl]carbonylmethyl]ammonium chloride

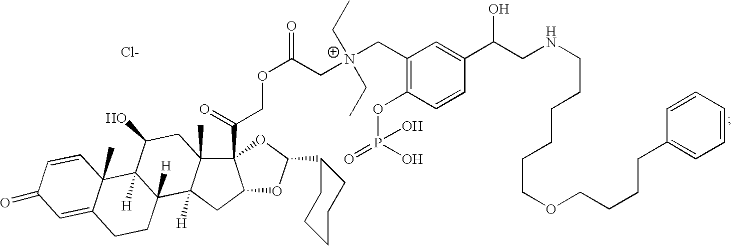

1-[5-[1-Hydroxy-2-[6-(4-phenylbutoxy)hexylamino]ethyl]-2-phosphonooxybenzyl]-3-[[[[[11β,16α]-[16,17-((R)-cyclohexylmethylene)bis(oxy)]-11-hydroxypregna-1,4-diene-3,20-dion-21-oxy]carbonyl]methyl]aminocarbonyl]pyridinium chloride

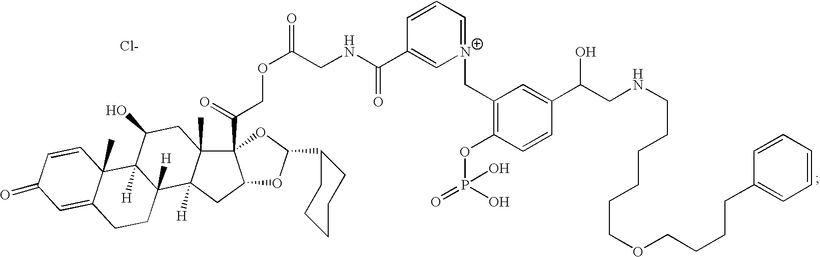

1-[5-[1-Hydroxy-2-[6-(4-phenylbutoxy)hexylamino]ethyl]-2-phosphonooxybenzyl]-1-methyl-4-[[11β,16α]-[[((R)-cyclohexylmethylene)bis(oxy)]-11-hydroxypregna-1,4-diene-3,20-dion-21-oxy]carbonyl]piperidinium acetate

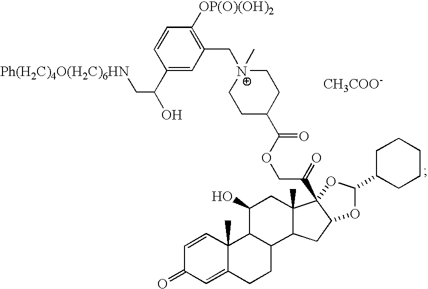

[5-[1-(R)-hydroxy-2-[6-(4-phenylbutoxy)hexylamino]ethyl]-2-phosphonooxybenzyl]-(diethyl)-[[11β,16α]-[16,17-((R)-cyclohexylmethylene)bis(oxy)]-11-hydroxypregna-1,4-diene-3,20-dion-21-oxy]carbonylmethyl]ammonium chloride

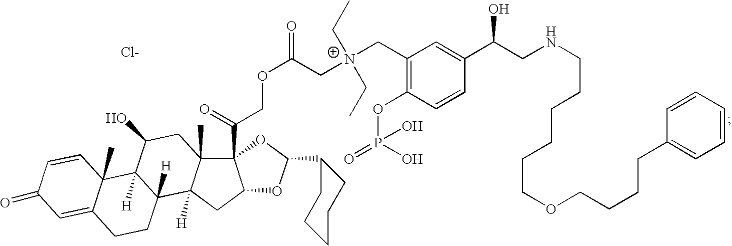

[5-[1-(S)-Hydroxy-2-[6-(4-phenylbutoxy)hexylamino]ethyl]-2-phosphonooxybenzyl]-(diethyl)-[[11β,16α]-[16,17-((R)-cyclohexylmethylene)bis(oxy)]-11-hydroxypregna-1,4-diene-3,20-dion-21-oxy]carbonylmethyl]ammonium chloride

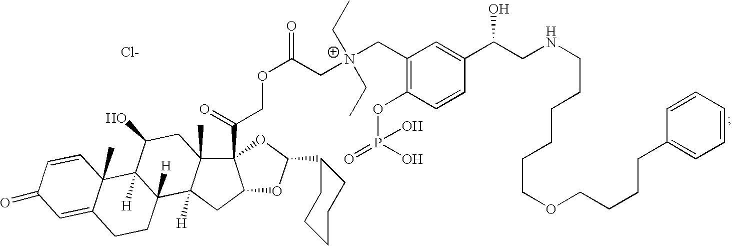

1-[5-[1-hydroxy-2-[6-(4-phenylbutoxy)hexylamino]ethyl]-2-phosphonooxybenzyl]-1-[[11β,16α]-[[15,16-((R)-cyclohexylmethylene)bis(oxy)]-11-hydroxypregna-1,4-diene-3,20-dion-21-oxy]carbonylmethyl]pyrrolidinium chloride

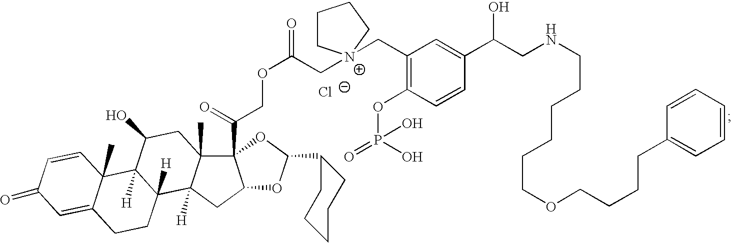

1-[5-[1-hydroxy-2-[6-(4-phenylbutoxy)hexylamino]ethyl]-2-phosphonooxybenzyl]-4-[[11,16α]-[[15,16-((R)-cyclohexylmethylene)bis(oxy)]-11-hydroxypregna-1,4-diene-3,20-dion-21-oxy]carbonylmethyl]-1-methylpiperazinium chloride

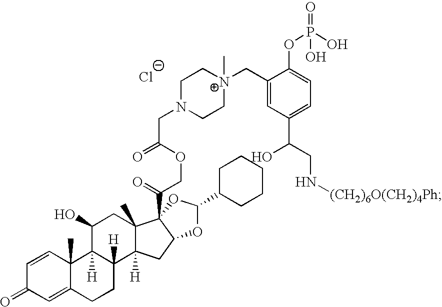

[5-[1-hydroxy-2-(1,1-dimethylethylamino)ethyl]-2-phosphonooxybenzyl]-(diethyl)-[[11β,16α]-[[15,16-((R)-cyclohexylmethylene)bis(oxy)]-11-hydroxypregna-1,4-diene-3,20-dion-21-oxy]carbonylmethyl]ammonium chloride

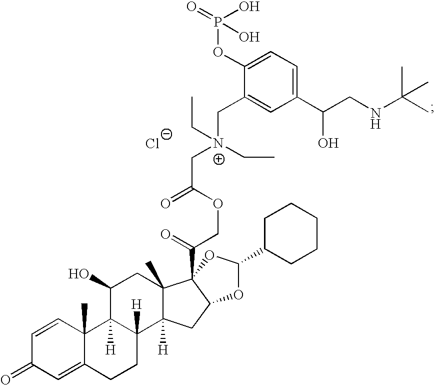

[5-[1-hydroxy-2-[6-(4-phenylbutoxy)hexylamino]ethyl]-2-phosphonooxybenzyl][4-[11β,16α]-[[15,16-((R)-cyclohexylmethylene)bis(oxy)]-11-hydroxypregna-1,4-diene-3,20-dion-21-oxy]carbonylphenyl]imidazolium chloride;

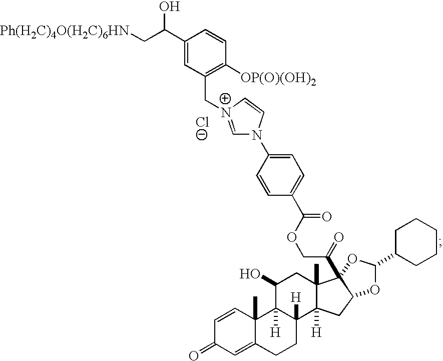

[5-[1-hydroxy-2-[6-(4-phenylbutoxy)hexylamino]ethyl]-2-phosphonooxybenzyl]-(dimethyl)-[5-amino-5-[[11,16α]-[[15,16-((R)-cyclohexylmethylene)bis(oxy)]-11-hydroxypregna-1,4-diene-3,20-dion-21-oxy]carbonyl]pentyl]ammonium chloride

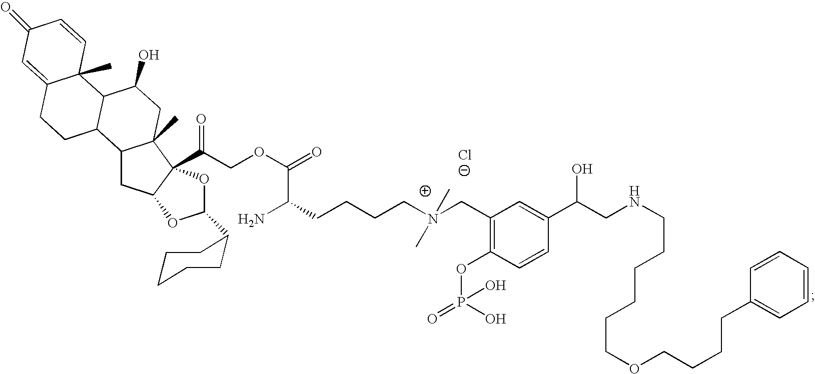

and pharmaceutically acceptable salts thereof or any subset thereof.

In one preferred embodiment, the compound of the invention is [5-[1-hydroxy-2-[6-(4-phenylbutoxy)hexylamino]ethyl]-2-phosphonooxybenzyl]-(diethyl)-[[11β,16α]-[[15,16-((R)-cyclohexylmethylene)bis(oxy)]-11-hydroxypregna-1,4-diene-3,20-dion-21-yl]carbonylmethyl]ammonium chloride

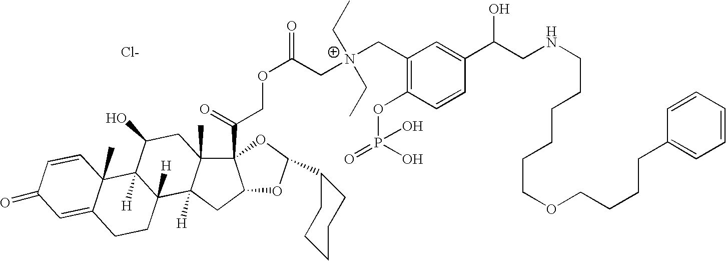

or a pharmaceutically acceptable salt thereof.

The compounds of Formula I, may be in the form of a salt, particularly a pharmaceutically acceptable salt thereof. Examples of pharmaceutically acceptable salts of the compounds of Formula I include salts derived from an appropriate base, such as an alkali metal or an alkaline earth (for example, Na⁺, Li⁺, K⁺, Ca²⁺ and Mg²⁺), ammonium and NR⁹₄⁺ (wherein R⁹ is C₁-C₄ alkyl). Pharmaceutically acceptable salts of a nitrogen atom or an amino group include (a) acid addition salts formed with inorganic acids, for example, hydrochloric acid, hydrobromic acid, sulfuric acid, sulfamic acids, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, isethionic acid, lactobionic acid, tannic acid, palmitic acid, alginic acid, polyglutamic acid, naphthalenesulfonic acid, methanesulfonic acid, p-toluenesulfonic acid, benzenesulfonic acid, naphthalenedisulfonic acid, naphthalene-1,5-disulfonic acid, polygalacturonic acid, malonic acid, sulfosalicylic acid, glycolic acid, 2-hydroxy-3-naphthoate, 1-hydroxy-2-naphthoate pamoate, salicylic acid, stearic acid, phthalic acid, mandelic acid, lactic acid, ethanesulfonic acid, lysine, arginine, glutamic acid, glycine, serine, threonine, alanine, isoleucine, leucine and the like; and (c) salts formed from elemental anions for example, chlorine, bromine, and iodine.

For therapeutic use, salts of active ingredients of the compounds of Formula I will be pharmaceutically acceptable, i.e. they will be salts derived from a pharmaceutically acceptable acid or base. However, salts of acids or bases which are not pharmaceutically acceptable may also find use, for example, in the preparation or purification of a pharmaceutically acceptable compound. All salts, whether or not derived from a pharmaceutically acceptable acid or base, are within the scope of the present invention.

Finally, it is to be understood that the compositions herein comprise compounds of the invention in their unionized, as well as zwitterionic form, and combinations with stoichiometric amounts of water as in hydrates.

The term “chiral” refers to molecules which have the property of non-superimposability of the mirror image partner, while the term “chiral” refers to molecules which are superimposable on their mirror image partner.

The term “stereoisomers” refers to compounds which have identical chemical constitution, but differ with regard to the arrangement of the atoms or groups in space. “Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

It is to be noted that all enantiomers, diastereomers, and racemic mixtures, tautomers, polymorphs, pseudopolymorphs of compounds within the scope of Formula I-1, I, II, or III and pharmaceutically acceptable salts thereof are embraced by the present invention. All mixtures of such enantiomers and diastereomers, including enantiomerically enriched mixtures and diastereomerically enriched mixtures are within the scope of the present invention. Enantiomerically enriched mixtures are mixtures of enantiomers wherein the ratio of the specified enantiomer to the alternative enantiomer is greater than 50:50. More particularly, an enantiomerically enriched mixture comprises at least about 75% of the specified enantiomer, and preferably at least about 85% of the specified enantiomer.

In one embodiment, the enantiomerically enriched mixture is substantially free of the other enantiomer. Similarly, diastereomerically enriched mixtures are mixtures of diastereomers wherein amount of the specified diastereomer is greater than the amount of each alternative diastereomer. More particularly, a diastereomerically enriched mixture comprises at least about 75% of the specified diastereomer, and preferably at least about 85% of the specified diastereomer. In one embodiment, the diastereomerically enriched mixture is substantially free of all other diastereomers.

For illustrative purposes, specific examples of enantiomers within the scope of the present invention include:
[5-[1-(R)-hydroxy-2-[6-(4-phenylbutoxy)hexylamino]ethyl]-2-phosphonooxybenzyl]-(diethyl)-[[11β,16α]-[[((R)-cyclohexylmethylene)bis(oxy)]-11-hydroxypregna-1,4-diene-3,20-dion-20-yl]methoxycarbonylmethyl]ammonium chloride

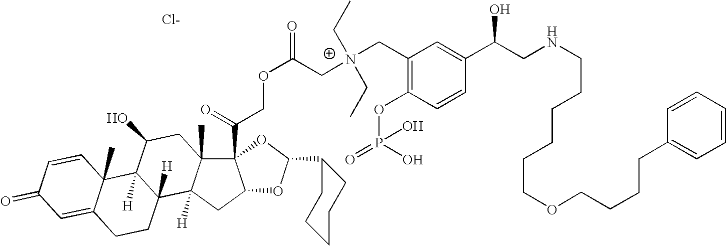

and
[5-[1-(S)-hydroxy-2-[6-(4-phenylbutoxy)hexylamino]ethyl]-2-phosphonooxybenzyl]-(diethyl)-[[11β,16α]-[[((R)-cyclohexylmethylene)bis(oxy)]-11-hydroxypregna-1,4-diene-3,20-dion-20-yl]methoxycarbonylmethyl]ammonium chloride

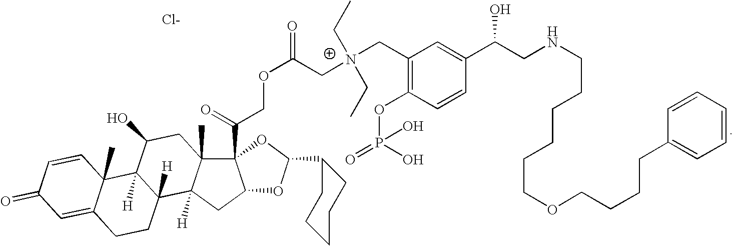

In one preferred embodiment, the present invention provides an enantiomerically enriched mixture comprising

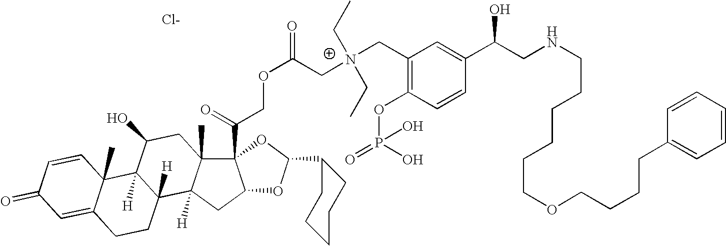

or a pharmaceutically acceptable salt thereof, as the predominant isomer.

A compound of Formula I and pharmaceutically acceptable salts thereof may exist as different polymorphs or pseudopolymorphs. As used herein, crystalline polymorphism means the ability of a crystalline compound to exist in different crystal structures. The crystalline polymorphism may result from differences in crystal packing (packing polymorphism) or differences in packing between different conformers of the same molecule (conformational polymorphism). As used herein, crystalline pseudopolymorphism also includes the ability of a hydrate or solvate of a compound to exist in different crystal structures. The pseudopolymorphs of the instant invention may exist due to differences in crystal packing (packing pseudopolymorphism) or due to differences in packing between different conformers of the same molecule (conformational pseudopolymorphism). The instant invention comprises all polymorphs and pseudopolymorphs of the compounds of Formula I and pharmaceutically acceptable salts thereof.

A compound of Formula I and pharmaceutically acceptable salts thereof may also exist as an amorphous solid. As used herein, an amorphous solid is a solid in which there is no long-range order of the positions of the atoms in the solid. This definition applies as well when the crystal size is two nanometers or less. Additives, including solvents, may be used to create the amorphous forms of the instant invention. The instant invention comprises all amorphous forms of the compounds of Formula I and pharmaceutically acceptable salts thereof.

The modifier “about” used in connection with a quantity is inclusive of the stated value and has the meaning dictated by the context (e.g., includes the degree of error associated with measurement of the particular quantity).

### Uses and Methods of Treatment

The compounds of the invention are useful as a medicament and more particularly, are useful for the treatment of clinical conditions for which a corticosteroid and/or selective β-agonists, and particularly β₂-agonists, are indicated. Such conditions may involve pulmonary inflammation and/or bronchoconstriction, and include diseases associated with reversible or irreversible airway obstruction. More particularly, such conditions include asthma, chronic obstructive pulmonary diseases (COPD), chronic bronchitis, bronchiectasis, emphysema, respiratory tract infection and upper respiratory tract diseases (e.g., rhinitis, including seasonal and allergic rhinitis).

Accordingly, in one aspect, the present invention provides a method for the treatment of a condition in a mammal, such as a human, for which a corticosteroid and/or β-agonist is indicated.

The terms “treating” and “treatment”, as used herein refers to reversing, alleviating, inhibiting the progress of, or preventing the disorder or condition or one or more symptoms of such disorder or condition.

All therapeutic methods described herein are carried out by administering an effective amount of a compound of the invention, i.e., a compound of Formula I-1, I, II or III or a pharmaceutically acceptable salt thereof, to a subject (typically mammal and preferably human) in need of treatment.

In one embodiment the invention provides a method for the treatment of pulmonary inflammation and bronchoconstriction in a mammal, particularly a human, in need thereof. In one particular embodiment the present invention provides a method for the treatment of a condition associated with reversible airway obstruction in a mammal, particularly a human in need thereof. In one embodiment the invention provides a method for the treatment of asthma in a mammal, particularly a human, in need thereof.

In one embodiment the invention provides a method for the treatment of chronic obstructive pulmonary disease in a mammal, particularly a human, in need thereof. In one embodiment the invention provides a method for the treatment of bronchitis, including chronic or wheezy bronchitis in a mammal, particularly a human, in need thereof. In one embodiment the invention provides a method for the treatment of bronchiectasis in a mammal, particularly a human, in need thereof. In one embodiment the invention provides a method for the treatment of emphysema in a mammal, particularly a human in need thereof. In one embodiment the invention provides a method for the treatment of a respiratory tract infection or upper respiratory tract disease in a mammal, particularly a human in need thereof.

There is also provided a compound of the invention for use in medical therapy, particularly for use in the treatment of condition in a mammal, such as a human, for which a corticosteroid and/or β-agonist is indicated. All therapeutic uses described herein are carried out by administering an effective amount of a compound of the invention to the subject in need of treatment. In one embodiment there is provided a compound of the invention for use in the treatment of pulmonary inflammation and bronchoconstriction in a mammal, particularly a human, in need thereof. In one particular embodiment there is provided a compound of the invention for use in the treatment of a condition associated with reversible airway obstruction in a mammal, particularly a human in need thereof. In one embodiment, there is provided a compound of the invention for use in the treatment of asthma in a mammal, particularly a human, in need thereof. In one embodiment there is provided a compound of the invention for use in the treatment of chronic obstructive pulmonary disease in a mammal, particularly a human, in need thereof. In one embodiment there is provided a compound for use in the treatment of bronchitis, including chronic bronchitis in a mammal, particularly a human, in need thereof. In one embodiment there is provided a compound for use in the treatment of bronchiectasis in a mammal, particularly a human, in need thereof. In one embodiment there is provided a compound for use in the treatment of emphysema in a mammal, particularly a human in need thereof. In one embodiment there is provided a compound of the invention for use in the treatment of a respiratory tract infection or upper respiratory tract disease in a mammal, particularly a human, in need thereof.

The present invention also provides the use of a compound of the invention in the manufacture of a medicament for the treatment of a condition in a mammal, such as a human, for which a corticosteroid and/or β-agonist is indicated. In one embodiment is provided the use of a compound of the invention in the manufacture of a medicament for the treatment of pulmonary inflammation and bronchoconstriction in a mammal, particularly a human, in need thereof. In one particular embodiment is provided the use of a compound of the invention in the manufacture of a medicament for the treatment of a condition associated with reversible airway obstruction in a mammal, particularly a human in need thereof. In one embodiment is provided a compound of the invention in the manufacture of a medicament for the treatment of asthma in a mammal, particularly a human, in need thereof. In one embodiment is provided the use of a compound of the invention in the manufacture of a medicament for the treatment of chronic obstructive pulmonary disease in a mammal, particularly a human, in need thereof. In one embodiment is provided the use of a compound of the invention in the manufacture of a medicament for the treatment of bronchitis, including chronic bronchitis in a mammal, particularly a human, in need thereof. In one embodiment is provided the use of a compound of the invention in the manufacture of a medicament for the treatment of bronchiectasis in a mammal, particularly a human, in need thereof. In one embodiment is provided the use of a compound of the invention for the manufacture of a medicament for the treatment of emphysema in a mammal, particularly a human in need thereof. In one embodiment is provided the use of a compound of the invention for the manufacture of a medicament for the treatment of a respiratory tract infection or upper respiratory tract disease in a mammal, particularly a human in need thereof.

The term “effective amount”, as used herein, is an amount of compound of the invention which is sufficient in the subject to which it is administered, to elicit the biological or medical response of a cell culture, tissue, system, mammal (including human) that is being sought, for instance by a researcher or clinician. The term also includes within its scope, amounts effective to enhance normal physiological function. In one embodiment, the effective amount is the amount needed to provide a desired level of drug in the secretions and tissues of the airways and lungs, or alternatively, in the bloodstream of a subject to be treated to give an anticipated physiological response or desired biological effect when such a composition is administered by inhalation. For example an effective amount of a compound of the invention for the treatment of a condition for which a corticosteroid and/or β-agonist is indicated is sufficient in the subject to which it is administered to treat the particular condition. In one embodiment an effective amount is an amount of a compound of the invention which is sufficient for the treatment of asthma, or COPD in a human.

The precise effective amount of the compounds of the invention will depend on a number of factors including but not limited to the species, age and weight of the subject being treated, the precise condition requiring treatment and its severity, the bioavailability, potency, and other properties of the specific compound being administered, the nature of the formulation, the route of administration, and the delivery device, and will ultimately be at the discretion of the attendant physician or veterinarian.

An estimated dose (for inhalation) of a compound of the invention for treatment of a 70 kg human may be in the range of from about 10 to about 5000 μg. The selection of the specific dose for a patient will be determined by the attendant physician, clinician or veterinarian of ordinary skill in the art based upon a number of factors including those noted above. In one particular embodiment, the dose of a compound of the invention for the treatment of a 70 kg human will be in the range of from about 50 to about 2500 μg. In one preferred embodiment the dose of a compound of the invention for the treatment of a 70 kg human will be in the range of from about 100 to about 1000 μg. Doses may be adjusted if the compound is administered via a different route. Determination of an appropriate dose for administration by other routes is within the skill of those in the art in light of the foregoing description and the general knowledge in the art.

Delivery of an effective amount of a compound of the invention may entail delivery of a single dosage form or multiple unit doses which may be delivered contemporaneously or separate in time over a designated period, such as 24 hours. Typically, a compound of the invention (alone or in the form of a composition comprising the same) will be administered four, three, two, or most preferably once per day (24 hours).

### Compositions

While it is possible for a compound of the invention to be administered alone, it is preferable to present it in the form of a composition, particularly a pharmaceutical composition (formulation). Thus, in another aspect, the invention provides compositions, and particularly pharmaceutical compositions (such as an inhalable pharmaceutical composition) comprising a compound of the invention as an active ingredient and a pharmaceutically acceptable excipient, diluent or carrier. The term “active ingredient” as employed herein refers to any of a compound of Formula I-1, I, II or III or a pharmaceutically acceptable salt of any of the foregoing. In a particular embodiment, the composition is a novel, efficacious, safe, nonirritating and physiologically compatible inhalable composition comprising the active ingredient. The composition is preferably suitable for treating asthma, bronchitis, or COPD.

Pharmaceutical compositions according to the invention include those suitable for oral administration; parenteral administration, including subcutaneous, intradermal, intramuscular, intravenous and intraarticular; and administration to the respiratory tract, including the nasal cavities and sinuses, oral and extrathoracic airways, and the lungs, including by use of aerosols which may be delivered by means of various types of dry powder inhalers, pressurized metered dose inhalers, softmist inhalers, nebulizers, or insufflators. The most suitable route of administration may depend upon, several factors including the patient and the condition or disorder being treated.

The formulations may be presented in unit dosage form or in bulk form as for example in the case of formulations to be metered by an inhaler and may be prepared by any of the methods well known in the art of pharmacy. All methods include the step of bringing the active ingredient into association with the carrier, diluent or excipient and optionally one or more accessory ingredients. In general the formulations are prepared by uniformly and intimately bringing into association the active ingredient with one or more liquid carriers, diluents or excipients or finely divided solid carriers, diluents or excipients, or both, and then, if necessary, shaping the product into the desired formulation.

In one preferred embodiment, the composition is an inhalable pharmaceutical composition which is suitable for inhalation and delivery to the endobronchial space. Typically, such composition is in the form of an aerosol comprising particles for delivery using a nebulizer, pressurized metered dose inhaler (pMDI), softmist inhaler, or dry powder inhaler (DPI).

Aerosols used to administer medicaments to the respiratory tract are typically polydisperse, that is they are comprised of particles of many different sizes. The particle size distribution is typically described by the Mass Median Aerodynamic Diameter (MMAD) and the Geometric Standard Deviation (GSD). For optimum drug delivery to the endobronchial space the MMAD is in the range from about 1 to about 10 μm and preferably from about 1 to about 5 μm, and the GSD is less than 3, and preferably less than about 2. Aerosols having a MMAD above 10 μm are generally too large when inhaled to reach the lungs. Aerosols with a GSD greater than about 3 are not preferred for lung delivery as they deliver a high percentage of the medicament to the oral cavity. To achieve these particle sizes the particles of the active ingredient as produced may be size reduced using conventional techniques such as micronisation. Non limiting examples of other processes or techniques that can be used to produce respirable particles include spray drying, precipitation, supercritical fluid, and freeze drying. The desired fraction may be separated out by air classification or sieving. In one embodiment, the particles will be crystalline.

Aerosol particle size distributions are determined using devices well known in the art. For example a multi-stage Anderson cascade impactor or other suitable method such as those specifically cited within the US Pharmacopoeia Chapter 601 as characterizing devices for aerosols emitted from metered-dose and dry powder inhalers.

Dry powder compositions for topical delivery to the lung by inhalation generally contain a mix of the active ingredient and a suitable powder base (carrier/diluent/excipient substance) such as mono-, di- or poly-saccharides (e.g., lactose or starch). Lactose is typically preferred. When a solid excipient such as lactose is employed, generally the particle size of the excipient will be much greater than the active ingredient to aid the dispersion of the formulation in the inhaler.

Non-limiting examples of dry powder inhalers include reservoir multi-dose inhalers and pre-metered multi-dose inhalers. A reservoir inhaler contains a large number of doses (e.g. 60) in one container. Prior to inhalation, the patient actuates the inhaler which causes the inhaler to meter one dose of medicament from the reservoir and prepare it for inhalation. In a pre-metered multi-dose inhaler, each individual dose has been manufactured in a separate container, and actuation of the inhaler prior to inhalation causes a new dose of drug to be released from its container and prepared for inhalation. During inhalation, the inspiratory flow of the patient accelerates the powder out of the device and into the oral cavity. Turbulent flow characteristics of the powder path cause the excipient-drug aggregates to disperse, and the particles of active ingredient are deposited deep in the lungs. In preferred embodiments, a compound of the invention is delivered as a dry powder using a dry powder inhaler wherein the particles emitted from the inhaler have an MMAD in the range of about 1 μm to about 5 μm and a GSD about less than 2.

Examples of suitable dry powder inhalers and dry powder dispersion devices for use in the delivery of compounds and compositions according to the present invention include but are not limited to those disclosed in U.S. Pat. No. 7,520,278; U.S. Pat. No. 7,322,354; U.S. Pat. No. 7,246,617; U.S. Pat. No. 7,231,920; U.S. Pat. No. 7,219,665; U.S. Pat. No. 7,207,330; U.S. Pat. No. 6,880,555; U.S. Pat. No. 5,522,385; U.S. Pat. No. 6,845,772; U.S. Pat. No. 6,637,431; U.S. Pat. No. 6,329,034; U.S. Pat. No. 5,458,135; U.S. Pat. No. 4,805,811.

In one embodiment, the pharmaceutical formulation according to the invention is a dry powder for inhalation which is formulated for delivery by a Diskus®-type device. The Diskus® device comprises an elongate strip formed from a base sheet having a plurality of recesses spaced along its length and a lid sheet hermetically but peelably sealed thereto to define a plurality of containers, each container having therein an inhalable formulation containing a predetermined amount active ingredient either alone or in admixture with one or more carriers or excipients (e.g., lactose) and/or other therapeutically active agents. Preferably, the strip is sufficiently flexible to be wound into a roll. The lid sheet and base sheet will preferably have leading end portions which are not sealed to one another and at least one of the leading end portions is constructed to be attached to a winding means. Also, preferably the hermetic seal between the base and lid sheets extends over their whole width. To prepare the dose for inhalation, the lid sheet may preferably be peeled from the base sheet in a longitudinal direction from a first end of the base sheet.

In another embodiment, the pharmaceutical formulation according to the invention is a dry powder for inhalation wherein the dry powder is formulated into microparticles as described in PCT Publication No. WO2009/015286 or WO2007/114881, both to NexBio. Such microparticles are generally formed by adding a counterion to a solution containing a compound of the invention in a solvent, adding an antisolvent to the solution; and gradually cooling the solution to a temperature below about 25° C., to form a composition containing microparticles comprising the compound. The microparticles comprising the compound may then be separated from the solution by any suitable means such as sedimentation, filtration or lyophilization. Suitable counterions, solvents and antisolvents for preparing microparticles of the compounds of the invention are described in WO2009/015286.

Spray compositions for topical delivery to the endobronchial space or lung by inhalation may for example be formulated as aqueous solutions or suspensions or as aerosols delivered from pressurized packs, such as metered dose inhalers, with the use of suitable liquefied propellants, softmist inhalers, or nebulizers. Such aerosol compositions suitable for inhalation can be either a suspension or a solution and generally contain the active ingredient together with a pharmaceutically acceptable carrier or diluent (e.g., water, saline, or ethanol) and optionally one or more therapeutically active agents.

Aerosol compositions for delivery by pressurized metered dose inhalers typically further comprise a pharmaceutically acceptable propellant. Examples of such propellants include fluorocarbon or hydrogen-containing chlorofluorocarbon or mixtures thereof, particularly hydrofluoroalkanes, e.g., dichlorodifluoromethane, trichlorofluoromethane, dichlorotetrafluoroethane, especially 1,1,1,2-tetrafluoroethane, 1,1,1,2,3,3,3,-heptafluoro-n-propane or a mixture thereof. The aerosol composition may be excipient free or may optionally contain additional formulation excipients well known in the art such as surfactants e.g., oleic acid or lecithin and cosolvents e.g., ethanol. Pressurized formulations will generally be retained in a canister (e.g., an aluminum canister) closed with a valve (e.g., a metering valve) and fitted into an actuator provided with a mouthpiece.

In another embodiment, a pharmaceutical composition according to the invention is delivered as a dry powder using a metered dose inhaler. Non-limiting examples of metered dose inhalers and devices include those disclosed in U.S. Pat. No. 5,261,538; U.S. Pat. No. 5,544,647; U.S. Pat. No. 5,622,163; U.S. Pat. No. 4,955,371; U.S. Pat. No. 3,565,070; U.S. Pat. No. 3,361,306 and U.S. Pat. No. 6,116,234. In a preferred embodiment, a compound of the invention is delivered as a dry powder using a metered dose inhaler wherein the emitted particles have an MMAD that is in the range of about 1 μm to about 5 μm and a GSD that is less than about 2.

In one embodiment is provided a pharmaceutical composition comprising an effective amount of a compound of the invention in a dosage form suitable for delivery via a nebulizer, metered dose inhaler, or dry powder inhaler. In one particular embodiment is provided a pharmaceutical composition comprising an effective amount of a compound of the invention in a dosage form suitable for aerosolization by metered-dose inhaler; or jet, ultrasonic, or vibrating porous plate nebulizer.

Such liquid inhalable solutions for nebulization may be generated by solubilizing or reconstituting a solid particle formulation or may be formulated with an aqueous vehicle with the addition of agents such as acid or alkali, buffer salts, and isotonicity adjusting agents. They may be sterilized by in process techniques such as filtration, or terminal processes such as heating in an autoclave or gamma irradiation. They may also be presented in non-sterile from.

Such formulations may be administered using commercially available nebulizers or other atomizer that can break the formulation into particles or droplets suitable for deposition in the nasal cavities or respiratory tract. Non-limiting examples of nebulizers which may be employed for the aerosol delivery of a composition of the invention include pneumatic jet nebulizers, vented or breath enhanced jet nebulizers, or ultrasonic nebulizers including static or vibrating porous plate nebulizers. A jet nebulizer utilizes a high velocity stream of air blasting up through a column of water to generate droplets. Particles unsuitable for inhalation impact on walls or aerodynamic baffles. A vented or breath enhanced nebulizer works the same as a jet nebulizer except that inhaled air passes through the primary droplet generation area to increase the output rate of the nebulizer while the patient inhales. In an ultrasonic nebulizer, vibration of a piezoelectric crystal creates surface instabilities in the drug reservoir that cause droplets to be formed. In porous plate nebulizers pressure fields generated by sonic energy force liquid through the mesh pores where it breaks into droplets by Rayleigh breakup. The sonic energy may be supplied by a vibrating horn or plate driven by a piezoelectric crystal, or by the mesh itself vibrating. Non-limiting examples of atomizers include any single or twin fluid atomizer or nozzle that produces droplets of an appropriate size. A single fluid atomizer works by forcing a liquid through one or more holes, where the jet of liquid breaks up into droplets. Twin fluid atomizers work by either forcing both a gas and liquid through one or more holes, or by impinging a jet of liquid against another jet of either liquid or gas.

The nebulizer which aerosolizes the formulation of the active ingredient is important in the administration of the active ingredient. Different nebulizers have differing efficiencies based their design and operation principle and are sensitive to the physical and chemical properties of the formulation. For example, two formulations with different surface tensions may have different particle size distributions. Additionally, formulation properties such as pH, Osmolality, and permeant ion content can affect tolerability of the medication, so preferred embodiments conform to certain ranges of these properties.

In a preferred embodiment, the formulation for nebulization is delivered to the endobronchial space as an aerosol having an MMAD between about 1 μm and about 5 μm and a GSD less than 2 using an appropriate nebulizer. To be optimally effective and to avoid upper respiratory and systemic side effects, the aerosol should not have a MMAD greater than about 5 μm and should not have a GSD greater than about 2. If an aerosol has an MMAD larger than about 5 kin or a GSD greater than about 2, a large percentage of the dose may be deposited in the upper airways decreasing the amount of drug delivered to the site of inflammation and bronchoconstriction in the lower respiratory tract. If the MMAD of the aerosol is smaller than about 1 μm, then the particles may remain suspended in the inhaled air and may then be exhaled during expiration.

Formulations suitable for oral administration may be presented as discrete units such as capsules, cachets or tablets, each containing a predetermined amount of the active ingredient; as a powder or granules; as a solution or suspension in an aqueous liquid or a non-aqueous liquid; or as an oil-in-water liquid emulsion or a water-in-oil liquid emulsion. The active ingredient may also be presented as a sachet, bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binders, lubricant, inert diluent, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Formulations for topical administration in the mouth, for example buccally or sublingually, include lozenges, comprising the active ingredient in a flavored base such as sucrose and acacia or tragacanth, and pastilles comprising the active ingredient in a base such as gelatin and glycerin or sucrose and acacia.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example saline or water-for-injection, immediately prior to use. Extemporaneous injection solutions and suspensions may be prepared from sterile powders, granules and tablets of the kind previously described.

In another aspect of the invention, the aerosolizable formulation of a compound of the invention delivers an effective amount of the compound ranging from about 1 to about 5000 μg to the lungs wherein the composition produces plasma concentrations of the β-agonist and/or corticosteroid of less than about 10 nanograms/mL one hour after administration of said composition. In a preferred embodiment of the invention, the plasma concentrations of the β-agonist and/or corticosteroid produced are less than about 5 nanograms/mL one hour after administration of the composition. In a particularly preferred embodiment of the invention, the plasma concentrations of the β-agonist and/or corticosteroid produced are less than about 2 nanograms/mL one hour after administration of the composition.

In another aspect, the invention provides a method of treating pulmonary inflammation and bronchoconstriction comprising treating a subject in need thereof with an effective amount of an inhalable pharmaceutical composition of a compound of the invention wherein the inhalable pharmaceutical composition produces plasma concentrations of the β-agonist and/or corticosteroid comprising the compound of the invention of less than 10 nanograms/mL one hour after administration of said composition. In a preferred embodiment of the method, the plasma concentrations of the β-agonist and/or corticosteroid produced are less than about 5 nanograms/mL one hour after administration of said formulation. In a particularly preferred embodiment of the method, the plasma concentrations of the β-agonist and/or corticosteroid produced are less than about 2 nanograms/mL one hour after administration of said formulation.

In another aspect, the invention provides a method of treating asthma, COPD, bronchitis, bronchiectasis, emphysema or rhinitis in a human subject comprising treating the subject with an effective amount of a inhalable pharmaceutical composition of a compound of the invention wherein the inhalable pharmaceutical composition produces plasma concentrations of the β-agonist and/or corticosteroid of less than 10 nanograms/mL one hour after administration of said composition. In a preferred embodiment of the method, the plasma concentrations of the β-agonist and/or corticosteroid produced are less than about 5 nanograms/mL one hour after administration of said formulation. In a particularly preferred embodiment of the method, the plasma concentrations of the β-agonist and/or corticosteroid produced are less than about 2 nanograms/mL one hour after administration of said formulation.

Preferred unit dosage formulations for the compounds of the invention are those containing an effective amount of the active ingredient or an appropriate fraction thereof.

It should be understood that in addition to the ingredients particularly mentioned above, the formulations of this invention may include other agents conventional in the art having regard to the type of formulation in question for example those suitable for oral administration may include flavoring agents.

As noted above, the compounds of the invention may be formulated and/or used in combination with other therapeutically active agents. Examples of other therapeutically active agents which may be formulated or used in combination with the compounds of the invention include but are not limited to anti-inflammatory agents, anticholinergic agents, β-agonists (including selective β₂-agonists), peroxisome proliferator-activated receptor (PPAR) gamma agonists, PPAR delta agonists, epithelial sodium channel blockers (ENaC receptor blockers), kinase inhibitors, antiinfective agents and antihistamines. The present invention thus provides, as another aspect, a composition comprising an effective amount of compound of the invention and another therapeutically active agent selected from anti-inflammatory agents, anticholinergic agents, β-agonists (including selective β₂-agonists), peroxisome proliferator-activated receptor (PPAR) gamma agonists, PPAR delta agonists, epithelial sodium channel blockers (ENaC receptor blockers), kinase inhibitors, antiinfective agents and antihistamines.

Suitable anti-inflammatory agents for use in combination with the compounds of the invention include corticosteroids and non-steroidal anti-inflammatory drugs (NSAIDs), particularly phosphodiesterase inhibitors. Examples of corticosteroids for use in the present invention include oral or inhaled corticosteroids or prodrugs thereof. Specific examples include but are not limited to ciclesonide, desisobutyryl ciclesonide, budesonide, flunisolide, mometasone and esters thereof (e.g., mometasone furoate), fluticasone propionate, fluticasone furoate, beclomethasone, methyl prednisolone, prednisolone, dexamethasone, 6a,9a-difluoro-17a-[(2-furanylcarbonyl)oxy]-11β-hydroxy-16a-methyl-3-oxo-androsta-1,4-diene-17β-carbothioic acid S-fluoromethyl ester, 6a,9a-difluoro-11β-hydroxy-16a-methyl-3-oxo-17a-propionyloxy-androsta-1,4-diene-17β-carbothioic acid S-(2-oxo-tetrahydro-furan-3S-yl) ester, beclomethasone esters (e.g., the 17-propionate ester or the 17,21-dipropionate ester, fluoromethyl ester, triamcinolone acetonide, rofleponide, or any combination or subset thereof. Preferred corticosteroids for formulation or use in combination with the compounds of the invention are selected from ciclesonide, desisobutyryl ciclesonide, budesonide, mometasone, fluticasone propionate, and fluticasone furoate, or any combination or subset thereof.

NSAIDs for use in the present invention include but are not limited to sodium cromoglycate, nedocromil sodium, phosphodiesterase (PDE) inhibitors (e.g., theophylline, PDE4 inhibitors, mixed PDE3/PDE4 inhibitors or mixed PDE4/PDE7 inhibitors), leukotriene antagonists, inhibitors of leukotriene synthesis (e.g., 5 LO and FLAP inhibitors), nitric oxide synthase (iNOS) inhibitors, protease inhibitors (e.g., tryptase inhibitors, neutrophil elastase inhibitors, and metalloprotease inhibitors) β2-integrin antagonists and adenosine receptor agonists or antagonists (e.g., adenosine 2a agonists), cytokine antagonists (e.g., chemokine antagonists) or inhibitors of cytokine synthesis (e.g., prostaglandin D2 (CRTh2) receptor antagonists).

The PDE4 inhibitor, mixed PDE3/PDE4 inhibitor or mixed PDE4/PDE7 inhibitor may be any compound that is known to inhibit the PDE4 enzyme or which is discovered to act as a PDE4 inhibitor, and which are selective PDE4 inhibitors (i.e., compounds which do not appreciably inhibit other members of the PDE family). Examples of specific PDE4 inhibitors for formulation and use in combination with the compounds of the present invention include but are not limited to roflumilast, pumafentrine, arofylline, cilomilast, tofimilast, oglemilast, tolafentrine, piclamilast, ibudilast, apremilast, 2-[4-[6,7-diethoxy-2,3-bis(hydroxymethyl)-1-naphthalenyl]-2-pyridinyl]-4-(3-pyridinyl)-1(2H)-phthalazinone (T2585), N-(3,5-dichloro-4-pyridinyl)-1-[(4-fluorophenyl)methyl]-5-hydroxy-α-oxo-1H-indole-3-acetamide (AWD-12-281, 4-[(2R)-2-[3-(cyclopentyloxy)-4-methoxyphenyl]-2-phenylethyl]-pyridine (CDP-840), 2-[4-[[[[2-(1,3-benzodioxol-5-yloxy)-3-pyridinyl]carbonyl]amino]methyl]-3-fluorophenoxy]-(2R)-propanoic acid (CP-671305), N-(4,6-dimethyl-2-pyridinyl)-4-[4,5,6,7-tetrahydro-2-(4-methoxy-3-methylphenyl)-5-(4-methyl-1-piperazinyl)-1H-indol-1-yl]-benzenesulfonamide, (2E)-2-butenedioate (YM-393059), 9-[(2-fluorophenyl)methyl]-N-methyl-2-(trifluoromethyl)-9H-purin-6-amine (NCS-613), N-(2,5-dichloro-3-pyridinyl)-8-methoxy-5-quinolinecarboxamide (D-4418), N-[(3R)-9-amino-3,4,6,7-tetrahydro-4-oxo-1-phenylpyrrolo[3,2,1-][1,4]benzodiazepin-3-yl]-3H-purin-6-amine (PD-168787), 3-[[3-(cyclopentyloxy)-4-methoxyphenyl]methyl]-N-ethyl-8-(1-methylethyl)-3H-purin-6-amine hydrochloride (V-11294A), N-(3,5-dichloro-1-oxido-4-pyridinyl)-8-methoxy-2-(trifluoromethyl)-5-quinolinecarboxamide (Sch351591), 5-[3-(cyclopentyloxy)-4-methoxyphenyl]-3-[(3-methylphenyl)methyl]-(3S,5)-2-piperidinone (HT-0712), 5-(2-((1r,4r)-4-amino-1-(3-(cyclopenyloxy)-4-methyoxyphenyl)cyclohexyl)ethynyl)-pyrimidine-2-amine (GSK-256066), cis-[4-cyano-4-(3-cyclopropylmethoxy-4-difluoromethoxyphenyl)cyclohexan-1-ol, and 4-[6,7-d]ethoxy-2,3-bis(hydroxymethyl)-1-naphthalenyl]-1-(2-methoxyethyl)-2(1H)-pyridinone (T-440), and any combination or subset thereof.

Leukotriene antagonists and inhibitors of leukotriene synthesis include zafirlukast, montelukast sodium, zileuton, and pranlukast.

Antichlolinergic agents for formulation or use in combination with the compounds of the invention include but are not limited to muscarinic receptor antagonists, particularly including pan antagonists and antagonists of the M₃ receptors. Exemplary compounds include the alkaloids of the belladonna plants, such as atropine, scopolamine, homatropine, hyoscyamine, and the various forms including salts thereof (e.g., anhydrous atropine atropine sulfate, atropine oxide or HCl, methylatropine nitrate, homatropine hydrobromide, homatropine methyl bromide, hyoscyamine hydrobromide, hyoscyamine sulfate, scopolamine hydrobromide, scopolamine methyl bromide) tolterodine, darifenacin, solifenacin, revatropate, or any combination or subset thereof.

Additional anticholinergics for formulation and use in combination with the methantheline, propantheline bromide, anisotropine methyl bromide or Valpin 50, aclidinium bromide, glycopyrrolate (Robinul), isopropamide iodide, mepenzolate bromide, tridihexethyl chloride, hexocyclium methylsulfate, cyclopentolate HCl, tropicamide, trihexyphenidyl CCl, pirenzepine, telenzepine, and methoctramine, or any combination or subset thereof.

Preferred anticholinergics for formulation and use in combination with the compounds of the invention include ipratropium (bromide), oxitropium (bromide) and tiotropium (bromide), or any combination or subset thereof.

Examples of β-agonists for formulation and use in combination with the compounds of the invention include but are not limited to salmeterol, R-salmeterol, and xinafoate salts thereof, albuterol or R-albuterol (free base or sulfate), formoterol (fumarate), fenoterol, terbutaline and salts thereof, and any combination or subset thereof.

Examples of PPAR gamma agonists for formulation and use in combination with the compounds of the invention include but are not limited to thiazolidinediones, rosiglitazone, pioglitazone, and troglitazone.

Examples of ENaC receptor blockers for formulation and use in combination with the compounds of the invention include but are not limited to amiloride and derivatives thereof such as those compounds described in U.S. Pat. No. 6,858,615 to Parion Sciences, Inc.

Examples of kinase inhibitors include inhibitors of NFkB, PI3K (phosphatidylinositol 3-kinase), p38-MAP kinase and Rho kinase.

Antiinfective agents for formulation and use in combination with the compounds of the invention include antivirals and antibiotics. Examples of suitable antivirals include Tamiflu® and Relenza®. Examples of suitable antibiotics include but are not limited to aztreonam (arginine or lysine), fosfomycin, and tobramycin, or any combination or subset thereof.

Antihistamines (i.e., H1-receptor antagonists) for formulation and use in combination with the compounds of the invention include hut are not limited to:
Ethanolamines such as diphenhydramine HCl, carbinoxamine maleate, doxylamine, clemastine fumarate, diphenylhydramine HCl and dimenhydrinate;
Ethylenediamines such as pyrilamine maleate (metpyramine), tripelennamine HCl, tripelennamine citrate, and antazoline;
Alkylamines such as pheniramine, chloropheniramine, bromopheniramine, dexchlorpheniramine, triprolidine and acrivastine;
Pyridines such as methapyrilene, piperazines such as hydroxyzine HCl, hydroxyzine pamoate, cyclizine HCl, cyclizine lactate, meclizine HCl and cetirizine HCl;
Piperidines such as astemisole, levocabastine HCl, loratadine, descarboethoxy loratadine, terfenadine, and fexofenadine HCl;
Tri- and Tetracyclics such as promethazine, chlorpromethazine trimeprazine and azatadine; and
Azelastine HCl, or any combination or subset thereof.

In one aspect, the present invention provides a composition comprising a compound of the invention and an anti-inflammatory agent. In one embodiment, the composition comprises a compound of the invention and a corticosteroid. In one particular embodiment, the composition comprises a compound of the invention and a corticosteroid selected from ciclesonide, desisobutyryl ciclesonide, budesonide mometasone, fluticasone propionate, and fluticasone furoate. In one particular embodiment, the composition comprises a compound of the invention and ciclesonide or desisobutyryl ciclesonide.

In one aspect, the present invention provides a composition comprising a compound of the invention and a PDE4 inhibitor.

In one aspect, the present invention provides a composition comprising a compound of the invention and a β2-agonist. In one embodiment, the composition comprises a compound of the invention and salmeterol, R-salmeterol or formoterol. In one particular embodiment, the composition comprises a compound of the invention and salmeterol or R-salmeterol.

In one aspect, the present invention provides a composition comprising a compound of the invention and an anticholinergic agent. In one embodiment, the composition comprises a compound of the invention and tiotropium.

In one aspect the present invention provides a composition comprising a compound of the invention and anti-histamine.

In the above-described methods of treatment and uses, a compound of the invention may be employed alone, or in combination with one or more other therapeutically active agents. Typically, any therapeutically active agent that has a therapeutic effect in the disease or condition being treated with the compound of the invention may be utilized in combination with the compounds of the invention, provided that the particular therapeutically active agent is compatible with therapy employing a compound of the invention. Typical therapeutically active agents which are suitable for use in combination with the compounds of the invention include the anti-inflammatory agents, anticholinergic agents, β-agonists, antiinfective agents and antihistamines described above.

In another aspect, the invention provides methods for treatment and uses as described above, which comprise administering an effective amount of a compound of the invention and at least one other therapeutically active agent. The compounds of the invention and at least one additional therapeutically active agent may be employed in combination concomitantly or sequentially in any therapeutically appropriate combination. The administration of a compound of the invention with one or more other therapeutically active agents may be by administration concomitantly in 1) a unitary pharmaceutical composition, such as the compositions described above, or 2) separate pharmaceutical compositions each including one or more of the component active ingredients. The components of the combination may be administered separately in a sequential manner wherein the compound of the invention is administered first and the other therapeutically active agent is administered second or vice versa.

When a compound of the invention is used in combination with another therapeutically active agent, the dose of each compound may differ from that when the compound of the invention is used alone. Appropriate doses will be readily determined by one of ordinary skill in the art. The appropriate dose of the compound of the invention, the other therapeutically active agent(s) and the relative timings of administration will be selected in order to achieve the desired combined therapeutic effect, and are within the expertise and discretion of the attendant physician, clinician or veterinarian.

In another aspect, the present invention provides methods for treating any of the conditions enumerated above, comprising administering an effective amount of a compound of the invention and an anti-inflammatory agent. In one embodiment, the method comprises administering an effective amount of a compound of the invention and a corticosteroid. In one particular embodiment, the method comprises administering an effective amount of a compound of the invention and a corticosteroid selected from ciclesonide, desisobutyryl ciclesonide, budesonide mometasone, fluticasone propionate, and fluticasone furoate. In one particular embodiment, the method comprises administering an effective amount of a compound of the invention and ciclesonide or desisobutyryl ciclesonide.

In one embodiment the present invention provides a method for treating any of the conditions enumerated above comprising administering an effective amount of a compound of the invention and a PDE4 inhibitor.

In one embodiment the present invention provides a method for treating any of the conditions enumerated above comprising administering an effective amount of a compound of the invention and a β-agonist, particularly a selective β₂-agonist. In one embodiment, the method comprises administering an effective amount of a compound of the invention and salmeterol, R-salmeterol or formoterol. In one particular embodiment, the method comprises administering an effective amount of a compound of the invention and salmeterol or R-salmeterol.

In one embodiment the present invention provides a method for treating any of the conditions enumerated above by administering an effective amount of a compound of the invention and an anticholinergic agent. In one embodiment, the method comprises administering an effective amount of a compound of the invention and tiotropium. In one embodiment the present invention provides a method for treating any of the conditions enumerated above by administering an effective amount of a compound of the invention and anti-histamine.

In another aspect the present invention provides a combination comprising a compound of the invention and an anti-inflammatory agent for the treatment of any condition enumerated above; and also the use of such combination for the manufacture of a medicament for the treatment of any of the conditions enumerated above. In one embodiment, the combination comprises a compound of the invention and a corticosteroid selected from ciclesonide, desisobutyryl ciclesonide, budesonide mometasone, fluticasone propionate, and fluticasone furoate. In one particular embodiment, the combination comprises a compound of the invention and ciclesonide or desisobutyryl ciclesomide.

In another aspect the present invention provides a combination comprising a compound of the invention and a PDE4 inhibitor for the treatment of any condition enumerated above; and also the use of such combination for the manufacture of a medicament for the treatment of any of the conditions enumerated above.

In another aspect the present invention provides a combination comprising a compound of the invention and a β-agonist for the treatment of any condition enumerated above; and also the use of such combination for the manufacture of a medicament for the treatment of any of the conditions enumerated above. In one embodiment the combination comprises a compound of the invention and salmeterol, R-salmeterol or formoterol. In one particular embodiment, the combination comprises a compound of the invention and salmeterol or R-salmeterol.

In another aspect the present invention provides a combination comprising a compound of the invention and an anticholinergic agent for the treatment of any condition enumerated above; and also the use of such combination for the manufacture of a medicament for the treatment of any of the conditions enumerated above. In one embodiment the combination comprises a compound of the invention and tiotropium.

In another aspect the present invention provides a combination comprising a compound of the invention and an antihistamine for the treatment of any condition enumerated above; and also the use of such combination for the manufacture of a medicament for the treatment of any of the conditions enumerated above.

The present invention also provides processes for preparing the compounds of the invention and to the synthetic intermediates useful in such processes, as described in detail below.

### Preparation of the Compounds of the Invention

Having generally described this invention, a further understanding can be obtained by reference to certain specific examples which are provided herein for purposes of illustration only and are not intended to be limiting unless otherwise specified.

Certain abbreviations and acronyms are used in describing the experimental details. Although most of these would be understood by one skilled in the art, Table 1 contains a list of many of these abbreviations and acronyms.

TABLE 1
List of abbreviations and acronyms.

| Abbreviation | Meaning |
|---|---|
| Boc | Tert-butoxycarbonyl |
| Boc₂O | di-tert-butyldicarbonate |
| CDI | carbonyldiimidazole |
| ACN | acetonitrile |
| DBU | 1,5-diazabicyclo[5.4.0]undecene-5 |
| DCM | dichloromethane |
| DIEA | N,N-diisopropylethylamine |
| DCC | dicyclohexylcarbodiimide |
| DMAP | 4-dimethylaminopyridine |
| DMDO | dimethyldioxirane |
| DMSO | dimethylsulfoxide |
| DMSO-d₆ | deuterated dimethylsulfoxide |
| DMF | dimethylformamide |
| Et | ethyl |
| EtOAc | ethyl acetate |
| Et₂O | diethyl ether |
| ESI | electrospray ionization |
| HATU | 2-(1H-7-Azabenzotriazol-1-yl)-1,1,3,3-tetramethyl uronium |
| | hexafluorophosphate |
| HPLC | High performance liquid chromatography |
| iPrOH | Isopropyl alcohol |
| Me | methyl |
| MeOH | methanol |
| m/z or m/e | mass to charge ratio |
| MH⁺ | mass plus 1 |
| MH⁻ | mass minus 1 |
| MS or ms | mass spectrum |
| Ms | methanesulfonate |
| Ph | phenyl |
| PMP | 1,2,2,6,6-pentamethylpiperidine |
| Py | Pyridyl/pyridine |
| rt or r.t. | room temperature |
| SCX | Strong cation exchange |
| TBAF | tetrabutylammonium fluoride |
| TBAI | tetra-butylammonium iodide |
| t-Bu | Tert-butyl |
| TEA | triethylamine |
| TBS | t-butyldimethylsilyl |
| TBSCl | t-butyldimethylsilyl chloride |
| TBSO | t-butyldimethylsilyloxy |
| TFA | trifluoroacetic acid |
| TfO⁻ | trifluoromethanesulfonate |
| Tf₂O | trifluoromethanesulfonyl anhydride |
| THF | tetrahydrofuran |
| TLC or tlc | thin layer chromatography |
| δ | parts per million down field from tetramethylsilane |

Compounds of the invention can be prepared according to the processes illustrated in Scheme 1.

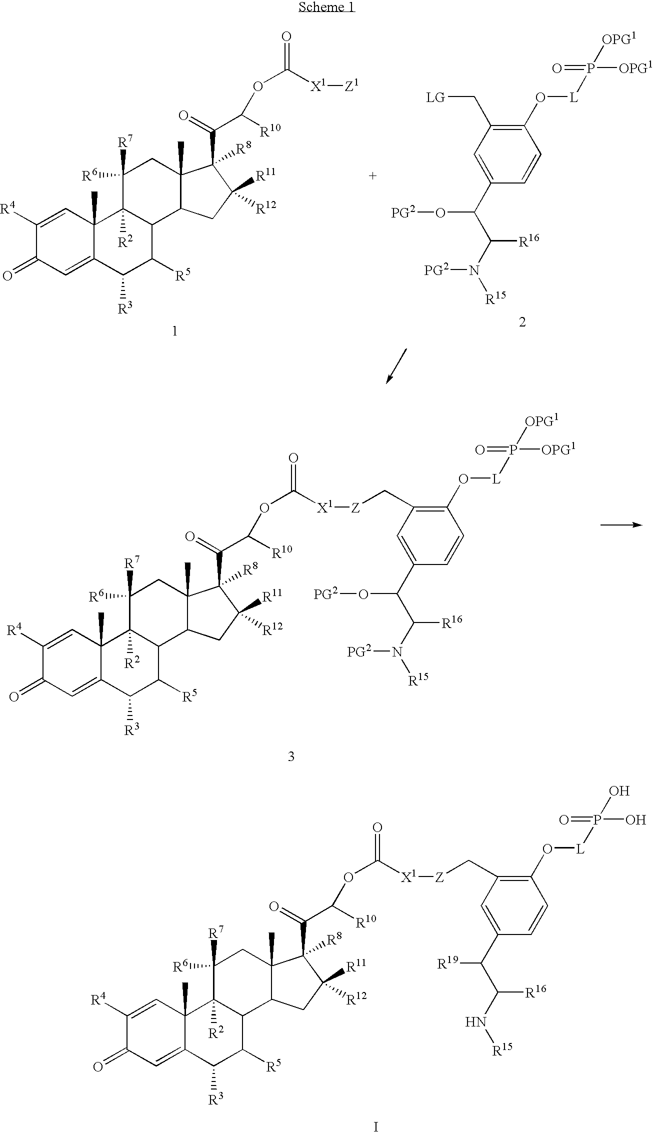

wherein:
Z¹ is NH₂, NH(C₁-C₆alkyl), NR¹⁷R¹⁸, SR¹⁷ or a 4-9 ring atom heterocyclyl wherein one ring atom is N or S;
each PG¹ is a phosphate protecting group such as methyl, ethyl, benzyl or t-butyl;
LG is a suitable leaving group, such as mesylate, triflate or iodide;
each PG² is H or Boc; and
all other variables are as defined herein.

Generally, the process comprises the steps of

a) coupling a compound of formula I with a compound of Formula 2 to prepare a compound of formula 3 or a pharmaceutically acceptable salt thereof; and
b) deprotecting the compound of formula 3 to prepare a compound of Formula I or a pharmaceutically acceptable salt thereof; and
c) optionally oxidizing the compound of Formula I wherein Z is NH₂, NH(C₁-C₆alkyl), NR¹⁷R¹⁸ or SR¹⁷ to prepare a compound of Formula I wherein Z is —NO— or —SO₂—.

More particularly, coupling a compound of formula I with a compound of formula 2 may be accomplished by activating the benzyl hydroxide of the protected, phosphorylated β-agonist of formula 3, optionally in the presence of a catalyst such as sodium iodide. The reaction may be carried out at an appropriate temperature based upon the leaving group, e.g., room temperature for mesylate or reduced temperature for the triflate. Suitable solvents include acetonitrile and methylene chloride.

The resulting compound of formula 3 may be deprotected using conventional processes, including mild acidolysis, either by brief treatment with HCl in dioxane or by low-temperature treatment with TFA in dichloromethane at about 0° C. The optimal method for removing the protecting groups may be based upon the definition of L. For example, in those embodiments wherein L is a bond, deprotection with HCl is preferred whereas in those embodiments wherein L is CH₂O, deprotection via trifluoroacetic acid may be preferred.

As will be apparent to those skilled in the art, the choice of protecting groups on the compound of formula 3 will be based at least in part on the steric bulk of the particular β-agonist side chain (R¹⁵) selected.

The foregoing process may be utilized to prepare the corresponding R-isomer of a compound of Formula II or III by substituting the R-enantiomer of the N-Boc-protected compound of formula 2 starting material for the racemate. Similarly the corresponding S-isomer of a compound of Formula II or III may be made by using the S-enantiomer of the N-Boc-protected compound of formula 2. The synthesis of an R-isomer and of an S-isomer of a compound of Formula II or III are each illustrated in the examples below. This same approach may be utilized to prepare enantiomerically enriched mixtures of any of the compounds of Formula I-1, I, II or III which contain a chiral center, and pharmaceutically acceptable salts thereof.

Compounds of formula 1 may be prepared as illustrated in Scheme 2.

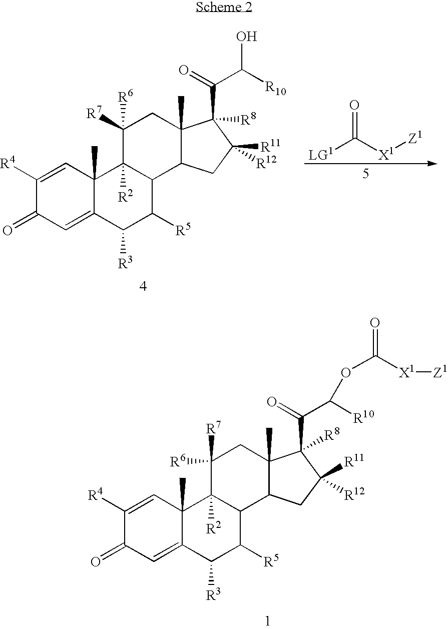

wherein

LG¹ is a suitable leaving group such as chloro or bromo or an activated ester such as 7-azabenzotriazol-1-yl;
Z¹ is NH₂, NH(C₁-C₆alkyl), NR¹⁷R¹⁸, SR¹⁷ or a 4-9 ring atom heterocyclyl wherein one ring atom is N or S; and
all other variables are as defined herein.

Generally, the process comprises reacting the compound of formula 4 with a compound of formula 5 to prepare the compound of formula 1.

More particularly, the 21-hydroxyl group of the compound of formula 4 may be derivatized with a variety of linkers through formation of an ester, carbamate or carbonate. As an example, N,N-dialkyl-a-aminoester was prepared by reacting the steroid with chloroacetyl chloride in DMF, followed by the nucleophilic substitution with a corresponding dialkylamine. Alternatively, HATU in presence of DIEA may be used as an activating reagent for 21-esterification. Carbamate linkers may be synthesized by forming the 21-chloroformate by reaction of phosgene with steroid, followed by the treatment with the appropriate amines. As another example, 21-hydroxyl moiety of steroid can be activated with p-nitrophenylchlorofortnate, followed by displacement with an alcohol yielding 21-carbonates. Compounds of formula 4 and 5 are either commercially available or may be prepared using conventional techniques.

Compounds of formula 2 may be prepared by either process illustrated in Scheme 3.

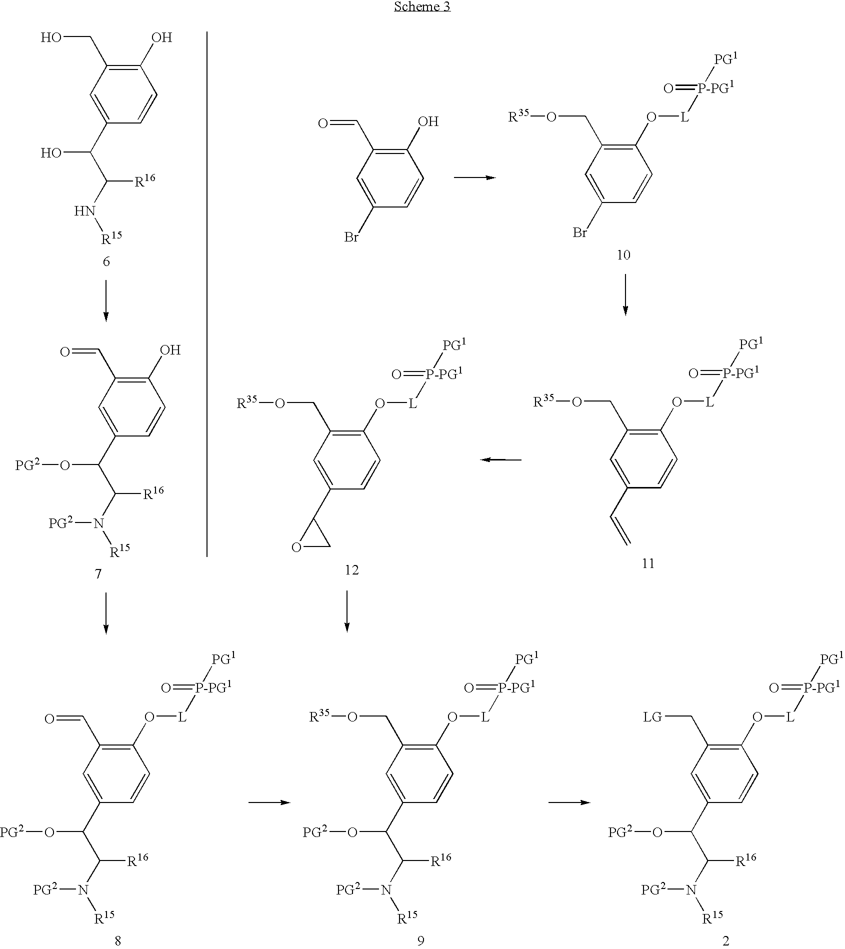

wherein
each PG¹ is an alcohol protecting group such as methyl, ethyl, butyl or t-butyl;
LG is a suitable leaving group, such as mesylate, triflate or iodide;
each PG² is H or Boc; and
R³⁵ is H or an alcohol protecting group such as tert-butyldimethylsilyl; and
all other variables are as defined herein.

In one embodiment, the process comprises the steps of:

a) oxidizing a compound of formula 6 to prepare a compound of formula 7;
b) phosphorylating the compound of formula 7 to prepare a compound of formula 8;
c) reducing the compound of formula 8 to prepare a compound of formula 9; and
d) installing a leaving group on the compound of formula 9 to prepare the compound of formula 2.

More particularly, the starting material compounds of formula 6 are either commercially available or may be prepared using conventional techniques. See, PCT Publication No. 2006/138212 to Baker et al., published 28 Dec. 2006. The compounds of formula 6 may be oxidized using conventional oxidation techniques and oxidizing agents to prepare compounds of formula 7. Suitable oxidation techniques include, for example, manganese(IV) oxide in chloroform. As will be apparent to those skilled in the art, it is desirable to install amine and/or alcohol protecting groups prior to oxidation. Suitable protecting groups include Boc. Methods are well known in the art for installing and removing such protecting groups and such conventional techniques may be employed in the instant reaction as well.

The compound of formula 7 may be phosphorylated using conventional techniques and phosphorylating agents. Examples of suitable phosphorylation techniques include but are not limited to reacting with di-t-butyl-phosphobromidate synthesized in situ in a one-pot procedure and alkylating at 50° C. with di-tert-butyl chloromethyl phosphate (Krise et al., J Med Chem (1999) 42:3094-3100).

The aldehyde moiety of the thus produced compound of formula 8 may be reduced using conventional techniques and reagents such as sodium borohydride at 0° C.

If desired, additional secondary hydroxyl protection can be introduced by reaction with excess of di-t-butyl-dicarbonate. The foregoing reduction may then be employed to prepare the primary alcohol analog of formula 9.

The installation of the leaving group on the compound of formula 9 may be accomplished using conventional techniques. For installation of the methanesulfonate leaving group, the foregoing protection strategy advantageously allows for quantitative sulfonylation carried out at room temperature, using methanesulfonyl chloride (MsCl) in the presence of 1,2,2,6,6-pentamethylpiperidine (PMP) to give the compound of formula 2 wherein LG is mesylate. In case of triflate leaving group the reaction may be carried out at 78° C. in order to minimize the formation of byproducts.

In another embodiment, the process comprises the steps of

a) phosphorylating and reducing 5-bromosalicylaldehyde to prepare a compound of formula 10;
b) reacting the compound of formula 10 under Suzuki reaction conditions to prepare a compound of formula 11;
c) reacting the compound of formula 11 with an epoxidation agent to prepare a compound of formula 12;
d) reacting the compound of formula 12 with an amine of formula H₂N—R¹⁵ to prepare a compound of formula 9; and
e) installing a leaving group on the compound of formula 9 to prepare the compound of formula 2.

In the preparation of compounds of Formula I wherein R¹⁵ is t-butyl, the steric bulk around the aminoalcohol moiety leads to a preference for the indirect synthetic approach illustrated in Scheme 3.

The syntheses starts with 5-bromosalicylaldehyde, which is phosphorylated using the techniques and reagents described above and reduced to form the alcohol. An alcohol protecting group is typically installed, such as by treatment with tert-butyldimethylsilyl chloride in the presence of imidazole, to prepare the compound of formula 10. Suzuki reaction conditions including the trivinylboroxine-pyridine complex in the presence of catalytic amounts of tricyclohexylphosphine and palladium (II) acetate may be used to introduce the vinyl substituent, thereby preparing the compound of formula 11. The compound of formula 11 then undergoes epoxidation and the epoxide then opened through nucleophilic substitution by treatment with and appropriate amine of formula NH₂—R¹⁵, in the presence of a Lewis acid such as lithium perchlorate. The epoxidation reaction may be accomplished by conventional means, including treatment with 2,2-dimethyldioxirane (DMDO) which may be conveniently generated in situ in a mixture of oxone and acetone. The nucleophilic substitution results in compounds of formula 9. Depending upon the definition of PG, the compounds of formula 9 may be acylated with, for example, di-tert-butyl dicarbonate, to install the Boc protecting group. The removal of the leaving group LG, in the compounds of formula 9 results in the compounds of formula 2, as described above.

In a particular embodiment, compounds of Formula 11:

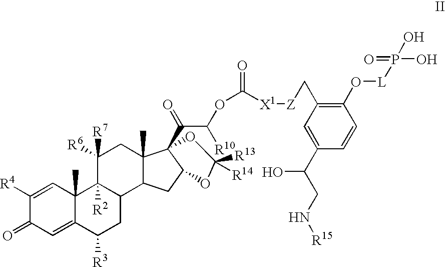

wherein:
X¹ is unsubstituted C₁alkylene;
Z is ^{⊕}(NR¹⁷R¹⁸)A⁽⁻⁾;
L is a bond; and
all other variables are as defined above,
   may be prepared by a process comprising the steps of:
   a) reducing the compound of formula 15 to prepare an activated phosphorylated β-agonist derivative of formula 16;
   b) activating the benzyl hydroxide of the compound of formula 16 and alkylating with the compound of formula 17 to prepare a compound of formula 18 (a protected derivative of the compound of Formula II); and
   c) deprotecting the compound of formula 18 to prepare a compound of Formula II-a.

This process is illustrated in Scheme 4.

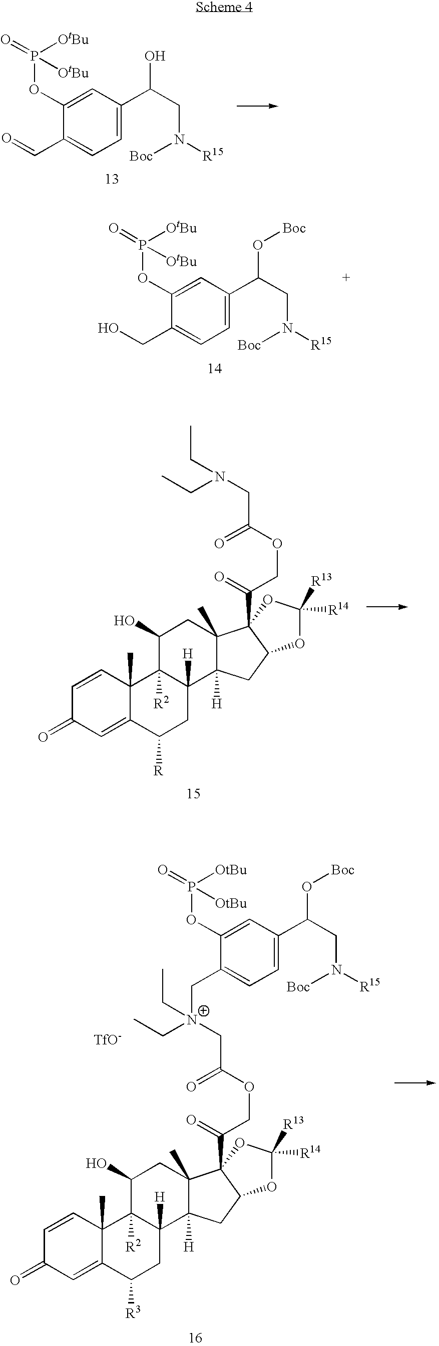

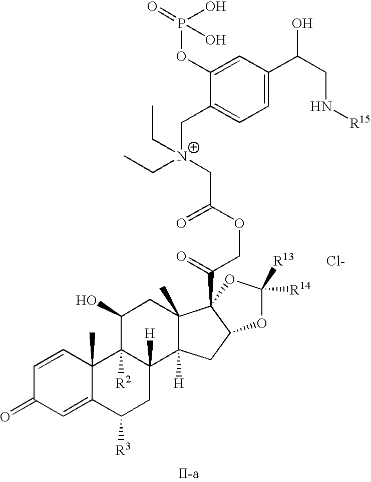

wherein all variables are as defined above.

According to this embodiment, the phosphorylated β-agonist derivative 13 may be prepared according to the process described above in Scheme 3. The 21-linked steroid derivative 15 may be prepared according to the process described above in Scheme 2.

The phosphorylated β-agonist derivative 14 may be coupled to the 21-linked steroid derivative 15 by activating the benzyl hydroxide of the protected, phosphorylated β-agonist derivative as the triflate and alkylating at −78° in CH₂Cl₂. The resulting protected product 16 may be deprotected using conventional processes, including the process described above, i.e., treatment with HCl in CH₂Cl₂.

The foregoing process may be utilized to prepare the corresponding R-isomer of a compound of Formula II by substituting the R-enantiomer of the N-Hoc-protected aldehyde 13 starting material for the racemic aldehyde. Similarly the corresponding S-isomer of a compound of Formula II may be made by using the S-enantiomer of the N-Hoc-protected aldehyde 13. The synthesis of an R-isomer and of an S-isomer of a compound of Formula II is illustrated in the examples below. This same approach may be utilized to prepare enantiomerically enriched mixtures of any of the compounds of Formula I-1, I, II or III which contain a chiral center, and pharmaceutically acceptable salts thereof.

### EXAMPLES

The foregoing may be better understood from the following examples, which are presented for the purposes of illustration and are not intended to limit the scope of the inventive concepts. The invention is defined solely by the claims which follow.

In the following examples, compounds are named using standard IUPAC naming principles where possible. The naming convention employed for the novel compounds are exemplified by the following name, [5-[1-hydroxy-2-[6-(4-phenylbutoxy)hexylamino]ethyl]-2-phosphonooxybenzyl]-(dimethyl)-[[[11β,16α]-[16,17-((R)-cyclohexylmethylene)bis(oxy)]-11-hydroxypregna-1,4-diene-3,20-dion-21-oxy]carbonyl]aminoethyl]ammonium chloride, which corresponds to the structure shown below.

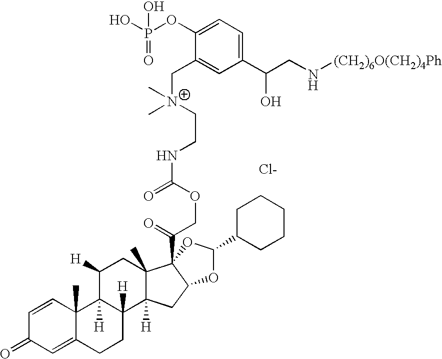

### Example 1

### [2-[4-(Di-tert-butoxyphosphoryloxy)-3-formylphenyl]-2-hydroxyethyl][6-(4-phenylbutoxy)hexyl]carbamic acid tert-butyl ester

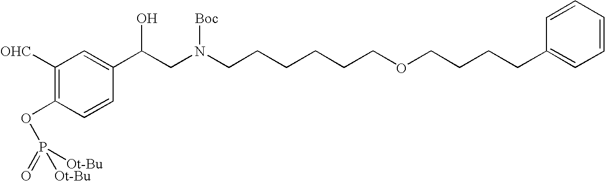

Benzyltriethylammonium chloride (334 mg, 1.46 mmol), dichloromethane (25 mL), and bromotrichloromethane (1.50 mL, 15.3 mmol), were added to a solution of sodium hydroxide (4.7 g, 120 mmol) in water (25 mL). To this biphasic mixtures vigorously stirred at 0° C., was added a solution of di-tert-butyl phosphite (2.92 mL, 14.7 mmol) in dichloromethane (25 mL) dropwise over 5 min. The reaction mixture was then allowed to warm up to room temperature while stirred vigorously for 2 h, at which point, a solution of 2-(3-formyl-4-hydroxyphenyl)-2-hydroxyethyl][6-(4-phenylbutoxy)hexyl]carbamic acid tert-butyl ester (6.03 g, 11.7 mmol) in dichloromethane (25 mL) and N,N-dimethylaminopyridine (143 mg, 1.7 mmol) was added. The mixture was then stirred for another 1 h, after which ethyl acetate (600 mL) was added and the aqueous layer was removed. The organic layer was then washed with 10% citric acid (2×100 mL), 2N NaOH (2×100 mL), dried over anhydrous sodium sulfate, filtered through a pad of activated basic alumina, and concentrated to give the title compound as clear oil.

(Yield: 7.95 g, 11.3 mmol, 95%). ³¹PNMR (CDCl₃): −15.107 ppm. LCMS: 100%, MNa⁺728.0 (exact mass 705.4 calcd for C₃₈H₆₀NO₉P). Anal. Calc: C, 64.66; H, 8.57; N, 1.98. Found: C, 64.09; H, 8.54; N, 2.02.

### Example 2

### [2-[4-(Di-tert-butoxyphosphoryloxy)-3-hydroxymethylphenyl]-2-hydroxyethyl][6-(4-phenylbutoxy)hexyl]carbamic acid tert-butyl ester

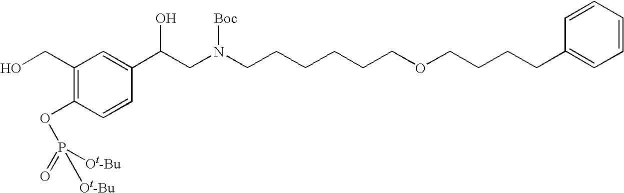

[2-[4-(Di-tert-butoxyphosphoryloxy)-3-formylphenyl]-2-hydroxyethyl][6-(4-phenylbutoxy)hexyl]carbamic acid tert-butyl ester was dissolved in THF (20 mL) and cooled to 0° C. followed by addition of NaBH₄ (354 mg, 9.36 mmol) in H₂O (4 mL). The resulting reaction mixture was stirred for 30 min then was added H₂O (50 mL). The aqueous was extracted with EtOAc (3×50 mL). The combined organic layers were washed with satd. NaHCO₃ (100 mL), brine (100 mL), dried over Na₂SO₄, and concentrated to give crude (4.69 g) alcohol title compound as a light yellow oil. ¹H NMR (CDCl₃): d 7.17-7.41 (m, 5H), 4.92 (m, 1H), 4.62 (bs, 2H), 3.39 (q, 2H), 2.64 (t 2H), 1.62 (m, 4H), 1.54 (s, 9H), 1.52 (s, 9H), 1.49 (s, 9H), 1.115-1.49 (m, 8H). ³¹PNMR (CDCl₃): −13.060 ppm. LCMS: 99%, MNa⁺ 730.0 (exact mass 707.4 calcd for C₃₈H₆₂NO₉P). Anal. Calc: C, 64.48; H, 8.83; N, 1.98. Found: C, 64.70; H, 8.84; N, 1.90.

### Example 3

### Methanesulfonic acid 5-[2-{tert-butoxycarbonyl-[6-(4-phenylbutoxy)hexyl]amino]-1-hydroxyethyl)-2-(di-tert-butoxy-phosphoryloxy)benzyl ester

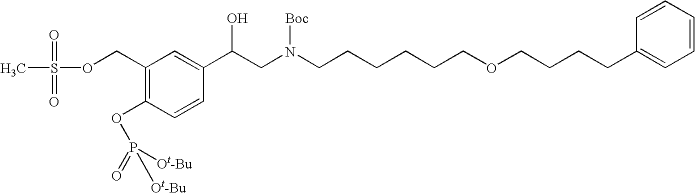

To a solution of [2-[4-(di-tert-butoxyphosphoryloxy)-3-hydroxymethylphenyl]-2-hydroxyethyl][6-(4-phenylbutoxy)hexyl]carbamic acid tert-butyl ester (described in Example 2) (1.20 g, 1.70 mmol) and 1,2,2,6,6-pentamethyl-piperidine (615 μL, 3.40 mmol) in dichloromethane (17 mL) at −78° C. was added a solution of methanesulfonic acid chloride (140 mL, 1.78 mmol) in dichloromethane (6 mL) over 5 min. Reaction stirred for 10 min at −78° C. Reaction solution was concentrated and purified by silica gel chromatography (gradient: 30% to 80% ethyl acetate in hexanes, both buffered with 1% triethylamine) to give the title compound as a clear oil (0.805 g, 1.02 mmol, 60%). ES/MS cacld. For C₃₉H₆₄NNaO₁₁PS 808.4, found m/z 808.3 (M+Na⁺)

### Example 4

### Carbonic acid 2-[tert-butoxycarbony[6-(4-phenylbutoxy)hexyl]amino]-1-[4-(di-tert-butoxyphosphoryloxy)-3-hydroxymethylphenyl]ethyl ester tert-butyl ester

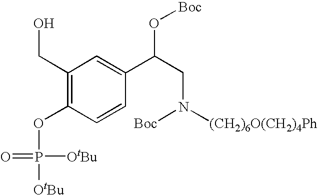

Solid (Boc)₂O (2.0 g, 9.35 mmol) DMAP (57 mg, 0.468 mmol), and pyridine (1.2 mL, 14.04 mmol) were added to a stirring solution of [2-[4-(di-tert-butoxyphosphoryloxy)-3-formylphenyl]-2-hydroxyethyl][6-(4-phenylbutoxy)hexyl]carbamic acid tert-butyl ester (described in Example 1) (3.3 g, 4.68 mmol) in CH₃CN (15 mL) at rt. The reaction mixture was stirred for 3 hr followed by addition of 10% (w/v) citric acid (50 mL). The aqueous was extracted with EtOAc (3×50 mL). The combined organic layers were washed with satd. NaHCO₃ (100 mL), brine (100 mL), dried over Na₂SO₄, and concentrated to give crude bis-Boc aldehyde. The crude was redissolved in THF (20 mL) and cooled to 0° C. followed by addition of NaBH₄ (354 mg, 9.36 mmol) in H₂O (4 mL). The resulting reaction mixture was stirred for 30 min then to this solution was added H₂O (50 mL). The aqueous layer was extracted with EtOAc (3×50 mL). The combined organic layers were washed with satd. NaBCO₃ (100 mL), brine (100 mL), dried over Na₂SO₄, and concentrated to give crude (4.69 g) alcohol as a light yellow oil. Chromatography afforded the alcohol title compound (3.0 g, 79% 2 steps) as a clear oil. ¹H NMR (400 MHz, CDCl₃) d 7.42 (d, 1H, J=15.2 Hz), 7.24-7.14 (m, 7H), 5.80 (m, 2H), 4.60 (s, 2H), 4.24 (m, 1H), 3.53 (m, 2H), 3.38 (m, 4H), 3.16 (m, 3H), 2.63 (m, 2H), 1.72-1.21 (m, 46H); ³¹P (400 MHz, CDCl₃) d −12.87.

### Example 5

### Methanesulfonic acid 5-[1-tert-butoxycarbonyloxy-2-[tert-butoxycarbonyl-[6-(4-phenylbutoxy)hexyl]amino]ethyl]-2-(di-tert-butoxyphosphoryloxy)benzyl ester

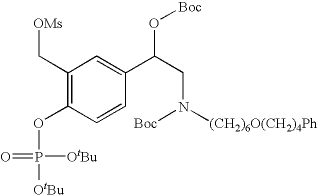

1,2,2,6,6-Pentamethylpiperidine (1.37 mL, 7.6 mmol) and methanesulfonyl chloride (0.443 mL, 5.7 mmol) were added to a stirring solution of carbonic acid [2-[tert-butoxycarbonyl[6-(4-phenylbutoxy)hexyl]amino]-1-[4-(di-tert-butoxyphosphoryloxy)-3-hydroxymethylphenyl]ethyl]ester tert-butyl ester (described in Example 4) (3.08 g, 3.8 mmol) in CH₂Cl₂ (10 mL) at rt. The reaction mixture was stirred for 30 min then quenched with 10% (w/v) citric acid (50 mL) and the aqueous layer was extracted with EtOAc (3×50 mL). The combined organic extracts were washed with satd. NaHCO₃ (100 mL), brine (100 mL), dried over Na₂SO₄, and concentrated to give crude mesylate (3.4 g). The crude title compound was used without purification in further synthesis.

### Example 6

### [2-[4-(Di-tert-butoxyphosphoryloxymethoxy)-3-formylphenyl]-2-hydroxyethyl]-[6-(4-phenylbutoxy)hexyl]carbamic acid tert-butyl ester

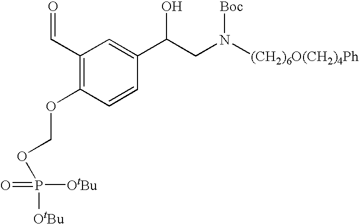

Sodium hydride (137 mg, 5.69 mmol) was cautiously added to a solution of [2-[4-(di-tert-butoxyphosphoryloxy)-3-formylphenyl]-2-hydroxyethyl][6-(4-phenylbutoxy)hexyl]carbamic acid tert-butyl ester (described in Example 1) (2.66 g, 5.17 mmol), di-tert-butyl chloromethyl phosphate (1.60 g, 6.20 mmol), and tetrabutylammonium iodide (153 mg, 0.40 mmol) in THF (50 mL) at 0° C. was cautiously added sodium hydride (137 mg, 5.69 mmol). Once bubbling ceased the reaction mixture was stirred at 50° C. overnight. Reaction mixture was cooled to it and 10% aqueous citric acid was cautiously added until bubbling ceased. The THF was removed by rotary evaporation. To this mixture was added 10% aqueous citric acid (100 mL) and the aqueous layer was washed/extracted with diethyl ether (3×100 ml). The combined organic layers were washed with 10% aqueous citric acid, water, and brine, dried over sodium sulfate, filtered and concentrated. The residue was purified by silica gel chromatography (R_{f}=0.25, 1:1 hexanes:ethyl acetate) to give the title compound as a clear oil (2.46 g, 3.34 mmol, 65%). ES/MS cacld. for C₃₉H₆₂NNaO₁₀P 758.4, found m/z=758.4 (M+Na⁺).

### Example 7

### [2-[4-(Di-tert-butoxyphosphoryloxyethoxy-3-hydroxymethylphenyl]-2-hydroxyethyl]-[6-(4-phenylbutoxy)hexyl]carbamic acid tent-butyl ester

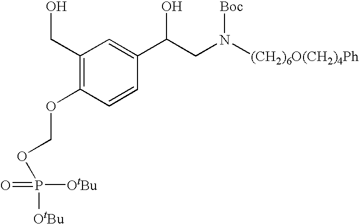

To a solution of [2-[4-(Di-tert-butoxyphosphoryloxymethoxy)-3-formylphenyl]-2-hydroxyethyl]-[6-(4-phenylbutoxy)hexyl]carbamic acid tert-butyl ester (described in Example 6) (2.46 g, 3.35 mmol) in THF (9 mL) at −78° C. was added sodium borohydride (400 mg, 10.5 mmol) followed by MeOH (0.9 mL). The reaction mixture was allowed to warm up to room temperature while stirred over 2 h. The solution was then diluted with dichloromethane and carefully quenched with 10% citric acid. The aqueous layer was extracted three times with dichloromethane and combined organic layers were washed with 10% citric acid, saturated sodium bicarbonate, brine dried over anhydrous sodium sulfate, filtered and concentrated to give the title compound as a clear oil (2.44 g, 3.31 mmol, 98%). ES/MS calcd. For C39H64NNaO10P 760.4, found m/z=760.4 (M+Na⁺).

### Example 8

### Methanesulfonic acid 5-[2-[tert-butoxycarbonyl-[6-(4-phenylbutoxy)hexyl]amino]-1-hydroxyethyl]-2-(di-tert-butoxyphosphoryloxymethoxy)-benzyl ester

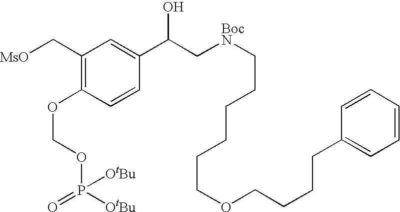

Methanesulfonyl chloride (27 mL, 0.347 mmol) was added dropwise over 5 min to a solution of [2-[4-(Di-tert-butoxyphosphoryloxynmethoxy)-3-hydroxymethylphenyl]-2-hydroxyethyl]-[6-(4-phenylbutoxy)hexyl]carbamic acid tert-butyl ester (described in Example 7) (233 mg, 0.315 mmol) and 1,2,2,6,6-pentamethyl-piperidine (114 μL, 0.630 mmol) in dichloromethane (3 mL) at −78° C. was added methanesulfonyl chloride (27 mL, 0.347 mmol) dropwise over 5 min. Reaction was stirred for 10 min at −78° C. Reaction solution was concentrated and purified by silica gel chromatography (gradient: 30% to 80% ethyl acetate in hexanes, both buffered with 1% triethylamine) to give mesylate the title compound as a clear oil (59 mg, 0.072 mmol, 23%). ES/MS cacld. For C₄₀H₆₆NNaO₁₂PS 838.4, found m/z=838.5 (M+Na⁺).

### Example 9

### Phosphoric acid 4-bromo-2-formylphenyl ester di-tert-butyl ester

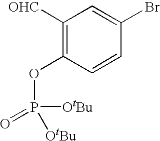

5-Bromosalicylaldehyde (8.04 g, 40 mmol) was phosphorylated analogously as described in Example 1, The crude product was purified by chromatography (9% ethyl acetate+1% triethylamine in hexane) yielding analytically pure title aldehyde as a yellowish solid (11.51 g, 73%). ¹HNMR (CDCl₃): 10.35 (s, 1H), 7.99 (d, 1H, J=2.4 Hz), 7.67 (dd, 1H, J=8.8 Hz, 2.4 Hz), 7.41 (d, 1H, J=8.8 Hz), 1.51 (s, 18H). ³¹PNMR (CDCl₃): −15.239 ppm.

LCMS: 99%, MNa⁺ 415 (exact mass 392.04 calcd for C₁₅H₂BrO₅P).

### Example 10

### Phosphoric acid 4-bromo-2-(tert-butyldimethylsilanyloxmethyl)phenyl ester di-tert-butyl ester

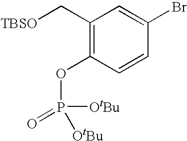

Phosphoric acid 4-bromo-2-formylphenyl ester di-tert-butyl ester (described in Example 9) was reduced to alcohol analogously as described in Example 2. The crude material was converted to the title compound by treatment with the slight excess of tert-butyldimethylsilyl chloride in DMF in presence of excess (5 equivalents) of imidazole. After the overnight reaction at room temperature the mixture was diluted with diethyl ether, washed extensively with 10% citric acid, brine and the organic phase was then dried with anhydrous magnesium sulfate, decanted and evaporated. The crude material was purified by chromatography using 10% ethyl acetate+1% triethylamine in hexane.

### Example 11

### Phosphoric acid di-tert-butyl ester 2-(tert-butyldimethylsilanyloxymethyl)-4-vinylphenyl ester

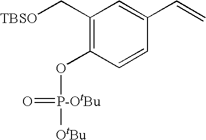

A two-neck, round bottomed flask, equipped with a reflux condenser was charged with the solution of phosphoric acid 4-bromo-2-(tert-butyldimethylsilanyloxymethyl)phenyl ester di-tert-butyl ester (described in Example 10) in a mixture of toluene (8 mL/mmol) and ethanol (1 mL/mmol) followed by adding a degassed 20% solution of potassium carbonate (8 mL/mmol). The biphasic mixture was vigorously stirred for 1 h while the stream of argon was passed through the flask. To this mixture, the trivinylboroxine-pyridine complex (1.5 equivalents) was added, followed by tricyclohexylphosphine (0.1 equivalent). The reaction mixture purged with argon once again for 30 minutes, then palladium (II) acetate (0.1 equivalents) was added, followed by vigorous stirring and heating under reflux under the positive pressure of argon for 4 h. After that time TLC analysis (chloroform/methanol 8:1) showed the complete consumption of starting material. The reaction mixture was diluted with ethyl acetate (3 times the original volume) and the organic phase was washed with water (3 times), 10% citric acid solution (twice) and brine and was dried over anhydrous MgSO₄. After filtration and evaporation of the solvent, the residue was purified by silica gel chromatography (ethyl acetate/hexanes 1:20 with 5% of triethylamine), yielding 80% of the desired title compound as a viscous oil. ¹H NMR (CDCl₃): 7.52 (s, 1H), 7.27 (d, 1H), 7.19 (d, 1H), 6.67 (dd, 1H), 5.66 (d, 1H), 5.17 (d, 1H), 4.71 (s, 2H), 1.48 (s, 18H), 0.95 (s, 9H), 0.10 (s, 6H). ³¹P NMR (CDCl₃): −14.18 ppm. LCMS: 95%, MNa⁺ 479 (exact mass 456.3 calcd for C₂₃H₄₁O₅PSi).

### Example 12

### Phosphoric acid di-tert-butyl ester 2-(tert-butyldimethylsilanyloxymethyl)-4-oxiranylphenyl ester

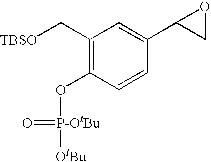

Oxone® (8 g, 13.1 mmol) was slowly added to a stirring solution of phosphoric acid di-tert-butyl ester 2-(tert-butyldimethylsilanyloxymethyl)-4-vinylphenyl ester (described in Example 11) (1.2 g, 2.63 mmol) in a CH₂C₁-/satd NaHCO₃ mixture (20 mL, 3:5) and acetone (10 mL) at 0° C. The pH of the mixture was adjusted to slightly above 7.5 with satd NaHCO₃ as needed. After stirring for 30 minutes at 0° C., then 90 minutes at room temperature the resulting suspension was extracted with CH₂Cl₂ (3×15 mL), the organic extract was dried over Na₂SO₄ and concentrated to give crude epoxide (1.3 g) as light yellow oil. Chromatography (3:1 hexanes/ethyl acetate, 0.5% Et₃N) afforded the title epoxide (0.804 g, 65%) as clear oil: ¹H NMR (400 MHz, DMSO-d₆) d 7.36 (s, 1H), 7.23 (m, 2H), 4.74 (s, 2H), 3.92 (dd, 1H, J=2.6, 4.1), 3.11 (dd, 1H, J=4.1, 5.3), 2.77 (dd, 1H, J=2.6, 5.3), 1.43 (s, 18H), 090 (s, 9H), 0.08 (s, 6H).

### Example 13

### Phosphoric acid di-tert-butyl ester 4-(2-tert-butylamino-1-hydroxyethyl)-2-(tert-butyldimethylsilanyloxymethyl)phenyl ester

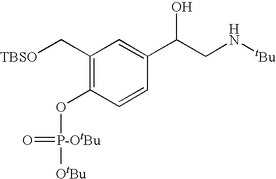

Solid LiClO₄ (180 mg, 1.7 mmol) was added at rt to a stirred solution of phosphoric acid di-tert-butyl ester 2-(tert-butyldimethylsilanyloxymethyl)-4-oxiranylphenyl ester (described in Example 12) (4 g, 8.5 mmol) in tert-butylamine (9 mL, 84 mmol). Stirring was continued for 48 h, and then the reaction mixture was diluted with ethyl acetate (20 mL). The organic layer was washed with water, brine, dried over Na₂SO₄ and concentrated to give crude aminoalcohol (5.3 g) as yellow oil. Chromatography (9:1, CH₂Cl₂/MeOH, 0.5% Et₃N) afforded the title compound (4.2 g, 91%) as light yellow oil. ¹H NMR (400 MHz, DMSO-d₆) d 7.45 (s, 1H), 7.23 (dd, 1H, J=2.1, 8.4), 7.18 (d, 1H, J=9.0), 4.75 (s, 2H), 4.49 (t, 1H, J=6.2), 3.17 (s, 1H), 2.58 (d, 2H, 1=6.3), 1.42 (m, 18H), 1.01 (d, 9H, J=14.4), 0.92 (s, 9H), 0.06 (s, 6H); ES/MS, calcd for C₂₇H₅₃NO₆PSi 546.34, found m/z 546.4 (M+H).

### Example 14

### Carbonic acid tert-butyl ester [2-tert-butylamino-1-[3-(tert-butyldimethylsilanyloxmethyl)-4-(di-tert-butoxyphosphoryloxy)phenyl]ethyl]ester

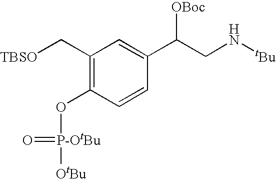

Solid (Boc)₂O (1.39 g, 6.4 mmol) was added at 0° C. to a stirred solution of phosphoric acid di-tert-butyl ester 4-(2-tert-butylamino-1-hydroxyethyl)-2-(tert-butyldimethylsilanyloxymethyl)phenyl ester (described in Example 13) (1.74 g, 3.19 mmol), PMP (1.7 mL, 9.6 mmol), and DMAP (39 mg, 0.319 mmol) in anhydrous CH₃CN (30 mL) at 0° C. After 90 minutes the reaction mixture was quenched with saturated NaHCO₃ (40 mL) and extracted with ethyl acetate (3×30 mL). The combined organic layers were washed with brine, dried over Na₂SO₄, and concentrated to give crude material (2.93 g) as white solid. Chromatography (1:3, hexanes/ethyl acetate, 0.5% Et₃N) afforded the title compound (1.21 g, 59%) as clear oil. ¹H NMR (400 MHz, DMSO-d₆) d 7.43 (s, 1H), 7.23 (m, 2H), 5.38 (dd, 1H, J=5.0, 7.7), 4.75 (s, 2H), 2.79 (m, 2H), 1.43 (s, 18H), 1.36 (s, 9H), 0.96 (s, 9H), 0.92 (s, 9H), 0.07 (m, 6H); ES/MS, calcd for C₃₂H₆₁NO₈PSi 646.39, found m/z 646.5 (M+H).

### Example 15

### Carbonic acid tert-butyl ester [2-tert-butylamino-1-[4-(di-tert-butoxyphosphoryloxy-3-hydroxymethylphenyl]ethyl]ester

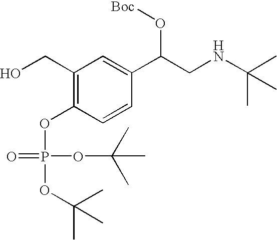

A 1.0M solution of TBAF in THF (1.4 mL, 1.4 mmol) was added to a stirred solution of carbonic acid tert-butyl ester [2-tert-butylamino-1-[3-(tert-butyldimethylsilanyloxymethyl)-4-(di-tert-butoxyphosphoryloxy)phenyl]ethyl]ester (described in Example 14) (0.9 g, 1.4 mmol) in anhydrous THF (14 mL) at rt. The resulting suspension was stirred for 1 hour, then quenched with satd NaHCO₃ (20 mL) and the aqueous layer was extracted with ethyl acetate (3×20 mL). The combined organic layers were washed with brine, dried over Na₂SO₄, and concentrated to give crude alcohol (1.01 g) as light yellow oil. Chromatography (1:3, hexanes/ethyl acetate, 0.5% Et₃N) afforded pure title compound (0.61 g, 82%) as a clear oil. ¹H NMR (400 MHz, DMSO-d₆) d 7.45 (s, 1H), 7.21 (m, 2H), 5.40 (dd, 1H, J=4.8, 8.0), 5.22 (t, 1H, J=5.6), 4.56 (d, 2H, J=5.5), 2.79 (ddd, 2H, J=6.5, 12.3, 17.1), 1.43 (m, 18H), 1.37 (s, 9H), 0.98 (s, 9H); ES/MS, calcd for C₂₆H₄₇NO₈P 532.30, found m/z=532.4 (M+H).

### Example 16

### Methanesulfonic acid 5-[2-(tert-butoxycarbonyl-tert-butylamino)-1-hydroxyethyl]-2-(di-tert-butoxyphosphoryloxy)benzyl ester

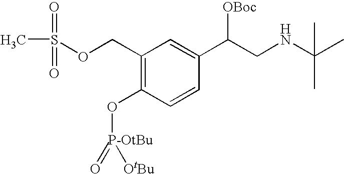

A solution of methanesulfonyl chloride (105 μL, 1.36 mmol) in CH₂Cl₂ (0.5 mL) was added dropwise at 0° C. to a stirred solution of carbonic acid tert-butyl ester [2-tert-butylamino-1-[4-(di-tert-butoxyphosphoryloxy)-3-hydroxymethylphenyl]ethyl]ester (described in Example 15) (0.6 g, 1.13 mmol) and PMP (817 μL, 4.52 mmol) in CH₂Cl₂ (12 mL) at 0° C. The reaction mixture was stirred for 30 minutes then quenched with satd NaHCO₃ (20 mL). The organic layer was separated, dried over Na₂SO₄, and concentrated to give crude mesylate (0.98 g) as light yellow oil. Chromatography (1:3, hexanes/ethyl acetate, 0.5% Et₃N) afforded the title compound (0.56 g, 76%) as a clear oil. ES/MS, calcd for C₂₇H₄₉NO₁₀PS 610.28, found m/z=610.4 (M+H).

### Example 17

### Phosphoric acid 4-bromo-2-formylphenoxymethyl ester di-tert-butyl ester

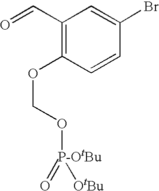

The title compound can be synthesized in a manner analogous to that described in Example 6, using 5-bromosalicaldehyde as a starting material.

### Example 18

### Phosphoric acid 4-bromo-2-(tert-butyldimethylsilanyloxy-methyl)-phenoxymethyl ester di-tert-butyl ester

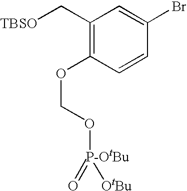

The title compound can be synthesized in a manner analogous to that described in Example 10, using the aldehyde prepared as described in Example 17a starting material.

### Example 19

### Phosphoric acid di-tert-butyl ester 2-(tert-butyldimethylsilanyloxymethyl)-4-vinylphenoxymethyl ester

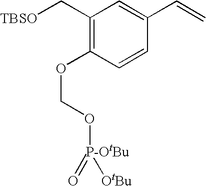

The title compound can be synthesized by the Suzuki vinylation analogous to that described in Example 11 using phosphoric acid 4-bromo-2-(tert-butyldimethylsilanyloxymethyl)-phenoxymethyl ester di-tert-butyl ester (described in Example 18) as a starting material.

### Example 20

### Phosphoric acid di-tert-butyl ester 2-(tert-butyldimethylsilanyloxymethyl)-4-oxiranylphenoxymethyl ester

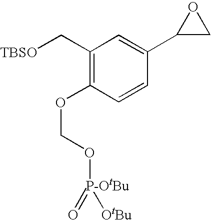

The title compound can be synthesized through epoxidation in a manner analogous to that described in Example 12, using phosphoric acid di-tert-butyl ester 2-(tert-butyldimethylsilanyloxymethyl)-4-vinylphenoxymethyl ester (described in Example 19) as a starting material.

### Example 21

### Phosphoric acid di-tert-butyl ester 4-(2-tert-butylamino-1-hydroxyethyl)-2-(tert-butyldimethylsilanyloxymethyl)phenoxymethyl ester

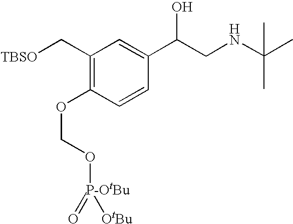

The title compound may be prepared by the aminolysis with t-butylamine in a manner analogous to that described in Example 13, using phosphoric acid di-tert-butyl ester 2-(tert-butyldimethylsilanyloxymethyl)-4-oxiranylphenoxymethyl ester (described in Example 20) as a substrate.

### Example 22

### Carbonic acid tert-butyl ester [2-tert-butylamino-1-[3-(tert-butyldimethylsilanyloxymethyl)-4-(di-ter-butoxyphosphoryloxymethoxy)phenyl]ethyl]ester

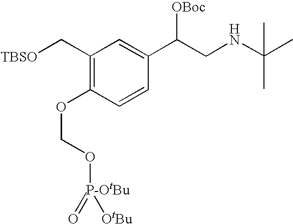

The O-acylation of phosphoric acid di-tert-butyl ester 4-(2-tert-butylamino-1-hydroxyethyl)-2-(tert-butyldimethylsilanyloxymethyl)phenoxymethyl ester (described in Example 21) can be accomplished in a manner analogous to that described in Example 14.

### Example 23

### Carbonic acid tert-butyl ester [2-tert-butylamino-1-[4-(di-tert-butoxyphosphoryloxmethoxy)-3-hydroxymethylphenyl]ethyl]ester

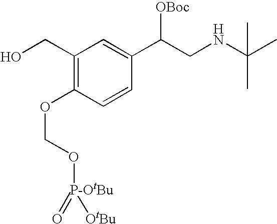

The TBS-removal from carbonic acid tert-butyl ester [2-tert-butylamino-1-[3-(tert-butyldimethylsilanyloxymethyl)-4-di-tert-butoxyphosphoryloxymethoxy)phenyl]ethyl]ester (described in Example 22) can be achieved in a manner analogous to that described in Example 15.

### Example 24

### Methanesulfonic acid 5-(1-tert-butoxycarbonyloxy-2-tert-butylaminoethyl)-2-(di-tert-butoxyphosphoryloxymethoxy)benzyl ester

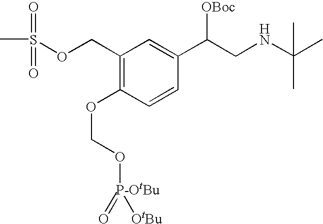

Title compound may be synthesized in a manner analogous to that described in Example 16, using the aminoalcohol carbonic acid tert-butyl ester [2-tert-butylamino-1-[4-(di-tert-butoxyphosphoryloxymethoxy)-3-hydroxymethylphenyl]ethyl]ester (described in Example 23) as a substrate.

### Example 25

### 1-Methyl-4-piperidinecarboxylic acid [[[11β,16α]-[[((R)-cyclohexylmethylene) bis(oxy)]-11-hydroxypregna-1,4-diene-3,20-dion-21-yl]ester

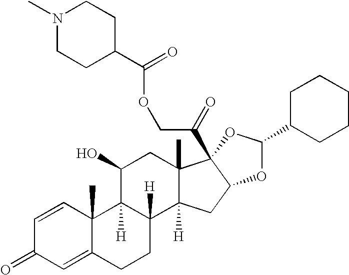

Neat DIEA (0.740 mL, 4.25 mmol) and HATU (0.970 g, 2.55 mmol) were added at rt to a stirred solution of 1-methylpiperidine-4-carboxylic acid (0.335 g, 2.34 mmol) in acetonitrile (10 mL). After 10 min des-CIC (desisobutyryl ciclesonide)(1.0 g, 2.12 mmol) was added in one portion. After stirring for 14 h the reaction mixture was poured into H₂O (50 mL), stirred 15 min and then extracted with DCM (3×10 mL). Combined DCM extracts were dried (MgSO₄), filtered and concentrated to provide crude ester (2.15 g) as a light yellow solid. Chromatography (DCM/MeOH gradient 1:0 to 4:1) afforded the title compound (1.106 g, 87% yield) as off-white solid. ¹H NMR (400 MHz, CDCl₃) d 7.30 (d, J=10.1 Hz, 1H), 6.15 (dd, J=10.1, 1.9 Hz, 1H), 5.90 (s, 1H), 4.95 (d, J=17.7 Hz, 1H), 4.82 (d, J=3.9 Hz, 1H), 4.77 (d, J=17.7 Hz, 1H), 4.68 (d, J=4.2 Hz, 1H), 4.37 (d, J=4.2 Hz, 1H), 4.33-4.26 (m, 1H), 2.73-2.67 (m, 2H), 2.55-2.48 (m, 1H), 2.42-2.21 (m, 2H), 2.12 (s, 3H), 2.10-1.87 (m, 4H), 1.86-1.77 (m, 4H), 1.74-1.45 (m, 11H), 1.37 (s, 3H), 1.23-0.80 (m, 10H); ES/MS cacld for C₃₅H₄₉NO₇ 595.4, found m/z=596.3 (M+H).

### Example 26

### Carbonic acid [[11β,16α]-[((R)-cyclohexylmethylene)bis(oxy)]-11-hydroxypregna-1,4-diene-3,20-dion-21-yl]ester 2-diethylaminoethyl ester

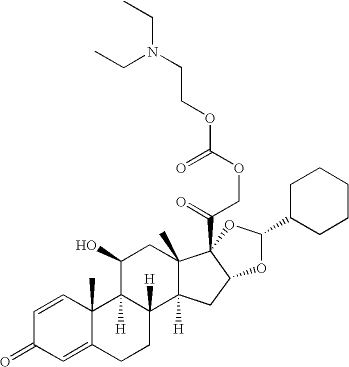

2-Diethylamino-ethanol (1.0 mL, 7.1 mmol) was dissolved in 7 mL of anhydrous THF. After flushing with N₂ and cooling to 0° C., 1.2 g of 1,1′-carbonyldiimidazole was added under a strong N₂ flow, followed by DIEA (1.11 mL, 10.7 mmol) added via syringe. The solution was allowed to warm to rt overnight, and then des-CIC (471 mg, 1.0 mmol) was added under N₂, and the reaction mixture was stirred at rt for 6 h. The solvent was evaporated, and the resulting residue was partitioned between EtOAc and water. After separation the aqueous layer was then extracted with EtOAc (twice), combined with extracts, washed with brine, dried over MgSO₄ and concentrated to give a clear residue. This material was purified by silica gel chromatography (0 to 50% MeOH in CH₂Cl₂ buffered with 0.5% TEA) to give 62 mg title compound as a white amorphous powder after concentration and lyophilization from water. ES/MS Calc. for C₃₅H₅₁NO₈=613.36, found m/z 614.2 [M+H].

### Example 27

### 4-Dimethylaminobutyric acid [[11β,16α]-[[((R)-cyclohexylmethylene)bis(oxy)]-11-hydroxypregna-1,4-diene-3,20-dion-21-yl]]ester

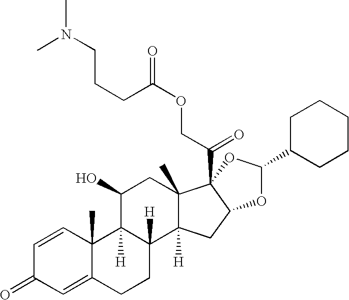

4-Dimethylamino-butyric acid hydrochloride (587 mg, 3.5 mmol) was suspended in 12 mL of anhydrous EtOAc in an oven-dried N₂ flushed, round bottom flask. After cooling to 0° C., DIEA (580 uL, 3.5 mmol) was added dropwise via syringe, followed by 3.5 mL DCC (1.0 M in CH₂Cl₂), and then pentafluorophenol (644 mg, 3.5 mmol). The reaction was allowed to warm to rt, and stirred overnight. The resulting opaque pink suspension was filtered and the filtrate used crude in the next step. Des-CIC (494 mg 1.05 mmol) and DIEA (232 uL, 1.4 mmol) were added at rt and the reaction mixture was stirred overnight, washed with water and brine, dried over MgSO₄, and concentrated to give white foam which was ˜90% pure according to LC/MS. TLC (70:30 EtOAc:Hexanes, product R_{f}=0.4). This material was purified by chromatography (0 to 100% EtOAc in hexanes), concentrated and lyophilized from a mixture of water and CH₃CN to give 545 mg (89%) of the title compound as a white amorphous powder. ES/MS Calc. for C₃₄H₄₉NO₇=583.35, found m/z 584.8 [M+H].

### Example 28

### Carbonic acid diethylaminoethyl ester [[11β,16α]-[((R-cyclohexylmethylene)bis(oxy)]-11-hydroxypregna-1,4-diene-3,20-dion-21-yl]ester 1-methyl-2-dimethylaminoethyl ester

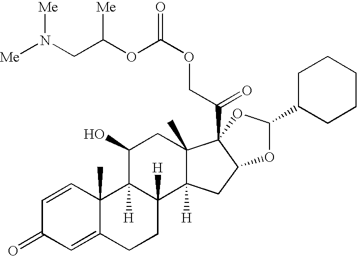

The title compound was prepared in an analogous manner to Example 26, using 580 μL of 1-dimethylamino-propan-2-ol. Purification (0 to 10% MeOH in CH₂Cl₂, followed by 20 to 85% EtOAc in hexanes) gave 85 mg of white amorphous powder after lyophilization. (14% yield). ES/MS Calc. for C₃₄H₄₉NO₈=599.35, found m/z 600.5 [M+H].

### Example 29

### Nicotinic acid [[11β,16α]-[[((R)-cyclohexylmethylene)bis(oxy)]-11-hydroxypregna-1,4-diene-3,20-dion-21-yl]]ester

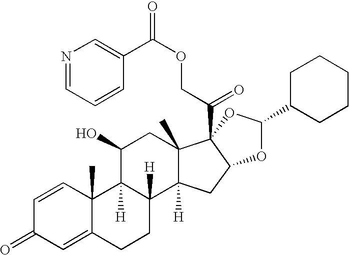

Desisobutyryl ciclesonide (750 mg, 1.6 mmol) was dissolved in 10 mL of dry CH₂Cl₂, followed by addition of TEA (550 μL, 5.4 mmol) via syringe, then nicotinoyl chloride (as hydrochloride; 313 mg, 1.76 mmol), and catalytic amount of DMAP. After stirring for 8 h at rt the reaction mixture was washed with water (twice), sat. NaHCO₃ (twice), brine and dried over anhydrous MgSO₄. The title compound (0.9 g) was obtained after decanting and evaporation, as a white amorphous solid (97% yield). ES/MS Calc. for C₃₄H₁₄₁N O₇=575.29, found m/z 576.3 [M+H].

### Example 30

### 4-Diethylaminomethylbenzoic acid [[11β,16α]-[[((R)-cyclohexylmethylene)bis(oxy)]-11-hydroxypregna-1,4-diene-3,20-dion-21-yl]]ester

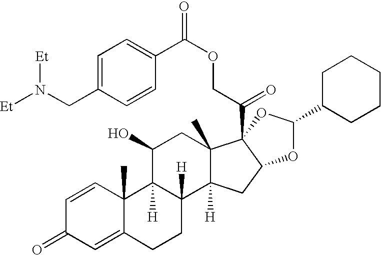

Desisobutyryl ciclesonide (1.0 g, 21.2 mmol) was dissolved in 50 mL of dry CH₂Cl₂ and cooled to 0° C. under N₂. 4-Chloromethyl-benzoyl chloride (442 mg, 2.34 mmol) and DIEA (527 uL, 3-18 mmol) were then added, and the reaction mixture was allowed to warm to it overnight. After diluting with water and separation, the organic layer was washed with water, satd. NaHCO₃ (twice), dried over MgSO₄ and concentrated to give the chloromethyl intermediate as a yellow foam (1.3 g). That material was then dissolved in anhydrous acetone, and diethylamine (1.8 mL, 16.7 mmol) together with NaI (313 mg, 2.09 mmol) were then added, and the reaction mixture heated under reflux for 4 hr. After cooling to rt, the acetone was evaporated, and the residue was partitioned between EtOAc and water. The organic layer was washed with water, and brine (twice), dried over MgSO₄ and concentrated to give a yellow residue, which was recrystallized from EtOH/H₂O to give the title compound as light tan needles. (870 mg, 63% yield). mp=123.5° C. (decomp). TLC (70:30 EtOAc hexanes) R_{f}=0.2. ES/MS Calc. for C₄₀₁H₅₃NO₇=659.38, found m/z 660.5 [M+H].

### Example 31

### 3-Diethylaminomethylbenzoic acid [[11β,16α]-[[((R)-cyclohexylmethylene)bis(oxy)]-11-hydroxypregna-1,4-diene-3,20-dion-21-yl]]ester

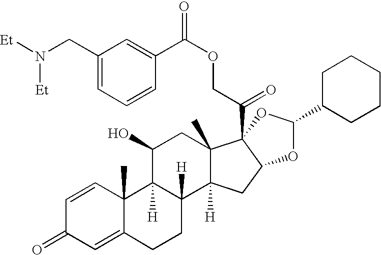

The title compound was prepared in a manner analogous to that described in Example 30, using 3-chloromethyl-benzoyl chloride (440 mg) as substrate. Crude product was purified by chromatography (0 to 100% EtOAc in hexanes) to provide a yellow solid (820 mg, 52% yield). ES/MS Calc. for C₄₀H₅₃NO₇=659.38, found m/z 660.5 [M+H].

### Example 32

### 4-Diethylaminoacetic acid [[11β,16α]-[[((R)-cyclohexylmethylene)bis(oxy)]-11-hydroxypregna-1,4-diene-3,20-dion-21-yl]]ester

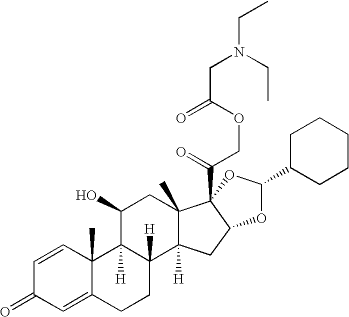

Chloroacetyl chloride (1.7 mL, 21.2 mmol) was added to a stirred solution of desisobutyryl ciclesonide (1 g, 2.1 mmol) in DMF (10 mL). The reaction mixture was stirred for 30 min at rt and then poured into H₂O (100 mL). The resulting suspension was filtered and filter cake was washed with H₂O (50 mL), then dried to give crude chloroester (1.22 g) as yellow solid. That material was redissolved in acetone (50 mL) followed by addition of NaI (334 mg, 2.2 mmol) and diethylamine (2.3 mL, 22 mmol). The resulting mixture was refluxed for 30 min, cooled, then poured into H₂O (100 mL). The aqueous layer was extracted with EtOAc (3×50 mL) and the combined organic layers were washed with brine (50 mL), dried over Na₂SO₄, and concentrated to give crude amino ester (1 g) as yellow oil. Crystallization from EtOAc and hexanes gave the title compound (0.72 g, 58% after 2 steps) as off-white crystals. ¹H NMR (400 MHz, CDCl₃) d 6.30 (dd, 1N, J=1.9, 10.1 Hz), 6.05 (s, 1H), 4.96 (d, 1H, J=17.6 Hz), 4.84 (d, 1H, J=5.0 Hz), 4.74 (d, 1H. J=17.6 Hz), 4.52 (brm, 1H), 4.35 (d, 1H, J=4.6 Hz), 3.46 (d, 24, J=2.1 Hz), 2.69 (q, 4H, J=7.1 Hz), 2.57 (m, 1H), 2.35 (m, 1H), 2.25-2.02 (m, 3H), 1.88 (dd, 1H, J=2.58, 14.03 Hz), 1.8-1.51 (m, 10H), 1.47 (s, 3H), 1.37-0.99 (m, 14H). 0.97 (s, 3H); ES/MS calcd for C₃₄H₅₀NO₇ 584.4, found m/z=584.4 (MH⁺)

### Example 33

### 3-(Pyridin-3-yl)acrylic acid [[11β,16a]-[[((R)-cyclohexylmethylene) bis(oxy)]-11-hydroxypregna-1,4-diene-3,20-dion-21-yl]]ester

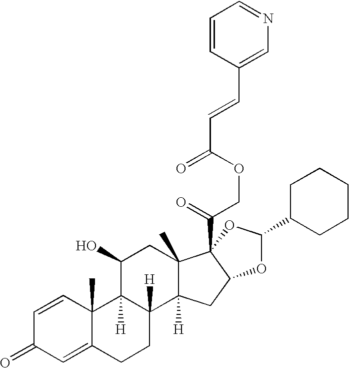

Diisopropylethyl amine (DIEA, 0.554 mL, 3.18 mmol) and HATU (0.970 g, 2.55 mmol) were added at rt to a stirred solution of 3-(pyridin-3-yl)acrylic acid (0.38 g, 2.55 mmol) in DMF (10 mL) at rt. After 10 min the desisobutyryl ciclesonide (1.0 g, 2.12 mmol) was added in one portion. After stirring for 14 h at it, the reaction mixture was poured into H₂O (50 mL) and then filtered to give crude ester (1.28 g) as a light yellow solid. Chromatography (1:3, hexanes/ethyl acetate, 0.5% Et₃N) afforded the title compound (0.461 g, 36%) as white solid. ¹H NMR (400 MHz, CDCl₃) d 8.80 (d, 1H, J=2.0 Hz), 8.64 (dd, 1H, J=1.5, 4.8 Hz), 7.88 (dt, 1H, J=1.9, 8.0 Hz), 7.78 (d, 1H, J=16.1 Hz), 7.37 (dd, 1H, J=4.8, 7.9 Hz), 7.30 (d, 1H, J=10.1 Hz), 6.64 (d, 1H, J=16.1 Hz), 6.31 (dd, 1H, J=1.9, 10.1 Hz), 6.05 (s, 1H), 4.97 (dd, 2H, J=16, 68 Hz), 4.88, (d, 1H, J=4.6 Hz), 4.56 (s, 114), 4.39 (d, 1H, J=4.6 Hz), 2.59 (m, 114), 2.36 (m, 1H), 2.28-1.86 (m, 5H), 1.85-1.55 (m, 9H), 1.37-1.05 (m, 8H), 1.02 (s, 3H), 0.89 (t, 2H, J=6.9); ES/MS cacld for C₃₆H₄₄NO₇ 602.3, found m/z 602.3 (MH⁺).

### Example 34

### 3-(Pyridin-3-yl)propionic acid [[11β,16α]-[[((R)-cyclohexylmethylene)bis(oxy)]-11-hydroxypregna-1,4-diene-3,20-dion-21-yl]]ester

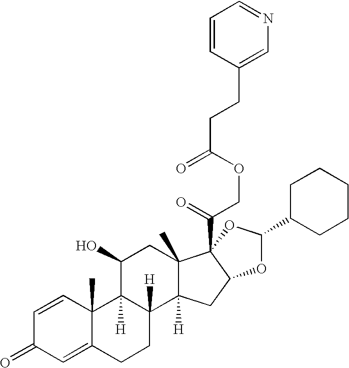

The title compound was synthesized in a manner analogous to that described in Example 33, using 3-(pyridin-3-yl)propanoic acid in place of 3-(pyridin-3-yl)acrylic acid. ¹H NMR (400 MHz, CDCl₃) d 8.49 (m, 2H), 7.55 (m, 1H), 7.28 (m, 2H), 6.29 (m, 1H), 6.03 (d, 1H, J=1.0 Hz), 4.84 (d, 1H, J=3.8 Hz), 4.80 (dd, 2H, J=17.6, 64 Hz), 4.52 (m, 1H), 4.33 (s, 1H), 3.01 (t, 2H, J=7.5 Hz), 2.78 (dd, 2H, J=17.6, 15.0 Hz), 2.57 (m, 1H), 2.34 (m, 1H), 2.05 (s, 4H), 1.97-0.87 (m, 23H); ES/MS cacld for C₃₆H₄₆NO₇ 604.3, found m/z=604.3 (MH⁺).

### Example 35

### (S)-2-Dimethylaminopropionic acid [[11β,16α]-[[((R)-cyclohexylmethylene)bis(oxy)]-11-hydroxypregna-1,4-diene-3,20-dion-21-yl]]ester

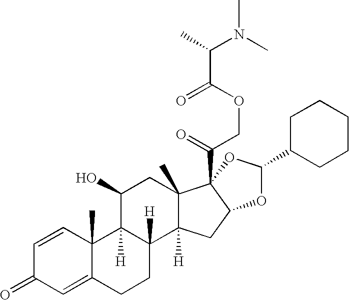

The title compound may be synthesized in a manner analogous to that described in Example 33, using (S)—N,N-dimethyl-alanine in place of 3-(pyridin-3-yl)acrylic acid.

### Example 36

### (S)-3-Dimethylaminopropionic acid [[11β,16α]-[[((R)-cyclohexylmethylene)bis(oxy)]-11-hydroxypregna-1,4-diene-3,20-dion-21-yl]]ester

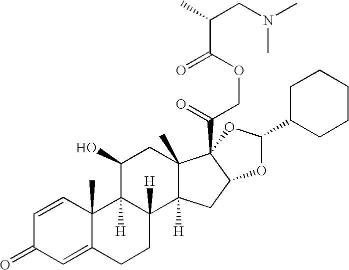

The title compound may be synthesized in a manner analogous to that described in Example 33, using (R)-3-dimethylamino-2-methyl-propionic acid in place of 3-(pyridin-3-yl)acrylic acid.

### Example 37

### 1-Dimethylaminomethylcyclopropanecarboxylic acid [[11β,16α]-[[((R)-cyclohexylmethylene)bis(oxy)]-11-hydroxypregna-1,4-diene-3,20-dion-21-yl]]ester

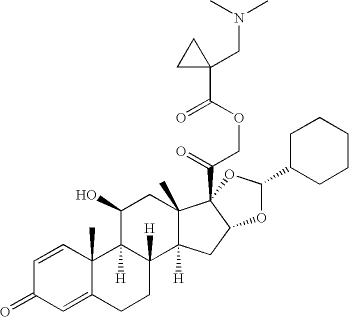

The title compound may be synthesized in a manner analogous to that described in Example 33, using 1-dimethylaminomethyl-cyclopropanecarboxylic acid in place of 3-(pyridin-3-yl)acrylic acid.

### Example 38

### (S) 1-Methylpyrrolidinecarboxylic acid [[11β,16α]-[[((R)-cyclohexylmethylene)bis(oxy)]-11-hydroxypregna-1,4-diene-3,20-dion-21-yl]]ester

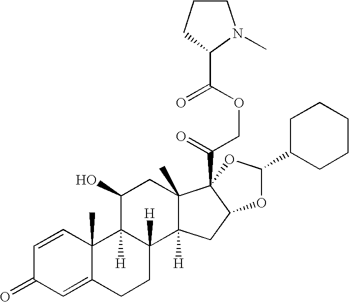

The title compound was synthesized in a manner analogous to that described in Example 33, using N-methyl-proline in place of 3-pyridin-3-yl-acrylic acid. ¹H NMR (400 MHz, CDCl3) d 7.27 (m, 1H), 6.29 (dd, 1H, J=1.9, 10.1 Hz), 6.03 (s, 1H), 5.00 (d, 1H, J=17.6 Hz), 4.82 (d, 1H, J=4.9 Hz), 4.72 (d, 1H, J=17.6 Hz), 4.50 (s, 1H), 4.33 (d, 1H, J=4.6 Hz), 3.17 (m, 1H), 3.07 (dd, 1H, J=7.5, 9.0 Hz), 2.57 (m, 1H), 2.46 (s, 3H), 2.36-0.88 (m, 33H); ES/MS cacld for C₃₄H₄₈NO₇ 582.3, found m/z=582.3 (MH⁺).

### Example 39

### 2-Dimethylaminoethylcarbamic acid [[11β,16α]-[[((R)-cyclohexylmethylene)bis(oxy)]-11-hydroxypregna-1,4-diene-3,20-dion-21-yl]]ester

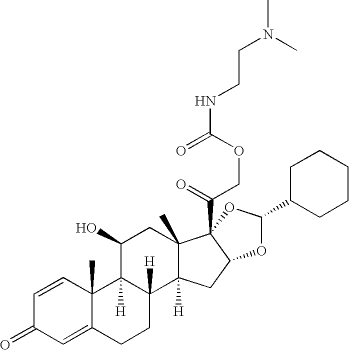

Phosgene (20% solution in toluene, 5.25 mL, 10.6 mmol) was added at rt to a stirred solution of des-CIC (1.0 g, 2.12 mmol) in THF (20 mL) at it. The resulting mixture was stirred for 30 min and then concentrated to give crude 21-chloroformate. That material was dissolved in CH₃CN (20 mL) and N,N-dimethylethylamine (0.463 mL, 4.24 mmol) was added at rt. The suspension was stirred for 1 hr then quenched with satd. NaHCO₃ (50 mL) and the resulting mixture was extracted with EtOAc (3×50 mL). The combined organic layers were washed with brine (50 mL), dried over Na₂SO₄, and concentrated to give crude carbamate (1.5 g) as yellow oil. Chromatography (9:1, CH₂Cl₂/MeOH, 0.5% Et₃N) afforded title compound (0.602 g, 48% from 2 steps) as a light yellow solid. ¹H NMR (400 MHz, CDCl₃) d 7.28 (d, 1H, J=9.3 Hz), 6.29 (dd, 1H, J=1.9, 10.1 Hz), 6.03 (s, 1H), 5.73 (m, 1H), 4.85 (m, 2H), 4.68 (d, 1H, J=18.0 Hz), 4.51 (s, 1H), 4.33 (d, 1H, J=4.5 Hz), 3.33 (dd, 2H, J=5.6, 11.4 Hz), 2.56 (m, 4H), 2.34 (m, 6H), 2.05 (m, 3H), 1.86 (m, 1H), 1.81-0.86 (m, 23H); ES/MS cacld for C₃₃H₄₉N₄O₇ 585.4, found m/=585.3 (MH⁺).

### Example 40

### 2-Dimethylaminoethyl(methyl)carbamic acid [[11β,16α]-[[((R) -cyclohexylmethylene)bis(oxy)]-11-hydroxypregna-1,4-diene-3,20-dion-21-yl]]ester

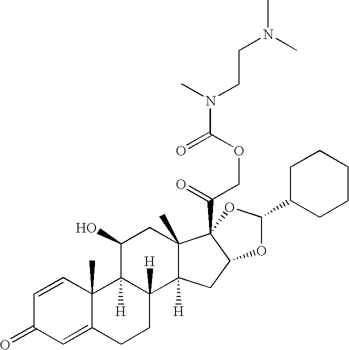

The title compound was synthesized in a manner analogous to that described in Example 39, using N,N,N′-trimethylethylene-1,2-diamine in place of N,N-dimethylethylamine. ¹H NMR (400 MHz, CDCl3) d 7.27 (m, 1H), 6.29 (dd, 1H, J=1.9, 10.1 Hz), 6.03 (m, 1H), 4.93 (d, 1H, J=17.9 Hz), 4.84 (s, 1H), 4.71 (s, 1H), 4.50 (s, 1H), 4.35 (d, 1H, J=4.6 Hz), 3.44 (m, 2H), 3.00 (d, 3H, J=21.1), 2.56 (m, 4H), 2.31 (m, 6H), 2.06 (m, 3H), 1.87 (dd, 1H, J=2.5, 14.0 Hz), 1.81-0.87 (m, 23H); ES/MS cacld for C₃₄H₅₁N₂O₇ 599.4, found m/z=599.4 (MH⁺).

### Example 41

### 4-Methylpiperazinecarboxylic acid [[11β,16α]-[[((R)-cyclohexylmethylene)bis(oxy)]-11-hydroxypregna-1,4-diene-3,20-dion-21-yl]]ester

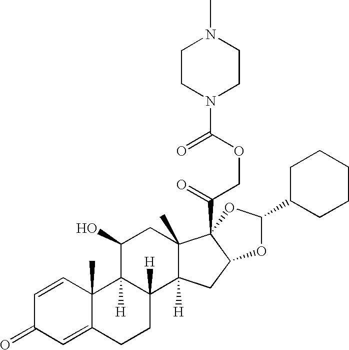

The title compound was synthesized in a manner analogous to that Example 39, using N-methyl piperazine in place of N,N-dimethyl-ethylamine. ¹H NMR (400 MHz, CDCl33) d 7.26 (m, 1H), 6.28 (d, 1H, J=10.1 Hz), 6.03 (s, 1H), 4.90 (d, 1H, J=18.0 Hz), 4.85 (d, 1H, J=4.7 Hz), 4.70 (d, 1H, J=18.0 Hz), 4.50 (s, 1H), 4.34 (d, 1H, J=4.6 Hz), 3.54 (brd, 2H, J=13.0 Hz), 2.56 (td, 1H, J=5.3, 13.5H), 2.40 (s, 3H), 2.36 (m, 1H), 2.31 (s, 3H), 2.04 (m, 4H), 1.86-0.87 (m, 26H); ES/MS cacld for C₃₄H₄₉N₂O₇ 597.4, found z/s=597.3 (MH⁺).

### Example 42

### Carbonate diethylaminoethyl ester [[6α,11β,16α]-[6,9-difluoro-11-hydroxy-16,17-(1-methylidene)bis(oxy)pregna-1,4-diene-3,20-dion-21-yl]]ester

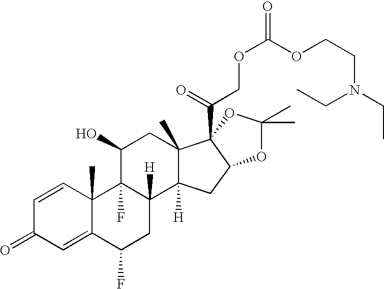

Pyridine (0.107 mL, 1.33 mmol) and p-nitrophenyl chloroformate (268 mg, 1.33 mmol) were added to a stirred solution of fluocinolone acetonide (300 mg, 0.662 mmol) in CH₂Cl₂ (6 mL) at 0° C. The reaction mixture was stirred for 90 min and then concentrated to give crude p-nitrophenylcarbonate. Chromatography (1:1, hexanes/ethyl acetate) afforded pure intermediate (340 mg. 83%) as white solid. That intermediate (300 mg, 0.486 mmol) was then dissolved in CH₂Cl₂ (5 mL), followed by addition of DMAP (71 mg, 0.583 mmol) and 2-diethylamino-ethanol (0.077 mL, 0.883 mmol) at 0° C. The resulting mixture was stirred for 4 days at it and then concentrated to give crude product. Chromatography (1:3, hexanes/ethyl acetate, buffered with 0.5% Et₃N) afforded the title compound (163 mg, 56%) as a white solid.

¹H NMR (400 MHz, DMSO-d₆) d 7.27 (dd, 1H, J=1.3, 10.2 Hz), 6.30 (dd, 1H, J=1.9, 10.2 Hz), 6.11 (s, 1H), 5.63 (m, 1H), 5.57 (m, 1H), 5.17 (d, 1H, J=18.1 Hz), 4.89 (d, 1H, J=4.4 Hz), 4.76 (d, 1H, J=18.1 Hz), 4.21 (m, 1H), 4.14 (t, 2H, J=6.0 Hz), 2.64 (m, 2H), 2.25 (d, 1H, J=10.9 Hz), 2.08 (m, 6H), 2.01 (m, 2H), 1.72 (d, 1H, J=12.9 Hz), 1.58 (m, 2H), 1.49 (s, 3H), 1.34 (s, 3H, J=10.2 Hz), 1.15 (m, 3H), 0.94 (t, 6H, J=7.1 Hz), 0.83 (s, 3H).

### Example 43

### 2-Diethylaminoacetic acid [[11β,16α]-[[((16,17-(butylidenedioxy)]-11-hydroxypregna-1,4-diene-3,20-dion-21-yl]]ester

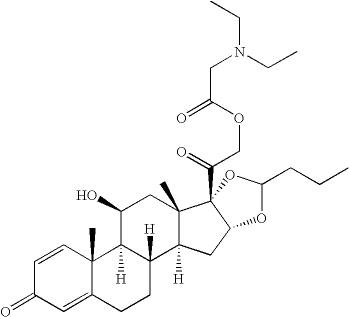

The title compound was synthesized in a manner analogous to that described in Example 32, using budesonide in place of desisobutyryl ciclesonide. ES/MS cacld for C₃₁H₄₆NO₇ 544.3, found m/z=544.3 (MH⁺).

### Example 44

### 3-(Pyridin-3-yl)acrylic acid [[11β,16α]-[[((16,17-(butylidenedioxyl)]-11-hydroxypregna-1,4-diene-3,20-dion-21-yl]]ester

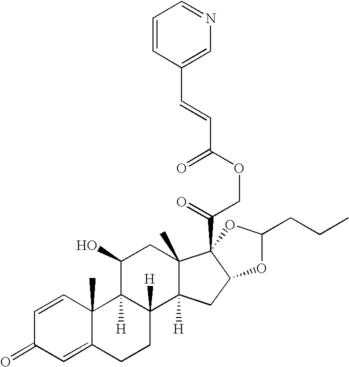

The title compound was synthesized in a manner analogous to that described in Example 33, using budesonide in place of desisobutyryl ciclesonide. ES/MS cacld for C₃₃H₄₀NO₇ 562.3, found m/z=562.3 (MH⁺).

### Example 45

### 4-Methylpiperazinecarboxylic acid [[11β,16α]-[[((16,17-(butylidenedioxy)]-11-hydroxypregna-1,4-diene-3,20-dion-21-yl]]ester

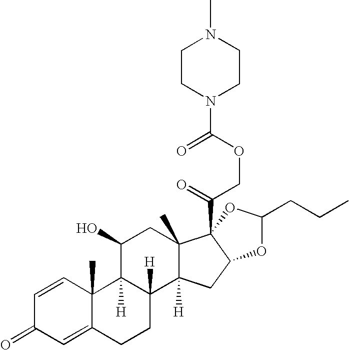

The title compound was synthesized in a manner analogous to that described in Example 41, using budesonide in place of desisobutyryl ciclesonide. ES/MS cacld for C₃₁H₄₅N₂O₇ 557.3, found m/z=557.3 (MH⁺).

### Example 46

### 1-[5-[1-Hydroxy-2-[6-(4-phenylbutoxy)hexylamino]ethyl]-2-phosphonooxybenzyl]-1-methyl-4-[[11β,16α]-[16,17-((R)-cyclohexylmethylene)bis(oxy)]-11-hydroxypregna-1,4-diene-3,20-dion-21-oxy]carbonyl]piperazinium chloride

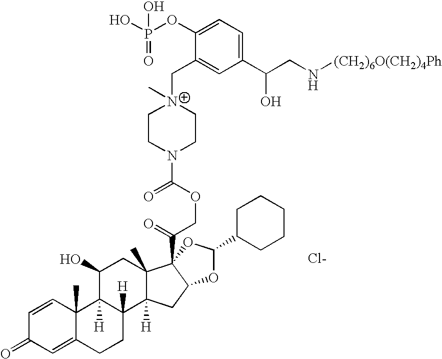

Solid NaI (47 mg, 0.316 mmol) was added to a stirred solution of methanesulfonic acid 5-[1-tert-butoxycarbonyloxy-2-[tert-butoxycarbonyl-[6-(4-phenylbutoxy)hexyl]amino]ethyl]-2-(di-tert-butoxyphosphoryloxy)benzyl ester (described in Example 5) (350 mg, 0.394 mmol) in CH₃CN (2 mL) at room temperature. The reaction mixture was stirred for 5 min followed by adding the steroid described in Example 41 (157 mg, 0.263 mmol) was added in one portion. The resulting suspension was stirred at rt and monitored by TLC and LC/MS. Once the steroid starting material was consumed (1 day) the reaction mixture was concentrated to give crude quaternary piperazinium salt. Chromatography (9:1, CH₂Cl₂/MeOH) afforded partially deprotected (mono-t-Bu-phosphate) intermediate (261 mg). It was redissolved in dioxane (2 mL) and 4 N HCl (2 mL, in dioxane) was added. The reaction mixture was stirred for 2 hr then concentrated to dryness. The residue was redissolved in minimum amount of CH₂Cl₂ (0.5 mL) and then dry Et₂O (50 mL) was added. Precipitate was filtered, washed with Et₂O (50 mL), and dried to give the title compound as a chloride salt (86 mg) as off white solid. ¹H NMR (400 MHz, DMSO) d 9.04 (m, 1H), 8.79 (m, 1H), 7.54 (m, 3H), 7.34 (d, 1H, J=10.1 Hz), 7.26 (m, 2H), 7.19 (m 3H), 6.17 (dd, 14H, J=1.8, 10.1 Hz), 5.92 (m, 1H), 5.00 (m, 2H), 4.75 (m, 4H), 4.39 (d, 1H, J=4.1 Hz), 4.32 (s, 1H), 3.99 (m, 2H), 3.82-3.31 (m, 14H), 3.18-2.85 (m, 8H), 2.57 (m, 2H), 2.31 (s, 1H), 2.27 (m, 1H), 2.13-1.94 (m, 2H), 1.71-0.80 (m, 34H); ³¹P NMR (400 MHz, DMSO-d₆) d −4.58; ES/MS calcd for C₅₉H₈₅N₃O₁₃P⁺ 1074.6, found m/z=1075.5 (M⁺).

### Example 47

### [5-[1-Hydroxy-2-[6-(4-phenylbutoxy hexylamino]ethyl]-2-phosphonooxybenzyl]-(dimethyl)-[[[11β,16α-[16,17-((R)-cyclohexylmethylene)bis(oxy)]-11-hydroxypregna-1,4-diene-3,20-dion-21-oxy]carbonyl]aminoethyl]ammonium chloride

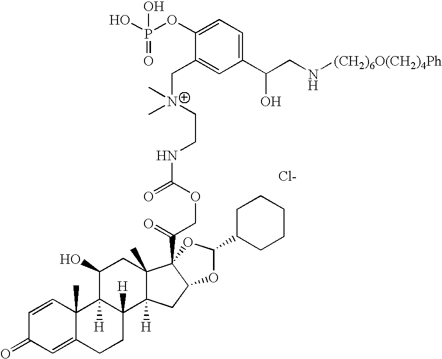

The title compound was synthesized in a manner analogous to that described in Example 46, using the steroid described in Example 39 as a substrate. ¹H NMR (400 MHz, DMSO) d 8.84 (s, 1H), 8.66 (s, 1H), 8.37 (s, 1H), 7.60 (d, 1H, J=8.7 Hz), 7.51 (s, 1H), 7.44 (d, 1H, J=9.7 Hz), 7.34 (d, 1H, J=10.1 Hz), 7.27 (m, 2H), 7.18 (m, 2H), 6.17 (dd, 1H, J=1.4, 10.1 Hz), 5.92 (s, 1H), 5.32 (d, H, J=12.4 Hz), 4.91 (m, 2H), 4.64 (m, 4H), 4.36 (d, 1H, J=4.2), 4.31 (m, 1H), 3.75-2.83 (m, 15H), 2.57 (m, 2H), 2.31 (m, 1H), 2.01 (m, 2H), 1.88-0.77 (m, 43H); ³¹P NMR (400 MHz, DMSO-d₆) d −4.01; ES/MS calcd for C₅₈H₈₅N₃O₁₃P⁺ 1062.6, found m/z=1062.6 (Me).

### Example 48

### 1-[5-[1-Hydroxy-2-[6-(4-phenylbutoxy)hexylamino]ethyl]-2-phosphonooxybenzyl]-3-[2-[[11β,16α]-[16,17-((R)-cyclohexylmethylene)bis(oxy)]-11-hydroxypregna-1,4-diene-3,20-dion-21-oxy]carbonyl ethen-1-yl]pyridinium chloride

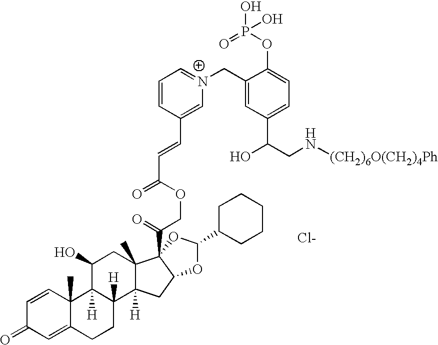

The title compound was synthesized in a mariner analogous to that described in Example 46, using the steroid described in Example 33 as a substrate. ¹H NMR (400 MHz, DMSO) d 9.66 (s, 1H), 9.09 (d, 1H, J=6.1 Hz), 8.99 (d, 1H, J=8.3 Hz), 8.88 (m, 1H), 8.66 (m, 1H), 8.21 (dd, 1H, J=6.2, 8.1 Hz), 7.84 (d, 1H, J=16.1 Hz), 7.65 (s, 1H), 7.46 (dd, 1H, J=1.8, 8.6 Hz), 7.37 (dd, 2H, J=9.3, 18.9 Hz), 7.25 (m, 2H), 7.17 (m, 3H, J=7.0 Hz), 6.25 (m, 1H), 6.18 (dd, 1H, J=1.9, 10.1 Hz), 5.90 (d, 2H, J=121.1 Hz), 5.16 (d, 1H, J=17.6 Hz), 4.99 (m, 2H), 4.72 (d, 1H, J=14.1 Hz), 4.43 (d, 1H, J=4.2 Hz), 4.33 (m, 1H), 3.86-3.21 (m, 9H), 3.12 (m, 1H), 2.94 (m, 3H), 2.57 (m, 2H), 2.32 (s, 3H), 2.29 (m, 1H), 2.08 (m, 2H), 1.88 (m, 2H), 1.25-0.87 (m, 31H); ³¹P NMR (400 MHz, DMSO) d −4.71; ES/MS calcd for C₆₁H₈₀N₂O₁₃P⁺ 1079.5, found m/z=1079.5 (M⁺).

### Example 49

### 1-[5-[1-Hydroxy-2-[6-(4-phenylbutoxy)hexylamino]ethyl]-2-phosphonooxybenzyl]-1-methyl-4-[[11β,16α]-[16,17-((R)-cyclohexylmethylene)bis(oxy)]-11-hydroxypregna-1,4-diene-3,20-dion-21-oxy]carbonyl]pyrrolidinium chloride

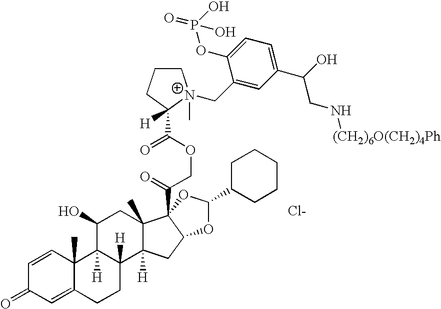

The title compound was synthesized in a manner analogous to that described in Example 46, using the steroid described in Example 38 as a substrate. ¹H NMR (400 MHz, DMSO-d₆) d 8.58 (m, 2H), 7.56 (m, 3H), 7.34 (d, 1H, J=10.1 Hz), 7.27 (m, 2H), 7.17 (d, 2H, J=7.5 Hz), 6.24 (s, 1H), 6.18 (d, 1H, J=8.9 Hz), 5.93 (m, 1H), 5.41-0.44 (m, 71H); ³¹P NMR (400 MHz, DMSO-d₆) d −5.02, −5.17; /MS calcd for C₅₉H₈₄N₂O₁₃P⁺1059.6, found m/z=1059.6 (M⁺).

### Example 50

### Valine [[11β,16α]-[16,17-((R)-cyclohexylmethylene)bis(oxy)]-11-hydroxypregna-1,4-diene-3,20-dion-21-yl]ester

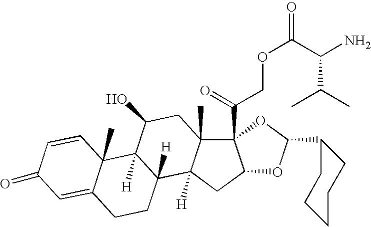

TEA (420 μL, 3 mmol) was added to a solution of Boc-valine (480 mg, 2.2 mmol) and HATU (837 mg, 2.2 mmol) in DMF (10 mL). After stirring for 10 min, des-CIC (940 mg, 2 mmol) was added and the resulting mixture was then stirred overnight, then poured into water (100 mL) and filtered. The precipitate was dissolved in ethyl acetate (100 mL) and washed with saturated sodium bicarbonate (50 mL), dried over magnesium sulfate, filtered and concentrated. The residue was purified by passing through a plug of silica gel to remove starting material (R_{f}=0.8 in ethyl acetate/hexanes 4:1 mixture) to give N-Boc-protected title compound (1.065 g). This solid was dissolved in DCM (8 mL) and cooled to 0° C. followed by the addition of TFA (6 mL) and the reaction mixture was allowed to warm up to rt and then stirred for additional 30 min, after which it was concentrated. The resulting residue was dissolved DCM (100 mL) and washed with sat. NaHCO₃ solution (2×50 mL), dried over MgSO₄, filtered and concentrated to give the title compound as a white solid (783 mg, 1.37 mmol, 69% after 2 steps). ES/MS calcd. for C₃₃H₄₈NO₇: 570.3; Found: 570.3 (M+H⁺).

### Example 51

### N-(Pyridin-3-carbonyl)valine [[11β,16α]-[16,17-((R)-cyclohexylmethylene)bis(oxy)]-11-hydroxypregna-1,4-diene-3,20-dion-21-yl]ester

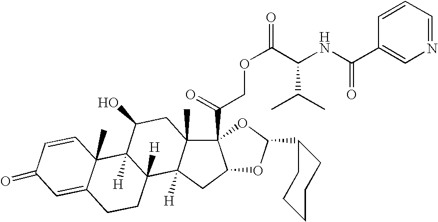

The title compound was synthesized in a manner analogous to that described in Example 29, using steroid described in Example 50 as a substrate. ES/MS calcd for C₃₉H₅₁N₂O₈: 675.4; Found: 675.3 (M+H⁺).

### Example 52

### [5-[1-hydroxy-2-[6-(4-phenylbutoxy)hexylamino]ethyl]-2-phosphonooxybenzyl]-(diethyl)-[[11β,16α]-[[15,16-((R)-cyclohexylmethylene)bis(oxy)]-11-hydroxypregna-1,4-diene-3,20-dion-21-yl]carbonylmethyl]ammonium chloride

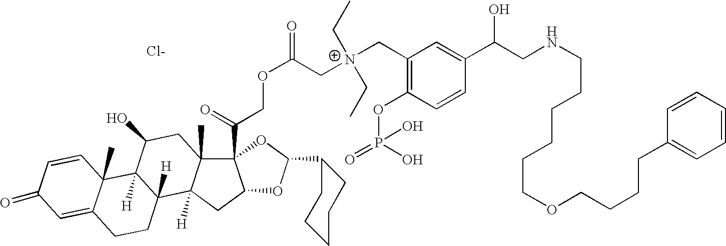

Method A. Solid NaI (76 mg, 0.51 mmol) was added to a stirred solution of methanesulfonic acid 5-[2-{tert-butoxycarbonyl-[6-(4-phenylbutoxy)hexyl]amino]-1-hydroxyethyl)-2-(di-tert-butoxy-phosphoryloxy)benzyl ester (described in Example 3) (3.1 g, 3.81 mmol) in CH₃CN (13 mL) at rt. The reaction mixture was stirred for 5 min and the compound prepare according to Example 32 (1.48 g, 2.54 mmol) was added in one portion. The resulting suspension was stirred at rt and monitored by TLC and LC/MS. Once the steroid starting material was consumed (usually after 5 days) the reaction mixture was concentrated and the residue was loaded onto a short plug of silica in minimal amount of CH₂Cl₂ and the plug was washed with EtOAc to remove impurities and then with 1:1 CH₂Cl₂:2-propanol to elute the desired intermediate. That intermediate was then redissolved in dioxane (5 mL) and 4 N HCl (5 mL, dioxane) was added. The reaction mixture was stirred for 2 hr and then concentrated to dryness. The residue was redissolved in minimum amount of CH₂Cl₂ (2 mL) then dry Et₂O (50 mL) was added to form a precipitate, which was filtered, washed with Et₂O (50 mL), and dried to give an off white solid. The solid was dissolved in an acetonitrile/water 1:1 mixture and the solution was passed through a short column packed with Dowex Cl⁻ resin (pretreated with 1N HCl and then washed with water and acetonitrile/water 1:1 to neutral pH) eluting with acetonitrile/water 1:1. The fractions containing the desired compound were frozen and lyophilized to give the title compound as a white solid (2.127 g, 1.88 mmol, 74%) ES/MS calcd for C₅₉H₈₆N₂O₁₃P⁺: 1061.6; Found: 1061.5 (M⁺).

Method B. 4-Diethylamino acetic acid [[11β,16α]-[[((R)-cyclohexylmethylene)bis(oxy)]-11-hydroxypregna-1,4-diene-3,20-dion-21-yl]]ester (described in Example 32) (796 mg, 1.36 mmol) and 1,2,2,6,6-pentamethylpiperidine (0.627 mL, 3.72 mmol) were added to a stirred solution of carbonic acid [2-[tert-butoxycarbonyl[6-(4-phenylbutoxy)hexyl]amino]-1-[4-(di-tert-butoxyphosphoryloxy)-3-hydroxymethylphenyl]ethyl]ester tert-butyl ester (described in Example 4) (1 g, 1.24 mmol) in DCM (10 mL). The resulting solution was cooled to −78° C. then a solution of Tf₂O (0.25 mL, 1.49 mmol) in DCM (1.5 mL) was added dropwise, followed by removal of the cooling bath to allow the reaction mixture to warm to rt over 1 hr. The reaction mixture was then concentrated to dryness, redissolved with ethyl acetate (30 mL) and the organic layer was washed with 10% citric acid (50 mL), saturated NaHCO₃ (50 mL), brine (50 mL), dried over Na₂SO₄, and concentrated to give crude residue that was dissolved in warm ethyl acetate and then passed through a plug of silica gel, eluting with ethyl acetate and then acetone. The acetone fractions were concentrated to give a mixture of the fully protected intermediate product and of unreacted steroid. The mixture was dissolved in DCM (5 mL) and anhydrous gaseous HCl was bubbled through the solution for about 1 min. The reaction mixture was stirred for 3 h at rt then diethyl ether (50 mL) was added. The resulting suspension was stirred for 30 min and the precipitate was filtered off to give the title compound (591 mg) in crude form. The steroid starting material was removed using an SCX column, followed by the ion exchange chromatography described in Method A.

### Example 53

### 1-[5-[1-Hydroxy-2-[6-(4-phenylbutoxy)hexylamino]ethyl]-2-phosphonooxybenzyl]-3-[[11β,16α]-[16,17-((R)-cyclohexylmethylene)bis(oxy)]-11-hydroxypregna-1,4-diene-3,20-dion-21-oxy]carbonyl]pyridinium chloride

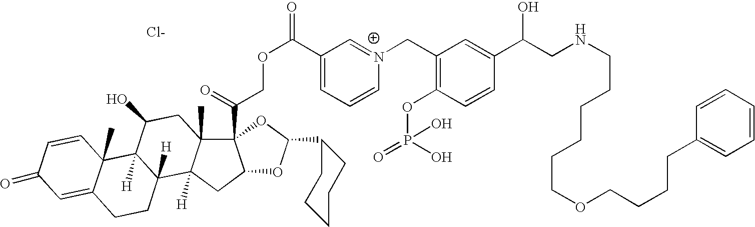

The title compound was synthesized in a manner analogous to that described in Example 46, using the steroid described in Example 29 as a substrate; ES/MS calcd for C₅₉H₇₉N₂O₁₃P⁺: 1053.5; Found: 1053.6 (M⁺).

### Example 54

### [5-[1-Hydroxy-2-[6-(4-phenylbutoxy)hexylamino]ethyl]-2-phosphonooxybenzyl]-(diethyl)-[3-[[11β,16α]-16,17-((R)-cyclohexylmethylene)bis(oxy)]-11-hydroxypregna-1,4-diene-3,20-dion-21-oxy]carbonyl]phenylmethyl]ammonium chloride

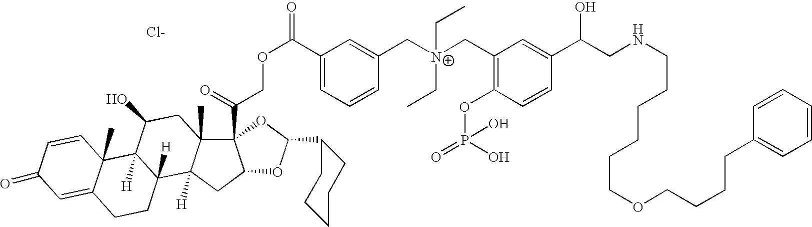

The title compound was synthesized in a manner analogous to that described in Example 52, using the steroid described in Example 31 as a substrate; ES/MS calcd for C₆₅H₉₀N₂O₁₃P⁺: 1137.6; Found: 1137.7 (M⁺).

### Example 55

### [5-[1-Hydroxy-2-[6-(4-phenylbutoxy)hexylamino]ethyl]-2-phosphonooxybenzyl]-(dimethyl)-[3-[[11β,16α]-16,17-((R)-cyclohexylmethylene)bis(oxy)]-11-hydroxypregna-1,4-diene-3,20-dion-21-(oxy)]carbonyl]propyl]ammonium chloride

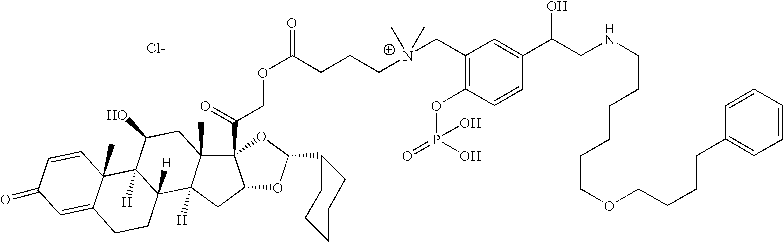

The title compound was synthesized in a manner analogous to that described in Example 46, using the steroid described in Example 27 as a substrate. ES/MS calcd for C₅₉H₈₆N₂O₃P⁺: 1061.6; Found: 1061.6 (M⁺).

### Example 56

### [5-[1-Hydroxy-2-[6-(4-phenylbutoxy)hexylamino]ethyl]-2-phosphonooxybenzyl]-(diethyl)-[[2-[[11β,16α]-[16,17-((R)-cyclohexylmethylene)bis(oxy)]-11-hydroxypregna-1,4-diene-3,20-dion-21-oxy]carbonyl]oxy]ethyl]ammonium chloride

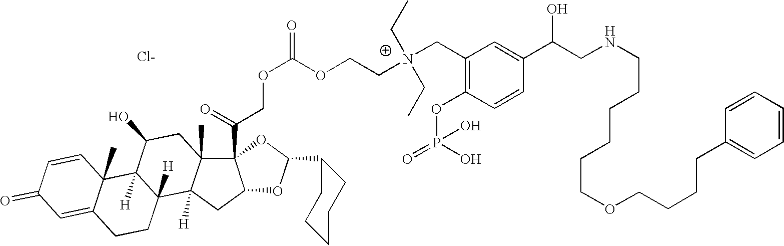

The title compound was synthesized in a manner analogous to that described in Example 52, using the steroid described in Example 26; ES/MS calcd for C₆₀H₈₈N₂O₁₄P⁺: 1091.6; Found: 1091.5 (M⁺).

### Example 57

### 1-[5-[1-Hydroxy-2-[6-(4-phenylbutoxy)hexylamino]ethyl]-2-phosphonooxybenzyl]-3-[[1-[[[11β,16α]-[16,17-((R)-cyclohexylmethylene)bis(oxy)]-11-hydroxypregna-1,4-diene-3,20-dion-21-oxy]carbonyl]-2-methylpropyl]aminocarbonyl]pyridinium chloride

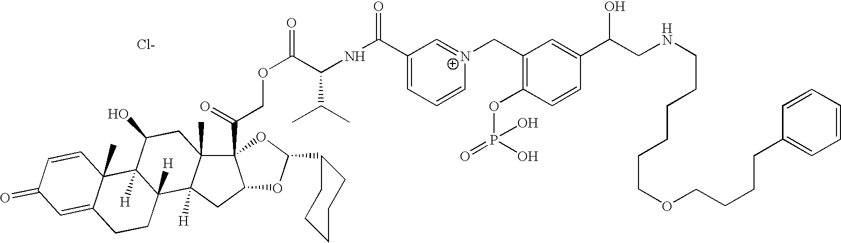

The title compound was synthesized in a manner analogous to that described in Example 46, using the steroid described in Example 51 as a substrate. ES/MS calcd for C₆₄H₈₇N₃O₁₄P: 1152.6; Found: 1152.5 (M⁴).

### Example 58

### [5-[1-hydroxy-2-[6-(4-phenylbutoxy)hexylamino]ethyl]-2-[(phosphonooxy)methoxy]benzyl]-(diethyl)-[[11β,16α]-[[15,16-((R)-cyclohexylmethylene)bis(oxy)]-11-hydroxypregna-1,4-diene-3,20-dion-21-oxy]carbonylmethyl]ammonium chloride

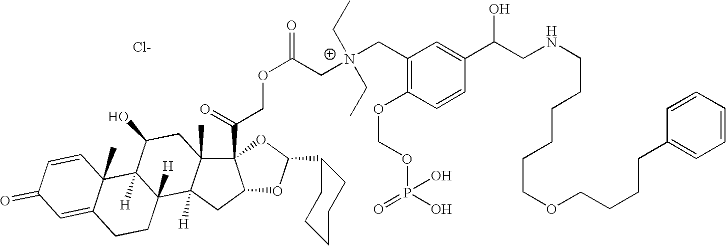

Solid NaI (2 mg, 9.6 μmol) was added to a stirred solution of methanesulfonic acid 5-[2-[tert-butoxycarbonyl-[6-(4-phenylbutoxy)hexyl]amino]-1-hydroxyethyl]-2-(di-tert-butoxyphosphoryloxymethoxy)benzyl ester (described in Example 8) (59 mg, 72 μmol) in CH₃CN (170 μL) at rt. The reaction mixture was stirred for 5 min and the product prepared according to Example 32 (28 mg, 48 μmol) was added in one portion. The resulting suspension was stirred at rt and monitored by TLC and LC/MS. Once the starting material was consumed (usually after 3 days) the reaction mixture was concentrated and the residue was loaded onto a short plug of silica in a minimal amount of DCM and the plug was washed with EtOAc to remove impurities then with 1:1 DCM/2-propanol 1:1 mixture to elute the protected product. That intermediate was redissolved in DCM (300 μL) and stirred at 0° C., followed by addition of TFA (300 μL) and the solution was allowed to warm to rt. After 2 hr the reaction mixture was concentrated to dryness and the residue was dissolved in a minimum amount of DCM (2 mL) followed by addition of dry Et₂O (50 mL). The precipitate was filtered, washed with Et₂O (50 mL), and dried to give an off white solid. The resulting solid was then purified by ion-exchange chromatography as described in Example 52, Method A to give the title compound as a white solid (40 mg, 34 μmol, 70%) ES/MS calcd for C₆₀H₈₈N₂O₁₄P⁺: 1091.6; Found: 1091.7 (M⁺).

### Example 59

### [5-[1-hydroxy-2-[6-(4-phenylbutoxy)hexylamino]ethyl]-2-[(phosphonooxy)methoxy]benzyl]-(diethyl)-[3-[[11β,16α]-[16,17-((R-cyclohexylmethylene)bis(oxy)]-11-hydroxypregna-1,4-diene-3,20-dion-21-oxy]carbonyl]pyridinium chloride

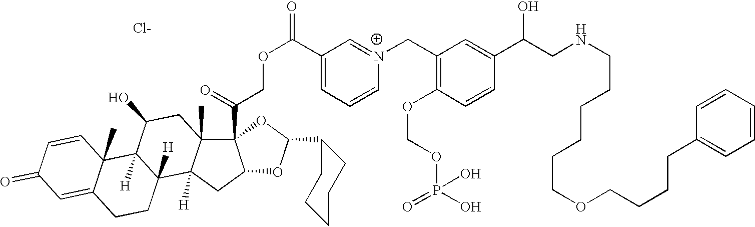

The title compound was synthesized in a manner analogous to that described in Example 58, using the steroid described in Example 29 as a substrate. ES/MS calcd for C₆₀H₈₀N₂O₁₄P⁺: 1083.5; Found: 1083.5 (M⁺).

### Example 60

### [5-[1-Hydroxy-2-[6-(4-phenylbutoxy)hexylamino]ethyl]-2-[(phosphonooxy)methoxy]benzyl]-(diethyl)-[[2-[[11β,16α]-[16,17-((R)-cyclohexylmethylene)bis(oxy)]-11-hydroxy-pregna-1,4-diene-3,20-dion-21oxy]carbonyl]oxy]ethyl]ammonium chloride

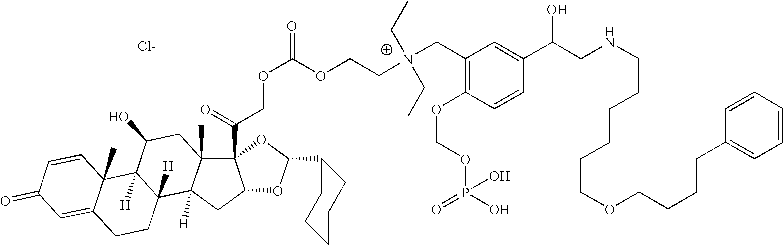

The title compound was synthesized in a manner analogous to that described in Example 58, using the steroid described in Example 26 as a substrate. ES/MS calcd for C₆₁H₉₀N₂O₁₅P⁺: 1121.6; Found: 1121.6 (M⁺).

### Example 61

### Glycine [[11β,16α-]-[16,17-((R)-cyclohexylmethylene)bis(oxy)]-11-hydroxypregna-1,4-diene-3,20-dion-21-yl]ester

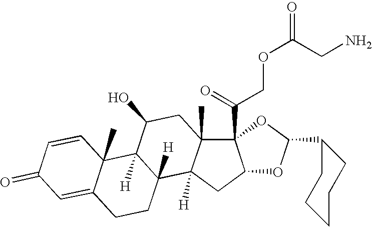

The title compound was synthesized in a manner analogous to that described in Example 50 using Boc glycine in place of Boc (L)-valine. ES/MS calcd for C₃₀H₄₂NO₇⁺: 1067.5; Found: 1067.2 (M+H⁺).

### Example 62

### Alanine [[11β,16α]-[16,17-((R)-cyclohexylmethylene)bis(oxy)]-11-hydroxypregna-1,4-diene-3,20-dion-21-yl]ester

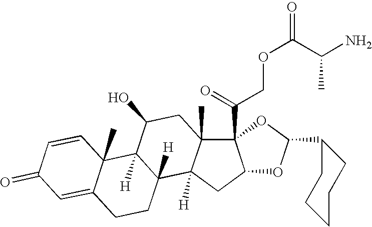

The title compound was synthesized in a manner analogous to that described in Example 50 using Boc (L)-alanine in place of Boc (L)-valine.

### Example 63

### 4-Aminobutanoic acid [[11β,16α]-[16,17-((R)-cyclohexylmethylene)bis(oxy)]-11-hydroxypregna-1,4-diene-3,20-dion-21-yl]ester

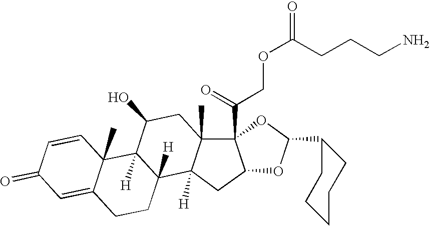

The title compound was synthesized in a manner analogous to that described in Example 50 using 4-Boc-aminobutanoic acid in place of Boc(L)-valine. ES/MS calcd for C₃₂H₄₆NO₇⁺: 556.3; Found: 555.8 (M+1H).

### Example 64

### 3-Amino-2-methylpropanoic acid [[11β,16α]-[16,17-((R)-cyclohexylmethylene)bis oxy]-11]-hydroxypregna-1,4-diene-3,20-dion-21-yl]ester

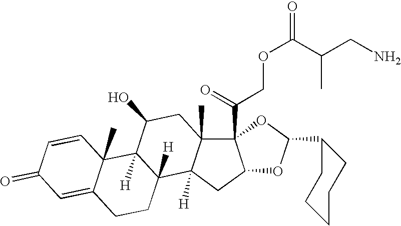

The title compound is synthesized in a manner analogous to that described in Example 50 using 3-Boc-amino-2-methylpropanoic acid in place of Boc (L)-valine. ES/MS calcd for C₃₂H₄₆NO₇⁺:556.3; Found: 555.8 (M+H>).

### Example 65

### N-(Pyridin-3-carbonyl)glycine [[11β,16α]-[16,17-((R)-cyclohexylmethylene)bis(oxy)]-11-hydroxypregna-1,4-diene-3,20-dion-21-yl]ester

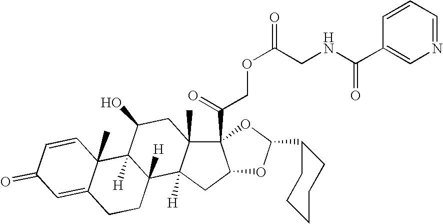

The title compound was synthesized in a manner analogous to that described in Example 29 using the compound prepared as described in Example 61 as a substrate. ES/MS calcd for C₃₆H₄₅N₂O₈⁺:633.3; Found: 632.7 (M+H⁺).

### Example 66

### N-(Pyridin-3-carbonyl)alanine [[11β,16α]-[16,17-((R)-cyclohexylmethylene)bis(oxy)]-11-hydroxypregna-1,4-diene-3,20-dion-21-yl]ester

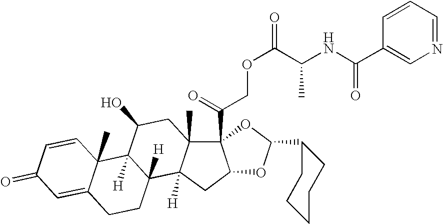

The title compound is synthesized in a manner analogous to that described in Example 29 and using the compound prepared as described in Example 62 as a substrate.

### Example 67

### 4-[(Pyridin-3-yl)carbonyl]aminobutanoic acid [[11β,16α]-[16,17-((R)-cyclohexylmethylene)bis(oxy)]-11-hydroxypregna-1,4-diene-3,20-dion-21-yl]ester

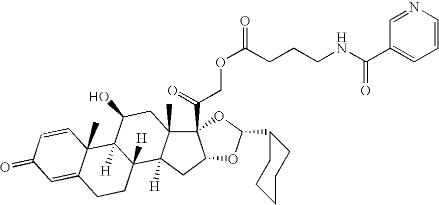

The title compound was synthesized in a manner analogous to that described in Example 29 and using the compound prepared as described in Example 63 as a substrate. ES/MS calcd for C₃₈H₄₉N₂O₈⁺: 661.3; Found: 660.7 (M+H⁺).

### Example 68

### 3-[(Pyridin-3-yl)carbonyl]amino-2-methylpropanoic acid [[11β,16α]-[16,17-((R)-cyclohexylmethylene)bis(oxy)]-11-hydroxypregna-1,4-diene-3,20-dion-21-yl]ester

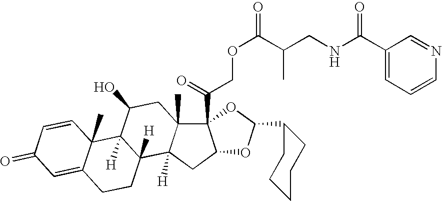

The title compound was synthesized in a manner analogous to that described in Example 29 using the compound prepared as described in Example 64 as a substrate. ES/MS calcd for C₃₈H₄₉N₂O₈⁺: 661.3; Found, 660.7 (M+H⁺).

### Example 69

### 1-[5-[1-Hydroxy-2-[6-(4-phenylbutoxy)hexylamino]ethyl]-2-phosphonooxybenzyl]-3-[[[[[11β,16α]-[16,17-((R)-cyclohexylmethylene)bis(oxy)]11-hydroxypregna-1,4-diene-3,20-dion-21-oxy]carbonyl]methyl]aminocarbonyl]pyridinium chloride

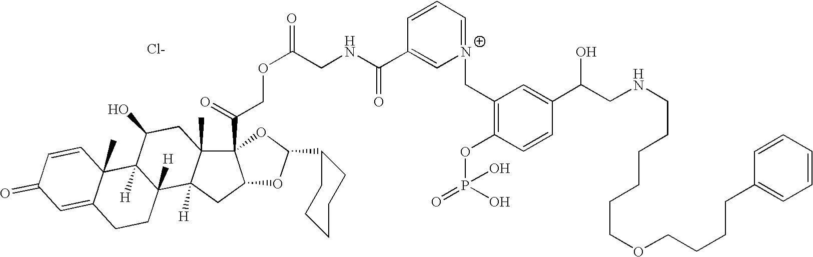

The title compound was synthesized in a manner analogous to that described in Example 46 but using the compound prepared as described in Example 65 as a substrate. ES/MS calcd for C₆₁H₈₁N₃O₁₄P⁺: 1110.5; Found: 1110.4 (M⁺). ¹H NMR (400 MHz, DMSO-d₆): d 11.12-10.88 (br s, 1H), 9.67 (s, 1H), 9.23-9.18 (m, 1H), 8.94-8.58 (m, 2H), 8.23 (dd, J=6.4, 8.0 Hz, 1H), 7.69 (s, 1H), 7.42 (s, 2H), 7.36-7.23 (m, 34), 7.21-7.13 (m, 3H), 6.24-6.18 (m, 1H) 6.17 (dd, J=10.09, 1.86 Hz, 1H), 5.94-5.86 (m, 2H), 5.06 (d, J=18.0 Hz, 1H), 4.96-4.88 (m, 2H), 4.87 (d, J=18.0 Hz, 1H) 4.73-4.70 (m, 1H), 4.39 (d, J=4.17 Hz, 1H), 4.34-4.27 (m, 1H), 4.18 (d, J=5.95 Hz, 2H), 3.40-3.05 (m, 4H), 3.35 (t, J=6.45 Hz, 2H), 3.33 (t, J=6.45 Hz, 2H), 3.04-2.86 (m, 3H), 2.69-2.44 (m, 2H), 2.35-2.25 (m, 1H), 2.14-1.95 (m, 2H), 1.89-0.84 (m, 35H)

### Example 70

### 1-[5-[1-Hydroxy-2-[6-(4-phenylbutoxy)hexylamino]ethyl]-2-phosphonooxybenzyl]-3-[[1-[[[11β,16α]-16,17-((R)-cyclohexylmethylene bis(oxy)-11-hydroxypregna-1,4-diene-3,20-dion-21-oxy]carbonyl]ethyl]aminocarbonyl]pyridinium chloride

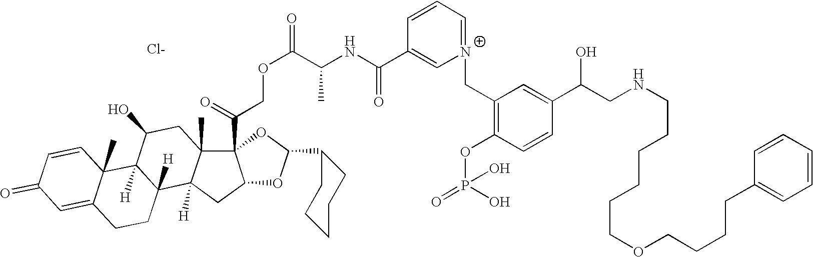

The title compound is synthesized in a manner analogous to that described in Example 46, but using the compound prepared as described in Example 66 as a substrate.

### Example 71

### 1-[5-[1-Hydroxy-2-[6-(4-phenylbutoxy)hexylamino]ethyl]-2-phosphonooxybenzyl]-3-[[3-[[[11β,16α]-[16,17-((R)-cyclohexylmethylene)bis(oxy)]-11-hydroxypregna-1,4-diene-3,20-dion-21-oxy]carbonyl]propyl]aminocarbonyl]pyridinium chloride

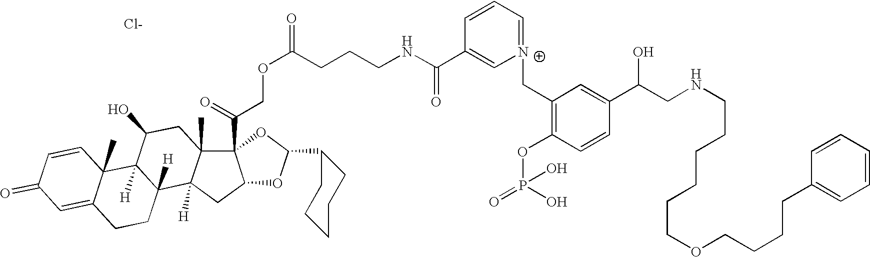

The title compound was synthesized in a manner analogous to that described in Example 46, but using the compound prepare as described in Example 67 as a substrate. ES/MS calcd for C₆₃H₈₅N₃O₄P⁺: 1138.6; Found: 1138.4 (M⁺).

### Example 72

### 1-[5-[1-Hydroxy-2-[6-(4-phenylbutoxy)hexylamino]ethyl]-2-phosphonooxybenzyl]-3-[[2-[[[11β,16α]-[16,17-((R)-cyclohexylmethylene)bis(oxy)]-11-hydroxypregna-1,4-diene-3,20-dion-21-oxy]carbonyl]-2-methylethyl]aminocarbonyl]pyridinium chloride

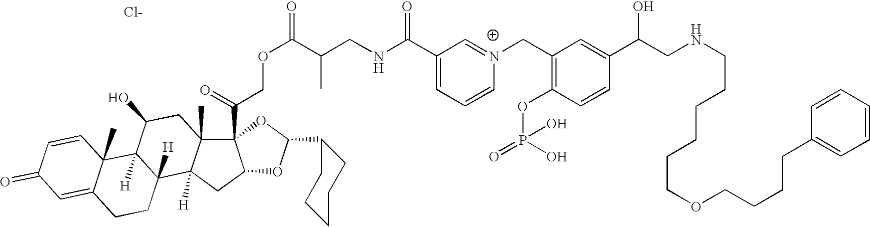

The title compound was synthesized in a manner analogous to that described in Example 46, but using the compound prepared as described in Example 68 as a substrate. ES/MS calcd for C₆₃H₅N₃O₁₄P⁺:1138.6; Found: 1138.6 (M⁺).

### Example 73

### 1-[5-[1-hydroxy-2-[6-(4-phenylbutoxy)hexylamino]ethyl]-2-phosphonooxybenzyl]-3-[[[11β,16α]-[16,17-((R)-cyclohexylmethylene)bis(oxy)]-11-hydroxypregna-1,4-diene-3,20-dion-21-oxy]carbonyl]methyl]pyridinium chloride

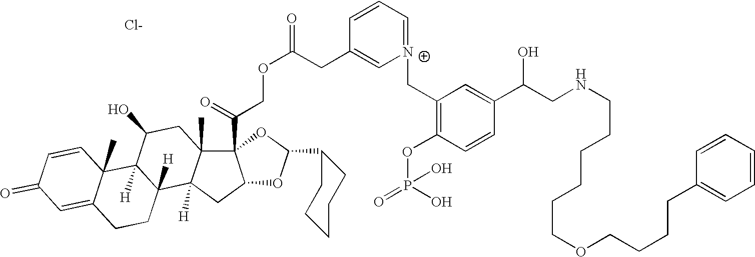

The title compound was synthesized in a manner analogous to that described in Example 46, but using the compound prepared as described in Example 76 as a substrate. ES/MS calcd for C₆₀H₈₀N₂O₁₃P⁺: 1067.5; Found: 1067.2 (M.

### Example 74

### 1-[5-[1-Hydroxy-2-[6-(4-phenylbutoxy)hexylamino]ethyl]-2-phosphonooxybenzyl]-3-[2-[[11β,16α]-[16,17-(R)-cyclohexylmethylene)bis(oxy)-hydroxypregna-1,4-diene-3,20-dion-21-(oxy)]carbonyl]ethyl]pyridinium chloride

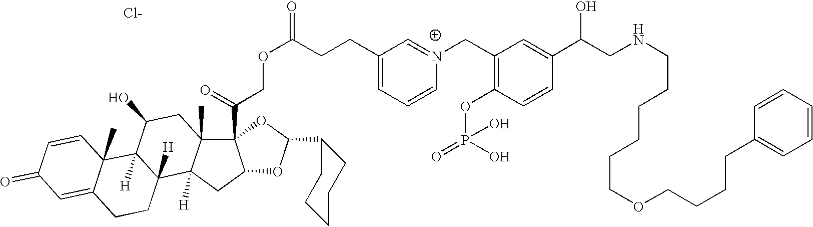

The title compound was synthesized in a manner analogous to that described in Example 46, but using the compound prepared as described in Example 34 as a substrate. ES/MS calcd. for C₆₁H₈₂N₂O₁₃P⁺: 1081.6; Found: 1081.4 (M⁺).

### Example 75

### N,N-Dimethylglycine [[11β,16α]-[16,17-((R)-cyclohexylmethlylene)bis(oxy)]-11-hydroxypregna-1,4-diene-3,20-dion-21-yl]ester

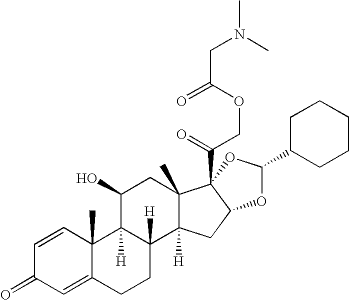

The title compound was synthesized in a manner analogous to that described in Example 32, but using dimethylamine in place of diethylamine. ES/MS calcd. for C₃₂H₄₆NO₇ 556.3, found m/z=556.4 (M+H⁺).

### Example 76

### 3-Pyridineacetic acid [[11β,16α]-[16,17-((R)-cyclohexylmethylene)bis(oxy)]-11-hydroxypregna-1,4-diene-3,20-dion-21-yl]ester

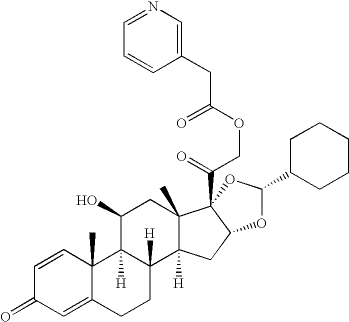

The title compound was synthesized in a manner analogous to that described in Example 33, but using pyridine-3-ylacetic acid in place of 3-(pyridin-3-yl)acrylic acid. ES/MS calcd for C₃₅H₄₄NO₇ 590.3, found m/z=589.7 (MH⁺).

### Example 77

### 1-[5-[1-Hydroxy-2-[6-(4-phenylbutoxy)hexylamino]ethyl]-2-phosphonooxybenzyl]-1-methyl-4-[[11β,16α]-[[((R)-cyclohexylmethylene)bis(oxy)]-11-hydroxypregna-1,4-diene-3,20-dion-21-oxy]carbonyl]piperidinium acetate

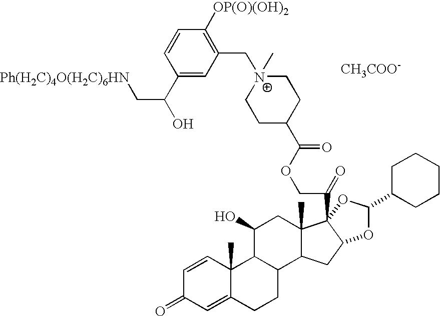

Solid Nat (24 mg, 0.158 nmol) was added to a stirred solution of carbonic acid [2-[tert-butoxycarbonyl[6-(4-phenylbutoxy)hexyl]amino]-1-[4-(di-tert-butoxyphosphoryloxy)-3-hydroxymethylphenyl]ethyl]ester tert-butyl ester (described in Example 4) (140 mg, 0.158 mmol) and the compound prepare as described in Example 25 (94 mg, 0.158 mmol) in CH₃CN (2 mL). After stirring the suspension at rt the for 15 h. the suspension was concentrated, dissolved in DCM (10 mL) and washed with water (10 mL×2) and brine (10 mL), dried (Na₂SO₄), and then concentrated to provide crude title compound (221 mg) as a yellow oil. The crude material was dissolved in DCM (2 mL) followed by addition of HCl (2 mL, 4 M in 1,4-dioxane, 8 mmol) with stirring. After stirring 1 h, Et₂O (10 mL) was added. After stirring an additional 1 h, the precipitate was filtered-off and washed with Et₂O which was also decanted. The solid was dried under reduced pressure to provide a crude product (178 mg) as a yellow solid. Chromatography (SCX column, DCM to MeOH gradient, then C18 column, H₂O with 0.1% AcOH to CH₃CN with 0.1% AcOH gradient) afforded the title compound (72 mg, 42% yield) as a white solid after lyophilization. ¹H NMR (400 MHz, CDCl₃) d 7.61-7.49 (m, 1H), 7.49-7.20 (m, 5H), 7.20-7.07 (m, 2H), 6.25-6.05 (m, 1H), 5.97-5.83 (m, 1H), 5.13-4.93 (m, 2H), 4.93-4.50 (m, 6H), 4.44-4.21 (m, 3H), 3.82-3.49 (m, 14H), 3.48-3.37 (m, 8H), 3.06-2.67 (m, 7H), 2.64-2.51 (m, 41), 2.37-2.22 (m, 2H), 2.22-1.92 (m, 4H), 1.89 (s, 1H), 1.87-1.72 (m, 2H), 1.72-1.39 (m, 11H), 1.36 (s, 2H), 1.32-1.22 (m, 3H), 1.22-0.74 (m, 7H); ³¹P NMR (400 MHz, DMSO-d₆) d −4.20, −4.40; ES/MS cacld for C₆₀H₈₆N₂O₁₃P⁺ 1073.6, found m/z=1072.4 (M+H).

### Example 78

### [5-[1-Hydroxy-2-[6-(4-phenylbutoxy)hexylamino]ethyl]-2-phosphonooxybenzyl]-(diethyl)-[[11β,16α]-[16,17-(propylmethylene)bis(oxy)]-11-hydroxypregna-1,4-diene-3,20-dion-21-oxy]carbonylmethyl]ammonium chloride

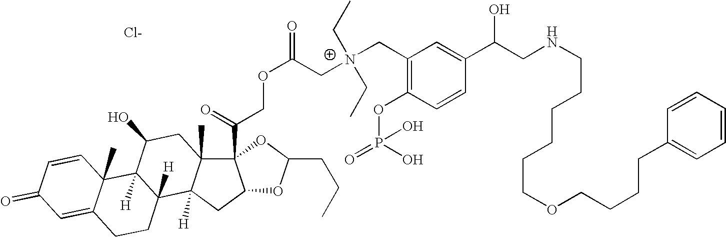

The title compound was synthesized in a manner analogous to that described in Example 52 Method A, using the compound prepared as described in Example 43 as a substrate. ES/MS calcd. for C₅₆H₈₂N₂O₁₃P⁺: 1021.6; Found: 1021.5 (M⁺).

### Example 79

### N,N-dimethylvaline [[11β,16α]-[16,17-((R)-cyclohexylmethylene)bis(oxy)]-11-hydroxypregna-1,4-diene-3,20-dion-21-yl]ester

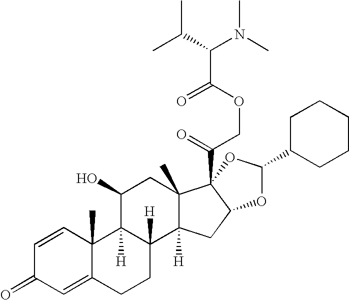

The title compound was synthesized in a manner analogous to that described in Example 33 using N,N-dimethyl-valine in place of 3-(pyridin-3-yl)acrylic acid. ES/MS calcd for C₃₅H₅₂NO₇ 598.4, found m/z=598.4 (MH⁺).

### Example 80

### [2-(3-Formyl-4-hydroxyphenyl)-2R-hydroxyethyl][6-(4-phenylbutoxy)hexyl]carbamic acid tert-butyl ester

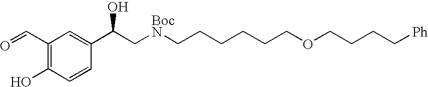

[2-(3-hydroxymethyl-4-hydroxyphenyl)-2R-hydroxyethyl][6-(4-phenylbutoxy)hexyl]carbamic acid tert-butyl ester (860 mg, 1.67 mmol) was dissolved in chloroform (15 mL) and activated manganese (TV) oxide (1.45 g, 85% w/w, 10 mmol) was added in portions with vigorous stirring. After 24 h at rt the slurry was filtered through a pad of Celite, followed by concentration of the filtrate combined with the chloroform washes and concentrated to give an oil (771 mg, 90%). ES/MS calcd. For C₃₀H₄₃NNaO₆ 536.3, found m/z=536.3 (M+Na⁺).

### Example 81

### [2-[4-(Di-tert-butoxy-phosphoryloxy)-3-hydroxymethylphenyl]-2R-hydroxyethyl][6-(4-phenylbutoxy)hexyl]carbamic acid tert-butyl ester

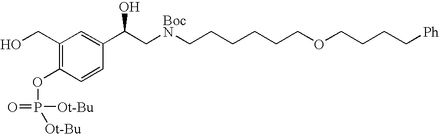

To a solution of sodium hydroxide (0.6 g, 15 mmol) in water (3 mL) was added benzyltriethylammonium chloride (42 mg, 0.188 mmol), DCM (3 mL), and bromotrichloromethane (0.195 mL, 1.95 mmol). To this vigorously stirred biphasic mixture at 0° C. was added a solution of di-tert-butyl phosphite (0.375 mL, 1.88 mmol) in DCM (3 mL) over 5 min. The reaction mixture was allowed to warm to rt and stirred vigorously for 2 h, at which point, a solution of [2-(3-formyl-4-hydroxyphenyl)-2R-hydroxyethyl]-[6-(4-phenylbutoxy)hexyl]carbamic acid tert-butyl ester (771 mg, 1.5 mmol) in DCM (3 mL) and dimethylaminopyridine (18 mg, 0.15 mmol) was added. The mixture stirred for 1 h. Ethyl acetate (60 mL) was added and the water layer was removed. The organic layer was washed with 10% citric acid (2×20 mL), 2N NaOH (2×20 mL), dried over sodium sulfate, filtered through a pad of activated basic alumina, and concentrated to give a clear oil. This clear oil was dissolved in THF (15 mL) and cooled to 0° C. followed by addition of NaBH₄ (114 mg, 3 mmol) in H₂O (1.5 mL). The resulting reaction mixture was stirred for 30 min and then was quenched with 10% citric acid (20 mL) followed by addition of DCM (60 mL). The aqueous layer was discarded and the organic layer was washed with saturated NaHCO₃ (30 mL), brine (300 mL), dried over Na₂SO₄, and concentrated to the title compound as a light yellow oil (995 mg, 93%). ¹H NMR (CDCl₃) selected signals: 7.17-7.41 (m, 8H), 4.92 (m, IX), 4.62 (bs, 2H), 3.39 (q, 2H), 2.64 (t 2H), 1.62 (m, 4H), 1.54 (s, 9H), 1.52 (s, 9H), 1.49 (s, 9H), 1.115-1.49 (m, 8H). ³¹P NMR (CDCl₃); -13.060 ppm. LCMS: 99%, MNa⁺ 730.0 (exact mass 707.4 calcd for C₃₉H₆₂NO₉P). Anal. Calc: C, 64.48; H, 8.83; N, 1.98. Found: C, 64.70; H, 8.84; N, 1.90.

### Example 82

### Methanesulfonic acid 5-[2-[tert-butoxycarbonyl[6-(4-phenylbutoxy)hexyl]amino]-1R-hydroxyethyl]-2-(di-tert-butoxyphosphoryloxy)benzyl ester

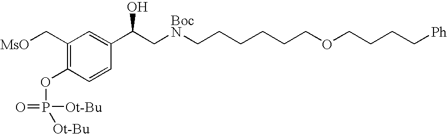

To a solution of [2-[4-(di-tert-butoxyphosphoryloxy)-3-hydroxymethylphenyl]-2R-hydroxyethyl]-[6-(4-phenylbutoxy)hexyl]carbamic acid tert-butyl ester (995 mg, 1.4 mmol) and 1,2,2,6,6-pentamethyl-piperidine (506 μL, 2.8 mmol) in DCM (14 mL) at −78° C. was added a solution of methanesulfonic acid chloride (0.1 mL, 1.4 mmol) in DCM (3 mL) over 5 mm. The reaction was stirred for 10 min at −78° C., concentrated and then purified by silica gel chromatography (gradient: 30% to 80% ethyl acetate in hexanes, both buffered with 1% TEA) to give the title compound as a clear oil (729 mg, 0.927 mmol, 66%). ES/MS calculated For C₃₉H₆₄NNaO₁₁PS 808.4, found m/z=808.3 (M+Na⁺).

### Example 83

### [5-[1-(R)-hydroxy-2-[6-(4-phenylbutoxy)hexylamino]ethyl]-2-phosphonooxybenzyl]-(diethyl)-[[11β,16α]-[16,17-((R-cyclohexylmethylene)bis(oxy)]-11-hydroxypregna-1,4-diene-3,20-dion-21-oxy]-carbonylmethyl]ammonium chloride

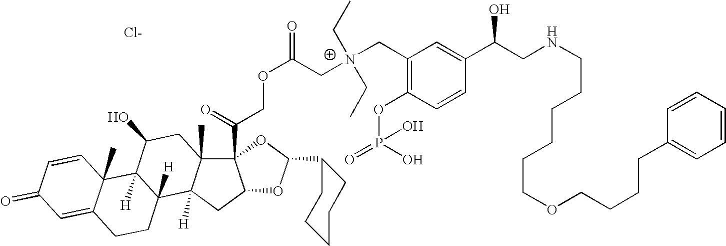

A mixture of NaI (128 mg, 0.856 mmol), methanesulfonic acid 5-(2-{tert-butoxycarbonyl-[6-(4-phenylbutoxy)hexyl]amino}-1R-hydroxyethyl)-2-(di-tert-butoxyphosphoryloxy)benzyl ester (729 mg, 0.927 mmol), and 16,17-(((R)-cyclohexylmethylene)bis(oxy))-11-hydroxy-21-(2-N,N-diethylamino-1-oxoethoxy)-pregna-1,4-diene-3,20-dione, [11β,16a] (416 mg, 0.713 mmol) in CH₃CN (3 mL) at room temperature was stirred for 2 days and concentrated. The title compound was obtained using a procedure analogous to that described in Example 52, Method A. The title compound was obtained as a white solid (300 mg, 0.264 mmol, 29%) ES/MS calcd. for C₅₉H₈₆N₂O₃P⁺: 1061.6; Found: 1061.5 (M⁺). ¹H NMR (400 MHz, DMSO-d₆): d 9.23-9.09 (m, 1H), 8.94-8.80 (m, 1H), 7.49 (s, 1H), 7.44 (s, 2H), 7.37-7.29 (m, 1H), 7.28-7.21 (m, 2H), 7.16-7.11 (m, 3H), 6.25-6.15 (m, 1H), 6.13 (dd, J=10.4, 1.6 Hz, 1H), 5.89 (s, 1H), 5.17-5.10 (m, 2H), 4.99-4.90 (m, 1H), 4.71 (s, 3H), 4.35 (dd, J=6.96, 2.38 Hz, 3H), 3.69-3.50 (m, 2H), 3.49-3.36 (m, 2H), 3.32 (t, J=6.4 Hz, 2H), 3.29 (t, J=6.4 Hz, 2H), 3.08-2.97 (m, 1H), 2.94-2.79 (m, 3H), 2.55 (t, J=7.50 Hz, 2H), 2.32-2.22 (m, 1H), 2.12-1.92 (m, 3H), 1.82-1.71 (m, 1H), 1.70-0.81 (m, 43H)

### Alternative Preparation of Compound of Example 83.

Mesylate derivative described in Example 5 (3.93 g; 4.43 mmol) was dissolved in anhydrous acetonitrile (8 mL) and the solution of sodium iodide (0.73 g; 4.88 mmol) in anhydrous acetonitrile (4 mL) was added with stirring at room temperature. After 1 h a solution of steroid described in Example 32 (2.07 g; 3.55 mmol) dissolved in anhydrous dichlormethane (6 mL) was added, followed by dropwise addition of the solution of silver trifluoromethanesulfonate (1.26 g; 4.9 mmol) dissolved in anhydrous acetonitrile (3 mL). The flask with reaction mixture was covered with aluminum foil to protect from light and the mixture was vigorously stirred at room temperature overnight. After 15 h the precipitate was filtered off and the filtration cake was washed with copious amount of dichloromethane. Combined filtrate and washes were concentrated, dissolved in minimum amount of diethyl ether and organic phase was extracted with 1N HCl (3 times), saturated sodium bicarbonate solution (3 times), finally with brine, followed by drying over anhydrous sodium sulfate. The residue obtained after filtration of the drying agent and concentration (6.03 g) was dissolved in diethyl ether (20 mL) and added dropwise to vigorously stirred hexanes (80 mL). After 30 min the supernatant was decanted, residue washed with hexanes and the ether/hexanes precipitation procedure repeated twice. Thus obtained residue was dried in vacuo yielding the protected intermediate as a foam (4.102 g; as trifluoromethanesulfonate; yield 76% vs. compound of the Example 32).

The bis-Boc-Bis-tBu-protected intermediate (4.02 g; 2.64 mmol) was dissolved in anhydrous dischloromethane (10 mL) and the solution of HCl (4N; from ampoule) in dioxane (10 mL) was added with vigorous stirring at room temperature. After 1 h diethyl ether (120 mL) was added and stirring continued for another 2 h. The precipitate formed was filtered off, washed with copious amount of diethyl ether and dried to provide 3.15 g of the crude material, which was purified by SCX chromatography (yielding 3.1 g) and subjected to ion-exchange chromatography on Dowex-Cl resin. Resin bed was activated by passing 1N MC1, rinsing with water to neutral pH of the eluent, followed by 2-propanol and dichloromethane. Material was loaded in dichloromethane and eluted with same solvent. Desired fractions were concentrated, evaporated with toluene, redissolved in minimum amount of dichloromethane and the final product was precipitated by addition of hexanes. Filtered-off and dried product (2.018 g; 70%) is a dihydrate of the title compound as determined by elemental analysis and Karl Fischer analysis.

Theory for C₅₉H₈₆ClN₂O₁₃P×2H₂O Formula Wt.: 1133.78—

Calc: C, 62.50; H, 8.00; Cl, 3.13; N, 2.47. K.F. −3.17%.

Found: C, 62.82; H, 7.44; Cl, 3.1; N, 2.50. K.F. −3.1-3.5%.

### Example 84

### [2-(3-Formyl-4-hydroxyphenyl)-2S-hydroxyethyl][6-(4-phenylbutoxy)hexyl]carbamic acid tert-butyl ester

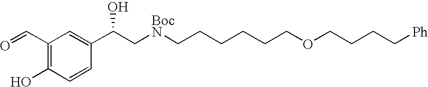

[2-(3-Hydroxymethyl-4-hydroxyphenyl)-2S-hydroxyethyl][6-(4-phenylbutoxy)hexyl]carbamic acid tert-butyl ester (928 mg, 1.8 mmol) was subjected to a procedure analogous to that described in Example 80 to yield the title compound as an oil (828 mg, 90%). ES/MS calcd. For C₃₀H₄₃NNaO₆ 536.3, found m/z=536.3 (M+Na⁺).

### Example 85

### [2-[4-(Di-tert-butoxy-phosphoryloxy)-3-hydroxymethylphenyl]-2S-hydroxyethyl][6-(4-phenylbutoxy)hexyl]carbamic acid tert-butyl ester

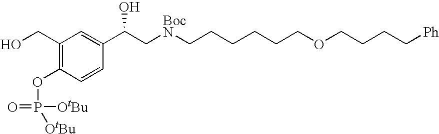

[2-(3-Formyl-4-hydroxyphenyl)-28-hydroxyethyl][6-(4-phenylbutoxy)hexyl]carbamic acid tert-butyl ester (described in Example 84) was subjected to a procedure analogous to that described in Example 81 to yield the title compound as a light yellow oil (940 mg, 85%). ¹H NMR (CDCl₃): 7.17-7.41 (m, 8H), 4.92 (m, 1H), 4.62 (bs, 2H), 3.39 (q, 2H), 2.64 (t 2H), 1.62 (m, 4H), 1.54 (s, 9H), 1.52 (s, 9H), 1.49 (s, 9H), 1.115-1.49 (m, 8H). ³¹PNMR (CDCl₃): −13.060 ppm. LCMS: 99%, MNa 730.0 (exact mass 707.4 calcd for C₃₈H₆₂NO₉P). Anal. Calc: C, 64.48; H, 8.83; N, 1.98. Found: C, 64.70; H, 8.84; N, 1.90.

### Example 86

### Methanesulfonic acid 5-[2-[tert-butoxycarbonyl[6-(4-phenylbutoxy)hexyl]amino]-1S-hydroxyethyl]-2-(di-tert-butoxyphosphoryloxy)benzyl ester

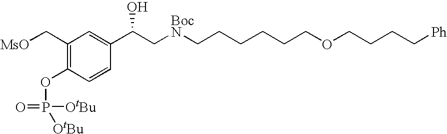

[2-[4-(Di-tert-butoxy-phosphoryloxy)-3-hydroxymethylphenyl]-2S-hydroxyethyl][6-(4-phenylbutoxy)hexyl]carbamic acid tert-butyl ester (described in Example 85) was subjected to a procedure analogous to that described in Example 82 to yield the title compound as a clear oil (546 mg, 0.694 mmol, 52%). ES/MS calcd. For C₃₉H₆₄NNaO₁₁PS 808.4, found m/z=808.3 (M+Na⁺).

### Example 87

### [5-[1-(S)-Hydroxy-2-[6-(4-phenylbutoxy)hexylamino]ethyl]-2-phosphonooxybenzyl]-(diethyl)-[[11β,16α]-[16,17-((R)-cyclohexylmethylene)bis(oxy)]-11-hydroxypregna-1,4-diene-3,20-dion-21-oxy]carbonylmethyl]ammonium chloride

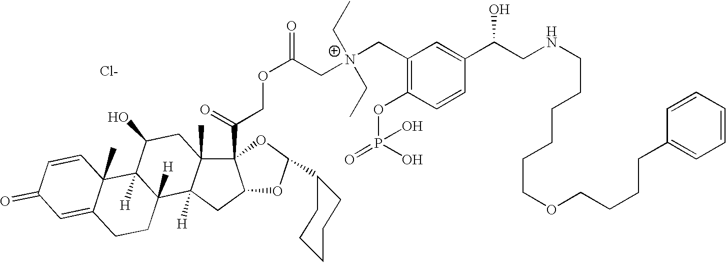

Methanesulfonic acid 5-[2-[tert-butoxycarbonyl[6-(4-phenylbutoxy)hexyl]amino]-1S-hydroxyethyl]-2-(di-tert-butoxyphosphoryloxy)benzyl ester (described in Example 86) was subjected to a procedure analogous to that described in Example 83 to yield the title compound as a white solid (184 ing, 0.264 mmol, 23%) ES/MS calcd. for C₅₉H₈₆N₂O₁₃P⁺:1061.6; Found: 1061.5 (M⁺).

### Example 88

### [5-[1-Hydroxy-2-[6-(4-phenylbutoxy)hexylamino]ethyl]-2-phosphonooxybenzyl]-(dimethyl)-[[2-[[11β,16α]-[[16,17-((R)-cyclohexylmethylene)bis(oxy)]-11-hydroxypregna-1,4-diene-3,20-dion-21-oxy]carbonyl]oxy]propyl]ammonium chloride

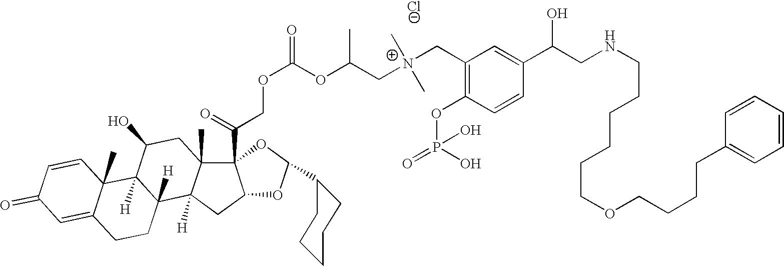

The title compound may be synthesized in manner analogous to Example 46 using the compound prepared as described in Example 28 as starting material.

### Example 89

### [5-[1-Hydroxy-2-[6-(4-phenylbutoxy)hexylamino]ethyl]-2-phosphonooxybenzyl]-(diethyl)-[4-[[11β,16α]-[[16,17-((r)-cyclohexylmethylene)bis(oxy)]-11-hydroxypregna-1,4-diene-3,20-dion-21-oxy]carbonyl]phenylmethyl]ammonium chloride

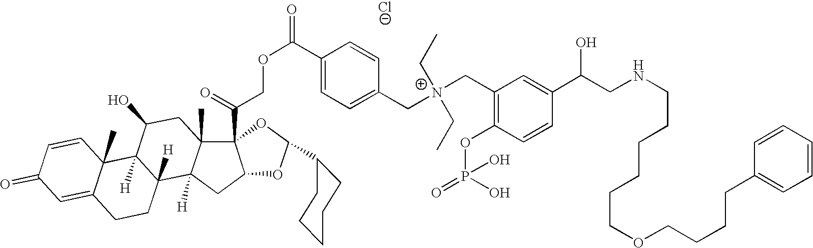

The title compound may be synthesized in a manner analogous to Example 46 using the compound prepared as described in Example 30 as starting material.

### Example 90

### [5-[1-Hydroxy-2-[6-(4-phenylbutoxy)hexylamino]ethyl]-2-phosphonooxybenzyl]-(diethyl)-[1-[[11β,16α]-[[16,17-((R)-cyclohexylmethylene)bis(oxy)]-11-hydroxypregna-1,4-diene-3,20-dion-21-oxy]-carbonyl]ethyl]ammonium chloride

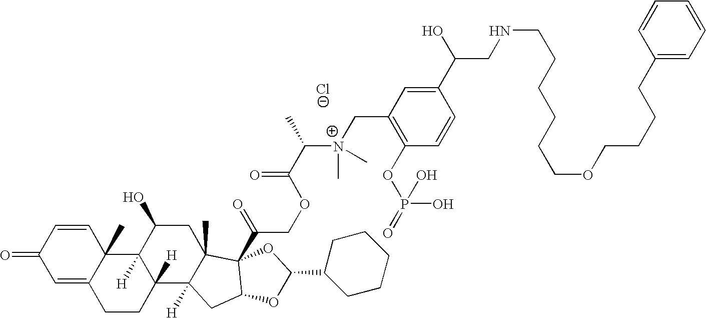

The title compound may be synthesized in a manner analogous to Example 52 Method B, using the compound prepared as described in Example 35 as a starting material.

### Example 91

### [5-[1-Hydroxy-2-[6-(4-phenylbutoxy)hexylamino]ethyl]-2-phosphonooxybenzyl]-(dimethyl)-[1-[[11β,16α]-[[16,17-((R)-cyclohexylmethylene) bis(oxy)]-11-hydroxypregna-1,4-diene-3,20-dion-21-oxy]carbonyl]propyl]ammonium chloride

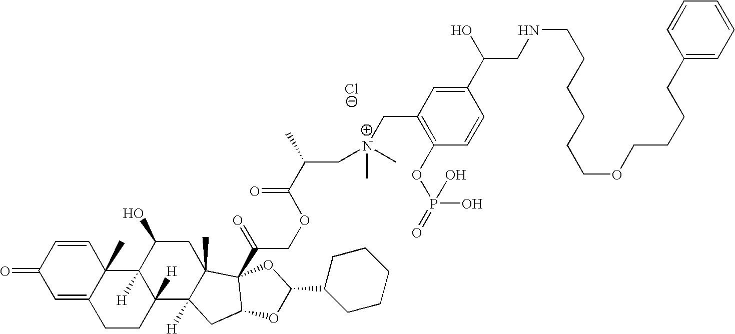

The title compound may be synthesized in a manner analogous to Example 46 using the compound prepared as described in Example 36 as a starting material.

### Example 92

### [5-[1-Hydroxy-2-[6-(4-phenylbutoxy)hexylamino]ethyl]-2-phosphonooxybenzyl]-(dimethyl)-[2-[[11β,16α]-[[16,17-((R)-cyclohexylmethylene bis(oxy)]-11-hydroxypregna-1,4-diene-3,20-dion-21-oxy]carbonyl]-2-cyclopropylethyl]ammonium chloride

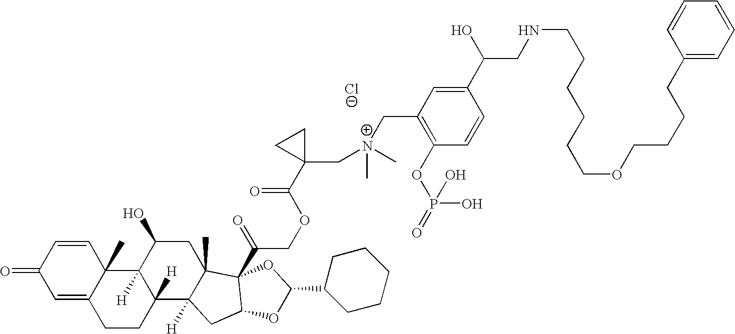

The title compound may be synthesized in an analogous manner to Example 46 using the compound prepared as described in Example 37 as a starting material.

### Example 93

### [5-[1-Hydroxy-2-[6-(4-phenylbutoxy)hexylamino]ethyl]-2-phosphonooxybenzyl]-(diethyl)-[[2-[[6α,11β,16α]-[6,9-difluoro-11-hydroxy-16,17-(1-methylethylidene his oxy)pregna-1,4-diene-3,20-dion-21-oxy]carbonyl]oxy]ethyl]ammonium chloride

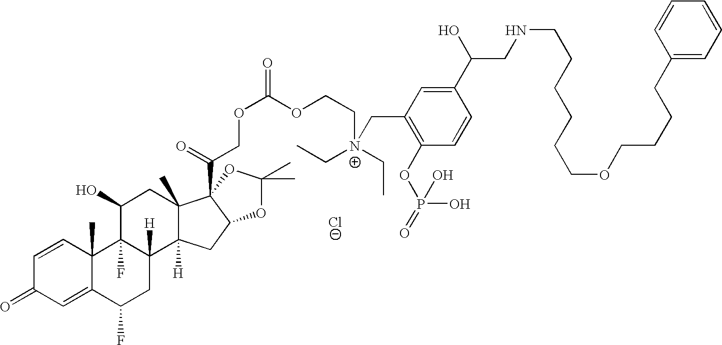

The title compound may be synthesized in a manner analogous to Example 52, Method B using the compound prepared as described in Example 42 as a starting material.

### Example 94

### 1-[5-[1-Hydroxy-2-[6-(4-phenylbutoxy)hexylamino]ethyl]-2-phosphonooxybenzyl]-3-[2-[[11β,16α]-[[16,17-(butylindinedioxy)]-11-hydroxypregna-1,4-diene-3,20-dion-21-oxy]carbonyl]ethen-1-yl]pyridinium chloride

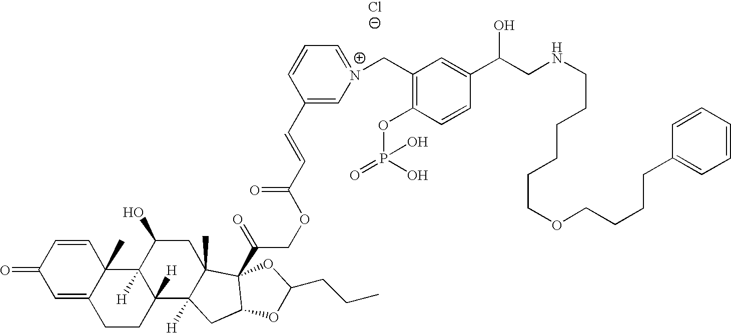

The title compound may be synthesized in a manner analogous to Example 46 using the compound prepared as described in Example 44 as a starting material.

### Example 95

### [5-[1-Hydroxy-2-[6-(4-phenylbutoxy)hexylamino]ethyl]-2-phosphonooxybenzyl]-(dimethyl)-[1-[[11β,16α]-[[16,17-((R)-cyclohexylmethylene) bis(oxy)]-11-hydroxypregna-1,4-diene-3,20-dion-21-oxy]carbonyl]-2-methylpropyl]ammonium chloride

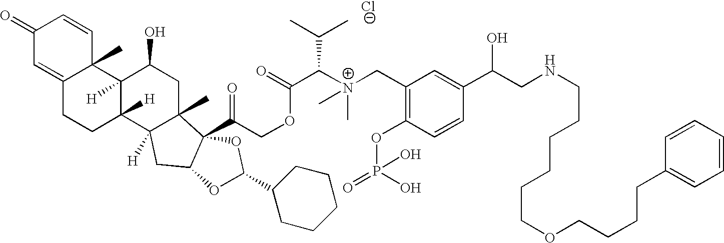

The title compound may be synthesized in a manner analogous to Example 52, Method B, using the compound prepared as described in Example 79 as a starting material.

### Example 96

### [5-[1-hydroxy-2-[6-(4-phenylbutoxy)hexylamino]ethyl]-2-phosphonooxybenzyl]-(dimethyl)-[[11β, 16α]-[[15,16-((R)-cyclohexylmethylene)bis(oxy)]-11-hydroxypregna-1,4-diene-3,20-dion-21-oxy]carbonylmethyl]ammonium chloride

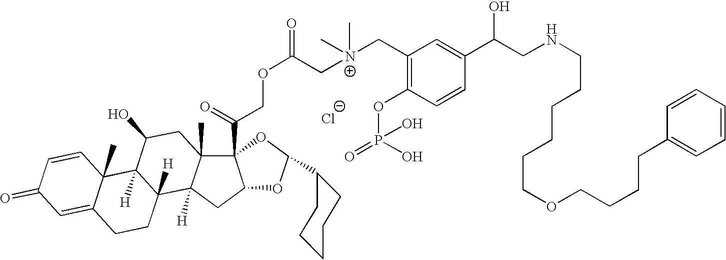

The title compound was synthesized in a manner analogous to Example 46, using the compound prepared as described in Example 75 as starting material. ES/MS cacld. for C₅₇H₈₂N₂O₁₃P⁺: 1033.6; Found: 1033.5 (M⁺).

### Example 97

### Pyrrolidine-1-acetic acid [[11β,16α]-[[((R)-cyclohexylmethylene)bis(oxy)]-11-hydroxypregna-1,4-diene-3,20-dion-21-yl]]ester

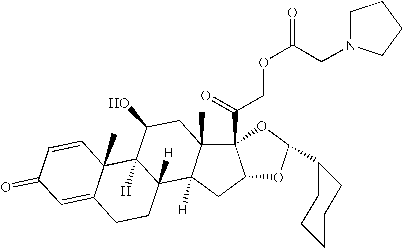

The title compound was synthesized as described in Example 32 using pyrrolidine in place of diethylamine. ES/MS cacld for C₃₄H₄₇NO₇ 582.3, found m/z=582.2 (MH⁺).

### Example 98

### 1-[5-[1-hydroxy-2-[6-(4-phenylbutoxy)hexylamino]ethyl]-2-phoshonooxybenzyl]-1-[[11β,16α]-[[15,16-((R)-cyclohexylmethylene)bis(oxy)]-11-hydroxypregna-1,4-diene-3,20-dion-21-oxy]carbonylmethyl]pyrrolidinium chloride

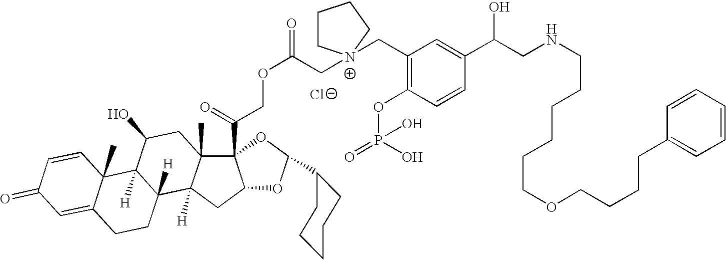

The title compound was synthesized in a manner analogous to Example 46, using the compound prepared as described in Example 97 as a substrate, ES/MS cacld. for C₅₉H₈₄N₂O₁₃P⁺: 1059.57; Found: 1059.57 (M⁺). ¹H NMR (400 MHz, DMSO-d₆): d 8.76-8.54 (m, 2H), 7.49-7.42 (m, 1H), 7.35-7.31 (m, 1H), 7.30-7.24 (m, 2H), 7.22-7.15 (m, 4H), 6.25-6.18 (m, 1H), 6.17 (dd, J=10.4, 1.6 Hz, 1H), 5.93 (s, 1H), 5.15 (d, J=17.8 Hz, 1H), 4.98-4.88 (m, 2H), 4.73-4.70 (m, 1H), 4.43 (d, J=4.4 Hz, 1H), 4.35-4.30 (m, 1H) 4.16-4.04 (m, 3H), 3.69-3.33 (m, 8H), 3.08-2.97 (m, 1H), 2.94-2.79 (m, 3H), 2.55 (t, J=7.50 Hz, 2H), 2.32-2.22 (m, 1H), 2.12-1.92 (m, 3H), 1.82-1.71 (m, 1H), 1.70-0.81 (m, 41H)

### Example 99

### 2-[(4-Methylpiperazin-1-yl)carbonylamino]acetic acid [[11β,16α]-[[((R)-cyclohexylmethylene)bis(oxy)]11-hydroxypregna-1,4-diene-3,20-dion-21-yl]]ester

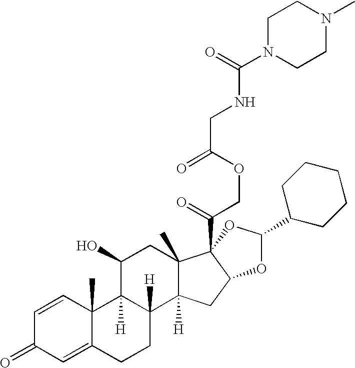

A solution of phosgene (20% in toluene, 2.5 mL, 4.73 mmol) and DIEA (1.6 mL, 9.46 mmol) were added to a stirred solution of compound prepared as described in Example 61 (500 mg, 0.946 mmol) in THF (10 mL) at rt. After stirring for 2 days additional 2.5 mL of phosgene was added. The resulting mixture was concentrated after 6 h then redissolved in acetonitrile (10 mL). Neat 1-methyl-piperazine (1.1 mL, 9.46 mmol) was added to that solution and mixture was stirred for 3 days at rt. The resulting suspension was quenched with satd. NaHCO₃ (20 mL) and the aqueous layer was extracted with EtOAc (3×10 mL). The combined organic layers were washed with brine (20 mL), dried over Na₂SO₄, and concentrated to give crude urea (1.2 g) as a brown oil. Chromatography (9-1, CH₂Cl₂/MeOH) afforded the title compound (145 mg, 23% 2 steps). ES/MS calcd for C₃₆H₅₂N₃O 654.4, found m/z=654.7 (MH⁺).

### Example 100

### 4-Methylpiperazine-1-acetic acid [[11β,16α]-[[((R)-cyclohexylmethylene)bis(oxy)]-11-hydroxypregna-1,4-diene-3,20-dion-21-yl]]ester

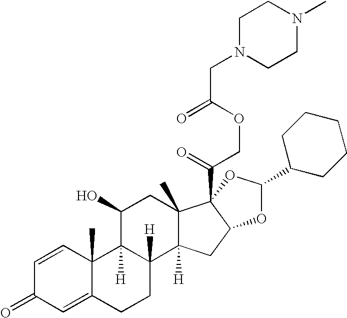

The title compound was synthesized in a manner analogous to that described in Example 32, using 1-methyl-piperazine in place of diethylamine. ES/MS calcd for C₃₅H₅₁N₂O₇, 611.4, found m/z=611.3 (MH⁺).

### Example 101

### 1-[5-[1-hydroxy-2-[6-(4-phenylbutoxy)hexylamino]ethyl]-2-phosphonooxybenzyl]-4-[[[11β,16α]-[[15,16-((R)-cyclohexylmethlylene)bis(oxy)]-11-hydroxypregna-1,4-diene-3,20-dion-21-oxy]carbonylmethyl]aminocarbonyl]-1-methylpiperazinium chloride

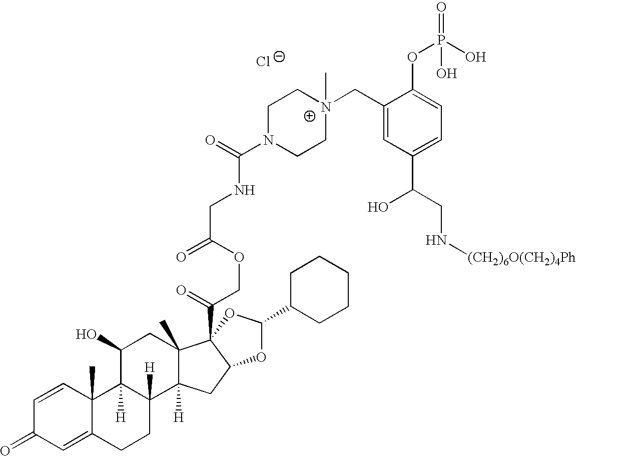

The title compound may be synthesized in a manner analogous to that described in Example 46, using the compound prepared as described in Example 99 as a substrate.

### Example 102

### 1-[5-[1-hydroxy-2-[6-(4-phenylbutoxy hexylamino]ethyl]-2-hydroxypregna-1,4-diene-3,20-dion-21-oxy]carbonylmethyl-1-methylpiperazinium chloride

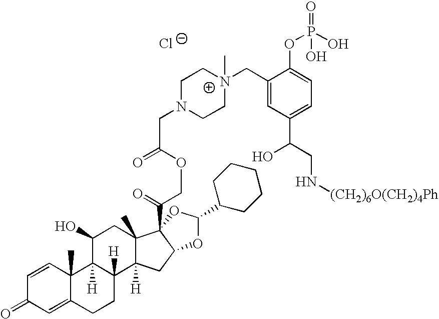

The title compound was synthesized in a manner analogous to that described in Example 46, using the compound prepared as described in Example 100 as a substrate. ES/MS calcd for C₆₀H₈₇N₃O₁₃P⁺ 1088.7, found m/z=1088.6 (M⁺). ¹H NMR (400 MHz, DMSO-d₆) d 9.17 (m, 11H), 8.82 (m, 1H), 7.49 (m, 2H), 7.29 (d, 1H, J=10.2 Hz), 7.19 (In, 1H), 7.09 (m, 2H), 6.27 (m, 1H), 6.10 (m, 1H), 5.84 (m, 1H), 4.98 (m, 2H), 4.79 (m, 1H), 4.63 (brs, 2H), 4.32 (d, 1H, J=4.0 Hz), 4.24 (brs, 1H), 3.44 (m, 3H), 3.25 (m, 3H), 2.93 (m, 6H), 2.41 (m, 1H), 2.22 (m, 1H), 1.97 (m, 2H), 1.84-0.69 (m, 53H)

### Example 103

### 1-[5-[1-hydroxy-2-[6-(4-phenylbutoxy)hexylamino]ethyl]-2-phosphonooxybenzyl]-4-[[11β,16α]-[[15,15-((R)-cyclohexylmethylene)bis(oxy)]-11-hydroxypregna-1,4-diene-3,20-dion-21-oxy]carbonyl]-1-methylpiperazinium chloride

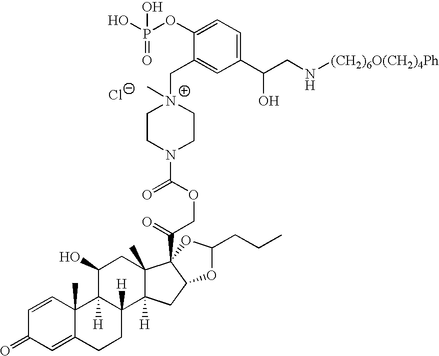

The title compound was synthesized in a manner analogous to that described in Example 46, using the compound prepared as described in Example 45 as a substrate. ES/MS calcd for C₅₆H₈₁N₃O₁₃P⁺: 1035.4, found m/z=1035.5 (M⁺).

### Example 104

### [5-[1-hydroxy-2-(1,1-dimethylethylamino)ethyl]-2-phosphonooxybenzyl]-(diethyl)-[[11β,16α]-[[15,16-((R)-cyclohexylmethylene)bis(oxy)]-11-hydroxypregna-1,4-diene-3,20-dion-21-oxy]carbonylmethyl]ammonium chloride

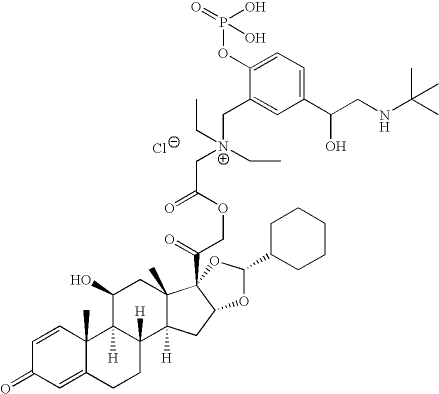

21-N,N-diethylglycyl-desisobutyryl ciclesonide (described in Example 32) (284 mg, 0.486 mmol) and PMP (0.264 mL, 1.46 mmol) were added to stirred solution of carbonic acid tert-butyl ester [2-tert-butylamino-1-[4-(di-tert-butoxyphosphoryloxy)-3′-hydroxymethylphenyl]ethyl]ester (described in Example 15) (310 mg, 0.583 mmol) in DCM (5 mL) at rt. The resulting mixture was cooled to −78° C. then a solution of Tf₂O (0.122 mL, 0.729 mmol) in DCM (0.7 mL) was added dropwise. Immediately that the cooling bath was removed and the solution was allowed to warn to rt over 40 min. The resulting suspension was concentrated to dryness and dissolved in EtOAc (10 mL). The organic layer was washed with 10% (w/v) citric acid (20 mL), satd. NaHCO₃ (20 mL), brine, dried over Na₂SO₄, and concentrated to give crude ammonium salt (686 mg) as a light yellow foamy solid. That intermediate was dissolved in EtOAc (4 mL) and the solution added dropwise into stirred hexane (50 mL). The supernatant was decanted and residue was dried and dissolved in DCM (15 mL). Dry, gaseous HCl was bubbled through the solution for 1 min and then resulting solution was stirred for 6 h at it. The reaction mixture was concentrated and purified by ion exchange chromatography, as described in Example 52, to give the title compound (307 mg, 66% after 2 steps) as an off-white solid. ES/MS calcd for C₄₇H₇₀N₂O₁₂P⁺, 885.5, found m/z=885.5 (M⁺). ¹H NMR (400 MHz, DMSO-d₆) d 9.36 (s, 1H), 8.61 (s, 1H), 7.51 (d, 2H, J=7.9 Hz), 7.44 (d, 1H, J=8.8 Hz), 7.30 (d, 1H, J=10. Hz), 6.30 (brs, 1H), 6.10 (d, 1H, J=10.1 Hz), 5.86 (s, 1H), 5.11 (m, 1H), 5.00 (m, 2H, J=19.3 Hz), 4.69 (m, 3H, J=20.6 Hz), 4.33 (m, 4H, J=16.5, 20.4 Hz), 3.43 (m, 5H), 2.99 (m, 1H), 2.86 (m, 1H), 2.25 (m, 1H), 2.09-0.74 (m, 43H).

### Example 105

### 3-Methylthiopropanoic acid [[11β,16α]-[[((R)-cyclohexylmethylene)bis(oxy)]-11-hydroxypregna-1,4-diene-3,20-dion-21-yl]methyl]ester

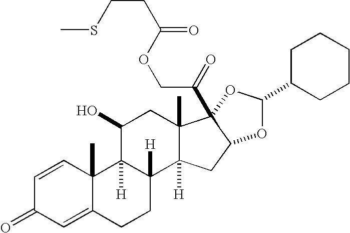

The title compound was synthesized in a manner analogous to that described in Example 25, using 3-(methylthio)propanoic acid in place of 1-methylpiperidine-4-carboxylic acid, to provide crude ester (1.42 g) as a light yellow solid. Chromatography (Hexanes/EtOAc gradient 1:0 to 1:1) afforded the title compound (0.52 g, 44% yield) as a white solid. ES/MS calcd. for C₃₂H₄₄O₇S 572.3, found m/z=572.7 (M+H).

### Example 106

### [5-[1-hydroxy-2-[6-(4-phenylbutoxy)hexylamino]ethyl]-2-phosphonooxybenzyl]-(methyl)-[[11β,16α]-[[15,16-((R)-cyclohexylmethylene)bis(oxy)]-11-hydroxypregna-1,4-diene-3,20-dion-21-oxy]carbonylethyl]thionium chloride

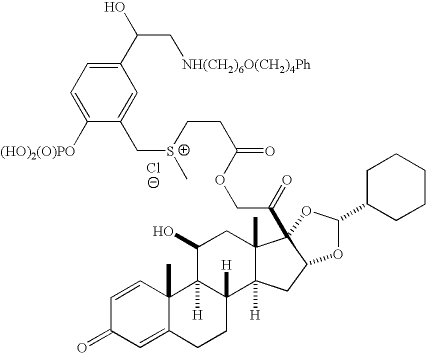

The title compound may be synthesized in a manner analogous to that described in Example 52, Method B, using the compound prepared as described in Example 105 as a substrate.

### Example 107

### 2-Methylthio-acetic acid [[11β,16α]-[((R)-cyclohexylmethylene)bis(oxy)]-11-hydroxypregna-1,4-diene-3,20-dion-21-yl]ester

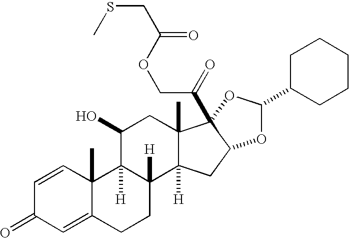

The title compound was synthesized in a manner analogous to that described in Example 25, using 2-(methylthio)acetic acid in place of 1-methylpiperidine-4-carboxylic acid, to provide crude ester (1.53 g) as a light yellow solid. Chromatography (Hexanes/EtOAc gradient 1:0 to 1:1) afforded the title compound (0.28 g, 24% yield) as a white solid. ES/MS calcd. for C₃₁H₄₂O₇S 558.7, found m/l=560.1 (M+H).

### Example 108

### [5-[1-hydroxy-2-[6-(4-phenylbutoxy)hexylamino]ethyl]-2-phosphonooxybenzyl]-(methyl)-[[11β,16α]-[[15,16-((R)-cyclohexylmethylene)bis(oxy]-11-hydroxypregna-1,4-diene-3,20-dion-21-oxy]carbonylethyl]thionium chloride

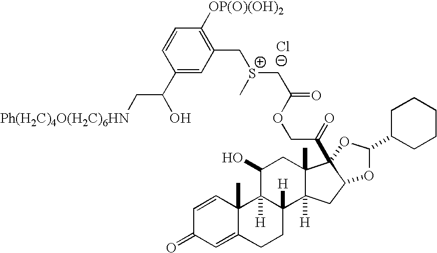

The title compound may be synthesized in a manner analogous to that described in Example 52, Method B, using the compound prepared as described in Example 107 as a substrate.

### Example 109

### 4-(Imidazol-1-yl)benzoic acid [11β,16α]-[[((R)-cyclohexylmethylene)bis(oxy)]-11-hydroxypregna-1,4-diene-3,20-dion-21-yl]ester

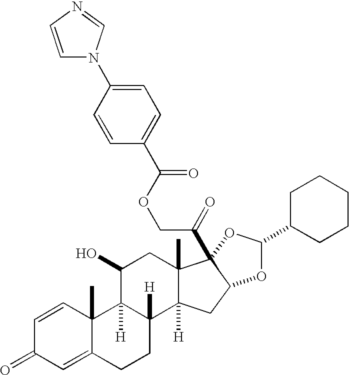

The title compound was synthesized as described in Example 25, substituting 1-methylpiperidine-4-carboxylic acid with 4-(imidazol-1-yl)benzoic acid, to provide crude ester (0.88 g) as a light yellow solid. Chromatography (Hexanes/EtOAc gradient 1:1 to 0:1) afforded the title compound (0.44 g, 33% yield) as white solid. ES/MS calcd. for C₃₈H₄₄N₂O₇ 640.8, found m/z=641.2 (M+H).

### Example 110

### [5-[1-hydroxy-2-[6-(4-phenylbutoxy)hexylamino]ethyl]-2-phosphonooxybenzyl][4-[11β,16α]-[[15,16-((R)-cyclohexylmethylene)bis(oxy)]-11-hydroxypregna-1,4-diene-3,20-dion-21-oxy]carbonylphenyl]imidazolium chloride

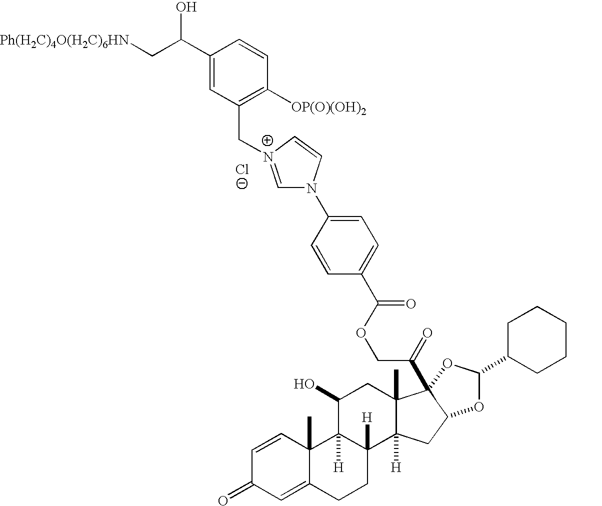

The title compound was synthesized in a manner analogous to that described in Example 77, using the compound prepared as described in Example 109 as a substrate to provide crude imidazolium salt (0.12 g) as a yellow solid. Chromatography (SCX column, gradient DCM to MeOH) afforded the title compound (0.07 g, 50% yield) as a white solid. ES/MS calcd. for C₆₃H₈₁N₃O₁₃P⁺ 1119.3, found m/z 1119.3 (M⁺). ¹H NMR (400 MHz, DMSO-d₆) d 8.88 (br, 1H), 8.65 (br, 1H), 8.42 (s, 1H), 8.21 (d, J=8.8 Hz, 2H), 8.07-7.99 (m, 3H), 7.58 (s, 1), 7.39 (s, 1H), 7.36-7.31 (m, 14), 7.27-7.22 (m, 2H), 7.17-7.13 (m, 4H), 6.23-6.09 (m, 1H), 5.92 (s, 1H), 5.51 (s, 2H), 5.30-5.22 (m, 1H), 5.12-5.00 (m, 1H), 4.94-4.87 (m, 2H), 4.72-4.70 (m, 1H), 4.48-4.44 (m, 1H), 4.34 (br, 1H), 3.13-2.83 (m, 4H), 2.58-2.52 (m, 2H), 2.33-2.26 (m, 1H), 2.14-1.82 (m, 3H), 1.73-1.42 (m, 23H), 1.38 (s, 3H), 1.31-0.95 (m, 14H), 0.88 (s, 3H).

### Example 111

### 2-Methyl-1-imidazolpropionic acid [11β,16α]-[[((R)-cyclohexylmethylene)bis(oxy)]-11-hydroxypregna-1,4-diene-3,20-dion-21-yl]ester

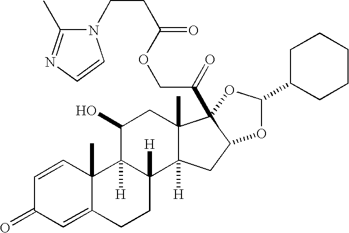

The title compound was synthesized in a manner analogous to that described in Example 25, using 3-(2-methyl-imidazol-1-yl)-propionic acid in place of 1-methylpiperidine-4-carboxylic acid to provide the crude ester (1.02 g) as a light yellow solid. Chromatography (EtOAc/DCM/MeOH gradient 1:0:0 to 0:1:0 to 0:9:1 then DCM/MeOH gradient 1:0 to 9:1) afforded the title compound (0.60 g, 48% yield) as a white solid. ES/MS calcd. for C39H₄₆N₂O₇ 606.8, found m/z=607.2 (M+H).

### Example 112

### 1-[5-[1-hydroxy-2-[6-(4-phenylbutoxy)hexylamino]ethyl]-2-phosphonooxybenzyl]-3-[[11β,16α]-[[15,16-((R)-cyclohexylmethylene)bis(oxy)]-11-hydroxypregna-1,4-diene-3,20-dion-21-oxy]carbonylethyl]-2-methylimidazolium chloride

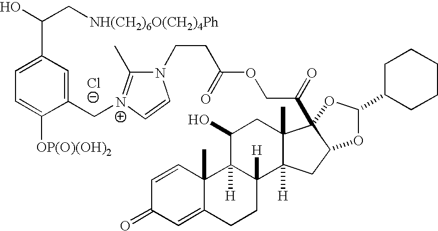

The title compound was synthesized in a manner analogous to that described in Example 77, using the compound prepared as described in Example 11 as a substrate to provide crude imidazolium salt (0.49 g) as a yellow solid. Chromatography (SCX column, gradient DCM to MeOH) afforded title compound (0.21 g, 46% yield) as a white solid. ES/MS calcd. for C₆₀H₈₃N₃O₁₃P⁺ 1084.6, found m/z=1084.6 (M⁺).

### Example 113

### 1-Imidazoleacetic acid [[11β,16⊕]-[[((R)-cyclohexylmethylene)bis(oxy)]-11-hydroxypregna-1,4-diene-3,20-dion-20-yl]methyl]ester

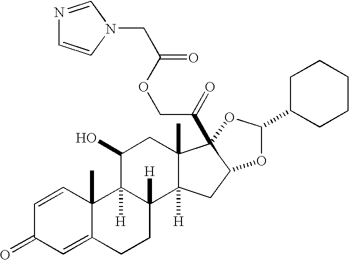

The title compound was synthesized in a manner analogous to that described in Example 25, using imidazol-1-yl-acetic acid in place of 1-methylpiperidine-4-carboxylic acid, to provide the crude ester (1.18 g) as a light yellow solid. Chromatography (EtOAc/DCM/MeOH gradient 1:0:0 to 0:1:0 to 0:9:1 then DCM/MeOH gradient 1:0 to 9:1) afforded the title compound (0.69 g, 58% yield) as white solid. ES/MS calcd. for C₃₃H₄₂N₂O₇ 578.3, found m/z=579.3 (M+H).

### Example 114

### 1-[5-[1-hydroxy-2-[6-(4-phenylbutoxy)hexylamino]ethyl-2-hydroxypregna-1,4-diene-3,20-dion-21-(oxy)]carbonylmethyl]imidazolium chloride

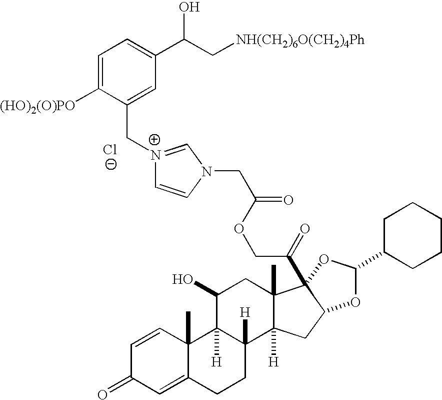

The title compound was synthesized in a manner analogous to that described in Example 77, using the compound prepared as described in Example 113 as a substrate to provide the crude imidazolium salt (0.12 g) as a yellow solid. Chromatography (SCX column gradient DCM to MeOH) afforded title compound (0.06 g, 34% yield) as a white solid. ES/MS calcd. for C₅₈H₇₉N₃O₁₃P⁺ 1056.5, found m/<=1056.5 (M⁺).

### Example 115

### 2-Ethylaminoacetic acid [[11β,16α]-[((R)-cyclohexylmethylene)bis(oxy)]-11-hydroxypregna-1,4-diene-3,20-dion-21-yl]ester

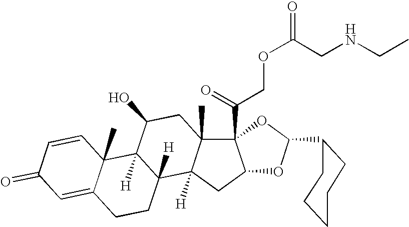

The title compound was synthesized in a manner analogous to that described in Example 50, using Boc-N-ethylglycine in stead of Boc-(R)-valine. ES/MS calcd for C₃₂H₄₆NO₇ 556.3, found me/=556.2 (M+H⁺).

### Example 116

### 1-[[5-[1-hydroxy-2-[(t-butoxycarbonyl)[6-(4-phenylbutoxy)hexyl]amino]ethyl]-2-(di-tert-butoxyphosphoryloxy)benzyl]amino]acetic acid [[11β,16α]-[[15,16-((R)-cyclohexylmethylene)bis(oxy)]-11-hydroxypregna-1,4-diene-3,20-dion-21-yl]ester

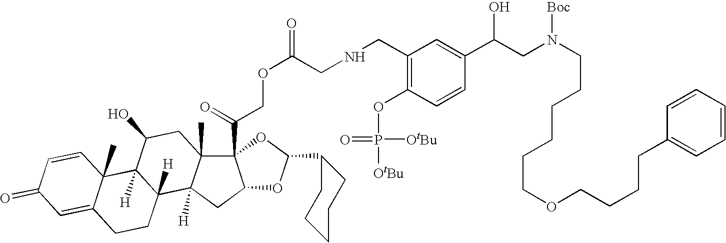

The compound prepared as described in Example 115 (0.831 g, 1.575 mmol) was added to a solution of [2-[4-(di-tert-butoxyphosphoryloxy)-3-formylphenyl]-2-hydroxyethyl][6-(4-phenylbutoxy)hexyl]carbamic acid tert-butyl ester (described in Example 1) (1.283 g, 1.818 mmol) in 1,2-dichloroethane (6 mL). Sodium triacetoxyborohydride (0.512 g, 2.416 mmol) was then added in one portion and the reaction mixture stirred overnight. It was quenched by the addition of saturated NaHCO₃ and layers separated. The aqueous layer was then extracted with EtOAc (3×). The combined organic extracts were washed with brine, dried over MgSO₄, filtered, and concentrated. The residue was purified by silica gel chromatography (gradient: 50% to 100% EtOAc in hexanes, column pretreated with TEA) to give the title compound as a white solid (0.783 g, 41%). ES/MS calcd for C₆₈H₁₀₂N₂O₁₅P 1217.7, found m/z=1217.6 (M+H⁺).

### Example 117

### 1-[5-[1-hydroxy-2-[[6-(4-phenylbutoxy)hexyl]amino]ethyl]-2-(2-phosphonooxy)benzyl]amino]acetic acid [[11β,16α]-[[15,16-((R) -cyclohexylmethylene)bis(oxy)]-11-hydroxypregna-1,4-diene-3,20-dion-21-yl]ester dihydrochloride

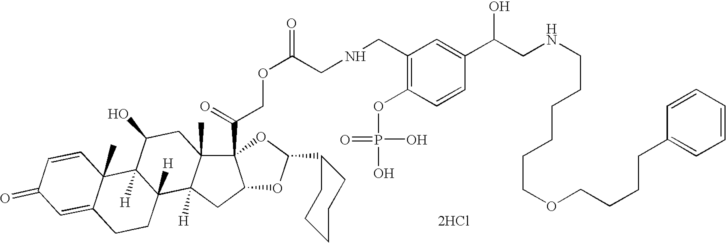

To a solution of compound described in Example 116 (0.146 g, 0.120 mmol) in DCM (0.25 mL), 4N HCl in dioxane (0.25 mL) was added dropwise over 5 min maintaining 0° C. The ice bath was removed and the mixture stirred at rt overnight. Et₂O, was added to precipitate the product and the suspension was centrifuged. The supernatant was removed and the solid was dissolved in DCM and the precipitation and centrifugation procedure was repeated to give title compound as a white solid (0.121 g, 94%). ES/MS calcd for C₅₅H₇₈N₂O₁₃P 1005.5, found m/z=1005.5 (M+H⁺).

### Example 118

### 1-[[5-[1-hydroxy-2-[t-butoxycarbonyl)[6-(4-phenylbutoxy)hexyl]amino]ethyl]-2-(di-tert-butoxyphosphoryloxy)benzyl](ethyl)amino]acetic acid [[11β,16α]-[[15,16-((R)-cyclohexylmethylene bis(oxy)]-11-hydroxypregna-1,4-diene-3,20-dion-21-yl]ester

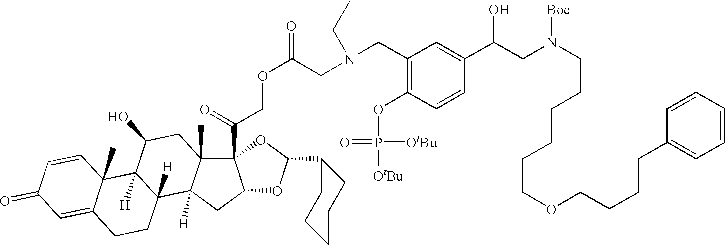

To a solution of the compound described in Example 116 (0.259 g, 0.821 mmol) in DMF (8.2 mL), PMP (1.5 mL, 8.3 mmol) was added, followed by iodoethane (0.43 mL, 5.35 mmol). The reaction mixture was heated at about 50° C. using an oil bath. After 4 h, water was added to the reaction mixture, which was then extracted with EtOAc (4×). The combined organic extracts were washed with water and brine, dried over MgSO₄, filtered, and concentrated. The residue was purified by silica gel chromatography (gradient: 0% to 100% EtOAc in hexanes) to give title compound as a white solid (0.192 g, 72%). ¹H NMR (400 MHz, CDCl₃): d 7.57-7.55 (m, 1H), 7.32-7.15 (m, 8H), 6.27 (dd, 1H, J=10.0, 2.0 Hz), 6.03-6.01 (m, 1H), 4.90-4.81 (m, 3H), 4.74-4.69 (m, 1H), 4.49-4.45 (m, 1H), 4.32 (dd, 1H, J=4.8, 2.0 Hz), 3.92-3.82 (m, 2H), 3.46-3.43 (m, 2H), 3.40 (t, 2H, J=6.4 Hz), 3.37 (t, 2H, J=6.4 Hz), 2.73 (quartet, 2H, J=7.2 Hz), 2.62 (t, 2H, J=7.5 Hz), 2.57-2.52 (m, 1H), 2.35-2.31 (m, 1H), 2.20-1.90 (m, 6H), 1.75-1.52 (m, 21H), 1.50 (s, 9H), 1.48 (s, 18H), 1.46-1.45 (m, 3H), 1.35-1.31 (m, 6H), 1.23-1.08 (m, 8H), 1.10 (t, 3H, J=7.2 Hz).

### Example 119

### 1-[5-[1-hydroxy-2-[[6-(4-phenylbutoxy)hexyl]amino]ethyl]-2-(2-phosphonooxy)benzyl](ethyl)amino]acetic acid [[11β,16α]-[[15,16-((R-cyclohexylmethylene)bis(oxy)]-11-hydroxypregna-1,4-diene-3,20-dion-21-yl]ester dihydrochloride

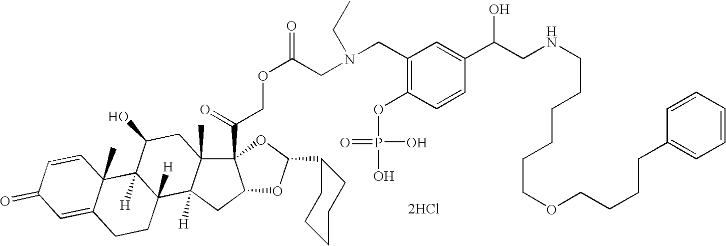

To a solution of the compound described in Example 118 (0.192 g, 0.154 mmol) in DCM (0.3 mL), a 4N solution of HCl in dioxane (0.3 mL) was added and stirred at 0° C. The ice bath was removed and the mixture stirred at rt for 1 h. Et₂O was added to precipitate the product and the suspension was centrifuged. The supernatant was removed and the remaining solid was dissolved in DCM and the precipitation/centrifugation procedure was repeated to give title compound as a white solid (0.124 g, 73%). ES/MS calcd for C₅₇H₈₂N₂O₁₃P 1033.6, found m/z=1033.5 (M+H⁺).

### Example 120

### 2-(t-Butoxycarbonylamino)-6-dimethylaminohexanoic acid [[11β,16α]-[16,17-((R)-cyclohexylmethylene)bis(oxy)]-11-hydroxypregna-1,4-diene-3,20-dion-21-yl]ester

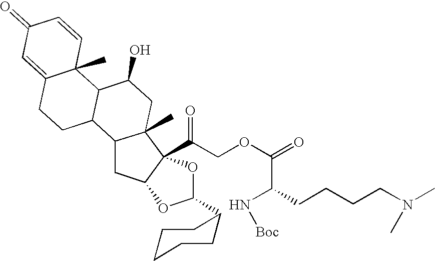

The title compound was synthesized in a manner analogous to that described in Example 25, using Boc-Lys(Me)₂-OH in place of 1-methylpiperidine-4-carboxylic acid. Chromatography on silica gel (DCM with increasing gradient of 2-propanol) gave the product in amorphous form in 79% yield. ES/MS calcd for C₄₁H₆₂N₂O₉ 726.4, found m/z=727.4 (M+H⁺).

### Example 121

### [5-[1-hydroxy-2-[6-(4-phenylbutoxy)hexylamino]ethyl]-2-phosphonooxybenzyl]-(dimethyl)-[5-amino-5-[[11β,16α]-[[15,16α-((R)-cyclohexylmethylene)bis(oxy)]-11-hydroxypregna-1,4-diene-3,20-dion-21-oxy]carbonyl]pentyl]ammonium chloride

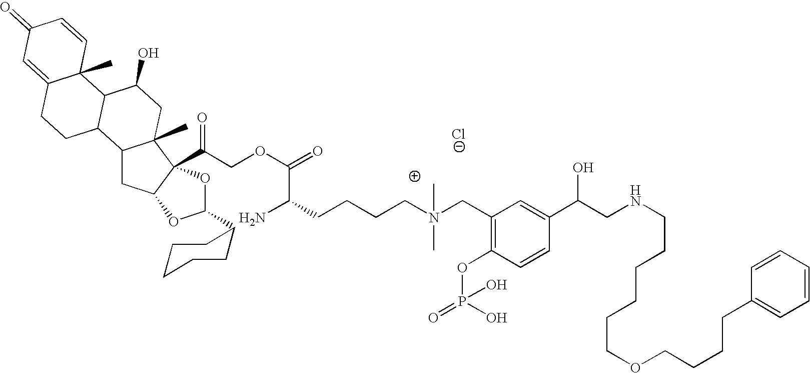

The title compound was synthesized in a manner analogous to that described in Example 46, using compound prepared as described in Example 120 as a substrate. Product was isolated as a hydrochloride, as indicated by elemental analysis, in overall 17% yield. ¹H NMR (400 MHz, DMSO-d₆): d 9.92 (b, 1H), 9.15 (b, 1H), 8.83 (b, 1H), 7.60-7.49 (m, 1H), 7.40-7.14 (m, 8H), 6.17 (dd, 1H, J=10.0, 2.0 Hz), 5.93 (bs, 1H), 5.15-4.91 (m, 3H), 4.71-4.54 (m, 2H), 4.42-4.16 (m, 3H), 3.39-3.30 (m, 3H), 3.10-2.99 (m, 4H), 2.60-2.54 (m, 1H), 2.10-1.77 (m, 9H), 1.73-1.44 (m, 30H), 1.38 (s, 3H), 1.33-1.27 (m, 3H), 1.24-0.92 (m, 12H), 0.86 (s, 3H).

³¹P NMR (DMSO-d₆): −6.658 ppm. ES/MS calcd for C₆₁H₉₁N₃O₁₃P 1104.6, found m/z=1105.6 (M+H⁺).

### Example 122

### N⁵-[bis(methylamino)methylene]-N²,N²-dimethyl-L-ornithine [[11β,16α]-[16,17-((R)-cyclohexylmethylene)bis(oxy)]-11-hydroxypregna-1,4-diene-3,20-dion-21-yl]ester

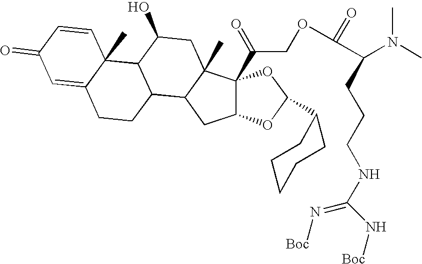

The title compound may be synthesized in a manner analogous to that described in Example 25, substituting N,N-dimethyl-Arg(Boc)₂ for 1-methylpiperidine-4-carboxylic acid.

### Example 123

### [5-[1-Hydroxy-2-[6-(4-phenylbutoxy)hexylamino]ethyl]-2-phosphonooxybenzyl]-(dimethyl)-[1-[[11β,16α]-[[16,17-((R)-cyclohexylmethylene)bis(oxy)]-11-hydroxypregna-1,4-diene-3,20-dion-21-oxy]carbonyl]-4-[[amino(imino)methyl]amino]-butyl]ammonium chloride

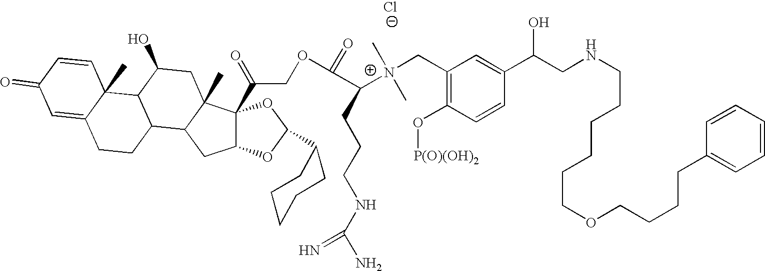

The title compound may be synthesized in a manner analogous to that described in Example 46, using compound prepared as described in Example 122 as a substrate.

### Example 124

### 4-Methylthiobenzoic acid [[11β,16α]-[[((R)-cyclohexylmethylene)bis(oxy)]-11-hydroxypregna-1,4-diene-3,20-dion-21-yl]ester

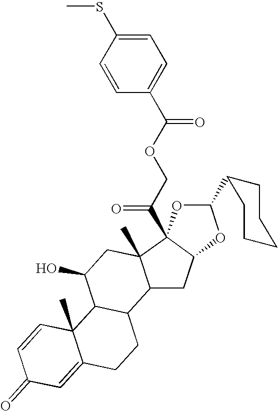

The title compound can be synthesized in a manner analogous to that described in Example 25, using 4-(methylthio)benzoic acid in place of 1-methylpiperidine-4-carboxylic acid.

### Example 125

### [5-[1-hydroxy-2-[6-(4-phenylbutoxy)hexylamino]ethyl]-2-phosphonooxybenzyl]-(methyl)-[4-[[11β,16α]-[15,16-((R)-cyclohexylmethylene)bis(oxy)-11-hydroxypregna-1,4-diene-3,20-dion-21-oxy]-carbonyl]phenyl]thionium

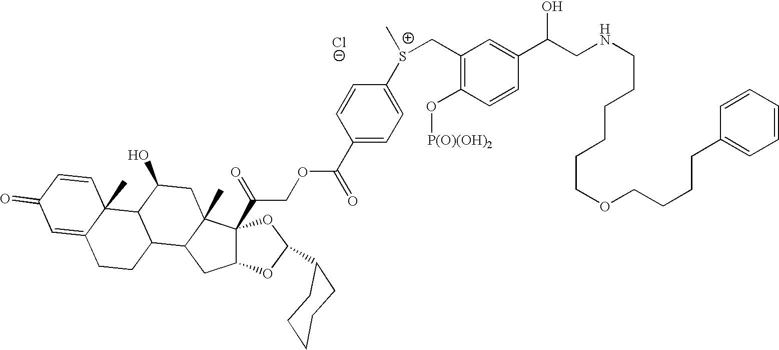

The title compound may be synthesized in a manner analogous to that described in Example 52 Method B, using the compound prepared as described in Example 124 as starting material.

### Example 126

### 3-Methylthiobenzoic acid [[11β,16α]-[[((R)-cyclohexylmethylene)bis(oxy)]-11-hydroxypregna-1,4-diene-3,20-dion-21-yl]ester

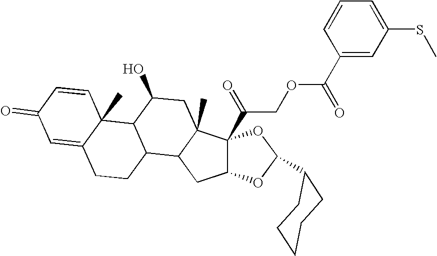

The title compound may be synthesized in a manner analogous to that described in Example 25, using 3-(methylthio)benzoic acid in place of 1-methylpiperidine-4-carboxylic acid.

### Example 127

### [5-[1-hydroxy-2-[6-(4-phenylbutoxy)hexylamino]ethyl]-2-phosphonooxybenzyl]-(methyl)-[3-[[11β,16α]-[[15,16-((R)-cyclohexylmethylene)bis(oxy)]-11-hydroxypregna-1,4-diene-3,20-dion-21-oxy]carbonyl]phenyl]thionium

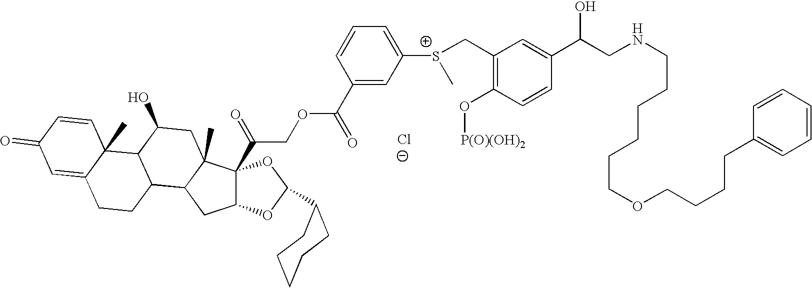

The title compound may be synthesized in a manner analogous to that described in Example 52 Method B, using the compound prepared as described in Example 126 as starting material.

### Example 128

### N-[(Pyridin-3-yl)-carbonyl]proline [[β,16α]-16,17-((R)-cyclohexylmethylene)bis(oxy)]-11-hydroxypregna-1,4-diene-3,20-dion-21-yl]ester

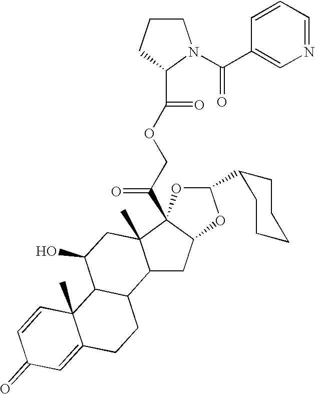

The title compound may be synthesized in a manner analogous to that described in Example 25, using nicotinoyl-Pro-OH in place of 1-methylpiperidine-4-carboxylic acid.

### Example 129

### 1-[5-[1-Hydroxy-2-[6-(4-phenylbutoxy)hexylamino]ethyl]-2-phosphonooxybenzyl]-3-[[2-[[11β,16α]-[16,17-((R)-cyclohexylmethylene)bis(oxy)]-11-hydroxypregna-1,4-diene-3,20-dion-21-oxy]carbonyl]pyrrolidin-1-ylcarbonyl]pyridinium chloride

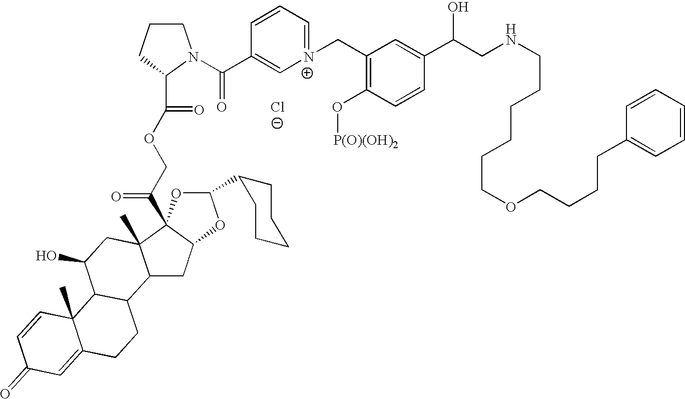

The title compound may be synthesized in a manner analogous to that described in Example 46, using compound prepared as described in Example 128 as a substrate

### Example 130

### N^{ζ},N^{ζ}-di-(t-Butoxycarbonyl)-N^{a}-[(pyridine-3-yl)carbonyl]arginine [[11β,16α]-[16,17-((R)-cyclohexylmethylene)bis(oxy)]-11-hydroxypregna-1,4-diene-3,20-dion-21-yl]ester

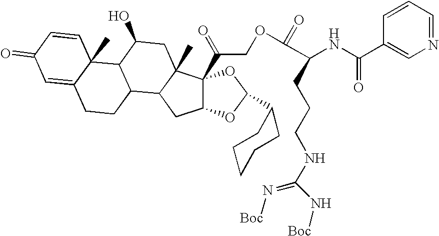

The title compound may be synthesized in a manner analogous to that described in Example 25, using nicotinoyl-Arg(Boc)₂-OH in place of 1-methylpiperidine-4-carboxylic acid.

### Example 131

### 1-[5-[1-Hydroxy-2-[6-(4-phenylbutoxy)hexylamino]ethyl]-2-phosphonooxybenzyl][[[1-[[11β,16α]-[[16,17-((R)-cyclohexylmethylene)bis(oxy)]-11-hydroxypregna-1,4-diene-3,20-dion-21-oxy]carbonyl]-4-[[amino(imino)methyl]amino]butyl]amino]carbonyl]pyridinium chloride

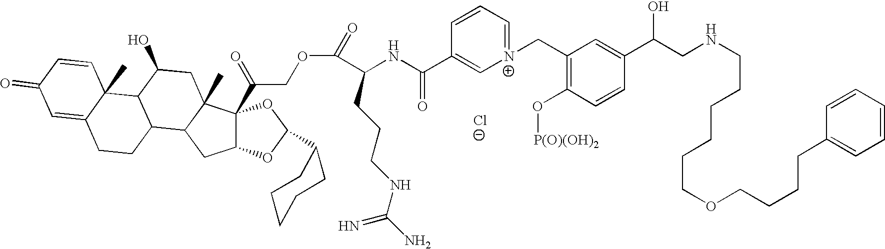

The title compound may be synthesized in a manner analogous to that described in Example 46, using compound prepared as described in Example 130 as a substrate.

### Example 132

### (Pyridin-4-yl)acetic acid [[11β,16a]-[[((R)-cyclohexylmethylene)bis(oxy)]-11-hydroxypregna-1,4-diene-3,20-dion-21-yl]]ester

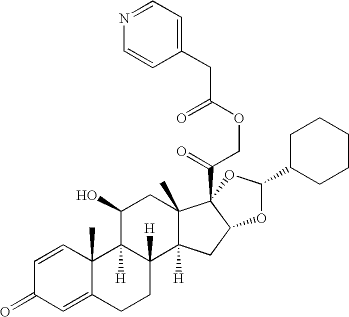

The title compound was synthesized in a manner analogous to that described in Example 33, using 4-pyridylacetic acid hydrochloride in place of 3-pyridine-3-yl-acrylic acid. NH NMR (400 MHz, CDCl₃) d 8.59 (dd, 2H, J=1.6, 4.4 Hz), 7.28-7.23 (m, 3H), 6.28 (dd, 1H, J=1.9, 10.1 Hz), 6.03 (s, 1H), 4.84 (m, 3H), 4.50 (s, 1H), 4.33 (d, 1N, J=4.6 Hz), 3.77 (s, 2H), 2.55 (m, 1H), 2.34 (m, 1H), 2.22-5304 (m, 3H), 1.81-1.57 (m, 9H), 1.45 (s, 3H), 1.32-0.87 (m, 12N); ES/MS calcd for C₃₅H₄₄NO₇ 590.3, found m/z=590.3 (MH⁺).

### Example 133

### 1-[5-[1-Hydroxy-2-[6-(4-phenylbutoxy)hexylamino]ethyl]-2-phosphonooxybenzyl]-4-[[11β,16α][[16,17-((R)-cyclohexylmethylene)bis(oxy)]-11-hydroxypregna-1,4-diene-3,20-dion-21-oxy]carbonylmethyl]pyridinium chloride

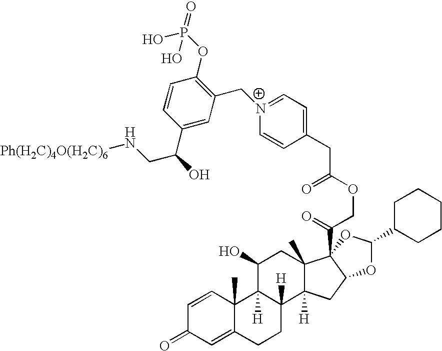

The title compound was synthesized in manner analogous to that described in Example 58, using the compound prepared as described in Example 132 and the compound prepared as described in Example 82 as starting material. ¹H NMR (400 MHz, DMSO-d6) d 9.27 (brs, 1H), 9.14 (d, 2H, J=4.0 Hz), 8.83 (brs, 1H), 8.10 (d, 2H, J=4.0 Hz), 7.68 (s, 1H), 7.46-7.24 (m, 5H), 7.20-7.14 (m, 2H), 6.16 (dd, H, J=1.5, 10.2 Hz), 5.91 (s, 1H), 5.85 (s, 2H), 5.05-4.83 (m, 3H), 4.68 (d, 1H, J=3.9 Hz), 4.37 (d, 1H, J=4.1 Hz), 4.29 (s, 2H), 3.35 (s, 4H), 3.13 (m, 1H), 3.01-2.85 (m, 3H), 2.57 (m, 2H), 2.28 (m, 1H), 2.08-1.94 (m, 2H), 1.80 (m, 2H), 1.16 (m, 40H); ³¹P NMR (400 Hz, DMSO-d6) d −5.85; ES/MS calcd for C₆₀H₈₀N₂O₁₃P⁺ 1067.5, found m/z=1068.5 (M⁺).

### Example 134

### [1-[5-[1-Hydroxy-2-[6-(4-phenylbutoxy)hexylamino]ethyl]-2-phosphonooxybenzyl]pyridin-4-yl]acetic acid [11β,16a]-[[((R-cyclohexylmethylene)bis(oxy)]-11-hydroxypregna-1,4-diene-3,20-dion-21-yl]]ester

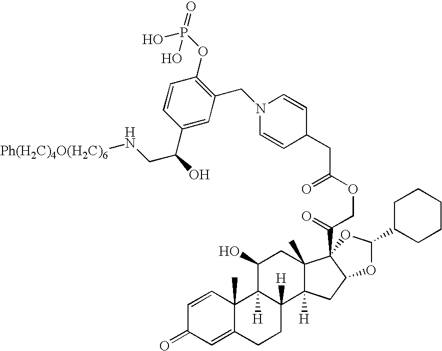

The title compound was prepared by dissolving Example 133 in CHCl₃. The organic layer was washed with 1N NaOH, washed with H₂O, washed with brine, dried over Na₂SO₄, and concentrated to give crude dihydropyridine. Recrystallization (CH₂Cl₉/Et₂O) afforded the title compound as brown solid. ¹H NMR (400 MHz, DMSO-d6) 7.46 (m, 1H), 7.36-7.09 (m, 9H), 6.13 (m, 2H), 5.89 (s, 1H), 4.92 (m, 3H), 4.75-4.57 (m, 4H), 4.48 (m, 1H), 4.35 (d, 1H, J=4.2 Hz), 4.28 (m, 1H), 3.29 (dd, 4H, J=9.6, 16.2 Hz), 2.55 (m, 2H), 2.25 (m, 1H), 2.08-1.93 (m, 2H), 1.89-1.76 (m, 2H), 1.08-0.77 (m, 45H); ³¹P NMR (400 MHz, DMSO-d6) d −4.30; ES/MS calcd for C₆₀H₈₀N₂O₁₃P⁺ 1067.5, found m/z=1067.5 (MH⁺).

### Example 135

### Stability of Steroid C-21 Esters, Carbonates and Carbamates by Rat Lung Homogenate

### Preparation of Rat Lung Homogenate

Lungs from Fischer 344 rats were obtained fresh by overnight delivery at 4° C. from BioReclamation Inc. (Hicksville, N.Y.). Lungs were weighed and homogenized in a 1:3 w/v ratio with sterile phosphate buffered saline (PBS, 10 mM, pH 7.4) in glass vials on ice. After centrifugation at 3,000×g for 10 min at 4° C. the supernatant was decanted into sterile conical tubes and placed on ice. The total protein content of the supernatant was determined by the bicinchoninic acid (BCA) method (Pierce Biotechnology, Rockford, Ill.), using bovine serum albumin (BSA) as the standard. Lung homogenates were prepared to a final concentration of 1 mg total protein/mL in 10 mM PBS, pH 7.4.

### In Vitro Metabolism of Steroid 21-Esters, Carbonates, Carbamates and Controls in Rat Lung Homogenate

Compounds were incubated with active or heat-inactivated rat lung homogenate in 10 mM PBS (pH 7.4). Heat-inactivation was achieved by incubation at 80° C. for 30 min, after which the homogenate was allowed to cool to room temperature, stored overnight at 4° C. The homogenate was used for the assay and standard curve preparations. Before use, each homogenate preparation was equilibrated for 15 min in a 37° C. water bath. The metabolism reactions were initiated by the addition of stock solutions of 21-derivatized steroids ciclesonide and desisobutyryl ciclesonide in 1 mM dimethyl sulfoxide (DMSO) to a final concentration of 900 nM. DMSO (2.7 uL) added to 3 mL of temperature-equilibrated homogenate served as a control. Aliquots (100 uL) of homogenate+compound were added to 400 μL quenching solution consisting of 100% HPLC-grade acetonitrile+500 ng/mL glyburide for the zero time point (n=3 for each time point).

The glyburide served as an internal LC/MS/MS standard. The remainder of each drug+homogenate solution was aliquoted into a 96-well tissue culture plates. After an additional 30 min and 120 min incubation at 37° C., 100 μL aliquots were added to 400 μL quenching solution. Denatured proteins in the quenching solution were separated by centrifugation at 3000×g for 2 min at 4° C., and 160 μL of the supernatants were transferred to new 96-well plate for analysis by LC/MS/MS. Collection plates were covered with plastic film and were kept on ice. For storage, covered plates were and kept stored at −80° C. until further use.

### Liquid Chromatography and Mass Spectrometry Analysis (LC/MS/MS)

An aliquot of 50 μL of each sample was diluted with 50 μL of water containing internal standard at 4° C. The diluted samples were then centrifuged for 20 minutes at 3000 rpm at 4° C. An aliquot of 20 μL of the solution was injected into the TSQ Ultra Quantum.

LC/MS/MS system. The compounds were separated by HPLC using a HyPurity C18 HPLC column (30×2.1 mm, 5μ) from ThermoHypersil. A Multiplex LX-2 HPLC system (Cohesive Technologies, Franklin, Mass.) with two identical Agilent 1100 series binary pumps (P/N G1312A) were used for elution and separation. Samples were maintained at 4° C. in an HTS Pal autosampler (LEAP Technologies, Carrboro, N.C.) in order to reduce any potential spontaneous hydrolysis of the compounds before injection onto the HPLC. The analytes were eluted using the following mobile phases; Mobile phase A contained 1% acetonitrile in 10 mM ammonium formate aqueous solution with 1% formic acid. Mobile phase B contained 80% acetonitrile in 10 mM ammonium formate with 1% formic acid. The HPLC elution program used to elute the analytes was as follows:

| | | Flow Rate | Mobile Phase | Mobile Phase |
|---|---|---|---|---|
| Time (sec) | Step Comments | (mL/min) | A (%) | B (%) |
| 90 | Sample | 0.50 | 100 | 0 |
| | Loading | | | |
| 150 | Ramp | 0.50 | 50 | 50 |
| 180 | Elution | 0.50 | 0 | 100 |
| 120 | Re-equilibrium | 0.50 | 100 | 0 |

The samples were further analyzed by tandem mass spectrometry using a TSQ Quantum Ultra triple quadrupole mass spectrometer (Thermo Finnigan, San Jose, Calif.) using a selective reaction monitoring (SRM) scan type. The mass spectrometry parameters used were as follows:

| | | | Sheath gas | Aux gas | Capillary |
|---|---|---|---|---|---|
| Ion | Source CID | Spray | pressure | pressure | temperature |
| source | (V) | voltage (V) | (Arb) | (Arb) | (° C.) |
| ESI+ | 10 | 4000 | 40 | 15 | 350 |

### Data Analysis

Nine-point standard curves for each test compound were prepared and analyzed in heat-inactivated lung homogenate. The concentration ranged from 1 nM to 10 M. The calibration curves of the steroid linkers, ciclesonide (CIC) and desisobutyryl ciclesonide (des-CIC) were evaluated by linear regression analysis. The data expressed in Table 2 represent the mean (n=3) percent remaining compound in both types of homogenate at 2 hours, 37° C. Table 1 also describes the values obtained for mean concentration remaining of the parent compound and des-CIC at 2 hours, 37° C.

### Results

The stability of the ester, carbonate, and carbamate components was determined as described in the preceding section. Table 2 shows the percentage of mean remaining parent compound in inactive or active Fischer 344 rat lung homogenate after incubation at 2 h at 37° C. and the mean concentration (nM) of parent and metabolite (des-ciclesonide) remaining at 2 h at 37° C.

TABLE 2

| | | Mean % | | Mean [Parent]/ | |
|---|---|---|---|---|---|
| | | compound | | [des-ClC] | |
| | | remaining at | | (nM) at | |
| | | 2 hrs, 37° C. | | 2 hrs, 37° C. | |
| | Controls | Inactive | Active | Inactive | Active |
| | ClC | 88 | 12 | 154/BLQ | 18/102 |
| | des-ClC | 71 | 98 | 84 | 119 |
| | Steroid | | | | |
| Linker | linkers | Inactive | Active | Inactive | Active |
| 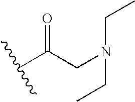 | Example 32 | 61 | 1 | 119/64 | 1/270 |
| 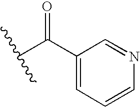 | Example 29 | 72 | 1 | 130/BLQ | 2/154 |
| 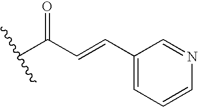 | Example 33 | 78 | 3 | 124/BLQ | 4/170 |
| 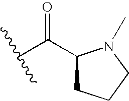 | Example 38 | 40 | 1 | 71/45 | 1/148 |
| 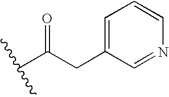 | Example 76 | 83 | 2 | 142/11 | 4/162 |
| 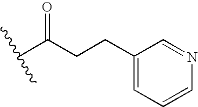 | Example 34 | 73 | 0 | 159/BLQ | 1/255 |
| 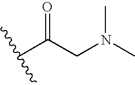 | Example 75 | 55 | 1 | 101/45 | 1/111 |
| 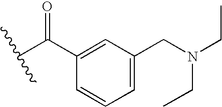 | Example 31 | 68 | 68 | 127/BLQ | 127/45 |
| 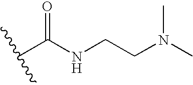 | Example 39 | 78 | 92 | 162/BLQ | 186/BLQ |
| 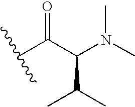 | Example 79 | 61 | 85 | 88/BLQ | 149/BLQ |
| 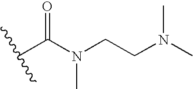 | Example 40 | 78 | 83 | 167/BLQ | 222/BLQ |
| 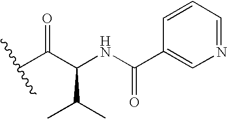 | Example 51 | 85 | 86 | 169/BLQ | 176/BLQ |
| 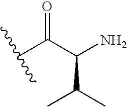 | Example 50 | 25 | 45 | 50/BLQ | 219/BLQ |
| 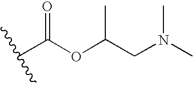 | Example 28 | 64 | 45 | 147/70 | 120/107 |
| 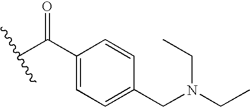 | Example 30 | 51 | 46 | 109/BLQ | 102/36 |
| 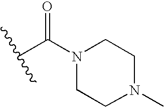 | Example 41 | 78 | 68 | 172/BLQ | 170/BLQ |

BLQ = below limit of quantitation;
LOQ (limit of quantitation) for des-ClC = 10 nM

### Example 136

### Pharmacokinetic Analysis of Drug Levels of Salmeterol, Desisobutyryl Ciclesonide (“des-ciclesonide”) and Compounds of Examples 52 and 83 in Lung Bronchoalveolar Lavage Fluid and Plasma After IT Administration in the Rat

### Dosing

Compounds of Examples 52 and 83 were formulated for intratracheal (IT) dosing in 10% EtOH, 90% Sterile Water, and dosed in male Sprague-Dawley rats at 3 mg/kg (Example 52) and 1 mg/kg (Example 83). Each dosing group consisted of 3 male, naïve purebred Sprague-Dawley rats. At dosing, the animals weighed ˜0.30 kg. The animals were fasted overnight prior to dose administration and up to 2 hr after dosing. The compounds were administered IT using a Penn Century Microsprayer (Model 1A-1B).

### Sample Collection and Analysis

### A. Plasma

Blood samples were collected at 0.5, 2, and 4 hours post-dose. Each blood sample (0.5-0.6 mL per sample) was collected via the orbital sinus (following anesthesia for BAL procedure) into tubes containing EDTA anti-coagulant into containers surrounded by dry ice at 0.5, 2 and 4 hr (mean, n=6). Blood samples were stored at −20±5° C. until shipped for analysis.

### B. BALF

The animals were anesthetized with an intramuscular (IM) injection of a ketamine/xylazine/acepromazine (80/10/2 mg/kg) cocktail at a dose volume of 1.1 mL/kg. A cannula (modified Bard® infant feeding tube) was inserted into the trachea. Warmed sterile saline was injected into the lungs. The lungs were gently massaged by palpation of the chest for approximately 45 seconds. The fluid (BALF) was recovered and placed on ice. The procedure was repeated two more times, and all three BALF samples pooled. The fluid was centrifuged under refrigerated conditions at 350 g for 10 minutes. The supernatant and cell pellet were collected and stored at approximately −70° C. until shipped for analysis.

### C. Lung Tissue Collection

Immediately following each BAL procedure, the lungs from each animal were removed, blotted dry, weighed, and stored frozen at −70° C. until shipped for analysis.

### D. Assay Methods

An LC/MS/MS method was used to measure the concentration of compound in plasma.

### Bioanalytical Method

### 1. Lung Homogenate

Add 3×w/v of 1×PBS buffer (90:10-PBS:ACN) to each lung tissue. Homogenize the sample w/Polytron (PT1200) and take 50 uL of supernatant and inject to LC/MSMS.

### 2. Sample Processing

An aliquot of 50 μL of each plasma sample was treated with 100 mL of acetonitrile (ACN) containing internal standard. After the protein precipitation, an aliquot of 100 μL of the supernatant was transferred to a clean 96-well plate and mixed with 100 μL of water. An aliquot of 30 μL of the above solution was injected to the TSQ Ultra Quantum LC/MS/MS system.

### 3. HPLC Condition

A HyPurity C18 HPLC column (30×2.1 mm, 5μ) from ThermoHypersil (Part #: 22105-032130) was used. Mobile phase A contained 1% acetonitrile in 10 mM ammonium formate aqueous solution with 1% formic acid. Mobile phase B contained 80% acetonitrile in 10 mM ammonium formate with 1% formic acid. An Agilent 1100 series binary pump (P/N G1312A Bin Pump) was used for elution and separation. HTS Pal autosampler from LEAP Technologies, Carrboro, N.C. was used.

### HPLC Elution Program:

| | | Flow Rate | Mobile Phase | Mobile Phase |
|---|---|---|---|---|
| Time (sec) | Step Comments | (mL/min) | A (%) | B (%) |
| 90 | Sample | 0.50 | 100 | 0 |
| | Loading | | | |
| 150 | Ramp | 0.50 | 50 | 50 |
| 180 | Elution | 0.50 | 0 | 100 |
| 120 | Re-equilibrium | 0.50 | 100 | 0 |

### 4. Mass Spectrometry

TSQ Quantum Ultra triple quadrupole mass spectrometer from Thermo Finnigan, San Jose, Calif. was used in selective reaction monitoring (SRM) operation mode, Tune file:

### ESI_tune112807_BL.

### Mass Spectrometry Parameters:

| | | | Sheath gas | Aux gas | Capillary |
|---|---|---|---|---|---|
| Ion | Source | Spray | pressure | pressure | temperature |
| source | CID (V) | voltage (V) | (Arb) | (Arb) | (° C.) |
| ESI+ | 10 | 4000 | 40 | 15 | 350 |

### SRM Channels:

| | | | Product | |
|---|---|---|---|---|
| | | Parent Mass | Mass | Collision |
| Analyte | ID | (m/z) | (m/z) | Energy (V) |
| Analyte | Example 83 | 1061.7 | 584.3 | 35 |
| Analyte | des-ciclesonide | 453.4 | 147.1 | 33 |
| Analyte | Example 32 | 584.5 | 86.1 | 35 |
| Analyte | salmeterol | 398.3 | 91.1 | 36 |
| Internal Standard | | 756.3 | 600.3 | 33 |
| Analyte | Example 52 | 1061.7 | 584.3 | 35 |
| Analyte | des-ciclesonide | 453.4 | 147.1 | 33 |
| Analyte | Example 32 | 584.5 | 86.1 | 35 |
| Analyte | salmeterol | 398.3 | 91.1 | 36 |
| Internal Standard | | 756.3 | 600.3 | 33 |

### Limits of Quantitation (LOQ) in Lung:

| Sample | | des- | | |
|---|---|---|---|---|
| Matrix | | ciclesonide | Example 32 | salmeterol |
| | Ex 52 | | | |
| Plasma (nM) | 5 | 5 | 2 | 1 |
| | Ex 83 | | | |
| Plasma (nM | 1 | 2 | 1 | 1 |

### Results

### Plasma and Lung Concentrations of Example 52 and Metabolite Following it Administration of the Compound of Example 52

| | Time | Plasma Conc | BALF Conc | Lung Conc |
|---|---|---|---|---|
| Analyte | (hr) | (nM) | (nM) | (nM) |
| Example 52 | 0.5 | 977 | | 237258 |
| Example 52 | 2.0 | 254 | | 316154 |
| Example 52 | 4.0 | 90.8 | | 189815 |
| Example 32 | 0.5 | 44.4 | | 1160 |
| Example 32 | 2.0 | 31.1 | | 1229 |
| Example 32 | 4.0 | 24.7 | | 851 |
| des-ciclesonide | 0.5 | 160 | | 9446 |
| des-ciclesonide | 2.0 | 115 | | 17920 |
| des-ciclesonide | 4.0 | 94 | | 10013 |
| salmeterol | 0.5 | BLQ | | 1829 |
| salmeterol | 2.0 | BLQ | | 1422 |
| salmeterol | 4.0 | BLQ | | 996 |

Plasma and Lung Concentrations of Example 83 and Metabolite Following IT Administration of the Compound of Example 83

| | Time | Plasma Conc | BALF Conc | Lung Conc |
|---|---|---|---|---|
| Analyte | (hr) | (nM) | (nM) | (nM) |
| Example 83 | 0.5 | 124 | 15391 | 71623 |
| Example 83 | 2.0 | 19.4 | 6112 | 17670 |
| Example 83 | 4.0 | 8.1 | 7704 | 33776 |
| Example 32 | 0.5 | 20.8 | 37.8 | 124.4 |
| Example 32 | 2.0 | 23.9 | 28.7 | 134.9 |
| Example 32 | 4.0 | 16.3 | 20.4 | 159.0 |
| des-ciclesonide | 0.5 | BLQ | 362 | 3989 |
| des-ciclesonide | 2.0 | BLQ | 363 | 3271 |
| des-ciclesonide | 4.0 | BLQ | 218.2 | 4159 |
| salmeterol | 0.5 | BLQ | BLQ | 515 |
| salmeterol | 2.0 | BLQ | BLQ | 434 |
| salmeterol | 4.0 | BLQ | BLQ | 705 |

### CONCLUSION

The results demonstrate that the compounds of Example 52 and 83 are metabolized to Salmeterol and Desisobutyryl Ciclesonide in the lung following IT administration.

### Example 137

### Drug Metabolism Studies Using Airway Epithelial Cells Cultured at an Air-Liquid Interface

Cryopreserved passage 1 cells were cultured in bronchial epithelial growth medium (Fulcher, M. L., et al., Well-differentiated human airway epithelial cell cultures. Methods Mol Med, 2005. 107: p. 183-206) on 100 mm Type I collagen-coated plastic dishes. At 70% confluence, passage 2 cells were transferred to type IV collagen-coated Millicell membranes (Millipore, Bedford, Mass.) in medium that supports growth at an air-liquid-interface (ALI) (Fulcher er al., 2005). Cells were maintained at an ALI and allowed to differentiate fully for approximately 28 days. Approximately 24 hrs prior to the start of the experiment, the apical surfaces of the cells were washed with sterile phosphate buffered saline (PBS, 10 mM, pH 7.4) and the basolateral media was replaced with fresh ALI media. Approximately 1 hour prior to the start of the experiment, the apical surfaces of the cells were washed once again with PBS and the basolateral media replaced with fresh ALI media. At time=0 hrs, the test article was diluted from a 10 mM stock solution in DMSO to a 40 μM solution in ALI media/PBS/10% EtOH/water (v/v). 50 μl of the resulting 40 μM solution was immediately added to the apical surface of the cells. 200 μl of the dosing solution was also added to 800 μl of 100% ACN and frozen immediately on dry ice. The remaining dosing solution was placed in the incubator with the cells. The dosing solution and cells were allowed to incubate for 10, 120 and 240 min at which points the apical surfaces of cells from 4 millicell cell culture inserts (n=4) were washed with 3×100 μl of PBS or 10% EtOH/water (v/v) per millicell. The three washes from each millicell cell culture insert were pooled. The entire basolateral medium from each millicell cell culture insert was also collected as were the airway epithelial cells which were excised from each millicell cell culture insert and added to 300 μl of 90% ACN/0.1% formic acid/9.9% water and immediately frozen on dry ice. The cells were thawed and lysed for 2 mins with a sonicator (Misonix, Fanningdale, N.Y.) set at 30 Amp. The cell suspension was then centrifuged at 18,000 g for 2 min and 50 μl of the supernatant was added to 200 μl of acetonitrile containing 100 ng ml⁻¹ glyburide. 50 μl of the pooled apical washes and basolateral medium was also added to 200 μl of ACN containing 100 ng ml⁻¹ glyburide. These samples were then frozen on dry ice and kept at −80° C. for their analysis by LC/MS/MS. At the same 10, 120 and 240 min time points, 200 μl of the dosing solution was added to 800 μl of ACN and immediately frozen on dry ice. These dosing solution samples were also kept at −80° C. for analysis by LC/MS/MS. Untreated control cells dosed at the apical surface with 50 μl of test article vehicle (ALI media/PBS/10% EtOH/water (v/v) were also included to provide apical, basolateral and cellular matrices for LC/MS/MS analytical standards.

The above samples were thawed prior to centrifugation for 10 minutes at 3000 rpm at 4° C. An aliquot of 150 μL of the above solution was mixed with 150 μL of water. 10 μL of the acetonitrile/water mix were injected into the Applied Biosystems/Sciex API 5000 LC/MS/MS system. The compounds were separated by HPLC using a Zorbax Extend C18 HPLC column (50×2.1 mm, 3.5 μl) from Agilent Technologies. An Aria Transcend duplexed HPLC system (Thermo Fisher, Franklin, Mass.) with two identical Agilent 1100 series binary pumps (P/N G1312B) were used for elution and separation. Samples were maintained at 4° C. in an HTS Pal autosampler (LEAP Technologies, Carrboro, N.C.) in order to reduce any potential spontaneous hydrolysis of the compounds before injection onto the HPLC. The analytes were eluted using the following mobile phases: Mobile phase A contained 1% acetonitrile in 10 mM ammonium formate aqueous solution with 1% formic acid. Mobile phase B contained 80% acetonitrile in 10 mM ammonium formate with 1% formic acid. The HPLC elution program used to elute the analytes was as follows:

| Time | | Flow Rate | Mobile Phase | Mobile Phase |
|---|---|---|---|---|
| (sec) | Step Comments | (mL/min) | A (%) | B (%) |
| 30 | Sample Loading | 0.50 | 90 | 10 |
| 150 | Ramp | 0.50 | 50 | 50 |
| 180 | Elution | 0.50 | 0 | 100 |
| 90 | Re-equilibrium | 0.50 | 90 | 10 |

The samples were further analyzed by tandem mass spectrometry using an ABI/Sciex API 5000 triple quadrupole mass spectrometer (Applied Biosystems, Foster City, Calif.) using a selective reaction monitoring (SRM) scan type. The mass spectrometry parameters used were as follows:

| | CAD | | Curtain | | | |
|---|---|---|---|---|---|---|
| | gas | Spray | gas | GS1 gas | GS21 gas | Capillary |
| Ion | pressure | voltage | pressure | pressure | pressure | temp |
| source | (arb) | (V) | (Arb) | (Arb) | (Arb) | (° C.) |
| ESI+ | 6 | 5000 | 25 | 40 | 60 | 350 |

Eleven-point standard curves for each test compound were prepared and analyzed in heat-inactivated lung homogenate, the concentration ranged from 1 nM to 10 μM. The calibration curves of the steroid linkers, ciclesonide (CIC) and desisobutyryl ciclesonide (des-CIC) were prepared by quadratic regression analysis.

The results are reported in Table 3 below.

TABLE 3

| | | Averaged (n = 4) pmoles present at 10 min/120 min/240 min | | | | |
|---|---|---|---|---|---|---|
| Example | | ALI | Parent | Steroid- | | |
| No. | | compartment* | Compound | linker | Salmeterol | Desciclesonide |
| 52 | ALI Media | Cellular | 85/55/25 | 9/305/211 | 7/48/93 | 21/155/250 |
| | | Apical | 912/305/37 | 15/89/52 | 14/23/18 | 494/119/77 |
| 83 | ALI Media | Cellular | 89/58/44 | 7/365/225 | 2/47/76 | 15/152/219 |
| | | Apical | 988/270/43 | 14/95/53 | 6/25/19 | 393/73/72 |
| 47 | PBS | Cellular | 20/40/8 | 0/0/4 | 3/15/15 | 3/6/4 |
| | | Apical | 383/312/35 | 0/0/0 | 0/0/0 | 5/2/1 |
| 56 | PBS | Cellular | 29/92/14 | 1/17/179 | 4/24/77 | 3/6/16 |
| | | Apical | 768/403/103 | 5/14/228 | 15/27/27 | 5/5/14 |
| 69 | 10% | Cellular | 206/244/57 | 3/26/66 | 6/23/29 | 12/44/125 |
| | EtOH/water | Apical | 950/554/204 | 33/483/621 | 15/45/42 | 47/51/59 |
| 74 | 10% | Cellular | 248/220/80 | 0/2/5 | 2/6/7 | 2/5/13 |
| | EtOH/water | Apical | 1112/935/288 | 20/123/133 | 8/18/17 | 4/7/8 |
| 110 | 10% | Cellular | 199/294/158 | 0/0/1 | 31/166/163 | 0/0/1 |
| | EtOH/water | Apical | 1495/1818/1200 | 0/2/2 | 24/30/25 | 0/0/0 |

Thus, for example, the amount of the parent compound of Example 52 decreased over time in both the apical and cellular compartments, while the amount of salmeterol and desciclesonide increased in the cellular compartment.

### Example 138

### In Vivo Efficacy of Compound from Example 52 in the Mouse Ovalbumin Model of Lung Inflammation

Mice (Balb/c) were immunized by intraperitoneal injection of ovalbumin (10 μg OVA suspended in 2 mg aluminum hydroxide) on day 0 and 7. One group was sensitized and treated with vehicle (NSV). One group was immunized with sterile water only and treated with vehicle, e.g. to serve as a nonsensitized (negative) control (Vehicle). Ciclesonide (positive control treatment; 1× per day (day 14 and 15; 3 mg/kg), Compound from Example 52 at 1, 3, and 6 mg/kg) or vehicle was delivered by intratracheal (IT) instillation 1×/day (day 14 and 15), 1 hour prior to OVA inhalation challenge. On days 14 and 15, the animals were exposed to OVA by inhalation (3 h; 5 mg/m³). Forty eight hours following the last OVA challenge (day 17) mice were sacrificed. Bronchoalveolar lavage (BAL) was taken from each animal to collect cells and fluid. Cell numbers and differentials were calculated from BAL.

### Example 139

### Eosinophil and Neutrophil Counts in Mouse Lung BAL After IT Administration of 0.6, 1, and 3 mpk of Compound from Example 52

| | |
|---|---|
| Neutrophils | Eosinophils |
| (×10⁴/mL) | (×10⁴/mL) |

| | Mean | SEM | Mean | SEM |
|---|---|---|---|---|
| NSV | 0.174 | 0.049 | 0.000 | 0.000 |
| Vehicle | 2.783 | 0.279 | 53.521 | 3.521 |
| Ciclesonide (3.0 mg/kg) | 0.479 | 0.103 | 6.017 | 1.023 |
| Example 52 (3.0 mg/kg) | 1.037 | 0.187 | 5.742 | 0.651 |
| Example 52 (1.0 mg/kg) | 0.534 | 0.089 | 16.332 | 2.913 |
| Example 52 (0.6 mg/kg) | 0.727 | 0.205 | 36.758 | 4.002 |

### Example 140

### LPS-Induced Airway Inflammation in Rats

Example 52 was evaluated in an LPS-induced airway inflammation model in rats. Male Fischer 344 rats (n 8/group) were treated with either vehicle or test article by intratracheal instillation in 400 III volume 1 hour prior to LPS challenge. Animals were then exposed to LPS by nose-only inhalation exposure for 10 minutes at 5 mg/m³ resulting in an estimated 5 μg lung deposition. Animals were sacrificed 4 hours after LPS exposure. Lungs were lavaged for bronchoalveolar lavage cell differentials and cytokine analysis by Luminex multiplex immunoassay. Example 52 and ciclesonide were shown to significantly inhibit LPS-induced airway inflammation measured as neutrophil influx and TNFα production at doses tested (p<−0.05 versus vehicle control).

| | Dose | Neutrophils | TNFα |
|---|---|---|---|
| Compound | (mg/kg) | (× 10⁴/ml) | (pg/ml) |
| Vehicle | | 35.1 ± 2.1 | 1116.9 ± 245.8 |
| Ciclesonide | 3 | 9.7 ± 0.89 | 629.2 ± 330.0 |
| Example 52 | 6 | 6.4 ± 0.41 | Not determined |
| Example 52 | 3 | 13.5 ± 0.92 | 307.0 ± 60.0 |
| Example 52 | 1 | Not determined | 361.7 ± 56.6 |
| Example 52 | 0.6 | Not determined | 293.6 ± 40.6 |

Mean ± s.e.m.

### Example 141

### Tobacco Smoke Model of Airway Inflammation in Ice

Example 52 was evaluated in a tobacco-smoke-induced airway inflammation model in female C3H mice. Vehicle and test articles are delivered by intratracheal instillation in 10% ethanol water to animals anesthetized with 3-5% isoflurane (n=8 animals per test article group). All compounds will be delivered on a daily (5 days per week) schedule for 3 weeks during the smoke exposures. Mice were exposed to cigarette smoke for 6 hours per day, 5 days per week for 3 weeks, in H1000 or H2000 chambers. Type 2R4F research cigarettes (Kentucky Tobacco Research and Development Center) were used in the study. Total particulate material (TPM) exposure was kept at 100 TPM/m³ for the first week to allow animals to reduce adverse effects during adaptation to smoke exposure. TPM exposure was maintained between 100 and 250 mg TPM/m³ during the remainder of the study. After 3 weeks, animals were euthanized and bronchoalveolar lavage fluid was obtained for cell differentials and cytokine analysis by Luminex multiplex immunoassay.

Inhibition of tobacco smoke-induced neutrophil influx into mouse airways was observed at p<0.05 versus vehicle control at 1.0 and 0.6 mg/kg doses of the compound of Example 52. Inhibition of neutrophil influx was not observed in other treatments groups, including ciclesonide, salmeterol xinafoate, and ciclesonide+salmeterol xinafoate combination. In a 20-plex Luminex assay for cytokines and growth factors, Example 52 exhibited inhibition of tobacco-smoke-induced IL-1α and MIP-1α production (p<0.05 versus vehicle control at 1.0 and 0.6 mg/kg doses).

| | Dose | | | |
|---|---|---|---|---|
| | (mg/kg) | Neutrophils | IL-1α | MIP-1α |
| Compound | i.t. | (×10⁴/ml) | (pg/ml) | (pg/ml) |
| Vehicle, no | | 0.04 ± 0.01 | 1.35 ± 0.47 | 3.92 ± 1.15 |
| smoke | | | | |
| Vehicle | | 14.8 ± 2.6 | 7.04 ± 1.03 | 11.61 ± 1.23 |
| Ciclesonide | 0.51 | 15.0 ± 1.5 | 5.50 ± 0.96 | 10.02 ± 1.75 |
| Ciclesonide | 0.31 | 13.9 ± 2.1 | 5.25 ± 0.85 | 8.31 ± 1.17 |
| Salmeterol | 0.57 | 10.9 ± 2.5 | 4.22 ± 0.73 | 6.09 ± 1.38 |
| Xinafoate | | | | |
| Ciclesonide + | 0.51 | 11.1 ± 1.9 | 4.92 ± 0.92 | 7.11 ± 1.64 |
| Salmeterol | 0.57 | | | |
| Xinafoate | | | | |
| Example 52 | 1.0 | 8.5 ± 1.3 | 2.74 ± 0.64 | 7.0 ± 1.40 |
| Example 52 | 0.6 | 7.7 ± 1.8 | 2.54 ± 0.59 | 4.34 ± 1.15 |

Mean ± s.e.m.

### Example 142

### Ragweed-Induced Bronchoconstriction in Dogs

The compound of Example 52 was assesed for bronchodilator activity in a ragweed-induced bronchoconstriction model in beagle dogs. Dogs were mechanically ventilated during each experiment. Airflow and tidal volume were measured using a differential pressure transducer located in front of the endotracheal tube. An esophageal balloon catheter placed in the esophagus was used to determine transpulmonary pressure. Pulmonary resistance and dynamic lung compliance were calculated from the simultaneous measurement of transpulmonary pressure and respiratory flow. The canine exposure system was designed to expose an anesthetized animal via an intubation tube. Dogs were administered vehicle or test article by inhalation 30 minutes before ragweed antigen exposure (n=4). Dogs were challenged with ragweed antigen (ragweed short, Ambrosia artemisifolia, Greer, Lenoir, N.C.) by inhalation (5 breaths). Immediately following ragweed challenge, changes in pulmonary resistance and compliance were measured for up to 30 minutes.

At doses of 20 μg/kg of the compound of Example 52, an inhibition of ragweed-induced increases in pulmonary resistance (p=0.0008 versus vehicle control) was observed. In comparison, an inhibition of increased pulmonary resistance change following antigen challenge (p=0.0008 versus vehicle control) was also observed at 10 mg/kg of salmeterol xinafoate (10 μg/kg). Significant inhibition of ragweed response was not observed with the compound of Example 52 at 6 μg/kg, salmeterol xinafoate at 3 μg/kg, or ciclesonide at 10 μg/kg.

| | | Pulmonary Resistance |
|---|---|---|
| Compound | Dose (μg/kg) | (% of Baseline) |
| Vehicle | | 367.2 ± 9.3 |
| Ciclesonide | 10 | 302.3 ± 24.8 |
| Salmeterol xinafoate | 10 | 177.8 ± 14.4 |
| Salmeterol xinafoate | 3 | 265.6 ± 38.8 |
| Example 52 | 20 | 181.2 ± 11.5 |
| Example 52 | 6 | 266.1 ± 19.6 |

Mean ± s.e.m.

### Example 143

### Ascaris suum-Induced Pulmonary Responses in Sheep

The compound of Example 52 was assessed for inhibition of early and late phase bronchoconstriction and development of airway hyperreactivity in sheep sensitized to Ascaris suum antigen as previously described (Abraham, W. M., A. Ahmed, I Serebrlakov, I. T. Lauredo, J. Bassuk, J. A. Adams, and M. A. Sackner. Am. J. Respir. Crit. Care. Med. 2006; 174:743-752. Early and late phase responses were measured as a function of increased pulmonary resistance during the 8 hour period following antigen. Airway hyperreactivity was evaluated as a function of PC400, the number of carbachol breath units required to induce a four-fold increase in bronchoconstriction measured 24 hours after antigen challenge. One breath unit is defined as one breath of a 1% w/v carbachol solution. Test article was administered either by a pre-dosing or duration of action protocol. In the pre-dosing regimen, animals were dosed once daily for four days, with the last dose administered 1 hr before antigen. In the duration of action regimen, animals were dosed once daily for four days, with the last dose administered 24 hours before antigen. Test article, Ascaris antigen, and carbachol were administered by nebulized aerosol to intubated sheep.

Using the predosing regimen, inhibition of late phase bronchoconstriction and development of airway hyperreactivity was observed with both the compound of Example 52 and ciclesonide. The results demonstrate the compound of Example 52 can provide steroid-dependent inhibition of the Ascaris response.

In the duration of action regimen a reduction of early phase bronchoconstriction and complete inhibition of late phase bronchoconstriction and development of airway hyperactivity were observed with the compound of Example 52. An inhibition of late phase bronchoconstriction and development of airway hyperreactivity was observed with a combination of ciclesonide+salmeterol. In contrast, reduced efficacy was observed with ciclesonide and salmeterol alone. These results suggest that the compound of Example 52 may possess advantageous anti-inflammatory properties compared to the combination of ciclesonide and salmeterol.

Ascaris suum Challenge

Predosing Regimen

Bronchoconstriction

Lung Resistance (% Increase)

| Time (hours) | Vehicle | Example 52 (4.7 mg) |
|---|---|---|
| Post-Ascaris | 380 | 220 |
| 1 | 231 | 118 |
| 2 | 130 | 69 |
| 3 | 53 | 34 |
| 4 | 21 | 9 |
| 5 | 69 | 24 |
| 6 | 105 | 34 |
| 6.5 | 109 | 19 |
| 7 | 139 | 23 |
| 7.5 | 122 | 28 |
| 8 | 107 | 14 |

Mean, n = 2

Hyperreactivity

PC400
(carbachol breath units)

| | Vehicle | Example 52 (4.7 mg) |
|---|---|---|
| Baseline | 23.0 | 27.5 |
| Post-Antigen | 13.0 | 26.0 |

Mean, n = 2

Bronchoconstriction

Lung Resistance (% Increase)

| Time (hours) | Vehicle | Ciclesonide (2.2 mg) |
|---|---|---|
| Post-Ascaris | 407 | 305 |
| 1 | 236 | 142 |
| 2 | 157 | 44 |
| 3 | 46 | 8 |
| 4 | 12 | 6 |
| 5 | 69 | 14 |
| 6 | 118 | 28 |
| 6.5 | 120 | 12 |
| 7 | 127 | 28 |
| 7.5 | 112 | 33 |
| 8 | 121 | 15 |

Mean, n = 2

Hyperreactivity

PC400
(carbachol breath units)

| | Vehicle | Ciclesonide (2.2 mg) |
|---|---|---|
| Baseline | 25.5 | 26.5 |
| Post-Antigen | 14.0 | 25.5 |

Mean, n = 2

Ascaris suum Challenge

Duration of Action Regimen

Bronchoconstriction

Lung Resistance (% Increase)

| Time (hours) | Vehicle | Example 52 (4.7 mg) |
|---|---|---|
| Post-Ascaris | 459 | 226 |
| 1 | 279 | 95 |
| 2 | 173 | 47 |
| 3 | 62 | 17 |
| 4 | 8 | 14 |
| 5 | 66 | 25 |
| 6 | 118 | 31 |
| 6.5 | 123 | 26 |
| 7 | 137 | 26 |
| 7.5 | 124 | 20 |
| 8 | 113 | 22 |

Mean, n = 4

Hyperreactivity

PC400
(carbachol breath units)

| | Vehicle | Example 52 (4.7 mg) |
|---|---|---|
| Baseline | 25 | 27.8 |
| Post-Antigen | 11.8 | 28 |

Mean, n = 4

Bronchoconstriction

Lung Resistance (% Increase)

| Time (hours) | Vehicle | Ciclesonide (2.2 mg) |
|---|---|---|
| Post-Ascaris | 373 | 416 |
| 1 | 232 | 224 |
| 2 | 140 | 146 |
| 3 | 55 | 69 |
| 4 | 11 | 21 |
| 5 | 58 | 44 |
| 6 | 116 | 66 |
| 6.5 | 119 | 71 |
| 7 | 125 | 65 |
| 7.5 | 130 | 62 |
| 8 | 111 | 50 |

Mean, n = 4

Hyperreactivity

PC400
(carbachol breath units)

| | Vehicle | Ciclesonide (2.2 mg) |
|---|---|---|
| Baseline | 24.5 | 28.3 |
| Post-Antigen | 13.5 | 21.5 |

Mean, n = 4

Bronchoconstriction

Lung Resistance (% Increase)

| Time (hours) | Vehicle | Salmeterol Xinafoate (2.5 mg) |
|---|---|---|
| Post-Ascaris | 390 | 370 |
| 1 | 239 | 213 |
| 2 | 147 | 114 |
| 3 | 72 | 64 |
| 4 | 14 | 25 |
| 5 | 62 | 54 |
| 6 | 121 | 86 |
| 6.5 | 115 | 85 |
| 7 | 121 | 83 |
| 7.5 | 121 | 76 |
| 8 | 118 | 72 |

Mean, n = 4

Hyperreactivity

PC400 (carbachol breath units)

| | Vehicle | Salmeterol Xinafoate (2.5 mg) |
|---|---|---|
| Baseline | 25.8 | 29.5 |
| Post- | 13.8 | 22.8 |
| Antigen | | |

Mean, n = 4

Bronchoconstriction

Lung Resistance (% Increase)

| | | Ciclesonide (2.2 mg) + Salmeterol |
|---|---|---|
| Time (hours) | Vehicle | Xinafoate (2.5 mg) |
| Post-Ascaris | 419 | 327 |
| 1 | 256 | 192 |
| 2 | 162 | 103 |
| 3 | 61 | 36 |
| 4 | 10 | 12 |
| 5 | 73 | 19 |
| 6 | 121 | 18 |
| 6.5 | 119 | 26 |
| 7 | 130 | 23 |
| 7.5 | 117 | 26 |
| 8 | 121 | 14 |

Mean, n = 4

Hyperreactivity

PC400 (carbachol breath units)

| | | Ciclesonide (2.2 mg) + Salmeterol |
|---|---|---|
| | Vehicle | Xinafoate (2.5 mg) |
| Baseline | 25.8 | 28.3 |
| Post- | 13.3 | 28.5 |
| Antigen | | |

Mean, n = 4

### Example 144

### Carbachol-Induced Bronchoconstriction in Sheep

The compound of Example 52 was assessed for inhibition of carbachol-induced bronchoconstiction as previously described (Abraham, W. M., A. Ahmed, I Serebrlakov, A. N. Carmillo, J. Ferrant, A. R. de Fougerolles, E. A. Garber, P. J. Gowals, V. E. Kotellansky, F. Taylor, R. R. Lobb. Am. J. Respir. Crit. Care. Med. 2004; 169:97-104). Bronchoconstriction was evaluated as a function of increased pulmonary resistance following carbachol challenge. Measurements of R_{L} are repeated immediately after inhalation of buffer and after each administration of 10 breaths of increasing concentrations of carbachol solution (0.25%, 0.5%, 1.0%, and 2.0% w/v). Test article was administered either by a pre-dosing or duration of action protocol. In the pre-dosing regimen, animals were dosed once daily for four days, with the last dose administered 1 hr before carbachol challenge. In the duration of action regimen, animals were dosed once daily for four days, with the last dose administered 24 hours before carbachol challenge. Test article and carbachol were administered by nebulized aerosol to intubated sheep.

Using the predosing regimen, inhibition of carbachol-induced bronchoconstriction was observed with both the compound of Example 52 and salmeterol xinafoate. The results demonstrate the beta-2 adrenergic receptor agonist sensitivity of the response. Inhibition (p<0.01) of carbachol-induced bronchoconstriction was also observed with the compound of Example 52 in the duration of action regimen. In contrast to the Ascaris response, inhibition of the carbachol-induced bronchoconstriction was not observed with the ciclesonide+salmeterol combination. These results suggest that the compound of Example 52 may possess advantageous bronchodilatory properties compared to the combination of ciclesonide+salmeterol.

### Carbachol Challenge

Predosing Regimen

| | Lung Resistance (% Increase) at 2% Carbachol | | |
|---|---|---|---|
| | Vehicle | Example 52 (4.7 mg) | Salmeterol (2.5 mg) |
| Baseline | 593 ± 59 | 335 ± 38 | 352 ± 27 |

Mean ± s.e.m, n = 4

Duration of Action Regimen

| | Lung Resistance (% Increase) at 2% Carbachol | | |
|---|---|---|---|
| | Vehicle | Example 52 (4.7 mg) | Salmeterol (2.5 mg) |
| Baseline | 595 ± 53 | 450 ± 36 | 594 ± 56 |

Lung Resistance (% Increase)
at 2% Carbachol

| | | Ciclesonide (2.2 mg) + Salmeterol |
|---|---|---|
| | Vehicle | Xinafoate (2.5 mg) |
| Baseline | 574 ± 19 | 501 ± 34 |

Mean ± s.e.m, n = 4

### Example 145

### Pharmaceutical Formulations

### DPI Formulation for Multidose Blister Strip or Capsule Based Inhaler

Target Unit Dose:
500 mcg micronized compound of Formula I (“API”)
15 mg lactose monohydrate for inhalation.

Micronize the API using a mill (e.g. Jet mill) to a mass median aerodynamic diameter T0 (MMAD) from about 1 to about 10 μm, and preferably a MMAD from about 1 to about 5 μm.

The lactose may be milled or sieved. Suitable commercial sources of lactose include DMV-Fonterra Excipients (Lactohale®) and Frieslandfoods Domo (Respotise®).

500 mg of API is blended with 15 g of lactose using an appropriate mixer (e.g. Turbula® Powder Blender). Additional fine lactose particles of less than 10 μm may be added. The blended product is filled into capsules or blister strips.

pMDI Liquid Suspension or Liquid Solution Formulation
Target Unit Dose:
250 mcg of micronized API
150 μl of propellant (e.g., HFA 134a or 227)

Each canister is to contain 120 dose equivalents of APT and propellant+10% overage. Each canister is filled with 33 mg of APT and sealed with a metering valve. The canister is then pressurized with 19.8 mL of propellant.

That which is claimed is:

1. A compound of Formula I: 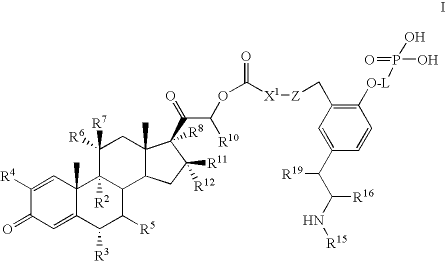
or a pharmaceutically acceptable salt thereof,
wherein:
R¹⁵ is a side chain radical of a β-agonist;
R¹⁶ is H, methyl or ethyl;
R¹⁹ is H, F, OH or methyl;
each R², R³, R⁴, and R⁵ are independently H, C₁-C₄alkyl or halo;
R⁶ and R⁷ are independently H or OH; or R⁶ and R⁷ taken together with the carbon to which they are attached form a >C═O group;
R⁸ is H, OH, O(CO)R⁹, or O(CO)OR⁹;
each R⁹ is independently C₁-C₄alkyl;
each R¹⁰ and R⁹ is independently H or C₁-C₄alkyl;
R¹² is H, OH, or C₁-C₄alkyl; or
R¹¹ and R¹² taken together with the carbon to which they are attached form a >═CH₉ group; or
R¹² and R⁸ taken together with the carbons to which they are attached form a 1,3-dioxolane ring represented by formula B: 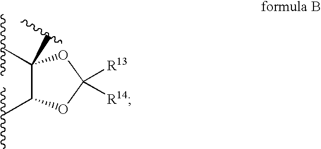
wherein one of R¹³ and R¹⁴ is H, methyl or ethyl and the other is H, C₁-C₁₀alkyl, C₂-C₁₀alkenyl, C₂-C₁₀alkynyl, optionally substituted C₃-C₁₀ carbocyclyl or optionally substituted 5-6 ring atom heterocycle wherein one or two ring atoms are selected from N, O and S, and wherein said carbocyclyl and said heterocyclyl are each optionally substituted 1, 2 or 3 times with a substituent selected from halo, C₁-C₄alkyl, and O—C₁-C₄alkyl;
Z is N(H), N(C₁-C₆alkyl), ^{⊕}(NR¹⁷R¹⁸)A⁽⁻⁾, N(O)R¹⁷ (N-oxide), S(O) (sulfoxide), S(═O)₂, ^{⊕}(SR¹⁷)A⁽⁻⁾, or a 4-9 ring atom heterocyclene wherein one ring atom is N, ^{⊕}(N)A⁽⁻⁾, ^{⊕}(N(C₁-C₆alkyl))A⁽⁻⁾ or ^{⊕}SA⁽⁻⁾, and the β-agonist moiety: 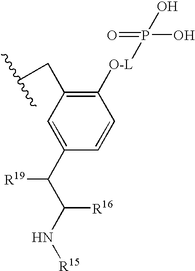
is bonded to said N, ^{⊕}N, ^{⊕}N(C₁-C₆alkyl) or ^{⊕}S of said heterocyclene;
X¹ is selected from a bond,
C₁-C₁₂alkylene, C₂-C₁₂alkenylene, C₂-C₂alkynylene,
O—C₁-C₁₂alkylene, O—C₂-C₁₂alkenylene, O—C₂-C₁₂alkynylene,
S—C₁-C₁₂alkylene, S—C)—C₁₋₂alkenylene, S—C₂-C₁₂alkynylene,
N(H)—C₁-C₁₂alkylene, N(H)—C₂-C₂alkenylene, N(H)—C₂-C₁₂alkynylene,
N(C₁-C₆alkyl)-C₁-C₁₂alkylene, N(C₁-C₆alkyl)-C₂-C₁₂alkenylene, N(C₁-C₆alkyl)-C₂-C₁₂alkynylene,
C₃-C₇-carbocyclene, C₃-C₇-carbocyclene-C₁-C₆alkylene, heterocyclene, heterocyclene-C₁-C₆alkylene, heterocyclene-N(H)C(O), wherein said heterocyclene is a 3-9 ring atom heterocyclene wherein 1 or 2 ring atoms are selected from N, O and S,
C₁-C₆alkylene-O—C₁-C₆alkylene, C₁-C₆alkylene-S—C₁-C₆alkylene, C₁-C₆alkylene-N(H)—C₁-C₆alkylene, C₁-C₆alkylene-N(C₁-C₃alkyl)-C₁-C₆alkylene,
C₁-C₆alkylene-C₃-C₇-carbocyclene-C₁-C₆alkylene, C₁-C₆alkylene-heterocyclene-C₁-C₆alkylene, wherein said heterocyclene is a 3-9 ring atom heterocyclene wherein 1 or 2 ring atoms are selected from N, O and S,
C₁-C₁₂alkylene-O, C₁-C₂alkylene-S, C₁-C₁₂alkylene-N(H), C₁-C₁₂alkylene-N(C₁-C₆alkyl), C₁-C₈alkylene-N(H)C(O), C₁-C₈alkylene-N(C₁-C₄alkyl)C(O), C₁-C₈alkylene-C(O)N(H), C₁-C₈alkylene-C(O)N(C₁-C₄alkyl),
CH-AA and C(H)(AA)-N(H)C(O), wherein AA is a proteinogenic amino acid side chain;
wherein each alkyl, alkylene, alkenylene, and alkynylene is optionally substituted 1 or 2 times with a substituent independently selected from halo, OH, OCH₃, NH₂, N(H)CH₃, and N(C₁₋₃)₂, and each carbocyclene and heterocyclene is optionally substituted 1, 2 or 3 times with a substituent independently selected from halo, and C₁-C₄alkyl;
wherein when Z is N(H), N(C₁-C₆alkyl), ^{⊕}(NR¹⁷R¹⁸)A⁽⁻⁾, N(O)R¹⁷ (N-oxide), S(O) (sulfoxide), S(═O)₂, or ^{⊕}(SR¹⁷)A⁽⁻⁾, then X¹ is neither a bond nor bound to Z through O, S, N(H), N(C₁-C₄alkyl), N(H)C(O), N(C₁-C₄alkyl)C(O), C(O)N(H) or C(O)N(C₁-C₄alkyl);
wherein each R¹⁷ and R¹⁸ are, independently, C₁-C₆alkyl, C₁-C₆alkenyl, C₁-C₆alkynyl, or C₃-C₇-carbocycle, wherein said alkyl, alkenyl, alkynyl is optionally substituted 1, 2 or 3 times with a substituent independently selected from halo, OH, and ═O, and the carbocycle is optionally substituted 1, 2 or 3 times with a substituent independently selected from halo, C₁-C₄alkyl, OH, and ═O;
L is a bond or —(CH₂O)—; and
A⁽⁻⁾ is a pharmaceutically acceptable negative counterion.

2. The compound according to claim 1 or a pharmaceutically acceptable salt thereof wherein R¹⁵ is
C₁-C₆alkyl;
C₆-C₁₀-carbocycle optionally substituted 1 or 2 times with halo, C₁-C₄alkyl, O—C₁-C₄alkyl, O—(CH₂)₄—NH₂, O—(CH₂)₄—N(H)C₁-C₄alkyl, O—(CH₂)₄—N(C₁-C₄alkyl)₂, O—C₁-C₄alkyl-C(O)—NH₂, O—C₁-C₄alkyl-C(O)—N(H)C₁-C₄alkyl, O—C₁-C₄alkyl-C(O)—N(C₁-C₄alkyl)₂1 or a group represented by formula i, ii, iii, iv, v, vi, vii, viii, or ix:
i: C₆alkylene-O—R²¹-Ph⁴;
ii: C₂-C₃alkylene-Ph¹-OR²-Ph⁴;
iii: C₂-C₃alkylene-Ph¹-N(H)—R²²-Ph²;
iv: C₂-C₃alkylene-Het-(R²³)-Ph³;
V: C₂-C₃alkylene-Ph¹-CO—C₂alkylene-C(O)N(H)—C₁-C₄alkylene-Ph³;
vi: C₂-C₃alkylene-Ph³;
vii: C₂-C₃alkylene-S(O)₂—C₂-C₄alkylene-O—C₂-C₄alkylene-Ph³;
viii: C₃-C₆alkylene-Ph¹-CO—C₂alkylene-C(O)N(H)—C₁₀-C₁₂ bicyclic carbocycle;
ix: C₃-C₆alkylene-Het-Ph⁴;
wherein:
R²¹ is C₂-C₆alkylene wherein one carbon of said alkylene is optionally replaced by O;
Ph⁴ is phenyl optionally substituted 1 or 2 times by halo, N(H)C(O)NH₂ or S-cyclopentyl,
Ph¹ is phenylene;
R²² is a bond or C₁-C₂alkylene optionally substituted once by OH or NH₂;
Ph² is phenyl optionally substituted 1 or 2 times by O-methyl,
—OCH₂C(CH₃)₂CH₂NH₂,
—SO₂—NH(C₆H₃)(CH₃)(C₇H₁₅) or 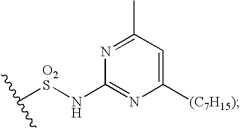
Het is 4-10 ring atom heterocyclene wherein 1, 2 or 3 ring atoms is/are N, O or S optionally substituted once by methyl;
R²³ is a C₂-C₄alkylene wherein one carbon of said alkylene is optionally replaced by O or —CO—C₂alkylene-C(O)N(H)—C₂-C₄alkylene; and
Ph³ is phenyl optionally substituted 1 or 2 times by halo or O-methyl.

3. The compound according to claim 1 or a pharmaceutically acceptable salt thereof, wherein R¹⁵ is C₁-C₆alkyl.

4. The compound according to claim 1 or a pharmaceutically acceptable salt thereof wherein R¹⁵ is a C₆-C₁₀ carbocycle optionally substituted 1 or 2 times with C₁-C₄alkyl, O—C₁-C₄alkyl, or O—C₁-C₄alkyl-C(O)—NH₂.

5. The compound according to claim 1 or a pharmaceutically acceptable salt thereof, wherein R1 is a group represented by formula i: C₆alkylene-O—R²¹-Ph⁴, wherein R²¹ is C₄alkylene and Ph⁴ is unsubstituted phenyl.

6. The compound according to claim 1 or a pharmaceutically acceptable salt thereof, wherein R¹⁵ is a group represented by formula ii: C₂-C₃alkylene-Ph¹-O—R²¹-Ph⁴, wherein R²¹ is C₄alkylene wherein one C is optionally replaced by O and Ph⁴ is unsubstituted phenyl.

7. The compound according to claim 1 or a pharmaceutically acceptable salt thereof wherein R¹⁵ is a group represented by formula iii: C₂-C₃alkylene-Ph¹-N(H)—R²²-Ph², wherein R²² is a bond or C₂ₐₗₖylene substituted once by OH or NH₂, Ph² is phenyl optionally substituted once by O-methyl or —OCH₂C(CH₃)₂CH₂NH₂.

8. The compound according to claim 1 or a pharmaceutically acceptable salt thereof, wherein R¹⁵ is a group represented by formula iv: C₂-C₃alkylene-Het-(R²³)-Ph³, wherein Het is a 9 or 10 ring atom heterocyclene wherein 1 or 2 ring atoms is N, O or S, R²³ is CH₂—O—CH₂— or —C(O)N(H)—CH₂—, and Ph³ is unsubstituted phenyl, or phenyl substituted twice by halo or O-methyl.

9. The compound according to claim 1 or a pharmaceutically acceptable salt thereof wherein R¹⁵ is a group represented by formula v: C₂-C₃alkylene-Ph¹-C₀-C₂alkylene-C(O)N(H)—C₁-C₄alkylene-Ph³, wherein Ph³ is phenyl substituted twice by halo or O-methyl.

10. The compound according to claim 1 or a pharmaceutically acceptable salt thereof, wherein R¹⁵ is a group represented by formula vi: C₂-C₃alkylene-Ph³, wherein Ph³ is phenyl substituted once by O-methyl.

11. The compound according to claim 1 or a pharmaceutically acceptable salt thereof wherein R¹⁵ is a group selected from 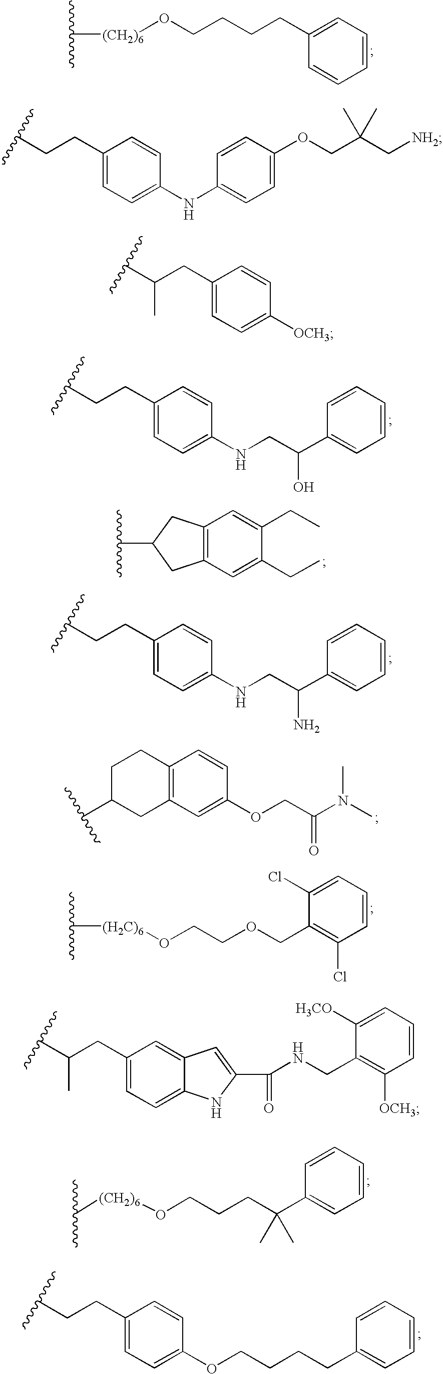 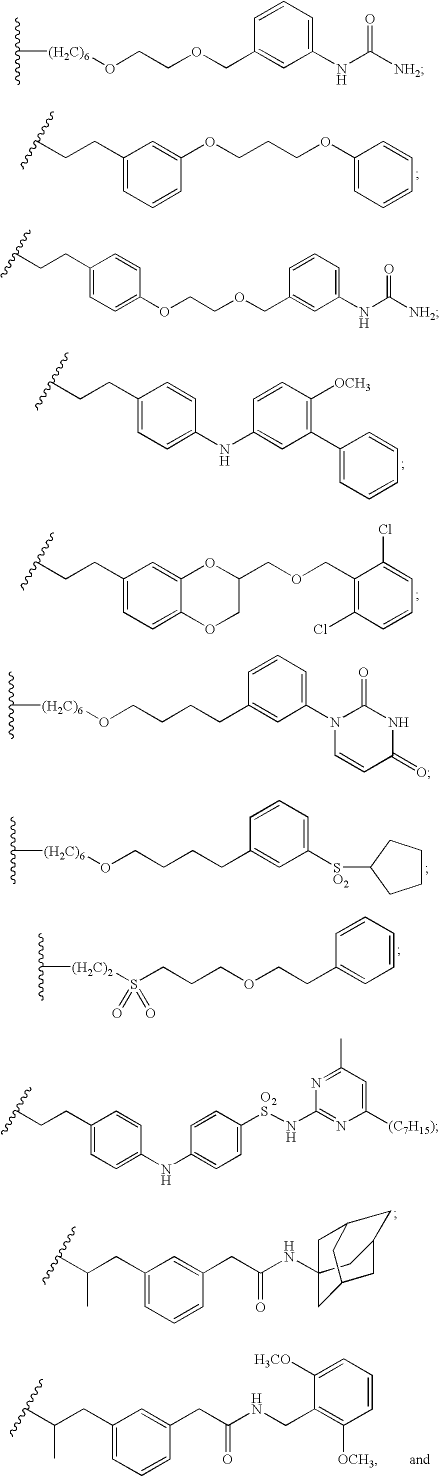 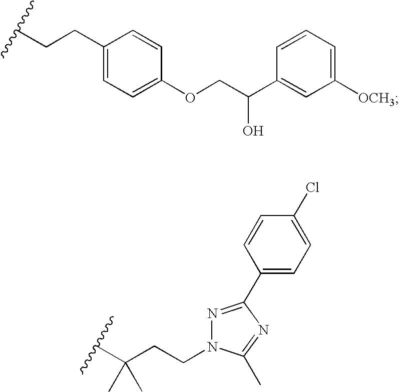
wherein the wavy bond indicates the point of attachment.

12. The compound according to claim 1 or a pharmaceutically acceptable salt thereof, wherein R², R³, R⁴, and R¹ are independently H, methyl, F or Cl.

13. The compound according to claim 1 or a pharmaceutically acceptable salt thereof, wherein R², R³, R⁴, and R⁵ are H.

14. The compound according to claim 1 or a pharmaceutically acceptable salt thereof, wherein R⁶ is H and R⁷ is OH.

15. The compound according to claim 1 or a pharmaceutically acceptable salt thereof, wherein R¹⁰ and R¹¹ are H.

16. The compound according to claim 1 or a pharmaceutically acceptable salt thereof, wherein R¹² and R⁸ taken together with the carbons to which they are attached form a 1,3-dioxolane ring represented by formula B:

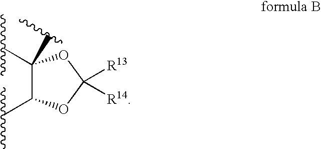

17. The compound according to claim 1 or a pharmaceutically acceptable salt thereof, wherein R¹² and R⁸ taken together with the carbons to which they are attached form a 1,3-dioxolane ring represented by formula B, and one of R¹³ and R¹⁴ is X, methyl or ethyl and the other is H, C₁-C₁₀alkyl, or C₃-C₁₀ carbocyclyl.

18. The compound according to claim 1 or a pharmaceutically acceptable salt thereof wherein R², R³, R⁴, R⁵, R⁷, R¹⁰, R¹¹, R¹², R⁸, R¹³, and R⁴ are defined as

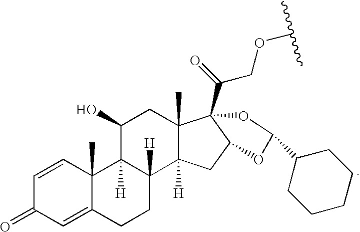

19. The compound according to claim 1 or a pharmaceutically acceptable salt thereof wherein Z is ^{⊕}(NR¹⁷R¹⁸)A⁽⁻⁾, ^{⊕}(SR¹⁷)A⁽⁻⁾, or a 4-9 ring atom heterocyclene wherein one ring atom is ^{⊕}(N)A⁽⁻⁾, ^{⊕}(N(C₁-C₆alkyl))A⁽⁻⁾ or ^{⊕}SA⁽⁻⁾, and the β-agonist moiety: 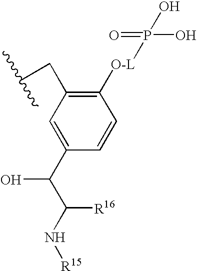
is bonded to said ^{⊕}N, ^{⊕}N(C₁-C₆alkyl) or ^{⊕}S of the heterocyclene.

20. The compound according to claim 1 or a pharmaceutically acceptable salt thereof, wherein Z is ^{⊕}(NR¹⁷R¹⁸)A⁽⁻⁾, wherein R¹⁷ and R¹⁸ are each independently, unsubstituted C₁-C₆alkyl, unsubstituted C₁-C₆alkenyl, unsubstituted C₁-C₆alkynyl or unsubstituted C₃-C₇-carbocycle.

21. The compound according to claim 1 or a pharmaceutically acceptable salt thereof, wherein Z is 5-6 ring atom heterocyclene wherein one ring atom is ^{⊕}(N)A⁽⁻⁾ or ^{⊕}(N(C₁-C₂alkyl))A⁽⁻⁾ and the β-agonist moiety is bonded to said ^{⊕}N or ^{⊕}N(C₁-C₂alkyl).

22. The compound according to claim 1 or a pharmaceutically acceptable salt thereof, wherein X¹ is a bond.

23. The compound according to claim 1 or a pharmaceutically acceptable salt thereof, wherein X¹ is selected from
C₁-C₆alkylene, C₂-C₆alkenylene, C₂-C₆alkynylene,
O—C₁-C₆alkylene, S—C₁-C₆alkylene, N(H)—C₁-C₆alkylene, N(H)—C₂-C₆alkenylene, N(C₁-C₄alkyl)-C₁-C₆alkylene,
C₃-C₆-carbocyclene, C₃-C₆-carbocyclene-C₁-C₄alkylene, and
C₁-C₄alkylene-N(H)C(O)—;
wherein each alkyl, alkylene, alkenylene, and alkynylene is optionally substituted 1 or 2 times with a substituent independently selected from halo, OH, OCH₃, NH₂, N(H)CH₃, and N(CH₃)₂ and each carbocyclene and heterocyclene is optionally substituted 1, 2 or 3 times with a substituent independently selected from halo, and C₁-C₄alkyl.

24. The compound according to claim 1 or a pharmaceutically acceptable salt thereof, wherein Z is ^{⊕}(NR¹⁷R¹⁸)A⁽⁻⁾ and X¹ is selected from C₁-C₆alkylene, C₂-C₆alkenylene, C₂-C₆alkynylene, O—C₁-C₆alkylene, N(H)—C₁-C₆alkylene, N(C₁-C₄alkyl)-C₁-C₆alkylene, phenylene, and C₃-C₆-carbocyclene-C₁-C₄alkylene, wherein each alkyl, alkylene, alkenylene, alkynylene, carbocyclene and phenylene of X¹ is unsubstituted.

25. The compound according to claim 1 or a pharmaceutically acceptable salt thereof, wherein Z is a 5-6 ring atom heterocyclene wherein one ring atom is ^{⊕}(N)A⁽⁻⁾ or ^{⊕}(N(C₁-C₆alkyl))A⁽⁻⁾, and the β-agonist moiety is bound to ^{⊕}N or ^{⊕}N(C₁-C₆alkyl), and X¹ is selected from a bond, C₁-C₆alkylene, C₁-C₆alkenylene, C₃-C₆-carbocyclene, C₃-C₆-carbocyclene-C₁-C₄alkylene, and C₁-C₄alkylene-N(H)C(O), wherein each alkyl, alkylene, alkenylene, alkynylene, carbocyclene and phenylene of X¹ are unsubstituted.

26. The compound according to claim 1 or a pharmaceutically acceptable salt thereof, wherein A⁽⁻⁾ is selected from chloride, bromide, sulfate, acetate, tartrate, fumarate and xinafoate.

27. The compound according to claim 1 or a pharmaceutically acceptable salt thereof, wherein L is a bond.

28. The compound according to claim 1 or a pharmaceutically acceptable salt thereof, which is a compound of Formula II; 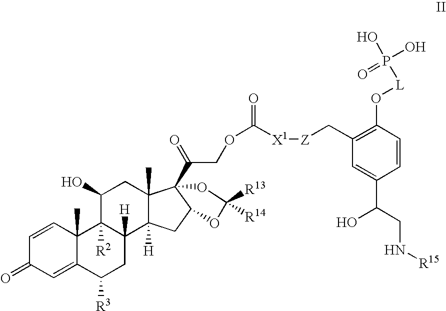
wherein all variables are defined as in any of claims 1-27.

29. The compound according to claim 1 or a pharmaceutically acceptable salt thereof, which is a compound of Formula III: 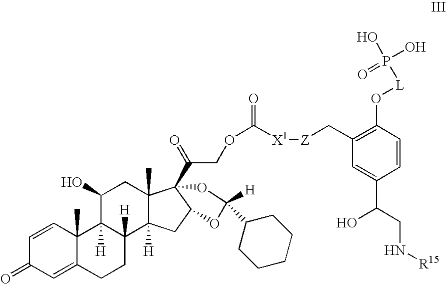
wherein all variables are defined as in any of claims 1-27.

30. The compound according to claim 1 or a pharmaceutically acceptable salt thereof, selected from
1-[5-[1-Hydroxy-2-[6-(4-phenylbutoxy)hexylamino]ethyl]-2-phosphonooxybenzyl]-3-[2-[11β,16α]-[16,17-((R)-cyclohexylmethylene)bis(oxy)]-11-hydroxypregna-1,4-diene-3,20-dion-21-oxy]carbonyl]ethen-1-yl]pyridinium chloride 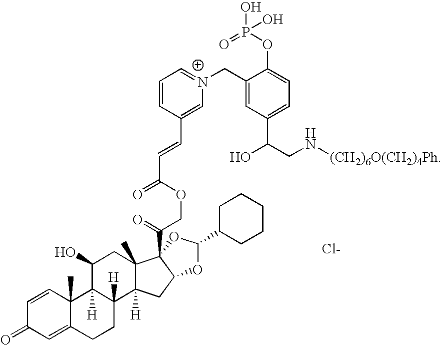
[5-[1-hydroxy-2-[6-(4-phenylbutoxy)hexylamino]ethyl]-2-phosphonooxybenzyl]-(diethyl)-[[11β,16α]-[[15,16-((R)-cyclohexylmethylene)bis(oxy)]-11-hydroxypregna-1,4-diene-3,20-dion-21-yl]carbonylmethyl]ammonium chloride 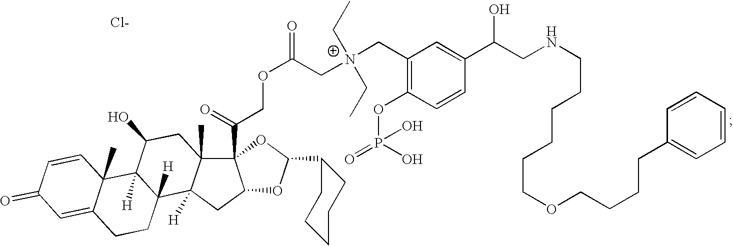
1-[5-[1-Hydroxy-2-[6-(4-phenylbutoxy)hexylamino]ethyl]-2-phosphonooxybenzyl]-3-[[[[[11β,16α]-[16,17-((R)-cyclohexylmethylene)bis(oxy)]-11-hydroxypregna-1,4-diene-3,20-dion-21-oxy]carbonyl]methyl]aminocarbonyl]pyridinium chloride 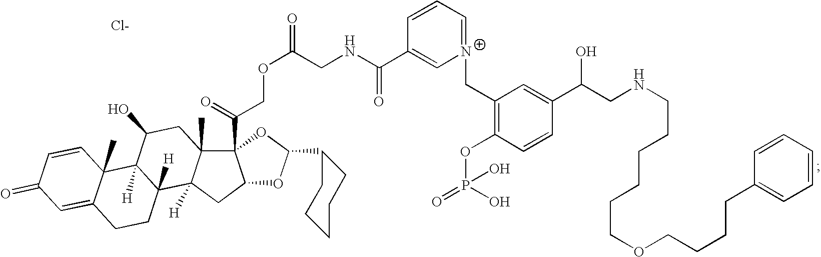
1-[5-[1-Hydroxy-2-[6-(4-phenylbutoxy)hexylamino]ethyl]-2-phosphonooxybenzyl]-1-methyl-4-[[11β,16α]-[[((R)-cyclohexylmethylene)bis(oxy)]-11-hydroxypregna-1,4-diene-3,20-dion-21-oxy]carbonyl]piperidinium acetate 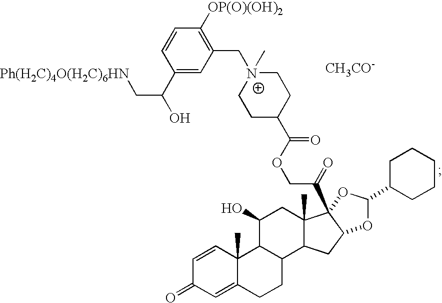
[5-[1-(R)-hydroxy-2-[6-(4-phenylbutoxy)hexylamino]ethyl]-2-phosphonooxybenzyl]-(diethyl)-[[11β,16α]-[16,17-((R)-cyclohexylmethylene)bis(oxy)]-1-hydroxypregna-1,4-diene-3,20-dion-21-oxy]carbonylmethyl]ammonium chloride 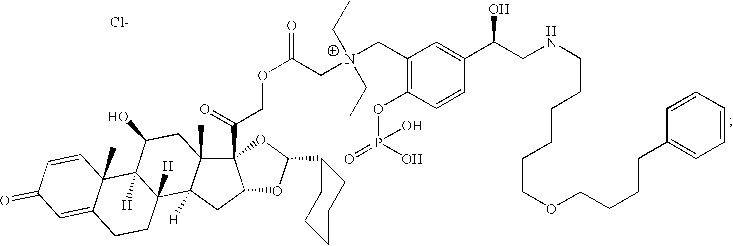
[5-[1-(S)-Hydroxy-2-[6-(4-phenylbutoxy)hexylamino]ethyl]-2-phosphonooxybenzyl]-(diethyl)-[[11β,16α-[16,17-((R)-cyclohexylmethylene)bis(oxy)]-1-hydroxypregna-1,4-diene-3,20-dion-21-oxy]carbonylmethyl]ammonium chloride 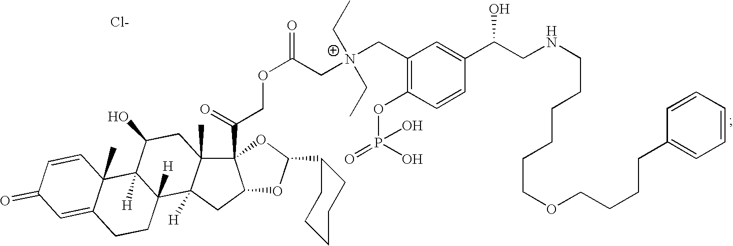
1-[5-[1-hydroxy-2-[6-(4-phenylbutoxy)hexylamino]ethyl]-2-phosphonooxybenzyl]-1-[[11β16α]-[[15,16-((R)-cyclohexylmethylene)bis(oxy)]-11-hydroxypregna-1,4-diene-3,20-dion-21-oxy]carbonylmethyl]pyrrolidinium chloride 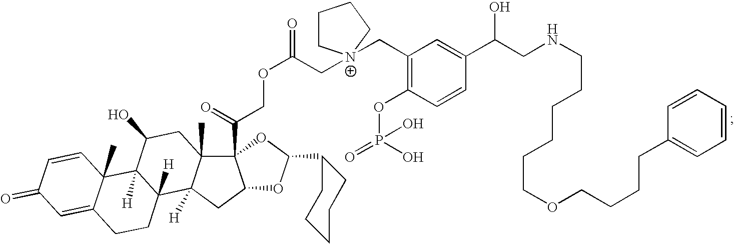
1-[5-[1-hydroxy-2-[6-(4-phenylbutoxy)hexylamino]ethyl]-2-phosphonooxybenzyl]-4-[[11β,16α]-[[15,16-((R)-cyclohexylmethylene)bis(oxy)]-11-hydroxypregna-1,4-diene-3,20-dion-21-oxy]carbonylmethyl]-1-methylpiperazinium chloride 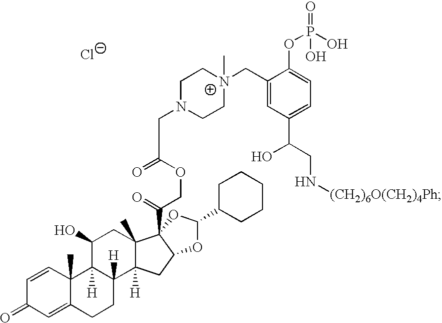
[5-[1-hydroxy-2-(1,1-dimethylethylamino)ethyl]-2-phosphonooxybenzyl]-(diethyl)-[[11,16α]-[[15,16-((R)-cyclohexylmethylene)bis(oxy)]-11-hydroxypregna-1,4-diene-3,20-dion-21-oxy]carbonylmethyl]ammonium chloride 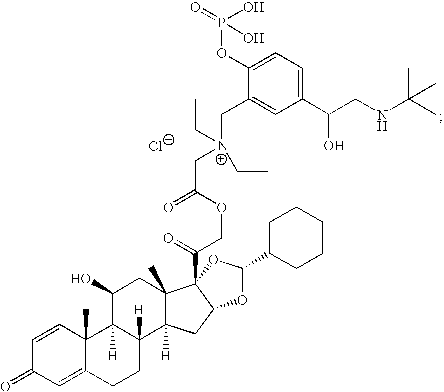
[5-[1-hydroxy-2-[6-(4-phenylbutoxy)hexylamino]ethyl]-2-phosphonooxybenzyl][4-[11β,16α]-[[15,16-((R)-cyclohexylmethylene)bis(oxy)]-11-hydroxypregna-1,4-diene-3,20-dion-21-oxy]carbonylphenyl]imidazolium chloride; and 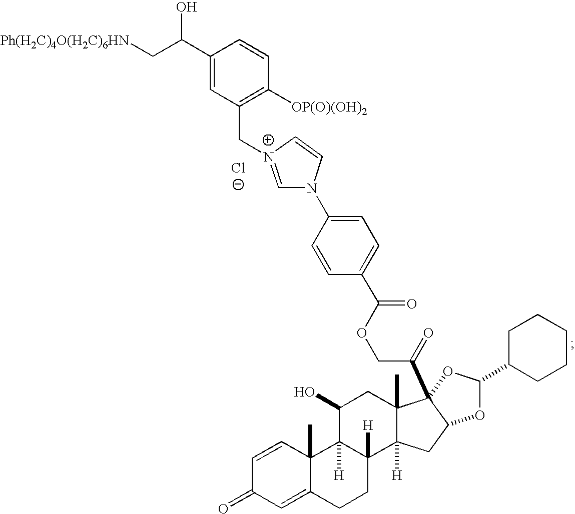
[5-[1-hydroxy-2-[6-(4-phenylbutoxy)hexylamino]ethyl]-2-phosphonooxybenzyl]-(dimethyl)-[5-amino-5-[[11β,16α]-[[15,16-((R)-cyclohexylmethylene)bis(oxy)]-11-hydroxypregna-1,4-diene-3,20-dion-21-oxy]carbonyl]pentyl]ammonium chloride 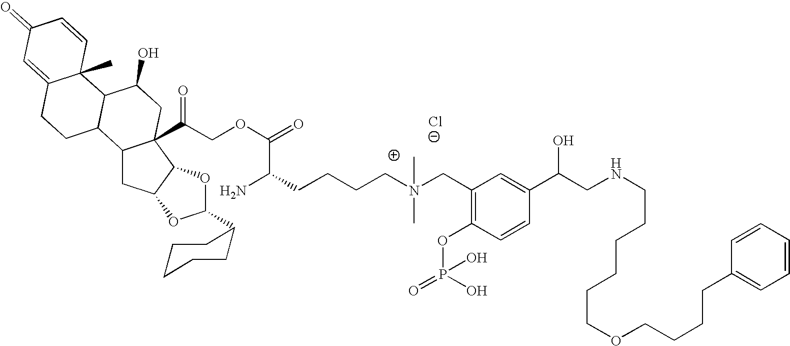
and
pharmaceutically acceptable salts thereof.

31. [5-[1-hydroxy-2-[6-(4-phenylbutoxy)hexylamino]ethyl]-2-phosphonooxybenzyl]-(diethyl)-[[11β,16α]-[[((R)-cyclohexylmethylene)bis(oxy)]-11-hydroxypregna-1,4-diene-3,20-dion-20-yl]methoxycarbonylmethyl]ammonium chloride 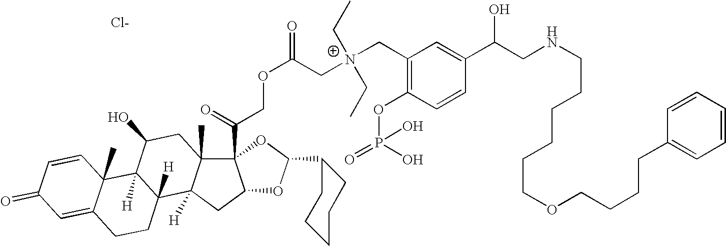
or a pharmaceutically acceptable salt thereof.

32. [5-[1-hydroxy-2-[6-(4-phenylbutoxy)hexylamino]ethyl]-2-phosphonooxybenzyl]-(diethyl)-[[11β,16α]-[[((R)-cyclohexylmethylene)bis(oxy)]-11-hydroxypregna-1,4-diene-3,20-dion-20-yl]methoxycarbonylmethyl]ammonium chloride

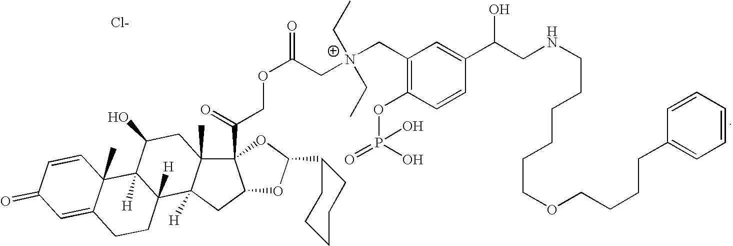

33. [5-[1-(R)-hydroxy-2-[6-(4-phenylbutoxy)hexylamino]ethyl]-2-phosphonooxybenzyl]-(diethyl)-[[11β,16α]-[[((R)-cyclohexylmethylene)bis(oxy)]-11-hydroxypregna-1,4-diene-3,20-dion-20-yl]methoxycarbonylmethyl]ammonium chloride 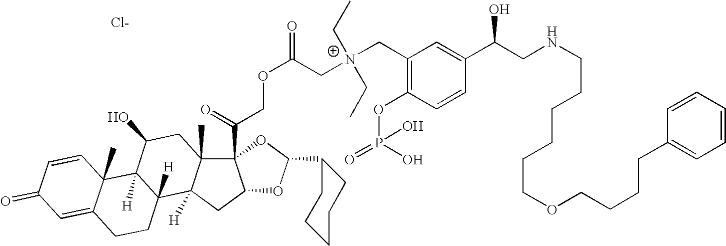
or a pharmaceutically acceptable salt thereof.

34. [5-[1-(R)-hydroxy-2-[6-(4-phenylbutoxy)hexylamino]ethyl]-2-phosphonooxybenzyl]-(diethyl)-[[11β,16α]-[[((R)-cyclohexylmethylene)bis(oxy)]-11-hydroxypregna-1,4-diene-3,20-dion-20-yl]methoxycarbonylmethyl]ammonium chloride

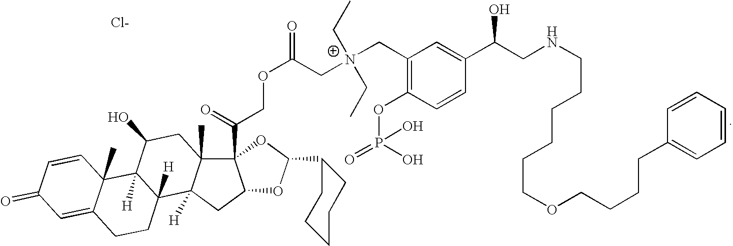

35. A composition comprising a compound according to claim 1 or a pharmaceutically acceptable salt thereof and a pharmaceutically acceptable excipient, diluent or carrier.

36. The composition according to claim 35, wherein the compound is [5-[1-hydroxy-2-[6-(4-phenylbutoxy)hexylamino]ethyl]-2-phosphonooxybenzyl]-(diethyl)-[[11β,16α]-[[((R)-cyclohexylmethylene)bis(oxy)]-11-hydroxypregna-1,4-diene-3,20-dion-20-yl]methoxycarbonylmethyl]ammonium chloride 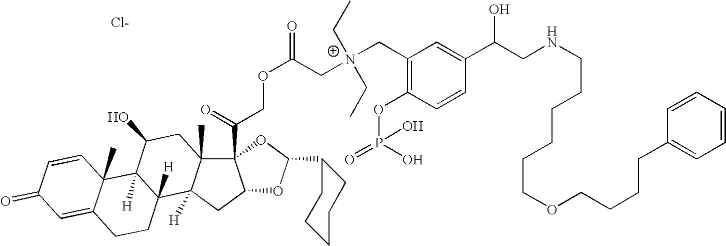
or a pharmaceutically acceptable salt thereof.

37. The composition according to claim 35, wherein the compound is [5-[1-(R)-hydroxy-2-[6-(4-phenylbutoxy)hexylamino]ethyl]-2-phosphonooxybenzyl]-(diethyl)-[[11β,16α]-[[((R)-cyclohexylmethylene)bis(oxy)]-11-hydroxypregna-1,4-diene-3,20-dion-20-yl]methoxycarbonylmethyl]ammonium chloride 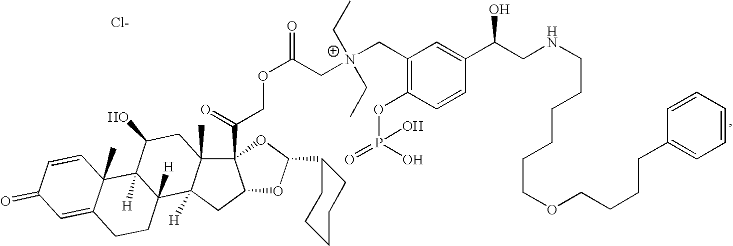
or a pharmaceutically acceptable salt thereof.

38. The composition according to claim 35, wherein said composition is suitable for inhalation.

39. The composition according to claim 35, wherein said composition is a solution for aerosolization and administration by nebulizer.

40. The composition according to claim 35, wherein said composition is suitable for administration by metered dose inhaler.

41. The composition according to claim 35 wherein said composition is a dry powder.

42. The composition according to claim 35 further comprising a therapeutically active agent selected from anti-inflammatory agents, anticholinergic agents, β-agonists, peroxisome proliferator-activated receptor agonists, epithelial sodium channel blockers, kinase inhibitors, antiinfective agents and antihistamines

43. The composition according to claim 42, wherein said therapeutically active agent is a corticosteroid.

44. The composition according to claim 43, wherein said corticosteroid is ciclesonide, desisobutyryl ciclesonide, budesonide mometasone, fluticasone propionate, or fluticasone furoate.

45. The composition according to claim 42, wherein said therapeutically active agent is a PDE4 inhibitor.

46. The composition according to claim 42, wherein said therapeutically active agent is tiotropium.

47. The composition according to claim 42, wherein said therapeutically active agent is salmeterol or R-salmeterol.

48. The composition according to claim 42, wherein said therapeutically active agent is a peroxisome proliferator-activated receptor gamma agonist.

49. A method comprising administering to a human, an effective amount of a compound according to claim 1 or a pharmaceutically acceptable salt thereof.

50. A method for the treatment of pulmonary inflammation or bronchoconstriction in a human in need thereof, comprising administering to said human an effective amount of a compound according to claim 1 or a pharmaceutically acceptable salt thereof.

51. A method for the treatment of a disease associated with reversible airway obstruction in a human in need thereof, comprising administering to said human an effective amount of a compound according to claim 1 or a pharmaceutically acceptable salt thereof.

52. A method for the treatment of asthma in a human in need thereof, comprising administering to said human an effective amount of a compound according to claim 1 or a pharmaceutically acceptable salt thereof.

53. A method for the treatment of COPD in a human in need thereof, comprising administering to said human an effective amount of a compound according to claim 1 or a pharmaceutically acceptable salt thereof.

54. A method for the treatment of bronchiectasis in a human in need thereof, comprising administering to said human an effective amount of a compound according to claim 1 or a pharmaceutically acceptable salt thereof.

55. A method for the treatment of emphysema in a human in need thereof, comprising administering to said human an effective amount of a compound according to claim 1 or a pharmaceutically acceptable salt thereof.

56. The method for the treatment of asthma or COPD in a human in need thereof, said method comprising administering to said human an effective amount of a compound according to claim 31.

57. The method for the treatment of asthma or COPD in a human in need thereof, said method comprising administering to said human an effective amount of a compound according to claim 33.

58. A method for delivering an effective amount of a steroid and a β-agonist to the lung of a human, said method comprising delivering an effective amount of a compound according to claim 1 to the lung of said human, wherein a phosphate group of said compound is cleaved by an endogenous enzyme and an ester group of said compound is cleaved by an endogenous esterase to deliver said steroid and said β-agonist.
